(19) United States
(12) Patent Application Publication
(10) Pub. No.: US 2007/0259881 A1
(43) Pub. Date: Nov. 8, 2007

(54) Substituted Imidazo Ring Systems and Methods

(76) Inventors: Joseph F. Dellaria JR., Woodbury, MN (US); Tushar A. Kshirsagar, Woodbury, MN (US); Shri Niwas, Maple Grove, MN (US); William H. Moser, St. Paul, MN (US); Joan T. Moseman, Lake Elmo, MN (US); Kyle J. Lindstrom, Houlton, WI (US); Azim A. Celebi, Clark, NJ (US); John F. Gerster, Woodbury, MN (US); Philip D. Heppner, Forest Lake, MN (US); Joshua R. Wurst, North St. Paul, MN (US)

Correspondence Address: 3M INNOVATIVE PROPERTIES COMPANY, PO BOX 33427, ST. PAUL, MN, 55133-3427, US

(21) Appl. No.: 11/570,715

(22) PCT Filed: Jun. 17, 2005
(86) PCT No.: PCT/US05/21435

§ 371 Date: Dec. 15, 2006

Related U.S. Application Data
(60) Provisional application No. 60/580,989, filed on Jun. 18, 2004.

(51) Int. Cl. A61K 31/44; A61K 31/437; A61K 31/496; A61P 31/12; A61P 35/00; C07D 401/02; C07D 401/14; C07D 471/02

(52) U.S. Cl. 514/253.03; 514/293; 514/303; 544/362; 546/118; 546/82

(57) ABSTRACT

Imidazo ring systems, which include, for example, imidazopyridine, imidazoquinoline, 6,7,8,9-tetrahydroimidazoquinoline, imidazonaphthyridine, and 6,7,8,9-tetrahydroimidazonaphthyridine compounds substituted at the 1-position and/or the 2-position, pharmaceutical compositions containing these compounds, methods of making these compounds, and methods of use of these compounds as immunomodulators, for inducing cytokine biosynthesis in animals and in the treatment of diseases including viral and neoplastic diseases are disclosed.

### CROSS REFERENCE TO RELATED APPLICATIONS

The present invention claims priority to U.S. Provisional Application Ser. Nos. 60/580,989, filed Jun. 18, 2004, which is incorporated herein by reference.

### BACKGROUND

In the 1950's the 1H-imidazo[4,5-c]quinoline ring system was developed, and 1-(6-methoxy-8-quinolinyl)-2-methyl-1H-imidazo[4,5-c]quinoline was synthesized for possible use as an antimalarial agent. Subsequently, syntheses of various substituted 1H-imidazo[4,5-c]quinolines were reported. For example, 1-[2-(4-piperidyl)ethyl]-1H-imidazo[4,5-c]quinoline was synthesized as a possible anticonvulsant and cardiovascular agent. Also, several 2-oxoimidazo[4,5-c]quinolines have been reported.

Certain 1H-imidazo[4,5-c]quinolin-4-amines and 1- and 2-substituted derivatives thereof were later found to be useful as antiviral agents, bronchodilators and immunomodulators. Subsequently, certain substituted 1H-imidazo[4,5-c]pyridin-4-amine, quinolin-4-amine, tetrahydroquinolin-4-amine, naphthyridin-4-amine, and tetrahydronaphthyridin-4-amine compounds as well as certain analogous thiazolo and oxazolo compounds were synthesized and found to be useful as immune response modifiers, rendering them useful in the treatment of a variety of disorders.

There continues to be interest in and a need for compounds that have the ability to modulate the immune response, by induction of cytokine biosynthesis or other mechanisms.

### SUMMARY

The present invention provides a new class of compounds that are useful in inducing cytokine biosynthesis in animals. Such compounds include an imidazo core of the following structure: 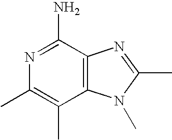
wherein the pendant bonds are used to indicate the atoms which are substituted by the substituents described below (and do not necessarily refer to methyl substituents, although they can).

Examples of such compounds include compounds of the following Formulas I through VII, VIII-1, VIII-2, VIII-3, VIII-4, IX-1, IX-2, IX-3, IX-4, X through XIV, XV-1, XV-2, XV-3, XV-4, XVI-1, XVI-2, XVI-3, XVI-4, XIXa, XIXb, XIXc, XIXd, XX, XXI, XXII, XXIII, and XXIV: 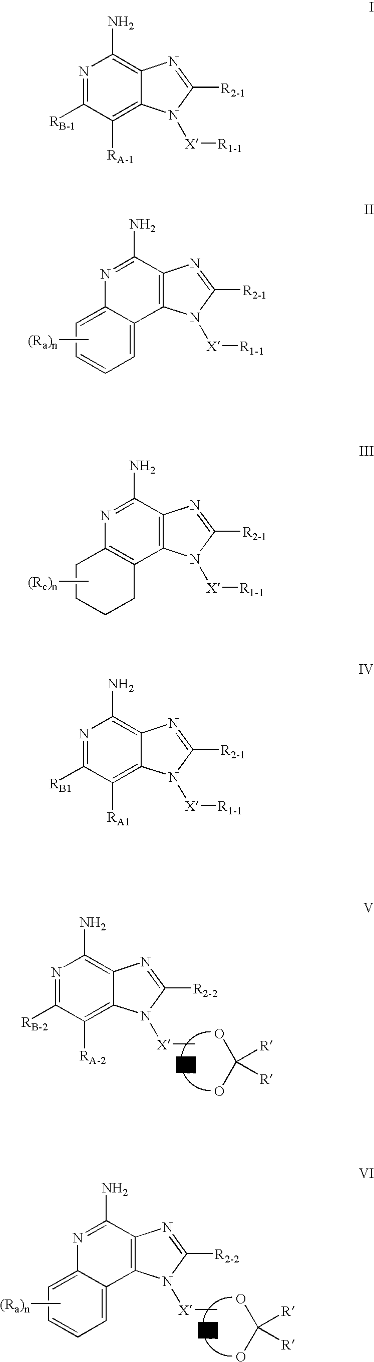 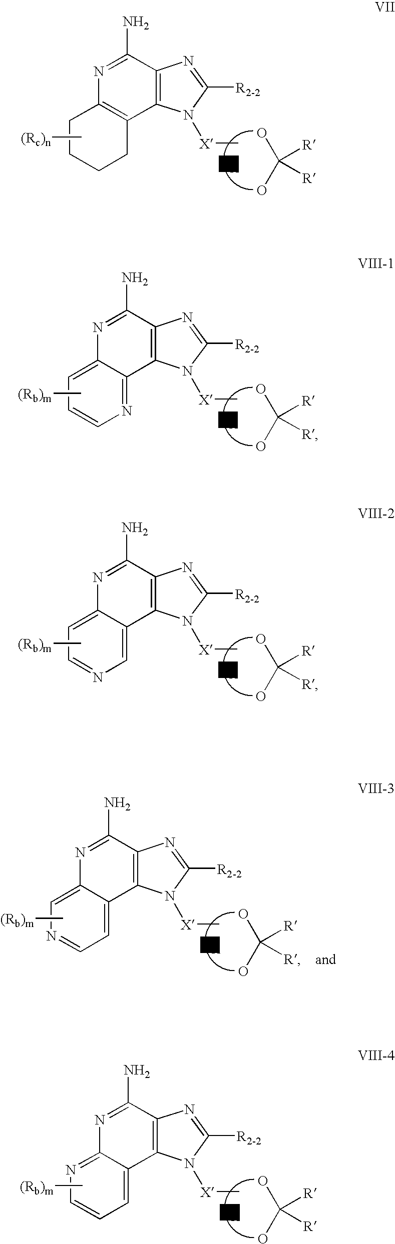 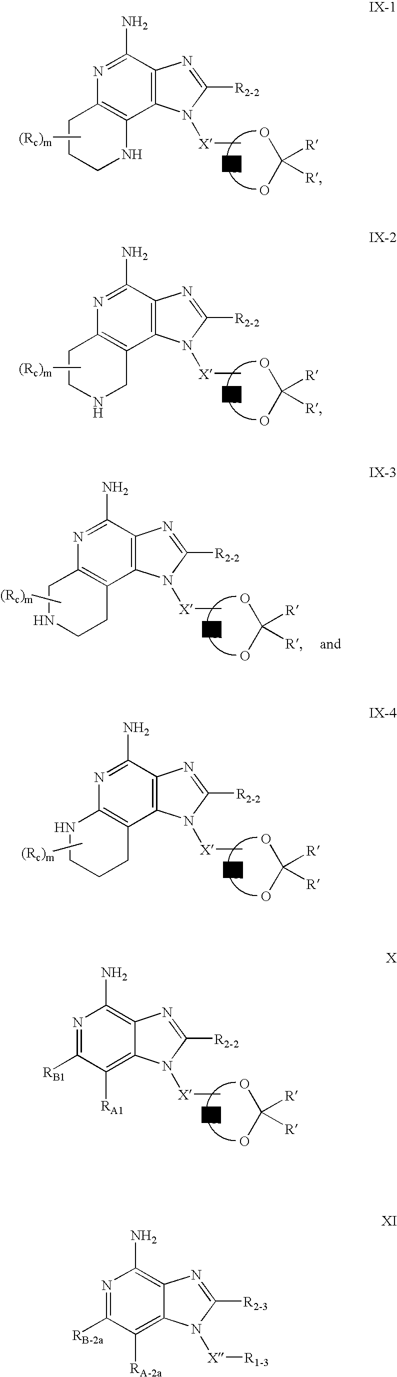 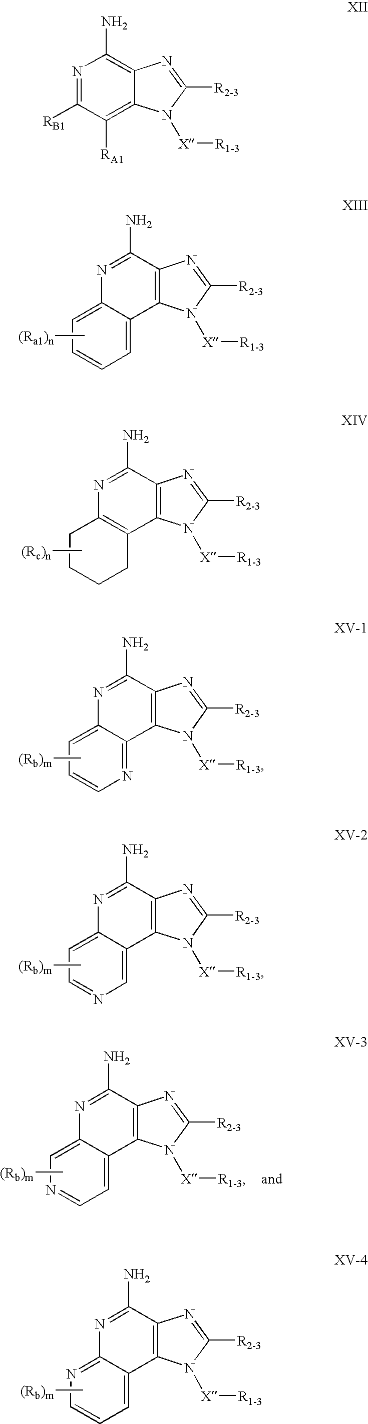 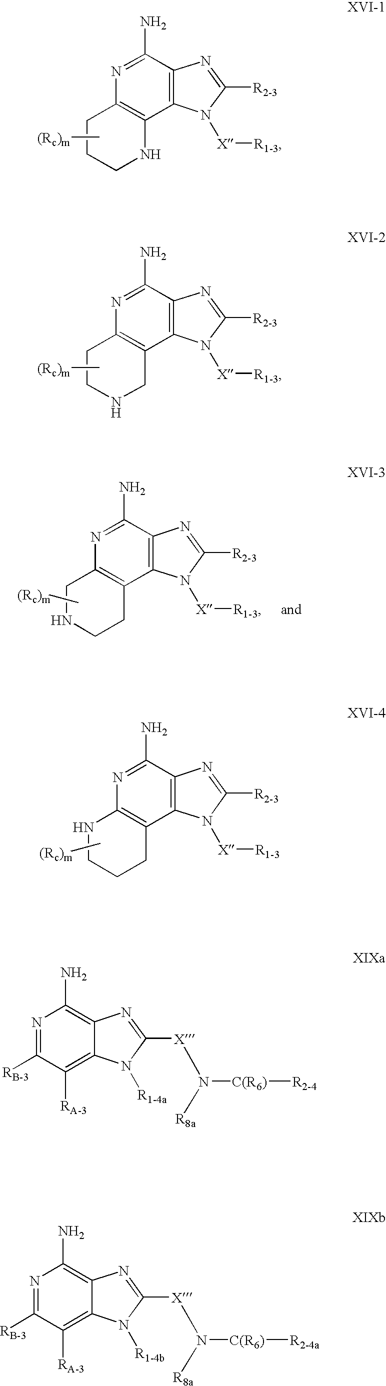 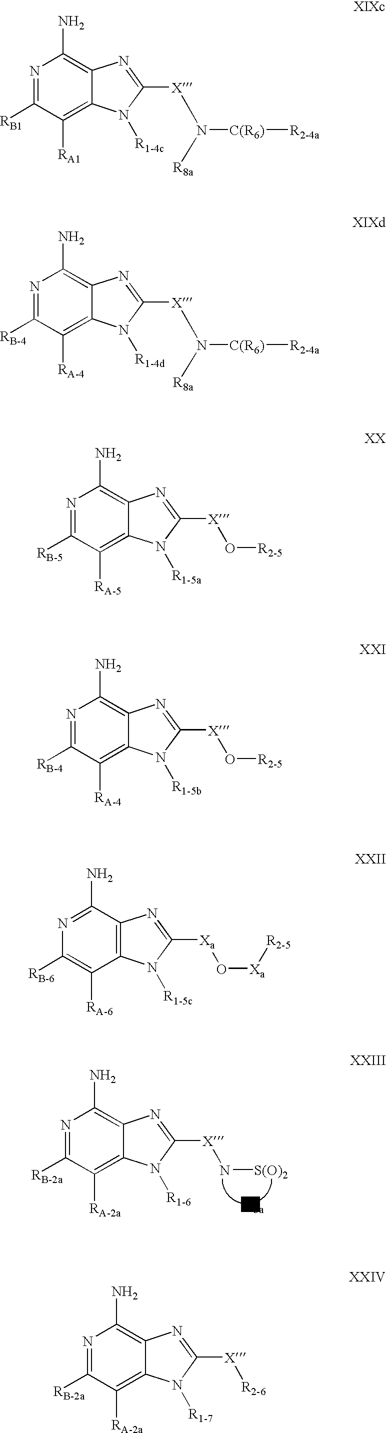
wherein R_{A1}, R_{B1}, R_{A-1}, R_{B-1}, R_{A-2}, R_{B-2}, R_{A-2a}, R_{B-2a}, R_{A-3}, R_{B-3}, R_{A-4}, R_{B-4}, R_{A-5}, R_{B-5}, R_{A-6}, R_{B-6}, R₁₋₁, R₁₋₃, R₁₋₄ₐ, R_{1-4b}, R_{1-4c}, R_{1-4d}, R₁₋₅ₐ, R_{1-5b}, R_{1-5c}, R₁₋₆, R₁₋₇, R₂₋₁, R₂₋₂, R₂₋₃, R₂₋₄, R₂₋₄ₐ, R₂₋₅, R₂₋₆, Rₐ, Rₐ₁, R_{b}, R_{c}, R₃ₐ, R₆, R₈ₐ, R′, R₁₁, Xₐ, X′, X″, X′″, m, and n are as defined below; and pharmaceutically acceptable salts thereof.

The present invention also provides compounds (which are prodrugs) of the following Formulas CI, CV, CXI, CXIX, CXX, CXXII, CXXIII, and CXXIV: 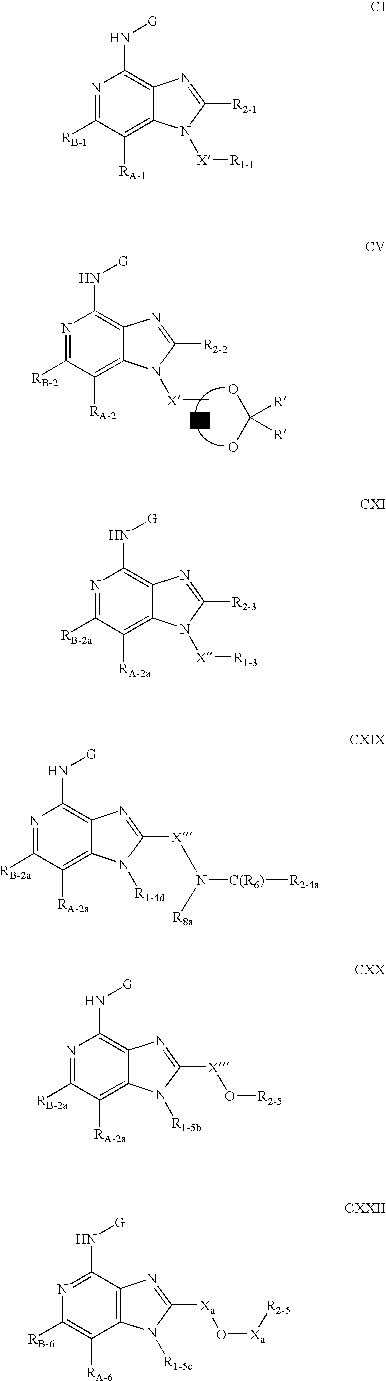 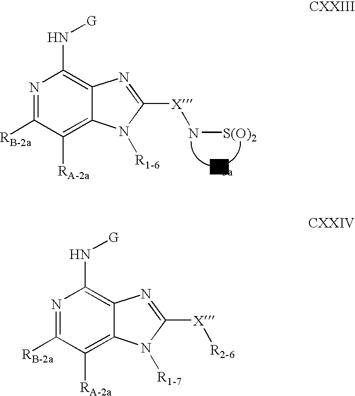
wherein G, R_{A-1}, R_{B-1}, R_{A-2}, R_{B-2}, R_{A-2a}, R_{B-2a}, R_{A-6}, R_{B-6}, R₁₋₁, R₁₋₃, R_{1-4d}, R_{1-5b}, R_{1-5c}, R₁₋₆, R₁₋₇, R₂₋₁, R₂₋₂, R₂₋₃, R₂₋₄ₐ, R₂₋₅, R₂₋₆, R₃ₐ, R₆, R₈ₐ, R′, R₁₁, Xₐ, X′, X″, and X′″ are as defined below; and pharmaceutically acceptable salts thereof.

The present invention also includes intermediates of the following Formulas XVII and XVIII: 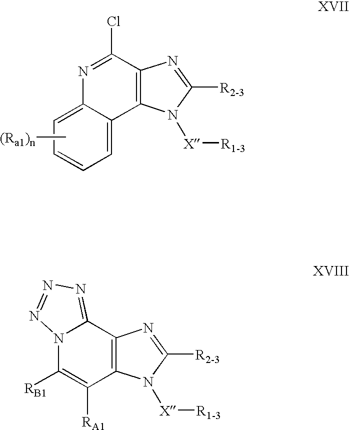
wherein Rₐ₁, n, R_{A1}, R_{B1}, X″, R₁₋₃, and R₂₋₃ are as defined below.

The compounds of Formulas I through VII, VIII-1, VIII-2, VIII-3, VIII-4, IX-1, IX-2, IX-3, IX-4, X through XIV, XV-1, XV-2, XV-3, XV-4, XVI-1, XVI-2, XVI-3, XVI-4, XIXa, XIXb, XIXc, XIXd, XX, XXI, XXII, XXIII, and XXIV, which include the above core, are useful as immune response modifiers due to their ability to induce cytokine biosynthesis (e.g., induce the synthesis of at least one cytokine) and otherwise modulate the immune response when administered to animals. This makes the compounds useful in the treatment of a variety of conditions such as viral diseases and tumors that are responsive to such changes in the immune response.

The invention further provides pharmaceutical compositions containing an effective amount of a compound of Formulas I through VII, VIII-1, VIII-2, VIII-3, VIII-4, IX-1, IX-2, IX-3, IX-4, X through XIV, XV-1, XV-2, XV-3, XV-4, XVI-1, XVI-2, XVI-3, XVI-4, XIXa, XIXb, XIXc, XIXd, XX, XXI, XXII, XXIII, or XXIV, or a compound of Formulas CI, CV, CXI, CXIX, CXX, CXXII, CXXIII, or CXXIV and methods of inducing cytokine biosynthesis in an animal, treating a viral infection and/or treating a neoplastic disease in an animal by administering an effective amount of a compound of Formulas I through VII, VIII-1, VIII-2, VIII-3, VIII-4, IX-1, IX-2, IX-3, IX-4, X through XIV, XV-1, XV-2, XV-3, XV-4, XVI-1, XVI-2, XVI-3, XVI-4, XIXa, XIXb, XIXc, XIXd, XX, XXI, XXII, XXIII, or XXIV, or a compound of Formulas CI, CV, CXI, CXIX, CXX, CXXII, CXXIII, or CXXIV to the animal.

In addition, methods of synthesizing compounds of Formulas I through VII, VIII-1, VIII-2, VIII-3, VIII-4, IX-1, IX-2, IX-3, IX-4, X through XIV, XV-1, XV-2, XV-3, XV-4, XVI-1, XVI-2, XVI-3, XVI-4, XIXa, XIXb, XIXc, XIXd, XX, XXI, XXII, XXIII, XXIV, CI, CV, CXI, CXIX, CXX, CXXII, CXXIII, and CXXIV and intermediates useful in the synthesis of these compounds are provided.

As used herein, “a”, “an”, “the”, “at least one”, and “one or more” are used interchangeably.

The terms “comprises” and variations thereof do not have a limiting meaning where these terms appear in the description and claims.

The above summary of the present invention is not intended to describe each disclosed embodiment or every implementation of the present invention. The description that follows more particularly exemplifies illustrative embodiments. In several places throughout the description, guidance is provided through lists of examples, which examples can be used in various combinations. In each instance, the recited list serves only as a representative group and should not be interpreted as an exclusive list.

### DETAILED DESCRIPTION OF ILLUSTRATIVE EMBODIMENTS OF THE INVENTION

The present invention provides compounds of the following Formulas I through VII, VIII-1, VIII-2, VIII-3, VIII-4, IX-1, IX-2, IX-3, IX-4, X through XIV, XV-1, XV-2, XV-3, XV-4, XVI-1, XVI-2, XVI-3, XVI-4, XIXa, XIXb, XIXc, XIXd, XX, XXI, XXII, XXIII, and XXIV: 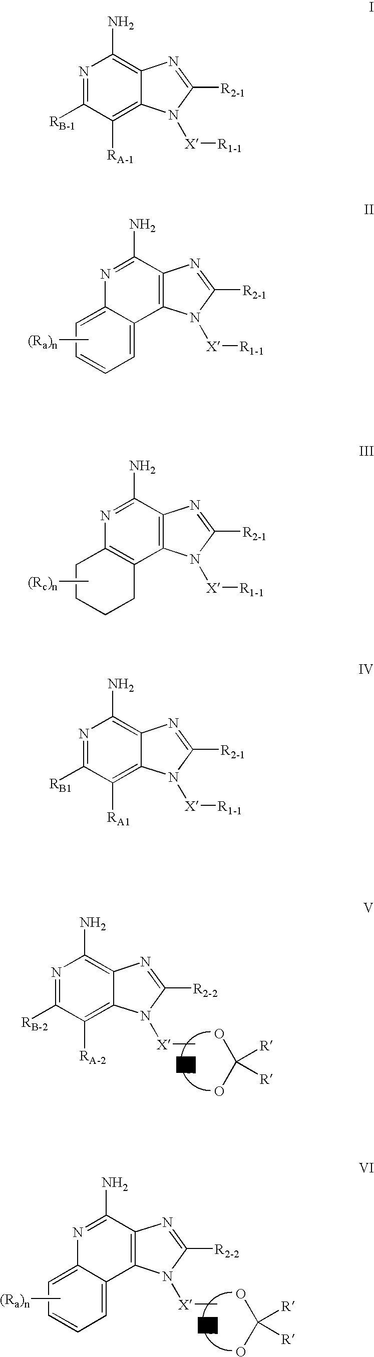 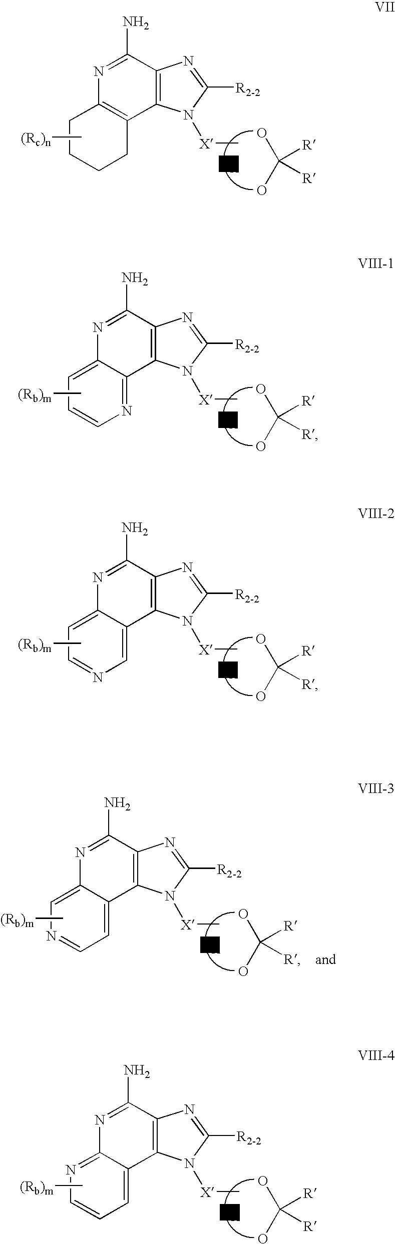 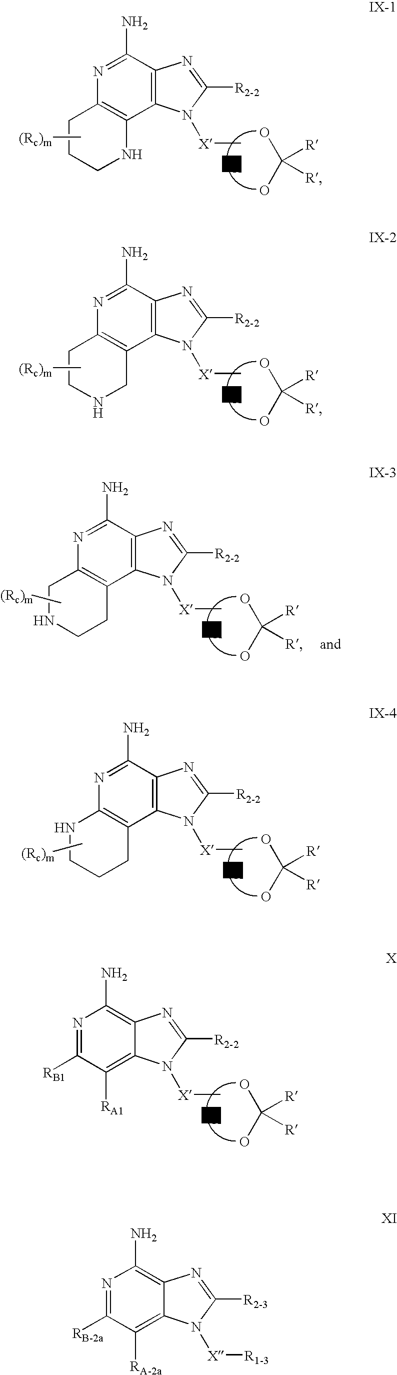 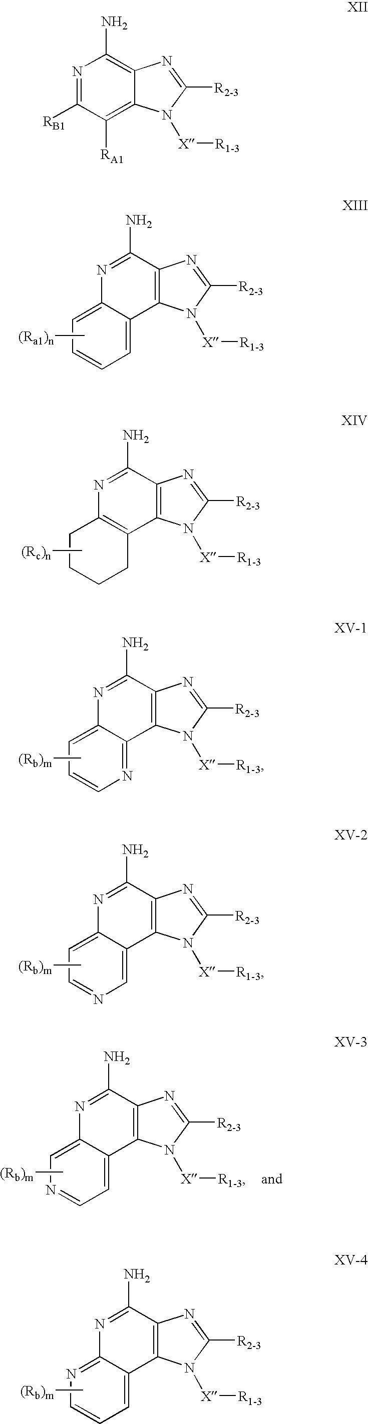 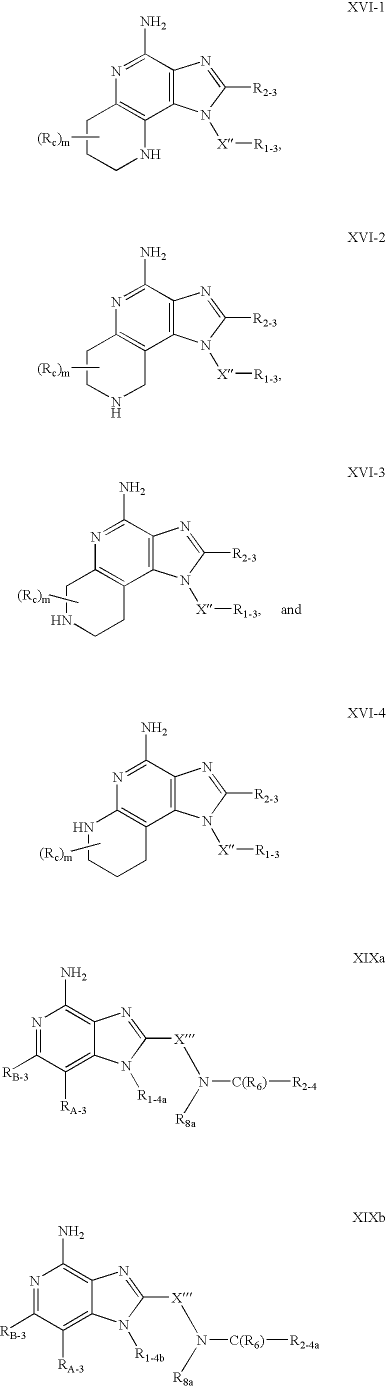 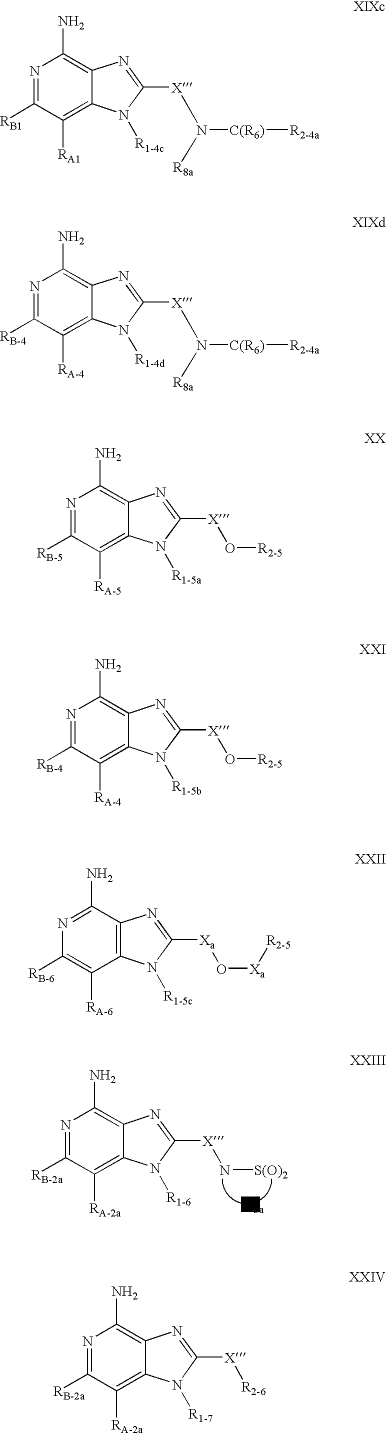
wherein R_{A1}, R_{B1}, R_{A-1}, R_{B-1}, R_{A-2}, R_{B-2}, R_{A-2a}, R_{B-2a}, R_{A-3}, R_{B-3}, R_{A-4}, R_{B-4}, R_{A-5}, R_{B-5}, R_{A-6}, R_{B-6}, R₁₋₁, R₁₋₃, R₁₋₄ₐ, R_{1-4b}, R_{1-4c}, R_{1-4d}, R₁₋₅ₐ, R_{1-5b}, R_{1-5c}, R₁₋₆, R₁₋₇, R₂₋₁, R₂₋₂, R₂₋₃, R₂₋₄, R₂₋₄ₐ, R₂₋₅, R₂₋₆, Rₐ, Rₐ₁, R_{b}, R_{c}, R₃ₐ, R₆, R₈ₐ, R′, R₁₁, Xₐ, X′, X″, X′″, m, and n are as defined below; and pharmaceutically acceptable salts thereof.

In one embodiment, there is provided a compound of the Formula (I): 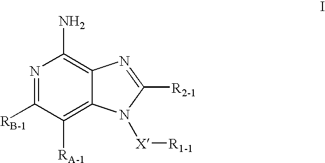
wherein:

R₁₋₁ is selected from the group consisting of —CH(CH₂OH)—OH, —CH(CH₂CH₂OH)—OH, and —CH(CH₂OH)₂;

X′ is selected from the group consisting of —CH(R₉)—, —CH(R₉)-alkylene-, and —CH(R₉)-alkenylene-; wherein the alkylene and alkenylene are optionally interrupted with one or more —O— groups;

R₂₋₁ is selected from the group consisting of hydroxyalkylenyl and alkoxyalkylenyl;

R_{A-1} and R_{B-1} are each independently selected from the group consisting of:
hydrogen,
halogen,
alkyl,
alkenyl,
alkoxy,
alkylthio, and
—N(R₉)₂;

or R_{A-1} and R_{B-1} taken together form either a fused aryl ring that is unsubstituted or substituted by one or more Rₐ groups, or a fused 5 to 7 membered saturated ring that is unsubstituted or substituted by one or more R_{c} groups;

Rₐ is selected from the group consisting of fluorine, alkyl, haloalkyl, alkoxy, and —N(R₉)₂;

R_{c} is selected from the group consisting of halogen, hydroxy, alkyl, alkenyl, haloalkyl, alkoxy, alkylthio, and —N(R₉)₂; and

R₉ is selected from the group consisting of hydrogen and alkyl;

or a pharmaceutically acceptable salt thereof.

In one embodiment, there is provided a compound of the Formula (II):

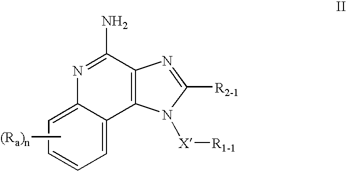

which is an embodiment of Formula I wherein n is an integer of 0 to 4, and Rₐ, n, X′, R₁₋₁, and R₂₋₁ are as defined in Formula I; or a pharmaceutically acceptable salt thereof.

In one embodiment, there is provided a compound of the Formula (III):

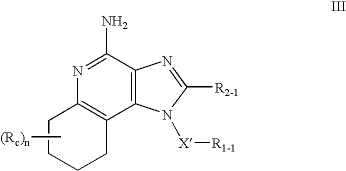

which is an embodiment of Formula I wherein n is an integer of 0 to 4, and R_{c}, n, X′, R₁₋₁, and R₂₋₁ are as defined in Formula I; or a pharmaceutically acceptable salt thereof.

In one embodiment, there is provided a compound of the Formula (IV): 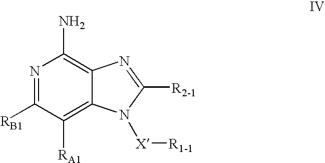
which is an embodiment of Formula I, wherein:

R_{A1} and R_{B1} are each independently selected from the group consisting of:
hydrogen,
halogen,
alkyl,
alkenyl,
alkoxy,
alkylthio, and
—N(R₉)₂; and
   X′, R₁₋₁, and R₂₋₁ are as defined in Formula I; or a pharmaceutically acceptable salt thereof.

In another embodiment, there is provided a compound of the Formula (V): 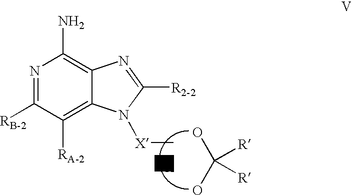
wherein:

R′ is selected from the group consisting of hydrogen, alkyl, alkoxy, and alkoxyalkylenyl, or the R′ groups join together to form a 5 to 7 membered saturated ring optionally substituted by phenyl or phenyl substituted with one or more substituents selected from the group consisting of alkyl, alkoxy, halogen, and trifluoromethyl;

X′ is selected from the group consisting of —CH(R₉)—, —CH(R₉)-alkylene-, and —CH(R₉)-alkenylene-; wherein the alkylene and alkenylene are optionally interrupted with one or more —O— groups;

R₁₁ is a straight chain C₂₋₃ alkylene;

R_{A-2} and R_{B-2} are each independently selected from the group consisting of:
hydrogen,
halogen,
alkyl,
alkenyl,
alkoxy,
alkylthio, and
—N(R₉)₂;

or R_{A-2} and R_{B-2} taken together form either a fused aryl ring that is unsubstituted or substituted by one or more Rₐ groups, or a fused 5 to 7 membered saturated ring that is unsubstituted or substituted by one or more R_{c} groups;

or R_{A-2} and R_{B-2} taken together form a fused heteroaryl or 5 to 7 membered saturated ring, containing one heteroatom selected from the group consisting of N and S, wherein the heteroaryl ring is unsubstituted or substituted by one or more R_{b} groups, and the 5 to 7 membered saturated ring is unsubstituted or substituted by one or more R_{c} groups;

Rₐ is selected from the group consisting of fluorine, alkyl, haloalkyl, alkoxy, and —N(R₉)₂;

R_{b} is selected from the group consisting of halogen, hydroxy, alkyl, haloalkyl, alkoxy, and —N(R₉)₂;

R_{c} is selected from the group consisting of halogen, hydroxy, alkyl, alkenyl, haloalkyl, alkoxy, alkylthio, and —N(R₉)₂;

R₂₋₂ is selected from the group consisting of
—R₄,
—X—R₄,
—X—Y—R₄, and
—X—R₅ₐ;

X is selected from the group consisting of alkylene, alkenylene, alkynylene, arylene, heteroarylene, and heterocyclylene wherein the alkylene, alkenylene, and alkynylene groups are optionally interrupted or terminated by arylene, heteroarylene or heterocyclylene and optionally interrupted by one or more —O— groups;

Y is selected from the group consisting of:
—S(O)₀₋₂—,
—S(O)₂—N(R₈)—,
—C(R₆)—,
—C(R₆)—O—,
—O—C(R₆)—,
—O—C(O)—O—,
—N(R₈)-Q′-,
—C(R₆)—N(R₈)—,
—O—C(R₆)—N(R₈)—,
—C(R₆)—N(OR₉)—,

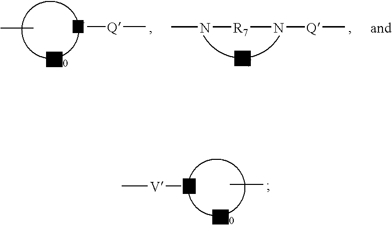

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups are unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;

R₅ₐ is selected from the group consisting of:

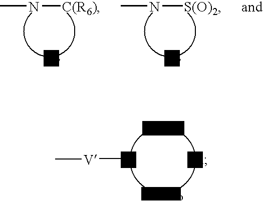

R₆ is selected from the group consisting of ═O and ═S;

R₇ is C₂₋₇ alkylene;

R₈ is selected from the group consisting of hydrogen, alkyl, alkoxyalkylenyl, and arylalkylenyl;

R₉ is selected from the group consisting of hydrogen and alkyl;

R₁₀ is C₃₋₈ alkylene;

A is selected from the group consisting of —O—, —C(O)—, —S(O)₀₋₂—, —CH₂—, and —N(R₄)—;

Q′ is selected from the group consisting of a bond, —C(R₆)—, —C(R₆)—C(R₆)—, —S(O)₂—, and —S(O)₂—N(R₈)—;

V′ is selected from the group consisting of —C(R₆)—, —O—C(R₆)—, and —S(O)₂—; and

a and b are each independently an integer from 1 to 6 with the proviso that a+b is ≦7;

or a pharmaceutically acceptable salt thereof.

In another embodiment, there is provided a compound of the Formula (VI): 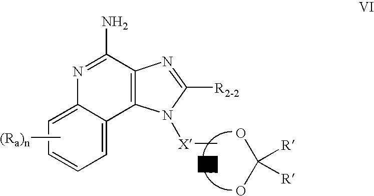
which is an embodiment of Formula V wherein n is an integer of 0 to 4, and Rₐ, X′, R₁₁, R′, and R₂₋₂ are as defined in Formula V; or a pharmaceutically acceptable salt thereof.

In another embodiment, there is provided a compound of the Formula (VII): 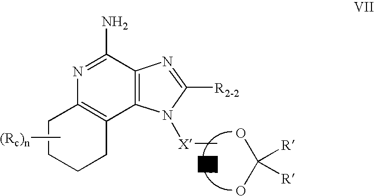
which is an embodiment of Formula V wherein n is an integer of 0 to 4, and R_{c}, X′, R₁₁, R′, and R₂₋₂ are as defined in Formula V; or a pharmaceutically acceptable salt thereof.

In another embodiment, there is provided a compound which is selected from the group consisting of the following formulas (VIII-1, VIII-2, VIII-3, and VIII-4): 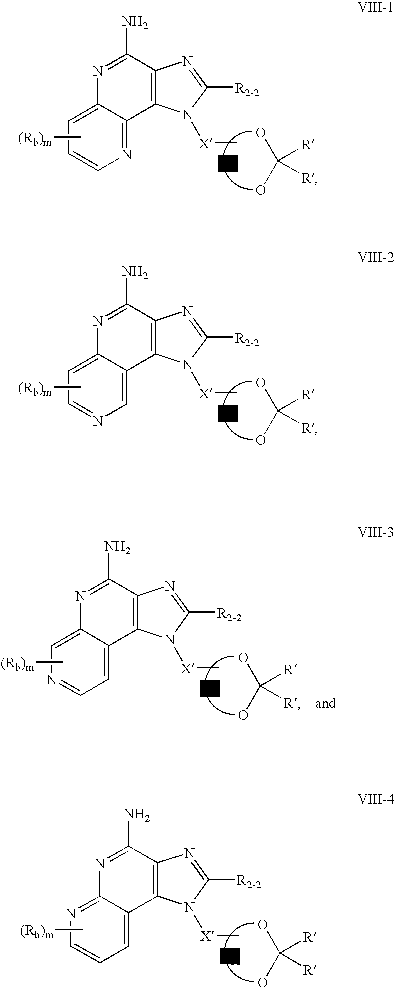
each of which is an embodiment of Formula V wherein m is an integer of 0 to 3, and R_{b}, m, X′, R₁₁, R′, and R₂₋₂ are as defined in Formula V; or a pharmaceutically acceptable salt thereof.

In another embodiment, there is provided a compound which is selected from the group consisting of the following formulas (IX-1, IX-2, IX-3, and IX-4): 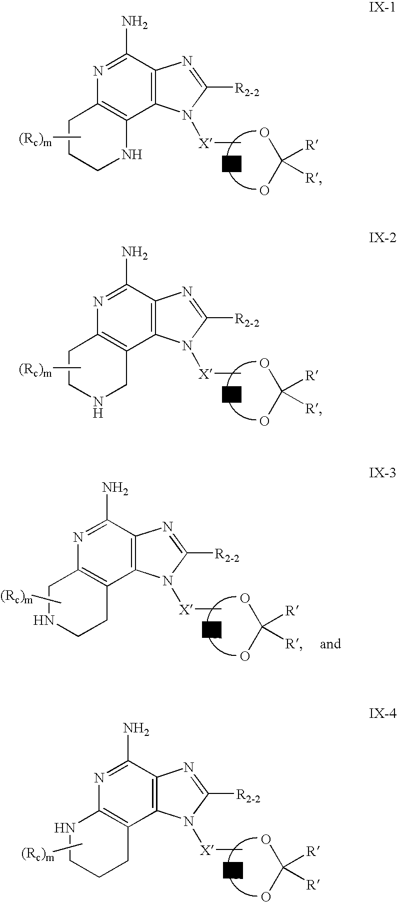
each of which is an embodiment of Formula V wherein m is an integer of 0 to 3, and R_{c}, m, X′, R₁₁, R′, and R₂₋₂ are as defined in Formula V; or a pharmaceutically acceptable salt thereof.

In another embodiment, there is provided a compound of the Formula (X): 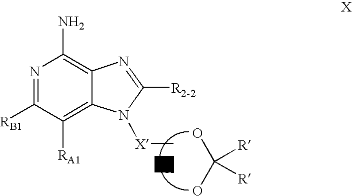
which is an embodiment of Formula V wherein:
R_{A1} and R_{B1} are each independently selected from the group consisting of:
hydrogen,
halogen,
alkyl,
alkenyl,
alkoxy,
alkylthio, and
—N(R₉)₂; and
   X′, R₁₁, R′, and R₂₋₂ are as defined in Formula V; or a pharmaceutically acceptable salt thereof.

In another embodiment, there is provided a compound of the Formula (XI): 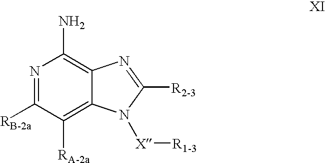
wherein:

R₁₋₃ is selected from the group consisting of:

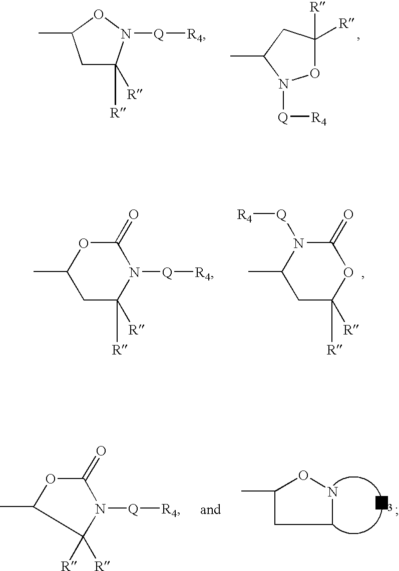

R₃ is C₃₋₅ alkylene;

R″ is selected from the group consisting of:
hydrogen,
alkyl,
alkenyl,
aryl,
arylalkylenyl,
heteroaryl,
heteroarylalkylenyl,
heterocyclyl,
heterocyclylalkylenyl, and
alkyl, alkenyl, aryl, arylalkylenyl, heteroaryl, heteroarylalkylenyl, heterocyclyl, or heterocyclylalkylenyl, substituted by one or more substituents selected from the group consisting of:
   hydroxy,
   alkyl,
   haloalkyl,
   hydroxyalkyl,
   alkoxy,
   dialkylamino,
   —S(O)₀₋₂-alkyl,
   —S(O)₀₋₂-aryl,
   —NH—S(O)₂-alkyl,
   —NH—S(O)₂-aryl,
   haloalkoxy,
   halogen,
   nitrile,
   nitro,
   aryl,
   heteroaryl,
   heterocyclyl,
   aryloxy,
   arylalkyleneoxy,
   —C(O)—O-alkyl,
   —C(O)—N(R₈)₂,
   —N(R₈)—C(O)-alkyl,
   —O—(CO)-alkyl, and
   —C(O)-alkyl;

or two R″ groups on the same carbon atom can join together to form a ring system selected from the group consisting of

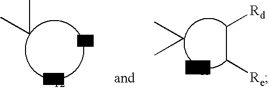

R_{d} and Rₑ are independently selected from the group consisting of hydrogen, halogen, hydroxy, alkyl, alkenyl, aryl, haloalkyl, alkoxy, alkylthio, and —N(R₉)₂; or R_{d} and Rₑ can join to form a fused aryl ring or fused 5-10 membered heteroaryl ring containing one to four heteroatoms;

A′ is selected from the group consisting of —O—, —S(O)₀₋₂—, —N(-Q-R₄)—, and —CH₂—;

R₁₂ is C₃₋₉ alkylene or C₃₋₉ alkenylene, optionally interrupted by one heteroatom;

R₁₃ is C₂₋₇ alkylene or C₂₋₇ alkenylene, optionally interrupted by one heteroatom;

X″ is selected from the group consisting of —CH(R₉)—, —CH(R₉)-alkylene-, and —CH(R₉)-alkenylene-;

R_{A-2a} and R_{B-2a} are each independently selected from the group consisting of:
hydrogen,
halogen,
alkyl,
alkenyl,
alkoxy,
alkylthio, and
—N(R₉)₂;

or R_{A-2a} and R_{B-2a} taken together form either a fused aryl ring that is unsubstituted or substituted by one or more Rₐ₁ groups, or a fused 5 to 7 membered saturated ring that is unsubstituted or substituted by one or more R_{c} groups;

or R_{A-2a} and R_{B-2a} taken together form a fused heteroaryl or 5 to 7 membered saturated ring containing one heteroatom selected from the group consisting of N and S, wherein the heteroaryl ring is unsubstituted or substituted by one or more R_{b} groups, and the 5 to 7 membered saturated ring is unsubstituted or substituted by one or more R_{c} groups;

Rₐ₁ is selected from the group consisting of halogen, alkyl, haloalkyl, alkoxy, and —N(R₉)₂;

R_{b} is selected from the group consisting of halogen, hydroxy, alkyl, haloalkyl, alkoxy, and —N(R₉)₂;

R_{c} is selected from the group consisting of halogen, hydroxy, alkyl, alkenyl, haloalkyl, alkoxy, alkylthio, and —N(R₉)₂;

R₂₋₃ is selected from the group consisting of:
—R₄,
—X—R₄,
—X—Y—R₄, and
—X—R₅ₐ;

X is selected from the group consisting of alkylene, alkenylene, alkynylene, arylene, heteroarylene, and heterocyclylene wherein the alkylene, alkenylene, and alkynylene groups are optionally interrupted or terminated by arylene, heteroarylene or heterocyclylene and optionally interrupted by one or more —O— groups;

Y is selected from the group consisting of:
—S(O)₀₋₂—,
—S(O)₂—N(R₈)—,
—C(R₆)—,
—C(R₆)—O—,
—O—C(R₆)—,
—O—C(O)—O—,
—N(R₈)-Q′-,
—C(R₆)—N(R₈)—,
—O—C(R₆)—N(R₈)—,
—C(R₆)—N(OR₉)—,

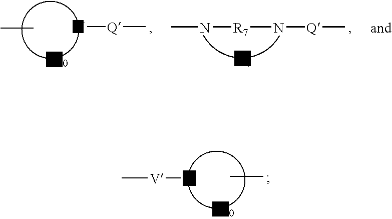

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups are unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;

R₅ₐ is selected from the group consisting of:

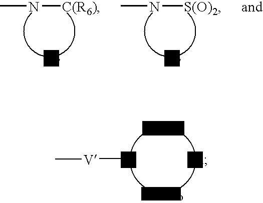

R₆ is selected from the group consisting of ═O and ═S;

R₇ is C₂₋₇ alkylene;

R₈ is selected from the group consisting of hydrogen, alkyl, alkoxyalkylenyl, and arylalkylenyl;

R₉ is selected from the group consisting of hydrogen and alkyl;

R₁₀ is C₃₋₈ alkylene;

A is selected from the group consisting of —O—, —C(O)—, —S(O)₀₋₂—, —CH₂—, and —N(R₄)—;

Q is selected from the group consisting of a bond, —C(R₆)—, —C(R₆)—C(R₆)—, —S(O)₂—, —C(R₆)—N(R₈)—W—, —S(O)₂—N(R₈)—, and —C(R₆)—O—, and —C(R₆)—N(OR₉)—;

Q′ is selected from the group consisting of a bond, —C(R₆)—, —C(R₆)—C(R₆)—, —S(O)₂—, and —S(O)₂—N(R₈)—;

V′ is selected from the group consisting of —C(R₆)—, —O—C(R₆)—, and —S(O)₂—;

W is selected from the group consisting of a bond, —C(O)—, and —S(O)₂—; and

a and b are each independently an integer from 1 to 6 with the proviso that a+b is ≦7;

or a pharmaceutically acceptable salt thereof.

In another embodiment, there is provided a compound of the Formula (XII): 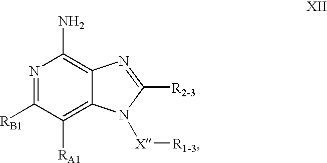
which is an embodiment of Formula XI, wherein:

R_{A1} and R_{B1} are each independently selected from the group consisting of:
hydrogen,
halogen,
alkyl,
alkenyl,
alkoxy,
alkylthio, and
—N(R₉)₂; and
   X″, R₁₋₃, and R₂₋₃ are as defined in Formula XI.

In another embodiment, there is provided a compound of the Formula (XIII): 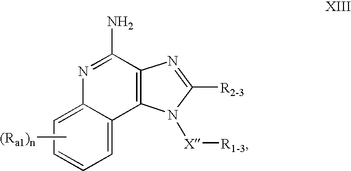
which is an embodiment of Formula XI, wherein n is an integer of 0 to 4, and Rₐ₁, X″, R₁₋₃, and R₂₋₃ are as defined in Formula XI.

In another embodiment, there is provided a compound of the Formula (XIV): 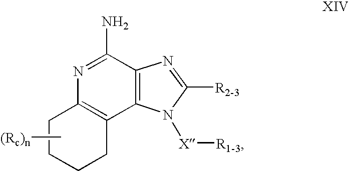
which is an embodiment of Formula XI, wherein n is an integer of 0 to 4, and R_{c}, X″, R₁₋₃, and R₂₋₃ are as defined in Formula XI.

In another embodiment, there is provided a compound selected from the group consisting of the following formulas (XV-1, XV-2, XV-3, and XV-4): 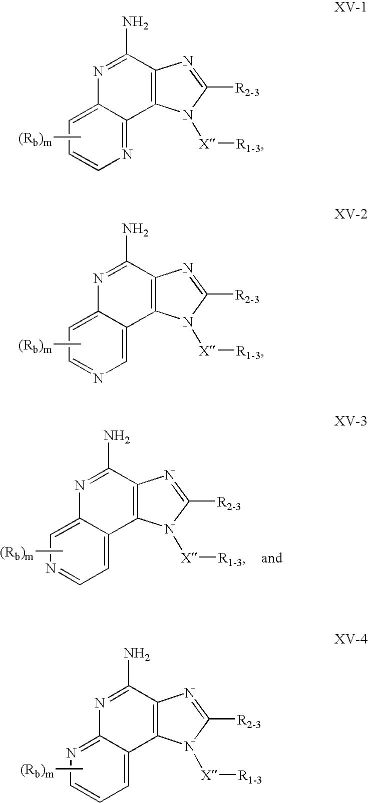
each of which is an embodiment of Formula XI, wherein m is an integer of 0 to 3, and R_{b}, X″, R₁₋₃, and R₂₋₃ are as defined in Formula XI.

In another embodiment, there is provided a compound selected from the group consisting of the following formulas (XVI-1, XVI-2, XVI-3, and XVI-4): 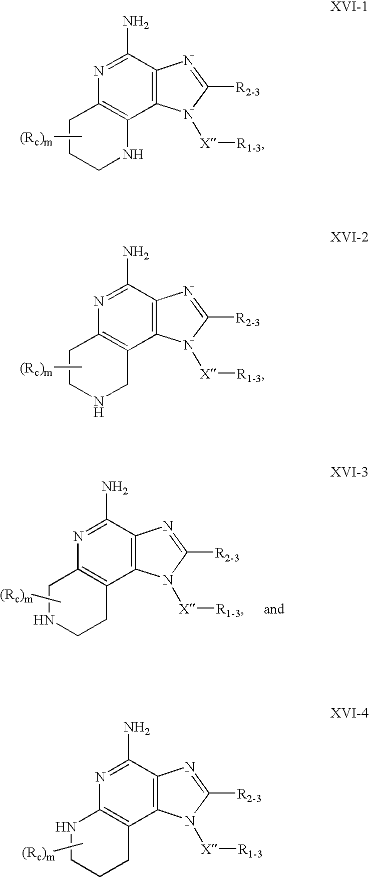
each of which is an embodiment of Formula XI, wherein m is an integer of 0 to 3, and R_{c}, X″, R₁₋₃, and R₂₋₃ are as defined in Formula XI.

In one embodiment, the present invention provides a compound of the following formula (XVII), which is useful, for example, as an intermediate for making compounds of Formulas XI, XIII, and XIV: 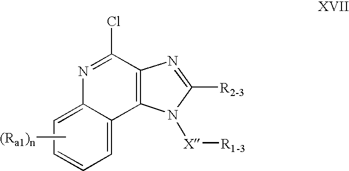
wherein Rₐ₁, n, X″, R₁₋₃, and R₂₋₃ are as defined in Formula XIII;
or a pharmaceutically acceptable salt thereof.

In one embodiment, the present invention provides a compound of the following formula (XVIII), which is useful, for example, as an intermediate for making compounds of Formula XII: 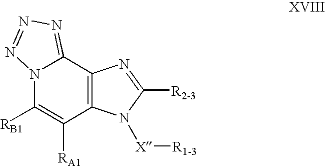
wherein R_{A1}, R_{B1}, X″, R₁₋₃, and R₂₋₃ are as defined in Formula XII;
or a pharmaceutically acceptable salt thereof.

In another embodiment, there is provided a compound of the Formula (XIXa): 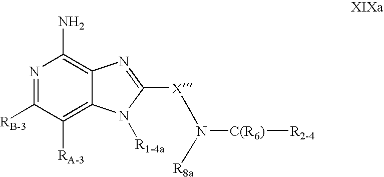
wherein:

X′″ is selected from the group consisting of C₁₋₄ alkylene and C₂₋₄ alkenylene;

R₂₋₄ is selected from the group consisting of C₃₋₆ alkyl, C₂₋₆ alkenyl,

C₂₋₆ alkynyl, aryl, arylC₁₋₄ alkylenyl, aryloxyC₁₋₄ alkylenyl, C₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl wherein the C₃₋₆ alkyl, C₂₋₆ alkenyl, C₂₋₆ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl groups are unsubstituted or substituted by one or more substituents independently selected from the group consisting of C₁₋₄ alkyl, C₁₋₄ alkoxy, C₁₋₄ alkanoyl, C₁₋₄ alkoxycarbonyl, hydroxyC₁₋₄ alkyl, haloC₁₋₄ alkyl, haloC₁₋₄ alkoxy, halogen, nitro, hydroxy, mercapto, cyano, amino, C₁₋₄ alkylamino, di(C₁₋₄ alkyl)amino, and in the case of C₃₋₆ alkyl, C₂₋₆ alkenyl, C₂₋₆ alkynyl, and heterocyclyl, oxo;

R_{A-3} and R_{B-3} form a fused aryl ring that is unsubstituted or substituted by one or more Rₐ₁ groups, or R_{A-3} and R_{B-3} form a fused 5 to 7 membered saturated ring that is unsubstituted or substituted by one or more R_{c} groups;

Rₐ₁ is selected from the group consisting of halogen, alkyl, haloalkyl, alkoxy, and —N(R₉)₂;

R_{c} is selected from the group consisting of halogen, hydroxy, alkyl, alkenyl, haloalkyl, alkoxy, alkylthio, and —N(R₉)₂;

R₁₋₄ₐ is selected from the group consisting of:
hydrogen,
alkyl,
alkenyl,
alkoxyalkylenyl,
aryl,
arylalkylenyl, 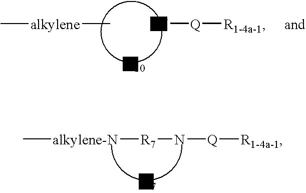
wherein the alkyl, alkenyl, alkoxyalkylenyl, aryl, and arylalkylenyl can be unsubstituted or substituted with one or more substituents selected from the group consisting of alkyl, alkoxy, hydroxyalkyl, haloalkyl, haloalkoxy, halogen, nitro, hydroxy, mercapto, cyano, amino, alkylamino, and dialkylamino;
with the proviso that when R₁₋₄ₐ includes a carbocyclic ring, then the ring carbon atom by which the ring is attached is otherwise unsubstituted or substituted by an atom other than O, S, or N;

R₁₋₄ₐ₋₁ is selected from the group consisting of alkyl, alkenyl, alkoxyalkylenyl, aryl, and arylalkylenyl, wherein the alkyl, alkenyl, alkoxyalkylenyl, aryl, and arylalkylenyl can be unsubstituted or substituted with one or more substituents selected from the group consisting of alkyl, alkoxy, hydroxyalkyl, haloalkyl, haloalkoxy, halogen, nitro, hydroxy, mercapto, cyano, amino, alkylamino, and dialkylamino;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—;

W is selected from the group consisting of a bond, —C(O)—, and —S(O)₂—;

R₆ is selected from the group consisting of ═O and ═S;

R₇ is C₂₋₇ alkylene;

R₈ₐ is selected from the group consisting of hydrogen and C₁₋₄ alkyl;

R₈ is selected from the group consisting of hydrogen, alkyl, alkoxyalkylenyl, and arylalkylenyl;

R₉ is selected from the group consisting of hydrogen and alkyl; and

R₁₀ is C₃₋₈ alkylene;

or a pharmaceutically acceptable salt thereof.

In another embodiment, there is provided a compound of the Formula (XIXb): 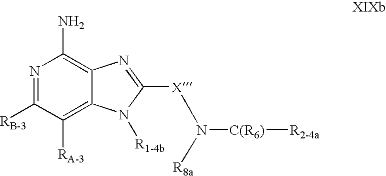
wherein:

X′″ is selected from the group consisting of C₁₋₄ alkylene and C₂₋₄ alkenylene;

R₂₋₄ₐ is selected from the group consisting of C₁₋₆ alkyl, C₂₋₆ alkenyl, C₂₋₆ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl wherein the C₁₋₆ alkyl, C₂₋₆ alkenyl, C₂₋₆ alkynyl, aryl, arylC₁₋₄ alkylenyl, aryloxyC₁₋₄ alkylenyl, C₁₋₄ alkylarylenyl, heteroaryl, heteroarylC₁₋₄alkylenyl, heteroaryloxyC₁₋₄ alkylenyl, C₁₋₄ alkylheteroarylenyl, and heterocyclyl groups are unsubstituted or substituted by one or more substituents independently selected from the group consisting of C₁₋₄ alkyl, C₁₋₄ alkoxy, C₁₋₄ alkanoyl, C₁₋₄ alkoxycarbonyl, hydroxyC₁₋₄ alkyl, haloC₁₋₄ alkyl, haloC₁₋₄ alkoxy, halogen, nitro, hydroxy, mercapto, cyano, amino, C₁₋₄ alkylamino, di(C₁₋₄ alkyl)amino, and in the case of C₂₋₆ alkyl, C₂₋₆ alkenyl, C₂₋₆ alkynyl, and heterocyclyl, oxo;

R_{A-3} and R_{B-3} form a fused aryl ring that is unsubstituted or substituted by one or more Rₐ₁ groups, or R_{A-3} and R_{B-3} form a fused 5 to 7 membered saturated ring that is unsubstituted or substituted by one or more R_{c} groups;

Rₐ₁ is selected from the group consisting of halogen, alkyl, haloalkyl, alkoxy, and —N(R₉)₂;

R_{c} is selected from the group consisting of halogen, hydroxy, alkyl, alkenyl, haloalkyl, alkoxy, alkylthio, and —N(R₉)₂;

R_{1-4b} is selected from the group consisting of:
pyridinylmethyl,
—X₁—Y₁—R₄, 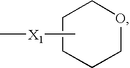
—X₂—Ar,
—X₂—Ar′—R₄,
—X₂—C(R₆)—O—R₄,
—X₂-alkylene-OH,
—X₂-alkynylene-R₄, 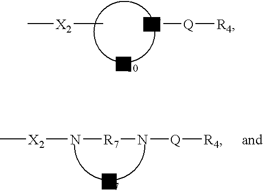
—X₁—R₅;

X₁ is selected from the group consisting of alkylene, alkenylene, alkynylene, arylene, heteroarylene, and heterocyclylene wherein the alkylene group can be optionally interrupted or terminated with arylene or heteroarylene and optionally interrupted by one or more —O— groups;

X₂ is selected from the group consisting of alkylene, alkenylene, alkynylene, arylene, heteroarylene, and heterocyclylene wherein the alkylene, alkenylene, and alkynylene groups are interrupted by one or more —O— groups and can be optionally interrupted or terminated with arylene, heteroarylene, or heterocyclylene;

Y₁ is selected from the group consisting of:
—S(O)₀₋₂—,
—O—C(R₆)—,
—O—C(O)—O—,
—N(R₈)-Q-,
—O—C(R₆)—N(R₈)—,

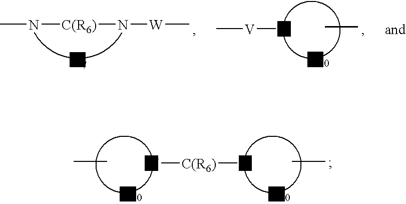

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups are unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;

R₅ is selected from the group consisting of:

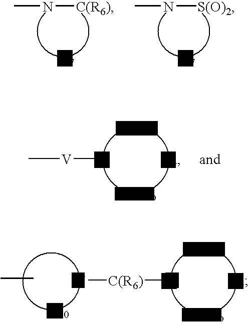

Ar is selected from the group consisting of aryl and heteroaryl both of which can be unsubstituted or can be substituted by one or more substituents independently selected from the group consisting of alkyl, alkoxy, haloalkyl, haloalkoxy, halogen, nitro, hydroxy, hydroxyalkyl, mercapto, cyano, carboxy, formyl, aryl, aryloxy, arylalkyleneoxy, heteroaryl, heteroaryloxy, heteroarylalkyleneoxy, heterocyclyl, heterocyclylalkylenyl, amino, alkylamino, and dialkylamino;

Ar′ is selected from the group consisting of arylene and heteroarylene both of which can be unsubstituted or can be substituted by one or more substituents independently selected from the group consisting of alkyl, alkoxy, haloalkyl, haloalkoxy, halogen, nitro, hydroxy, hydroxyalkyl, mercapto, cyano, carboxy, formyl, aryl, aryloxy, arylalkyleneoxy, heteroaryl, heteroaryloxy, heteroarylalkyleneoxy, heterocyclyl, heterocyclylalkylenyl, amino, alkylamino, and dialkylamino;

A is selected from the group consisting of —O—, —C(O)—, —CH₂—, —S(O)₀₋₂—, and —N(R₄)—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—;

V is selected from the group consisting of —O—C(R₆)— and —N(R₈)—C(R₆)—;

W is selected from the group consisting of a bond, —C(O)—, and —S(O)₂—;

a and b are each independently an integer from 1 to 6 with the proviso that a+b is ≦7;

R₆ is selected from the group consisting of ═O and ═S;

R₇ is C₂₋₇ alkylene;

R₈ₐ is selected from the group consisting of hydrogen and C₁₋₄ alkyl;

R₈ is selected from the group consisting of hydrogen, alkyl, alkoxyalkylenyl, and arylalkylenyl;

R₉ is selected from the group consisting of hydrogen and alkyl; and

R₁₀ is C₃₋₈ alkylene;

with the proviso that when X₁ is interrupted with one —O— group, then Y₁ is other than —S(O)₀₋₂—;

or a pharmaceutically acceptable salt thereof.

In another embodiment, there is provided a compound of the Formula (XIXc): 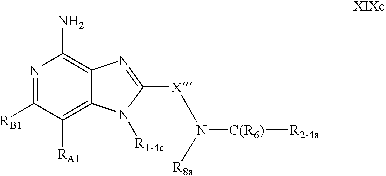
wherein:

X′″ is selected from the group consisting of C₁₋₄alkylene and C₂₋₄ alkenylene;

R₂₋₄ₐ is selected from the group consisting of C₁₋₆ alkyl, C₂₋₆ alkenyl, C₂₋₆ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄alkylenyl, C₁₋₄ alkylheteroarylenyl, and heterocyclyl wherein the C₁₋₆ alkyl, C₂₋₆ alkenyl, C₂₋₆ alkynyl, aryl, arylC₁₋₄ alkylenyl, aryloxyC₁₋₄alkylenyl, C₁₋₄alkylarylenyl, heteroaryl, heteroarylC₁₋₄alkylenyl, heteroaryloxyC₁₋₄ alkylenyl, C₁₋₄ alkylheteroarylenyl, and heterocyclyl groups are unsubstituted or substituted by one or more substituents independently selected from the group consisting of C₁₋₄ alkyl, C₁₋₄ alkoxy, C₁₋₄ alkanoyl, C₁₋₄ alkoxycarbonyl, hydroxyC₁₋₄ alkyl, haloC₁₋₄ alkyl, haloC₁₋₄ alkoxy, halogen, nitro, hydroxy, mercapto, cyano, amino, C₁₋₄ alkylamino, di(C₁₋₄ alkyl)amino, and in the case of C₂₋₆ alkyl, C₂₋₆ alkenyl, C₂₋₆ alkynyl, and heterocyclyl, oxo;

R_{A1} and R_{B1} are each independently selected from the group consisting of:
hydrogen,
halogen,
alkyl,
alkenyl,
alkoxy,
alkylthio, and
—N(R₉)₂;

R_{1-4c} is selected from the group consisting of:
—R₄ₐ,
—X₃—Y₃—R₄ₐ,
—X₂—R₄ₐ,
—X₂—Y₂—R₄ₐ, and
—X₂—R₅₋₁;

X₂ is selected from the group consisting of alkylene, alkenylene, alkynylene, arylene, heteroarylene, and heterocyclylene wherein the alkylene, alkenylene, and alkynylene groups are interrupted by one or more —O— groups and can be optionally interrupted or terminated with arylene, heteroarylene, or heterocyclylene;

X₃ is selected from the group consisting of alkylene, arylene, heteroarylene, and heterocyclylene wherein the alkylene group can be optionally interrupted or terminated by arylene, heteroarylene or heterocyclylene and optionally interrupted by one or more —O— groups;

Y₂ is selected from the group consisting of:
—S(O)₀₋₂—,
—C(R₆)—O—,
—O—C(R₆)—,
—O—C(O)—O—,
—N(R₈)-Q-, and
—O—C(R₆)—N(R₈)—,

Y₃ is selected from the group consisting of:

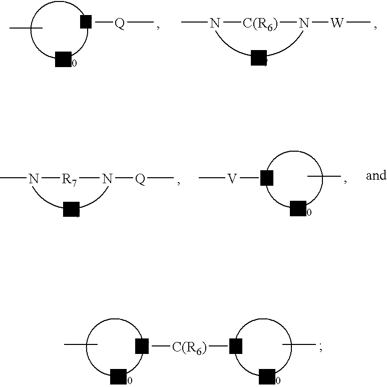

R₄ₐ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkylenyl, haloalkylenyl, haloalkyleneoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of heterocyclyl, oxo;

with the proviso that when R_{1-4c} is —R₄ₐ, and R₄ₐ includes a carbocyclic ring or heterocyclic ring containing one heteroatom, then the ring carbon atom by which the ring is attached is otherwise unsubstituted or substituted by an atom other than O, S, or N;

with the further proviso that R_{1-4c} is other than an unsubstituted or substituted isoxazolylalkylenyl, dihydroisoxazolylalkylenyl, or oxadiazolylalkylenyl group;

R₅₋₁ is selected from the group consisting of:

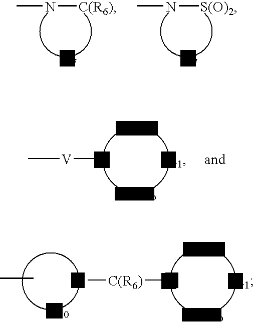

R₆ is selected from the group consisting of ═O and ═S;

R₇ is C₂₋₇ alkylene;

R₈ₐ is selected from the group consisting of hydrogen and C₁₋₄ alkyl;

R₈ is selected from the group consisting of hydrogen, alkyl, alkoxyalkylenyl, and arylalkylenyl;

R₉ is selected from the group consisting of hydrogen and alkyl;

R₁₀ is independently C₃₋₈ alkylene;

A₁ is selected from the group consisting of —O—, —C(O)—, —CH₂—, —S(O)₀₋₂—, and —N(R₄ₐ)—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—;

V is selected from the group consisting of —O—C(R₆)— and —N(R₈)—C(R₆)—;

W is selected from the group consisting of a bond, —C(O)—, and —S(O)₂—; and

a and b are each independently an integer from 1 to 6 with the proviso that a+b is ≦7;

or a pharmaceutically acceptable salt thereof.

In another embodiment, there is provided a compound of the Formula (XIXd): 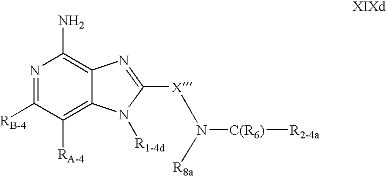
wherein:

X′″ is selected from the group consisting of C₁₋₄ alkylene and C₂₋₄ alkenylene;

R₂₋₄ₐ is selected from the group consisting of C₁₋₆ alkyl, C₂₋₆ alkenyl, C₂₋₆ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl wherein the C₁₋₆ alkyl, C₂₋₆ alkenyl, C₂₋₆ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl groups are unsubstituted or substituted by one or more substituents independently selected from the group consisting of C₁₋₄ alkyl, C₁₋₄ alkoxy, C₁₋₄ alkanoyl, C₁₋₄ alkoxycarbonyl, hydroxyC₁₋₄ alkyl, haloC₁₋₄ alkyl, haloC₁₋₄ alkoxy, halogen, nitro, hydroxy, mercapto, cyano, amino, C₁₋₄ alkylamino, di(C₁₋₄ alkyl)amino, and in the case of C₂₋₆ alkyl, C₂₋₆ alkenyl, C₂₋₆ alkynyl, and heterocyclyl, oxo;

R_{A-4} and R_{B-4} taken together form a fused heteroaryl or 5 to 7 membered saturated ring containing one heteroatom selected from the group consisting of N and S, wherein the heteroaryl ring is unsubstituted or substituted by one or more R_{b} groups, and the 5 to 7 membered saturated ring is unsubstituted or substituted by one or more R_{c} groups;

R_{b} is selected from the group consisting of halogen, hydroxy, alkyl, haloalkyl, alkoxy, and —N(R₉)₂;

R_{c} is selected from the group consisting of halogen, hydroxy, alkyl, alkenyl, haloalkyl, alkoxy, alkylthio, and —N(R₉)₂;

R_{1-4d} is selected from the group consisting of:
—R_{4b},
—X—R_{4b},
—X—Yₐ—R_{4b}, and
—X—R₅₋₂;

X is selected from the group consisting of alkylene, alkenylene, alkynylene, arylene, heteroarylene, and heterocyclylene wherein the alkylene, alkenylene, and alkynylene groups can be optionally interrupted or terminated by arylene, heteroarylene or heterocyclylene and optionally interrupted by one or more —O— groups;

Yₐ is selected from the group consisting of:
—S(O)₀₋₂—,
—C(R₆)—,
—C(R₆)—O—,
—O—C(R₆)—,
—O—C(O)—O—,
—N(R₈)-Q-,
—O—C(R₆)—N(R₈)—,
—C(R₆)—N(OR₉)—,

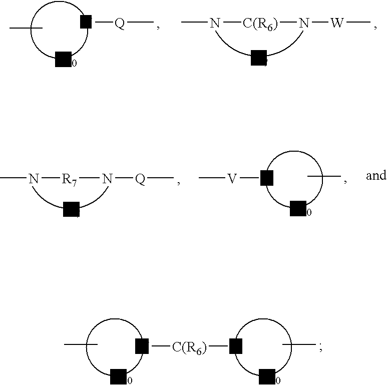

R_{4b} is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkylenyl, haloalkylenyl, haloalkyleneoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl and heterocyclyl, oxo;

with the proviso that when R_{1-4d} is —R_{4b} or —X—R_{4b}, and R_{4b} or X—R_{4b} includes a carbocyclic ring or heterocyclic ring containing one heteroatom, then the ring carbon atom by which the ring is attached is otherwise unsubstituted or substituted by an atom other than O, S, or N;

with the further proviso that R_{1-4d} is other than an unsubstituted or substituted isoxazolylalkylenyl, dihydroisoxazolylalkylenyl, or oxadiazolylalkylenyl group;

R₅₋₂ is selected from the group consisting of:

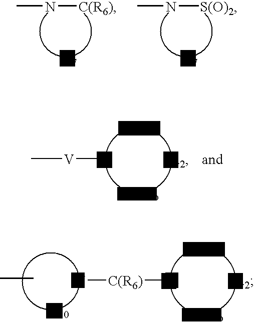

R₆ is selected from the group consisting of ═O and ═S;

R₇ is C₂₋₇ alkylene;

R₈ₐ is selected from the group consisting of hydrogen and C₁₋₄ alkyl;

R₈ is selected from the group consisting of hydrogen, alkyl, alkoxyalkylenyl, and arylalkylenyl;

R₉ is selected from the group consisting of hydrogen and alkyl;

R₁₀ is C₃₋₈ alkylene;

A₂ is selected from the group consisting of —O—, —C(O)—, —CH₂—, —S(O)₀₋₂—, and —N(R_{4b})—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—;

V is selected from the group consisting of —O—C(R₆)— and —N(R₈)—C(R₆)—;

W is selected from the group consisting of a bond, —C(O)—, and —S(O)₂—; and

a and b are each independently an integer from 1 to 6 with the proviso that a+b is ≦7;

with the proviso that when X is interrupted with one —O— group, then Y is other than —S(O)₀₋₂—;

or a pharmaceutically acceptable salt thereof.

In another embodiment, there is provided a compound of the Formula (XX): 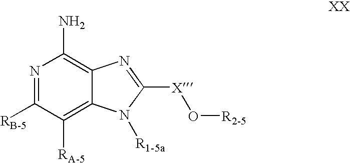
wherein:

X′″ is selected from the group consisting of C₁₋₄ alkylene and C₂₋₄ alkenylene;

R₁₋₅ₐ is selected from the group consisting of:
hydrogen,
alkyl,
alkoxyalkylenyl,
hydroxyalkoxyalkylenyl,
alkenyl,
alkynyl,
aryl,
arylalkylenyl,
alkylarylenyl,
heteroaryl,
heteroarylalkylenyl,
alkylheteroarylenyl,
heterocyclyl,
—X₃—O—C(R₆)—R₁₋₄ₐ₋₁,
X₃—O—C(R₆)—O—R₁₋₄ₐ₋₁, and
—X₃—O—C(R₆)—N(R₈)—R₁₋₄ₐ₋₁,
wherein the alkyl, aryl, arylalkylenyl, alkylarylenyl, heteroaryl, heteroarylalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, heteroaryl, tetrahydropyranyl, amino, alkylamino, dialkylamino, and in the case of heterocyclyl, oxo;

with the proviso that when R₁₋₅ₐ includes a carbocyclic ring or heterocyclic ring containing one heteroatom, then the ring carbon atom by which the ring is attached is otherwise unsubstituted or substituted by an atom other than O, S, or N;

with the further proviso that R₁₋₅ₐ is other than an unsubstituted or substituted isoxazolylalkylenyl, dihydroisoxazolylalkylenyl, or oxadiazolylalkylenyl group;

R₂₋₅ is selected from the group consisting of:
—Ar,
—Ar′—Y″—R₄₋₁, and
—Ar′—X′″—Y″—R₄₋₁,

R_{A-5} and R_{B-5} are each independently selected from the group consisting of:
hydrogen,
halogen,
alkyl,
alkenyl,
alkoxy,
alkylthio, and
—N(R₉)₂,

or R_{A-5} and R_{B-5} taken together form a fused aryl ring that is unsubstituted or substituted by one or more Rₐ₁ groups,

or R_{A-5} and R_{B-5} taken together form a fused 5 to 7 membered saturated ring, unsubstituted or substituted by one or more R_{c} groups;

Rₐ₁ is selected from the group consisting of halogen, alkyl, haloalkyl, alkoxy, and —N(R₉)₂;

R_{c} is selected from the group consisting of halogen, hydroxy, alkyl, alkenyl, haloalkyl, alkoxy, alkylthio, and —N(R₉)₂;

Ar is selected from the group consisting of aryl and heteroaryl both of which can be unsubstituted or can be substituted by one or more substituents independently selected from the group consisting of alkyl, alkoxy, haloalkyl, haloalkoxy, halogen, nitro, hydroxy, hydroxyalkyl, mercapto, cyano, carboxy, formyl, aryl, aryloxy, arylalkyleneoxy, heteroaryl, heteroaryloxy, heteroarylalkyleneoxy, heterocyclyl, heterocyclylalkylenyl, amino, alkylamino, and dialkylamino;

Ar′ is selected from the group consisting of arylene and heteroarylene both of which can be unsubstituted or can be substituted by one or more substituents independently selected from the group consisting of alkyl, alkoxy, haloalkyl, haloalkoxy, halogen, nitro, hydroxy, hydroxyalkyl, mercapto, cyano, carboxy, formyl, aryl, aryloxy, arylalkyleneoxy, heteroaryl, heteroaryloxy, heteroarylalkyleneoxy, heterocyclyl, heterocyclylalkylenyl, amino, alkylamino, and dialkylamino;

X₃ is selected from the group consisting of alkylene, arylene, heteroarylene, and heterocyclylene wherein the alkylene group can be optionally interrupted or terminated by arylene, heteroarylene or heterocyclylene and optionally interrupted by one or more —O— groups;

Y″ is selected from the group consisting of:
—S(O)₀₋₂—,
—S(O)₂—N(R₈ₐ)—,
—C(R₆)—,
—C(R₆)—O—,
—O—C(R₆)—,
—O—C(O)—O—,
—N(R₈ₐ)-Qₐ-,
—C(R₆)—N(R₈₁)—,
—O—C(R₆)—N(R₈ₐ)—, and
—C(R₆)—N(OR₉)—;

R₁₋₄ₐ₋₁ is selected from the group consisting of alkyl, alkenyl, alkoxyalkylenyl, aryl, and arylalkylenyl, wherein the alkyl, alkenyl, alkoxyalkylenyl, aryl, and arylalkylenyl can be unsubstituted or substituted with one or more substituents selected from the group consisting of alkyl, alkoxy, hydroxyalkyl, haloalkyl, haloalkoxy, halogen, nitro, hydroxy, mercapto, cyano, amino, alkylamino, and dialkylamino;

R₄₋₁ is selected from the group consisting of C₁₋₆ alkyl, C₂₋₆ alkenyl, C₂₋₆ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl wherein the C₁₋₆ alkyl, C₂₋₆ alkenyl, C₂₋₆ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl groups are unsubstituted or substituted by one or more substituents independently selected from the group consisting of C₁₋₄ alkyl, C₁₋₄ alkoxy, hydroxyC₁₋₄ alkyl, haloC₁₋₄ alkyl, haloC₁₋₄ alkoxy, halogen, nitro, hydroxy, mercapto, cyano, amino, C₁₋₄ alkylamino, di(C₁₋₄ alkyl)amino, and in the case of C₂₋₆ alkyl, C₂₋₆ alkenyl, C₂₋₆ alkynyl, and heterocyclyl, oxo;

with the proviso that when Y″ is —S(O)₂—N(R₈ₐ)— or —C(R₆)—N(R₈ₐ)—, then R₄₋₁ can also be hydrogen;

R₆ is selected from the group consisting of ═O and ═S;

R₈ₐ is selected from the group consisting of hydrogen and C₁₋₄ alkyl.

R₈ is selected from the group consisting of hydrogen, alkyl, alkoxyalkylenyl, and arylalkylenyl;

R₉ is selected from the group consisting of hydrogen and alkyl;

Qₐ is selected from the group consisting of a bond, —C(R₆)—, —C(R₆)—C(R₆)—, —S(O)₂—, —C(R₆)—N(R₈ₐ)—W—, —S(O)₂—N(R₈ₐ)—, —C(R₆)—O—, and —C(R₆)—N(OR₉)—; and

W is selected from the group consisting of a bond, —C(O)—, and —S(O)₂—;

or a pharmaceutically acceptable salt thereof.

In another embodiment, there is provided a compound of the Formula (XXI): 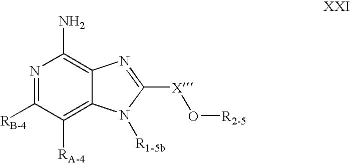
wherein:

X′″ is selected from the group consisting of C₁₋₄ alkylene and C₂₋₄ alkenylene;

R_{1-5b} is selected from the group consisting of:
—R_{4c},
—X—R_{4c},
—X—Y′—R_{4c}, and
—X—R₅₋₃;

R₂₋₅ is selected from the group consisting of:
—Ar,
—Ar′-Y″—R₄₋₁, and
—Ar′—X′″—Y″—R₄₋₁;

R_{A-4} and R_{B-4} taken together form a fused heteroaryl or 5 to 7 membered saturated ring containing one heteroatom selected from the group consisting of N and S, wherein the heteroaryl ring is unsubstituted or substituted by one or more R_{b} groups, and the 5 to 7 membered saturated ring is unsubstituted or substituted by one or more R_{c} groups;

R_{b} is selected from the group consisting of halogen, hydroxy, alkyl, haloalkyl, alkoxy, and —N(R₉)₂;

R_{c} is selected from the group consisting of halogen, hydroxy, alkyl, alkenyl, haloalkyl, alkoxy, alkylthio, and —N(R₉)₂;

Ar is selected from the group consisting of aryl and heteroaryl both of which can be unsubstituted or can be substituted by one or more substituents independently selected from the group consisting of alkyl, alkoxy, haloalkyl, haloalkoxy, halogen, nitro, hydroxy, hydroxyalkyl, mercapto, cyano, carboxy, formyl, aryl, aryloxy, arylalkyleneoxy, heteroaryl, heteroaryloxy, heteroarylalkyleneoxy, heterocyclyl, heterocyclylalkylenyl, amino, alkylamino, and dialkylamino;

Ar′ is selected from the group consisting of arylene and heteroarylene both of which can be unsubstituted or can be substituted by one or more substituents independently selected from the group consisting of alkyl, alkoxy, haloalkyl, haloalkoxy, halogen, nitro, hydroxy, hydroxyalkyl, mercapto, cyano, carboxy, formyl, aryl, aryloxy, arylalkyleneoxy, heteroaryl, heteroaryloxy, heteroarylalkyleneoxy, heterocyclyl, heterocyclylalkylenyl, amino, alkylamino, and dialkylamino;

X is selected from the group consisting of alkylene, alkenylene, alkynylene, arylene, heteroarylene, and heterocyclylene wherein the alkylene, alkenylene, and alkynylene groups can be optionally interrupted or terminated by arylene, heteroarylene or heterocyclylene and optionally interrupted by one or more —O— groups;

Y′ is selected from the group consisting of:
—S(O)₀₋₂—,
—C(R₆)—,
—C(R₆)—O—,
—O—C(R₆)—,
—O—C(O)—O—,
—N(R₈)-Q-,
—O—C(R₆)—N(R₈)—,
—C(R₆)—N(OR₉)—,

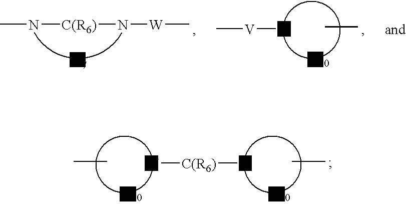

Y″ is selected from the group consisting of:
—S(O)₀₋₂—,
—S(O)₂—N(R₈ₐ)—,
—C(R₆)—,
—C(R₆)—O—,
O—C(R₆)—,
—O—C(O)—O—,
—N(R₈ₐ)-Qₐ-,
—C(R₆)—N(R₈ₐ)—,
—O—C(R₆)—N(R₈ₐ)—, and
—C(R₆)—N(OR₉)—;

R_{4c} is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, and in the case of heterocyclyl, oxo;

R₄₋₁ is selected from the group consisting of C₁₋₆ alkyl, C₂₋₆ alkenyl, C₂₋₆ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl wherein the C₁₋₆ alkyl, C₂₋₆ alkenyl, C₂₋₆ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl groups are unsubstituted or substituted by one or more substituents independently selected from the group consisting of C₁₋₄ alkyl, C₁₋₄ alkoxy, hydroxyC₁₋₄ alkyl, haloC₁₋₄ alkyl, haloC₁₋₄ alkoxy, halogen, nitro, hydroxy, mercapto, cyano, amino, C₁₋₄ alkylamino, di(C₁₋₄ alkyl)amino, and in the case of C₂₋₆ alkyl, C₂₋₆ alkenyl, C₂₋₆ alkynyl, and heterocyclyl, oxo;

with the proviso that when Y″ is —S(O)₂—N(R₈ₐ)— or —C(R₆)—N(R₈ₐ)—, then R₄₋₁ can also be hydrogen;

R₅₋₃ is selected from the group consisting of:

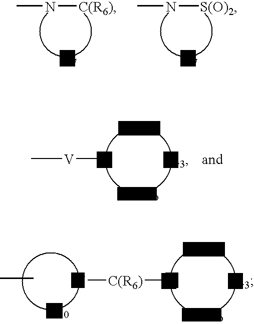

R₆ is selected from the group consisting of ═O and ═S;

R₇ is C₂₋₇ alkylene;

R₈ₐ is selected from the group consisting of hydrogen and C₁₋₄ alkyl;

R₈ is selected from the group consisting of hydrogen, alkyl, alkoxyalkylenyl, and arylalkylenyl;

R₉ is selected from the group consisting of hydrogen and alkyl;

R₁₀ is C₃₋₈ alkylene;

A₃ is selected from the group consisting of —O—, —C(O)—, —S(O)₀₋₂—, —CH₂—, and —N(R_{4c})—;

Qₐ is selected from the group consisting of a bond, —C(R₆)—, —C(R₆)—C(R₆)—, —S(O)₂—, —C(R₆)—N(R₈ₐ)—W—, —S(O)₂—N(R₈ₐ)—, —C(R₆)—O—, and —C(R₆)—N(OR₉)—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—;

V is selected from the group consisting of —O—C(R₆)— and —N(R₈)—C(R₆)—;

W is selected from the group consisting of a bond, —C(O)—, and —S(O)₂—; and

a and b are each independently an integer from 1 to 6 with the proviso that a+b is ≦7;

with the proviso that when R_{1-5b} includes a carbocyclic ring or heterocyclic ring containing one heteroatom, then the ring carbon atom by which the ring is attached is otherwise unsubstituted or substituted by an atom other than O, S, or N;

with the further proviso that R_{1-5b} is other than an unsubstituted or substituted isoxazolylalkylenyl, dihydroisoxazolylalkylenyl, or oxadiazolylalkylenyl group;

with the proviso that when X is interrupted with one —O— group, then Y′ is other than —S(O)₀₋₂—;

or a pharmaceutically acceptable salt thereof.

In another embodiment, there is provided a compound of the Formula (XXII): 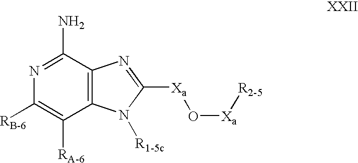
wherein:

Xₐ is C₁₋₂ alkylene;

R_{1-5c} is selected from the group consisting of:
—R_{4c},
—X₃—R_{4c},
—X₃—Y′″—R_{4c}, and
—X₃—R₅₋₃;

R₂₋₅ is selected from the group consisting of:
—Ar;
—Ar′—Y″—R₄₋₁, and
—Ar′—X′″—Y″—R₄₋₁;

R_{A-6} and R_{B-6} are each independently selected from the group consisting of:
hydrogen,
halogen,
alkyl,
alkenyl,
alkoxy,
alkylthio, and
—N(R₉)₂;

or R_{A-6} and R_{B-6} taken together form either a fused aryl ring that is unsubstituted or substituted by one or more Rₐ₁ groups, or a fused 5 to 7 membered saturated ring that is unsubstituted or substituted by one or more R_{c} groups;

or R_{A-6} and R_{B-6} taken together form a fused heteroaryl or 5 to 7 membered saturated ring, containing one heteroatom selected from the group consisting of N and S, wherein the heteroaryl ring is unsubstituted or substituted by one or more R_{b} groups, and the 5 to 7 membered saturated ring is unsubstituted or substituted by one or more R_{c} groups;

Rₐ₁ is selected from the group consisting of halogen, alkyl, haloalkyl, alkoxy, and —N(R₉)₂;

R_{b} is selected from the group consisting of halogen, hydroxy, alkyl, haloalkyl, alkoxy, and —N(R₉)₂;

R_{c} is selected from the group consisting of halogen, hydroxy, alkyl, alkenyl, haloalkyl, alkoxy, alkylthio, and —N(R₉)₂;

Ar is selected from the group consisting of aryl and heteroaryl both of which can be unsubstituted or can be substituted by one or more substituents independently selected from the group consisting of alkyl, alkoxy, haloalkyl, haloalkoxy, halogen, nitro, hydroxy, hydroxyalkyl, mercapto, cyano, carboxy, formyl, aryl, aryloxy, arylalkyleneoxy, heteroaryl, heteroaryloxy, heteroarylalkyleneoxy, heterocyclyl, heterocyclylalkylenyl, amino, alkylamino, and dialkylamino;

Ar′ is selected from the group consisting of arylene and heteroarylene both of which can be unsubstituted or can be substituted by one or more substituents independently selected from the group consisting of alkyl, alkoxy, haloalkyl, haloalkoxy, halogen, nitro, hydroxy, hydroxyalkyl, mercapto, cyano, carboxy, formyl, aryl, aryloxy, arylalkyleneoxy, heteroaryl, heteroaryloxy, heteroarylalkyleneoxy, heterocyclyl, heterocyclylalkylenyl, amino, alkylamino, and dialkylamino;

X₃ is selected from the group consisting of alkylene, arylene, heteroarylene, and heterocyclylene wherein the alkylene group can be optionally interrupted or terminated by arylene, heteroarylene or heterocyclylene and optionally interrupted by one or more —O— groups;

Y′″ is selected from the group consisting of:
—S(O)₀₋₂—,
—O—C(R₆)—,
—O—C(O)—O—,
—N(R₈)-Q-,
—O—C(R₆)—N(R₈)—,

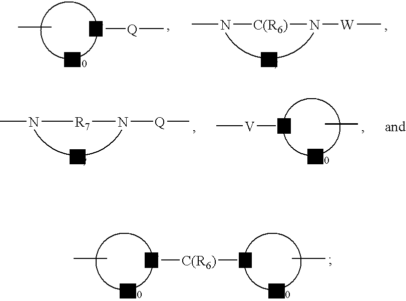

with the proviso that when X₃ is interrupted with one —O— group, then Y′″ is other than —S(O)₀₋₂—;

with the further proviso that when R_{A-6} and R_{B-6} taken together form a fused heteroaryl or 5 to 7 membered saturated ring, containing one heteroatom selected from the group consisting of N and S, wherein the heteroaryl ring is unsubstituted or substituted by one or more R_{b} groups, and the 5 to 7 membered saturated ring is unsubstituted or substituted by one or more R_{c} groups, then Y′″ can also be selected from the group consisting of —C(R₆)—, —C(R₆)—O—, and —C(R₆)—N(OR₉)—;

X′″ is selected from the group consisting of a C₁₋₄ alkylene and C₂₋₄ alkenylene;

Y″ is selected from the group consisting of:
—S(O)₀₋₂—,
—S(O)₂—N(R₈ₐ)—,
—C(R₆)—,
—C(R₆)—O—,
—O—C(R₆)—,
—O—C(O)—O—,
—N(R₈ₐ)-Qₐ-,
—C(R₆)—N(R₈ₐ)—,
—O—C(R₆)—N(R₈ₐ)—, and
—C(R₆)—N(OR₉)—,

R_{4c} is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently-selected from the group consisting of alkyl, alkoxy, hydroxyalkyl, halo alkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, and in the case of heterocyclyl, oxo;

R₄₋₁ is selected from the group consisting of C₁₋₆ alkyl, C₂₋₆ alkenyl, C₂₋₆ alkynyl, aryl, arylC₁₋₄ alkylenyl, aryloxyC₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl wherein the C₁₋₆ alkyl, C₂₋₆ alkenyl, C₂₋₆ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl groups are unsubstituted or substituted by one or more substituents independently selected from the group consisting of C₁₋₄ alkyl, C₁₋₄ alkoxy, hydroxyC₁₋₄ alkyl, haloC₁₋₄ alkyl, haloC₁₋₄ alkoxy, halogen, nitro, hydroxy, mercapto, cyano, amino, C₁₋₄ alkylamino, di(C₁₋₄ alkyl)amino, and in the case of C₂₋₆ alkyl, C₂₋₆ alkenyl, C₂₋₆ alkynyl, and heterocyclyl, oxo;

with the proviso that when Y″ is —S(O)₂—N(R₈ₐ)— or —C(R₆)—N(R₈ₐ)—, then R₄₋₁ can also be hydrogen;

R₅₋₃ is selected from the group consisting of:

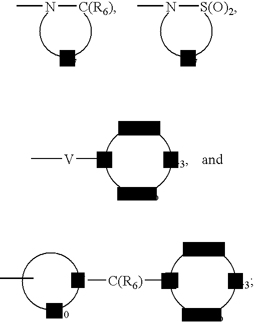

R₆ is selected from the group consisting of ═O and ═S;

R₇ is C₂₋₇ alkylene;

R₈ₐ is selected from the group consisting of hydrogen and C₁₋₄ alkyl;

R₈ is selected from the group consisting of hydrogen, alkyl, alkoxyalkylenyl, and arylalkylenyl;

R₉ is selected from the group consisting of hydrogen and alkyl;

R₁₀ is C₃₋₈ alkylene;

A₃ is selected from the group consisting of —O—, —C(O)—, —S(O)₀₋₂—, —CH₂—, and —N(R_{4c})—;

Qₐ is selected from the group consisting of a bond, —C(R₆)—, —C(R₆)—C(R₆)—, —S(O)₂—, —C(R₆)—N(R₈ₐ)—W—, S(O)₂—N(R₈ₐ)—, —C(R₆)—O—, and —C(R₆)—N(OR₉)—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—;

V is selected from the group consisting of —O—C(R₆)— and —N(R₈)—C(R₆)—;

W is selected from the group consisting of a bond, —C(O)—, and —S(O)₂—; and

a and b are each independently an integer from 1 to 6 with the proviso that a+b is ≦7;

with the proviso that when R_{1-5c} includes a carbocyclic ring or heterocyclic ring containing one heteroatom, then the ring carbon atom by which the ring is attached is otherwise unsubstituted or substituted by an atom other than O, S, or N;

with the further proviso that R_{1-5c} is other than an unsubstituted or substituted isoxazolylalkylenyl, dihydroisoxazolylalkylenyl, or oxadiazolylalkylenyl group;

or a pharmaceutically acceptable salt thereof.

In another embodiment, there is provided a compound of the following Formula (XXIII): 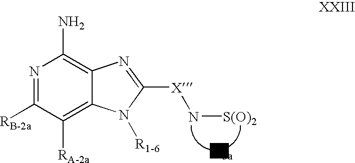
wherein:

X′″ is selected from the group consisting of C₁₋₄ alkylene and C₂₋₄ alkenylene;

R₃ₐ is C₂₋₅ alkylene;

R_{A-2a} and R_{B-2a} are each independently selected from the group consisting of:
hydrogen,
halogen,
alkyl,
alkenyl,
alkoxy,
alkylthio, and
—N(R₉)₂;

or R_{A-2a} and R_{B-2a} taken together form either a fused aryl ring that is unsubstituted or substituted by one or more Rₐ₁ groups, or a fused 5 to 7 membered saturated ring that is unsubstituted or substituted by one or more R_{c} groups;

or R_{A-2a} and R_{B-2a} taken together form a fused heteroaryl or 5 to 7 membered saturated ring, containing one heteroatom selected from the group consisting of N and S, wherein the heteroaryl ring is unsubstituted or substituted by one or more R_{b} groups, and the 5 to 7 membered saturated ring is unsubstituted or substituted by one or more R_{c} groups;

Rₐ₁ is selected from the group consisting of halogen, alkyl, haloalkyl, alkoxy, and —N(R₉)₂;

R_{b} is selected from the group consisting of halogen, hydroxy, alkyl, haloalkyl, alkoxy, and —N(R₉)₂;

R_{c} is selected from the group consisting of halogen, hydroxy, alkyl, alkenyl, haloalkyl, alkoxy, alkylthio, and —N(R₉)₂;

R₁₋₆ is selected from the group consisting of:
—R₄ₐ,
—X₃—R₄ₐ,
—X₃—Yₐ—R₄ₐ, and
—X₃—R₅₋₁;

X₃ is selected from the group consisting of alkylene, arylene, heteroarylene, and heterocyclylene wherein the alkylene group can be optionally interrupted or terminated by arylene, heteroarylene or heterocyclylene and optionally interrupted by one or more —O— groups;

Yₐ is independently selected from the group consisting of:
—S(O)₀₋₂—,
—C(R₆)—
—C(R₆)—O—,
—O—C(R₆)—,
—O—C(O)—O—,
—N(R₈)-Q-,
—O—C(R₆)—N(R₈)—,
—C(R₆)—N(OR₉)—,

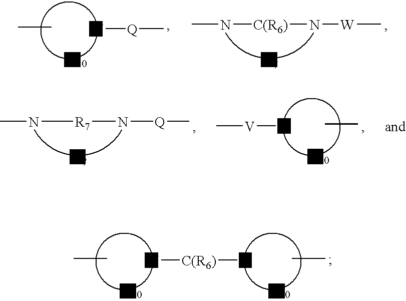

R₄ₐ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of heterocyclyl, oxo;

R₅₋₁ is selected from the group consisting of:

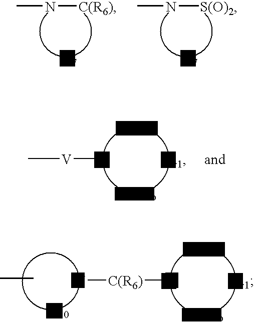

R₆ is selected from the group consisting of ═O and ═S;

R₇ is C₂₋₇ alkylene;

R₈ is selected from the group consisting of hydrogen, alkyl, alkoxyalkylenyl, and arylalkylenyl;

R₉ is selected from the group consisting of hydrogen and alkyl;

R₁₀ is C₃₋₈ alkylene;

A₁ is selected from the group consisting of —O—, —C(O)—, —CH₂—, —S(O)₀₋₂—, and —N(R₄ₐ)—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—;

V is selected from the group consisting of —O—C(R₆)— and —N(R₈)—C(R₆)—;

W is selected from the group consisting of a bond, —C(O)—, and —S(O)₂—; and

a and b are each independently an integer from 1 to 6 with the proviso that a+b is ≦7;

with the proviso that when X₃ is interrupted with one —O— group, then Yₐ is other than —S(O)₀₋₂—;

with the further proviso that when R₁₋₆ includes a carbocyclic ring or heterocyclic ring containing one heteroatom, then the ring carbon atom by which the ring is attached is otherwise unsubstituted or substituted by an atom other than O, S, or N;

with the further proviso that R₁₋₆ is other than an unsubstituted or substituted isoxazolylalkylenyl, dihydroisoxazolylalkylenyl, or oxadiazolylalkylenyl group;

or a pharmaceutically acceptable salt thereof.

In another embodiment, a compound of the Formula (XXIV): 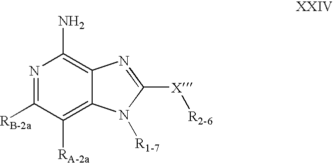
wherein:

X′″ is selected from the group consisting of C₁₋₄ alkylene and C₂₋₄ alkenylene;

R₂₋₆ is selected from the group consisting of:

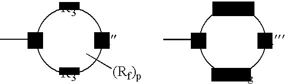

R₃′ is C₁₋₃ alkylene;

A″ is selected from the group consisting of —O—, —NH—, and —CH₂—;

R_{f} is selected from the group consisting of C₁₋₄ alkyl, phenyl, arylC₁₋₄ alkylenyl, hydroxy, hydroxyC₁₋₄ alkyl, C₁₋₄alkoxycarbonyl, carboxy, C₁₋₄ alkylcarbonylamino, pyrrolidinyl, and —C(O)N(R₉ₐ)₂;

p is 1 or 2;

R₉ₐ is selected from the group consisting of hydrogen and C₁₋₄ alkyl;

f and g are independently an integer from 1 to 3;

A′″ is selected from the group consisting of —S— and —N(-Q″-R₂₋₄ₐ)—;

R₂₋₄ₐ is selected from the group consisting of C₁₋₆ alkyl, C₂₋₆ alkenyl, C₂₋₆ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl wherein the C₁₋₆ alkyl, C₂₋₆ alkenyl, C₂₋₆ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl groups are unsubstituted or substituted by one or more substituents independently selected from the group consisting of C₁₋₄ alkyl, C₁₋₄ alkoxy, C₁₋₄ alkanoyl, C₁₋₄ alkoxycarbonyl, hydroxyC₁₋₄ alkyl, haloC₁₋₄ alkyl, haloC₁₋₄ alkoxy, halogen, nitro, hydroxy, mercapto, cyano, amino, C₁₋₄ alkylamino, di(C₁₋₄ alkyl)amino, and in the case of C₂₋₆ alkyl, C₂₋₆ alkenyl, C₂₋₆ alkynyl, and heterocyclyl, oxo;

R_{A-2a} and R_{B-2a} are each independently selected from the group consisting of:
hydrogen,
halogen,
alkyl,
alkenyl,
alkoxy,
alkylthio, and
—N(R₉)₂;

or R_{A-2a} and R_{B-2a} taken together form either a fused aryl ring that is unsubstituted or substituted by one or more Rₐ₁ groups, or a fused 5 to 7 membered saturated ring that is unsubstituted or substituted by one or more R_{c} groups;

or R_{A-2a} and R_{B-2a} taken together form a fused heteroaryl or 5 to 7 membered saturated ring, containing one heteroatom selected from the group consisting of N and S, wherein the heteroaryl ring is unsubstituted or substituted by one or more R_{b} groups, and the 5 to 7 membered saturated ring is unsubstituted or substituted by one or more R_{c} groups;

Rₐ₁ is selected from the group consisting of halogen, alkyl, haloalkyl, alkoxy, and —N(R₉)₂;

R_{b} is selected from the group consisting of halogen, hydroxy, alkyl, haloalkyl, alkoxy, and —N(R₉)₂;

R_{c} is selected from the group consisting of halogen, hydroxy, alkyl, alkenyl, haloalkyl, alkoxy, alkylthio, and —N(R₉)₂;

R₁₋₇ is selected from the group consisting of
hydrogen,
alkyl,
alkoxyalkylenyl,
hydroxyalkoxylalkyleneyl,
alkenyl,
alkynyl,
aryl,
arylalkylenyl,
alkylarylenyl,
heteroaryl,
heteroarylalkylenyl,
alkylheteroarylenyl,
heterocyclyl, and
—X₄—Y₄—R₄ₐ;

wherein alkyl, aryl, arylalkylenyl, alkylarylenyl, heteroaryl, heteroarylalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, heteroaryl, heterocyclyl, amino, alkylamino, dialkylamino, and in the case of heterocyclyl, oxo;

with the proviso that when R₁₋₇ includes a carbocyclic ring or heterocyclic ring containing one heteroatom, then the ring carbon atom by which the ring is attached is otherwise unsubstituted or substituted by an atom other than O, S, or N;

with the further proviso that R₁₋₇ is other than an unsubstituted or substituted isoxazolylalkylenyl, dihydroisoxazolylalkylenyl, or oxadiazolylalkylenyl group;

X₄ is selected from the group consisting of alkylene, alkenylene, alkynylene, arylene, heteroarylene, and heterocyclylene wherein the alkylene group can be optionally interrupted or terminated by arylene, heteroarylene or heterocyclylene and optionally interrupted by one or more —O— groups;

Y₄ is selected from the group consisting of:

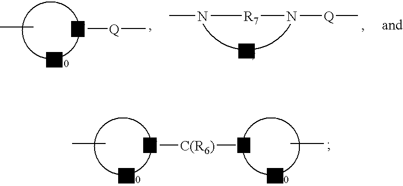

R₄ₐ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of heterocyclyl, oxo;

R₆ is selected from the group consisting of ═O and ═S;

R₇ is C₂₋₇ alkylene;

R₈ is selected from the group consisting of hydrogen, alkyl, alkoxyalkylenyl, and arylalkylenyl;

R₉ is selected from the group consisting of hydrogen and alkyl;

R₁₀ is C₃₋₈ alkylene;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—;

Q″ is selected from the group consisting of a bond, —C(R₆)—, —S(O)₂—, —S(O)₂—N(R₈)—, and —C(R₆)—O—; and

W is selected from the group consisting of a bond, —C(O)—, and —S(O)₂—;

or a pharmaceutically acceptable salt thereof.

The present invention also provides compounds (which are prodrugs) of the following Formulas CI, CV, CXI, CXIX, CXX, CXXII, CXXIII, and CXXIV: 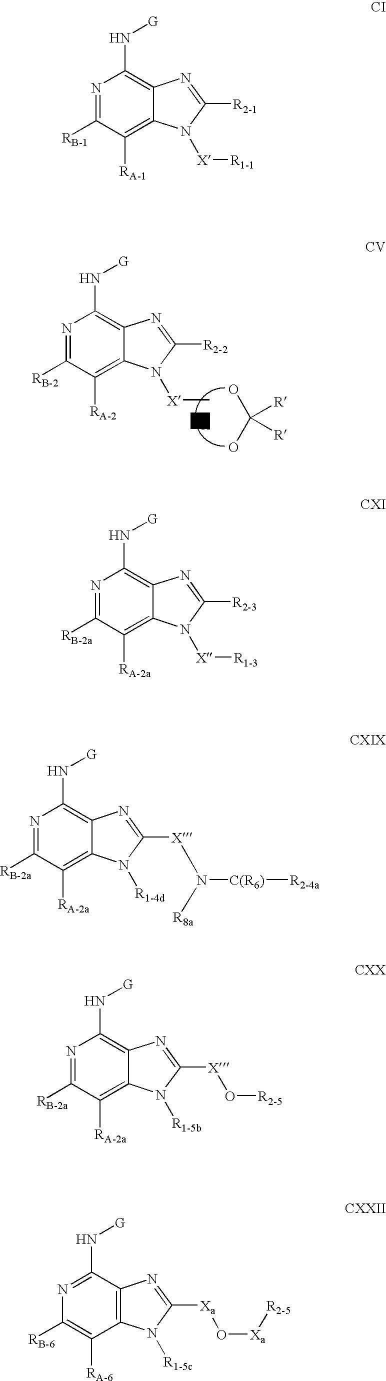 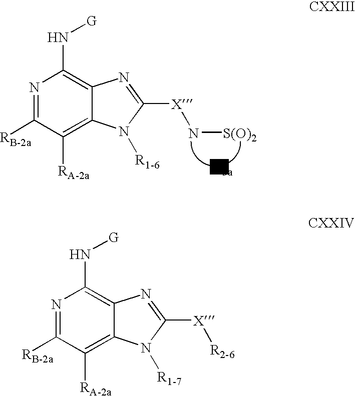
wherein G, R_{A-1}, R_{B-1}, R_{A-2}, R_{B-2}, R_{A-2a}, R_{B-2a}, R_{A-6}, R_{B-6}, R₁₋₁, R₁₋₃, R_{1-4d}, R_{1-5b}, R_{1-5c}, R₁₋₆, R₁₋₇, R₂₋₁, R₂₋₂, R₂₋₃, R₂₋₄ₐ, R₂₋₅, R₂₋₆, R₃ₐ, R₆, R₈ₐ, R′, R₁₁, Xₐ, X′, X″, and X′″ are as defined below; and pharmaceutically acceptable salts thereof.

In one embodiment, there is provided a compound of the Formula (CI): 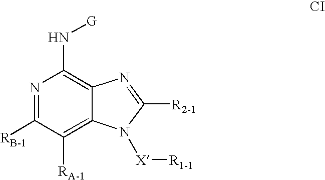
wherein:

G is selected from the group consisting of:
—C(O)—R′″,
α-aminoacyl,
α-aminoacyl-α-aminoacyl,
—C(O)—O—R′″,
—C(O)—N(R″″)R′″,
—C(═NY₅)—R′″,
—CH(OH)—C(O)—OY₅,
—CH(OC₁₋₄ alkyl)Y₀,
—CH₂Y₆, and
—CH(CH₃)Y₆;

R′″ and R″″ are independently selected from the group consisting of C₁₋₁₀ alkyl, C₃₋₇ cycloalkyl, and benzyl, each of which may be unsubstituted or substituted by one or more substitutents selected from the group consisting of halogen, hydroxy, nitro, cyano, carboxy, C₁₋₆ alkyl, C₁₋₄ alkoxy, aryl, heteroaryl, arylC₁₋₄ alkylenyl, heteroarylC₁₋₄ alkylenyl, haloC₁₋₄ alkylenyl, haloC₁₋₄ alkoxy, —O—C(O)—CH₃, —C(O)—O—CH₃, —C(O)—NH₂, —O—CH₂—C(O)—NH₂, —NH₂, and —S(O)₂—NH₂, with the proviso that R″″ can also be hydrogen;

α-aminoacyl is an acyl group derived from an amino acid selected from the group consisting of racemic, D-, and L-amino acids;

Y₅ is selected from the group consisting of hydrogen, C₁₋₆ alkyl, and benzyl;

Y₀ is selected from the group consisting of C₁₋₆ alkyl, carboxyC₁₋₆ alkylenyl, aminoC₁₋₄ alkylenyl, mono-N—C₁₋₆ alkylaminoC₁₋₄ alkylenyl, and di-N,N—C₁₋₆ alkylaminoC₁₋₄ alkylenyl;

Y₆ is selected from the group consisting of mono-N—C₁₋₆ alkylamino, di-N,N—C₁₋₆ alkylamino, morpholin-4-yl, piperidin-1-yl, pyrrolidin-1-yl, and 4-C₁₋₄ alkylpiperazin-1-yl; and

R₁₋₁, X′, R₂₋₁, R_{A-1} and R_{B-1} are as defined in Formula I above;

or a pharmaceutically acceptable salt thereof.

In another embodiment, there is provided a compound of the Formula (CV): 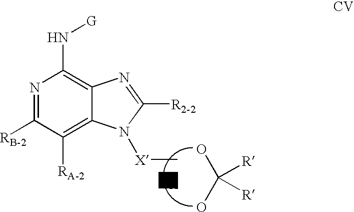
wherein G is as defined in Formula CI above; and
R′, R₁₁, X′, R₂₋₂, R_{A-2} and R_{B-2} are as defined in Formula V above;
or a pharmaceutically acceptable salt thereof.

In another embodiment, there is provided a compound of the Formula (CXI): 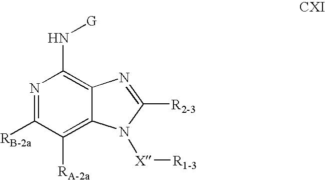
wherein G is as defined in Formula CI above; and
R₁₋₃, X″, R₂₋₃, R_{A-2a} and R_{B-2a} are as defined in Formula XI above;
or a pharmaceutically acceptable salt thereof.

In another embodiment, there is provided a compound of the Formula (CXIX): 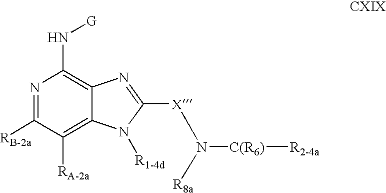
wherein G is as defined in Formula CI above;
R_{1-4d}, X′″, R₂₋₄ₐ, R₆, and R₈ₐ are as defined in Formula XIXd above; and
R_{A-2a} and R_{B-2a} are as defined in Formula XI above;
or a pharmaceutically acceptable salt thereof.

In another embodiment, there is provided a compound of the Formula (CXX): 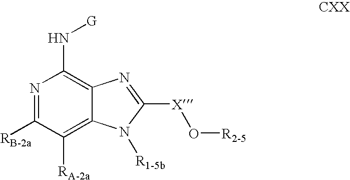
wherein G is as defined in Formula CI above;
R_{1-5b}, X′″, and R₂₋₅ are as defined in Formula XXI above; and
R_{A-2a} and R_{B-2a} are as defined in Formula XI above;
or a pharmaceutically acceptable salt thereof.

In another embodiment, there is provided a compound of the Formula (CXXII): 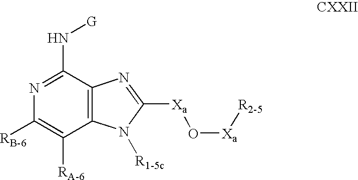
wherein G is as defined in Formula CI above; and
R_{1-5c}, Xₐ, R₂₋₅, R_{A-6} and R_{B-6} are as defined in Formula XXII above;
or a pharmaceutically acceptable salt thereof.

In another embodiment, there is provided a compound of the Formula (CXXIII): 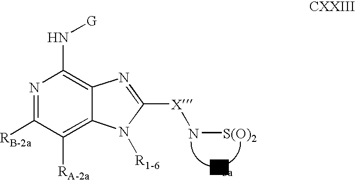
wherein G is as defined in Formula CI above; and
R₁₋₆, X′″, R₃ₐ, R_{A-2a} and R_{B-2a} are as defined in Formula XXIII above;
or a pharmaceutically acceptable salt thereof.

In another embodiment, there is provided a compound of the Formula (CXXIV): 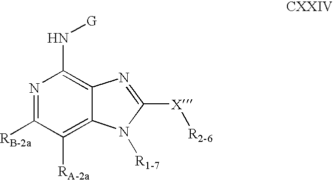
wherein G is as defined in Formula CI above; and
R₁₋₇, X′″, R₂₋₆, R_{A-2a} and R_{B-2a} are as defined in Formula XXIV above;
or a pharmaceutically acceptable salt thereof.

For any of the compounds presented herein, each one of the following variables (e.g., X, X′, X₁, Y, Y′, Y₁, R_{A-1}, R_{B-1}, R_{A-2}, R_{B-2}, R₁₋₁, R₁₃, R₂₁, R₂₋₃, Q, R₄, n, and so on) in any of its embodiments can be combined with any one or more of the other variables in any of their embodiments and associated with any one of the formulas described herein, as would be understood by one of skill in the art. Each of the resulting combinations of variables is an embodiment of the present invention.

For certain embodiments of Formula I, the fused aryl ring or fused 5 to 7 membered saturated ring is unsubstituted.

For certain embodiments, including any one of the above embodiments of Formula II or Formula III, n is 0.

For certain embodiments of Formula IV, R_{A1} and R_{B1} are each methyl.

For certain embodiments, including any one of the above embodiments of Formulas I through IV, R₁₋₁ is

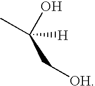

For certain embodiments, including any one of the above embodiments of Formulas I through IV which do not exclude this definition, R₁₋₁ is

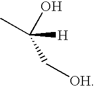

For certain embodiments, including any one of the above embodiments of Formulas I through IV which do not exclude this definition, R₁₋₁ is —CH(CH₂OH)₂.

For certain embodiments, including any one of the above embodiments of Formulas I through IV, X′ is —CH₂—.

For certain embodiments, including any one of the above embodiments of Formulas I through IV, R₂₋₁ is alkoxyalkylenyl.

For certain embodiments, including any one of the above embodiments of Formulas I through IV, R₂₋₁ is C₁₋₄ alkyl-O—C₁₋₄ alkylenyl. For certain of these embodiments, R₂₋₁ is ethoxymethyl or 2-methoxyethyl.

For certain embodiments, including any one of the above embodiments of Formulas I through IV which do not exclude this definition, R₂₋₁ is hydroxyC₁₋₄ alkylenyl or C₁₋₄ alkyl-O—C₁₋₄ alkylenyl. For certain of these embodiments, R₂₋₁ is hydroxymethyl, 2-hydroxyethyl, methoxymethyl, ethoxymethyl or 2-methoxyethyl.

For certain embodiments of Formula V, Y can also be 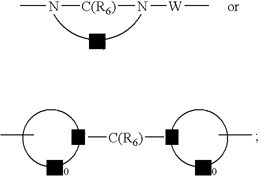
R₅ can also be 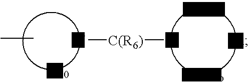
Q′ can also be —C(R₆)—N(R₈)—W— wherein W is selected from the group consisting of a bond, —C(O)—, and —S(O)₂— or —C(R₆)—N(OR₉)—; and V′ can also be —N(R₈)—C(R₆)—.

For certain embodiments, including any one of the above embodiments of Formula V, the fused aryl ring, fused 5 to 7 membered saturated ring, fused heteroaryl ring, or fused 5 to 7 membered saturated ring containing one heteroatom is unsubstituted.

For certain embodiments, including any one of the above embodiments of Formula VI or Formula VII, n is 0.

For certain embodiments, including any one of the above embodiments of Formulas VIII-1, VIII-2, VIII-3, VIII-4, IX-1, IX-2, IX-3, and IX-4, m is 0.

For certain embodiments of Formula X, R_{A1} and R_{B1} are each methyl.

For certain embodiments, including any one of the above embodiments of Formulas V through VII, VIII-1, VIII-2, VIII-3, VIII-4, IX-1, IX-2, IX-3, IX-4, and X, 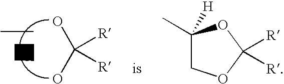
In certain of these embodiments, R′ is C₁₋₃ alkyl.

For certain embodiments, including any one of the above embodiments of Formulas V through VII, VIII-1, VIII-2, VIII-3, VIII-4, IX-1, IX-2, IX-3, IX-4, and X, which does not exclude this definition, 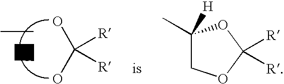
In certain of these embodiments, R′ is C₁₋₃ alkyl.

For certain embodiments, including any one of the above embodiments of Formulas V through VII, VIII-1, VIII-2, VIII-3, VIII-4, IX-1, IX-2, IX-3, IX-4, and X, which does not exclude this definition, 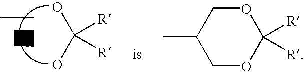
In certain of these embodiments, R′ is C₁₋₃ alkyl.

For certain embodiments, including any one of the above embodiments of Formulas V through VII, VIII-1, VIII-2, VIII-3, VIII-4, IX-1, IX-2, IX-3, IX-4, and X, XI is —CH₂—.

For certain embodiments, including any one of the above embodiments of Formulas V through VII, VIII-1, VIII-2, VIII-3, VIII-4, IX-1, IX-2, IX-3, IX-4, and X, R₂₋₂ is hydrogen, C₁₋₄ alkyl, hydroxyC₁₋₄ alkylenyl, or C₁₋₄ alkyl-O—C₁₋₄ alkylenyl. For certain of these embodiments, R₂₋₂ is hydrogen, methyl, ethyl, n-propyl, n-butyl, hydroxymethyl, 2-hydroxyethyl, methoxymethyl, ethoxymethyl or 2-methoxyethyl.

For certain embodiments, including any one of the above embodiments of Formulas V through VII, VIII-1, VIII-2, VIII-3, VIII-4, IX-1, IX-2, IX-3, IX-4, and X, R₂₋₂ is hydrogen, C₁₋₄ alkyl, or C₁₋₄ alkyl-O—C₁₋₄ alkylenyl. For certain of these embodiments, R₂₋₂ is hydrogen, methyl, ethyl, n-propyl, n-butyl, ethoxymethyl or 2-methoxyethyl.

For certain embodiments of Formula XI, Y can also be 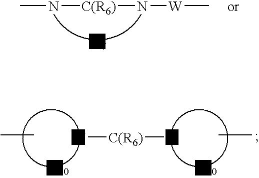
R₅ can also be 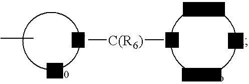
Q′ can also be —C(R₆)—N(R₈)—W— or —C(R₆)—N(OR₉)—; and V′ can also be —N(R₈)—C(R₆)—.

For certain embodiments, including any one of the above embodiments of Formula XI, Rₐ₁ is selected from the group consisting of fluorine, alkyl, haloalkyl, alkoxy, and —N(R₉)₂.

For certain embodiments of Formula XII, R_{A1} and R_{B1} are each methyl.

For certain embodiments, including any one of the embodiments of Formulas XIII, XIV, or XVII, n is 0.

For certain embodiments, including any one of the embodiments of Formulas XV-1, XV-2, XV-3, XV-4, XVI-1, XVI-2, XVI-3, or XVI-4, m is 0.

For certain embodiments, including any one of the embodiments of Formulas XI, through XIV, XV-1, XV-2, XV-3, XV-4, XVI-1, XVI-2, XVI-3, XVI-4, XVII, or XVIII, X″ is —CH₂—.

For certain embodiments, including any one of the embodiments of Formulas XI, through XIV, XV-1, XV-2, XV-3, XV-4, XVI-1, XVI-2, XVI-3, XVI-4, XVII, or XVIII, R₂₋₃ is hydrogen, alkoxyalkylenyl, hydroxyalkylenyl, —R₄, —X—R₄, or —X—Y—R₄; X is C₁₋₂ alkylene; Y is —S(O)₀₋₂—, —S(O)₂—N(R₈)—, —C(R₆)—, —C(R₆)—O—, —O—C(R₆)—, —O—C(O)—O—, —N(R₈)-Q′-, —C(R₆)—N(R₈)—, —O—C(R₆)—N(R₈)—, or —C(R₆)—N(OR₉)—; and R₄ is alkyl.

For certain embodiments, including any one of the embodiments of Formulas XI, through XIV, XV-1, XV-2, XV-3, XV-4, XVI-1, XVI-2, XVI-3, XVI-4, XVII, or XVIII, R₂₋₃ is hydrogen, C₁₋₄ alkyl, hydroxyC₁₋₄ alkylenyl, or C₁₋₄ alkyl-O—C₁₋₄ alkylenyl. For certain of these embodiments, R₂₋₃ is methyl, ethyl, n-propyl, n-butyl, ethoxymethyl, 2-methoxyethyl, hydroxymethyl, or 2-hydroxyethyl.

For certain embodiments, including any one of the embodiments of Formulas XI, through XIV, XV-1, XV-2, XV-3, XV-4, XVI-1, XVI-2, XVI-3, XVI-4, XVII, or XVIII, R₂₋₃ is hydrogen, C₄ alkyl, or C₁₋₄ alkyl-O—C₁₋₄ alkylenyl.

For certain embodiments, including any one of the embodiments of Formulas XI, through XIV, XV-1, XV-2, XV-3, XV-4, XVI-1, XVI-2, XVI-3, XVI-4, XVII, or XVIII, which does not exclude this definition, R₂₋₃ is hydrogen, alkoxyalkylenyl, —R₄, —X—R₄, or —X—Y—R₄; X is C₁₋₂ alkylene; Y is —S(O)₀₋₂—, —S(O)₂—N(R₈)—, —C(R₆)—, —C(R₆)—O—, —O—C(R₆)—, —O—C(O)—O—, —N(R₈)-Q-, —C(R₆)—N(R₈)—, —O—C(R₆)—N(R₈)—, or —C(R₆)—N(OR₉)—; and R₄ is alkyl. For certain of these embodiments, Q is Q′.

For certain embodiments, including any one of the embodiments of Formulas XI, through XIV, XV-1, XV-2, XV-3, XV-4, XVI-1, XVI-2, XVI-3, XVI-4, XVII, or XVIII, R″ is selected from the group consisting of alkyl, aryl, and heteroaryl wherein aryl is unsubstituted or substituted by halogen or haloalkyl, Q is a bond, and R₄ in R₃ is hydrogen, C₁₋₄ alkyl, or benzyl.

For certain embodiments, including any one of the embodiments of Formulas XI, through XIV, XV-1, XV-2, XV-3, XV-4, XVI-1, XVI-2, XVI-3, XVI-4, XVII, or XVIII, R₁₋₃ is selected from the group consisting of:

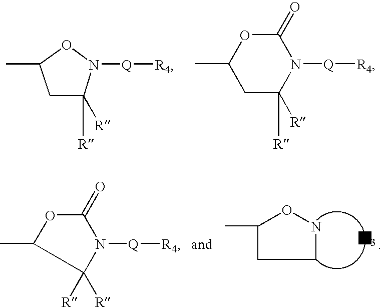

For certain embodiments, including any one of the embodiments of Formulas XI, through XIV, XV-1, XV-2, XV-3, XV-4, XVI-1, XVI-2, XVI-3, XVI-4, XVII, or XVIII, R₁₋₃ is selected from the group consisting of: 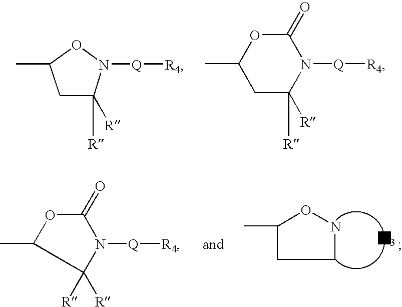
and Rₐ₁ is selected from the group consisting of fluorine, alkyl, haloalkyl, alkoxy, and —N(R₉)₂.

For certain embodiments, including any one of the above embodiments of Formula XIXa, R₂₋₄ is selected from the group consisting of C₃₋₆ alkyl optionally substituted by C₁₋₄ alkyl or C₁₋₄ alkoxy; aryl optionally substituted by C₁₋₄ alkyl, halogen, haloC₁₋₄ alkyl, haloC₁₋₄ alkoxy, or C₁₋₄ alkoxy; arylC₁₋₄ alkylenyl; heteroarylC₁₋₄ alkylenyl; and heteroarylC₃₋₆ cycloalkyl. For certain embodiments, including any one of the above embodiments of Formula XIXa, R₂₋₄ is C₃₋₆ cycloalkyl. For certain of these embodiments, R₂₋₄ is cyclopropyl.

For certain embodiments, including any one of the above embodiments of Formula XIXa, R₁₋₄ₐ is alkyl or hydroxyalkyl. For certain of these embodiments, R₁₋₄ₐ is 2-methylpropyl or 2-hydroxy-2-methylpropyl.

For certain embodiments, including any one of the above embodiments of Formulas XIXa or XIXb, R_{A-3} and R_{B-3} form a fused benzene ring that is unsubstituted or substituted by one or more Rₐ₁ groups.

For certain embodiments, including any one of the above embodiments of Formulas XIXa or XIXb, R_{A-3} and R_{B-3} form a fused benzene ring that is unsubstituted.

For certain embodiments, including any one of the above embodiments of Formulas XIXa or XIXb which does not exclude this definition, R_{A-3} and R_{B-3} form a fused cyclohexene ring that is unsubstituted or substituted by one or more R_{c} groups.

For certain embodiments, including any one of the above embodiments of Formula XIXb, R_{1-4b} is selected from the group consisting of:
—X₁—Y₁—R₄,
—X₂—Ar,
—X₂—Ar′—R₄,
—X₂—C(R₆)—O—R₄,
—X₂-alkylene-OH,
—X₂-alkynylene-R₄, 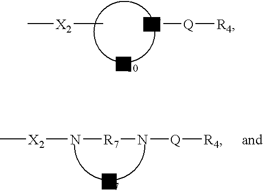
—X₁—R₅.

For certain embodiments, including any one of the above embodiments of Formula XIXb, R_{1-4b} is selected from the group consisting of —X₁—Y—R₄, —X₁—R₅, and 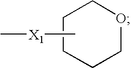
wherein X₁ is alkylene; Y, is —N(R₈)—C(O)—, —N(R₈)—S(O)₂—, —N(R₈)—C(O)—N(R₈)—, —N(R₈)—C(S)—N(R₈)—, or —N(R₈)—S(O)₂—N(R₈)—; R₄ is alkyl, aryl, or heteroaryl; and R₅ is 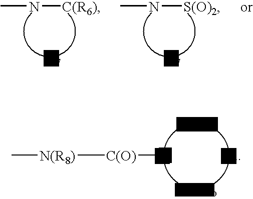
In certain of these embodiments, R_{1-4b} is tetrahydro-2H-pyran-4-ylmethyl. In certain other of these embodiments, R₅ is

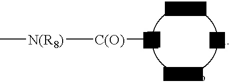

For certain embodiments, including the above embodiment of Formula XIXc, R_{A1} and R_{B1} are each methyl.

For certain embodiments, including any one of the above embodiments of Formula XIXc, R_{1-4c} is alkyl or hydroxyalkyl. For certain of these embodiments, R_{1-4c} is 2-methylpropyl, or 2-hydroxy-2-methylpropyl.

For certain embodiments, including the above embodiment of Formula XIXd, R_{A-4} and R_{B-4} taken together form a fused pyridine ring selected from the group consisting of: 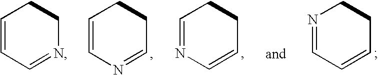
wherein the ring is unsubstituted or substituted by one or more R_{b} groups, and wherein the highlighted bond indicates the position where the ring is fused.

For certain embodiments, including any one of the above embodiments of Formula XIXd, R_{A-4} and R_{B-4} taken together form a fused pyridine ring, wherein the ring is unsubstituted.

For certain embodiments, including any one of the above embodiments of Formula XIXd, the fused pyridine ring is 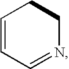
wherein the highlighted bond indicates the position where the ring is fused.

For certain embodiments, including any one of the above embodiments of Formula XIXd which does not exclude this definition, R_{A} and R_{B} taken together, form a fused piperidine ring selected from the group consisting of: 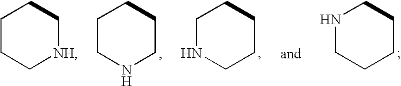
wherein the ring is unsubstituted or substituted by one or more R_{c} groups; and wherein the highlighted bond indicates the position where the ring is fused. For certain of these embodiments, the fused piperidine ring is 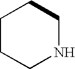
wherein the ring is unsubstituted or substituted by one or more R_{c} groups; and wherein the highlighted bond indicates the position where the ring is fused. For certain of these embodiments, the ring is unsubstituted.

For certain embodiments, including any one of the above embodiments of Formula XIXd, Yₐ is selected from the group consisting of:
—S(O)₀₋₂—,
—C(R₆)—,
—C(R₆)—O—,
—O—C(R₆)—,
—O—C(O)—O—,
—N(R₈)-Q-,
—O—C(R₆)—N(R₈)—,
—C(R₆)—N(OR₉)—, and

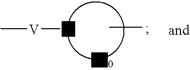

R₅₋₂ is

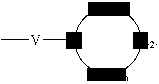

For certain embodiments, R₅₋₂ is also selected from the group consisting of:

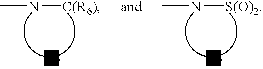

For certain embodiments, including any one of the above embodiments of Formula XIXd, R_{1-4d} is alkyl or hydroxyalkyl.

For certain embodiments, including any one of the above embodiments of Formula XIXd, R_{1-4d} is 2-methylpropyl or 2-hydroxy-2-methylpropyl.

For certain embodiments, including any one of the above embodiments of Formulas XIXa, XIXb, XIXc, and XIXd, X′″ is C₁₋₄ alkylene. For certain of these embodiments, X′″ is —CH₂—.

For certain embodiments, including any one of the above embodiments of Formulas XIXb, XIXc, and XIXd, R₂₋₄ₐ is selected from the group consisting of C₁₋₆ alkyl optionally substituted by C₁₋₄ alkyl or C₁₋₄ alkoxy; aryl optionally substituted by C₁₋₄ alkyl, halogen, haloC₁₋₄ alkyl, haloC₁₋₄ alkoxy, or C₁₋₄ alkoxy; arylC₁₋₄ alkylenyl; heteroarylC₁₋₄ alkylenyl; and heteroarylC₃₋₆ cycloalkyl.

For certain embodiments, including any one of the above embodiments of Formulas XIXb, XIXc, and XIXd, R₂₋₄ₐ is C₁₋₆ alkyl. For certain of these embodiments, R₂₋₄ₐ is methyl or cyclopropyl.

For certain embodiments of Formula XX, R₂₋₅ can also be —Ar′—R₅.

For certain embodiments, including any one of the above embodiments of Formula XX, R_{A-5} and R_{B-5} form a fused aryl ring that is unsubstituted or substituted by one or more Rₐ₁ groups.

For certain embodiments, including any one of the above embodiments of Formula XX, R_{A-5} and R_{B-5} form a fused aryl ring that is unsubstituted.

For certain embodiments, including any one of the above embodiments of Formula XX which does not exclude this definition, R_{A-5} and R_{B-5} form a fused 5 to 7 membered saturated ring, unsubstituted or substituted by one or more R_{c} groups.

For certain embodiments, including any one of the above embodiments of Formula XX which does not exclude this definition, R_{A-5} and R_{B-5} are each independently selected from the group consisting of:
hydrogen,
halogen,
alkyl,
alkenyl,
alkoxy,
alkylthio, and
—N(R₉)₂.
   For certain of these embodiments, R_{A-5} and R_{B-5} are each methyl.

For certain embodiments, including any one of the above embodiments of Formula XX, R₁₋₅ₐ is alkyl or hydroxyalkyl. For certain of these embodiments, R₁₋₅ₐ is 2-methylpropyl or 2-hydroxy-2-methylpropyl.

For certain embodiments of Formula XXI, R₂₋₅ can also be —Ar′—R₅.

For certain embodiments, including any one of the above embodiments of Formula XXI, R_{A-4} and R_{B-4} taken together form a fused heteroaryl ring containing one heteroatom selected from the group consisting of N and S, wherein the heteroaryl ring is unsubstituted or substituted by one or more R_{b} groups.

For certain embodiments, including any one of the above embodiments of Formula XXI, R_{A-4} and R_{B-4} taken together form a fused pyridine ring that is unsubstituted or substituted by one or more R_{b} groups.

For certain embodiments, including any one of the above embodiments of Formula XXI, R_{A-4} and R_{B-4} taken together form a fused pyridine ring that is unsubstituted.

For certain embodiments, including any one of the above embodiments of Formula XXI, the fused pyridine ring is 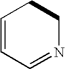
wherein the highlighted bond indicates the position where the ring is fused.

For certain embodiments, including any one of the above embodiments of Formula XXI which does not exclude this definition, R_{A-4} and R_{B-4} taken together form a fused 5 to 7 membered saturated ring containing one heteroatom selected from the group consisting of N and S, wherein the ring is unsubstituted or substituted by one or more R_{c} groups.

For certain embodiments, including any one of the above embodiments of Formula XXI, R_{1-5b} is selected from the group consisting of:
alkyl,
arylalkylenyl,
heterocyclylalkylenyl,
aryloxyalkylenyl,
hydroxyalkylenyl,
aminoalkylenyl,
haloalkylenyl,
alkylsulfonylalkylenyl,
—X—Y′—R_{4c}, and
—X—R₅₋₃;

wherein:
X is alkylene;
Y′ is selected from the group consisting of:
   —N(R₈)—C(O)—,
   —N(R₈)—S(O)₂—,
   —N(R₈)—C(O)—N(R₈)—,
   —N(R₈)—C(S)—N(R₈)—, and
   —N(R₈)—S(O)₂—N(R₈)—;
R_{4c} is selected from the group consisting of alkyl, aryl, and heteroaryl; and
R₅₋₃ is selected from the group consisting of: 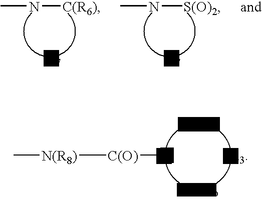
   In certain of these embodiments, R₅₋₃ is

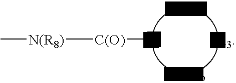

For certain embodiments, including any one of the above embodiments of Formula XXI, R_{1-5b} is alkyl or hydroxyalkylenyl. For certain of these embodiments, R_{1-5b} is 2-methylpropyl, butyl, or 2-hydroxy-2-methylpropyl. For certain embodiments, R_{1-5b} is 3-methoxypropyl.

For certain embodiments, including any one of the above embodiments of Formulas XX and XXI, X′″ is C₁₋₄ alkylene. For certain of these embodiments, X′″ is —CH₂—.

For certain embodiments of Formula XXII, R₂₋₅ can also be —Ar′—R₅.

For certain embodiments, including any one of the above embodiments of Formula XXII, R_{A-6} and R_{B-6} taken together form a fused aryl ring that is unsubstituted or substituted by one or more Rₐ₁ groups.

For certain embodiments, including any one of the above embodiments of Formula XXII, R_{A-6} and R_{B-6} taken together form a fused aryl ring that is unsubstituted.

For certain embodiments, including any one of the above embodiments of Formula XXII, the fused aryl ring is a fused benzene ring.

For certain embodiments, including any one of the above embodiments of Formula XXII which does not exclude this definition, R_{A-6} and R_{B-6} taken together form a fused 5 to 7 membered saturated ring that is unsubstituted or substituted by one or more R_{c} groups.

For certain embodiments, including any one of the above embodiments of Formula XXII which does not exclude this definition, R_{A-6} and R_{B-6} taken together form a fused heteroaryl ring containing one heteroatom selected from the group consisting of N and S, wherein the heteroaryl ring is unsubstituted or substituted by one or more R_{b} groups.

For certain embodiments, including any one of the above embodiments of Formula XXII which does not exclude this definition, R_{A-6} and R_{B-6} taken together form a fused pyridine ring that is unsubstituted or substituted by one or more R_{b} groups.

For certain embodiments, including any one of the above embodiments of Formula XXII which does not exclude this definition, R_{A-6} and R_{B-6} taken together form a fused pyridine ring that is unsubstituted.

For certain embodiments, including any one of the above embodiments of Formula XXII which does not exclude this definition, the fused pyridine ring is 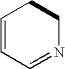
wherein the highlighted bond indicates the position where the ring is fused.

For certain embodiments, including any one of the above embodiments of Formula XXII which does not exclude this definition, R_{A-6} and R_{B-6} taken together form a fused 5 to 7 membered saturated ring containing one heteroatom selected from the group consisting of N and S, wherein the ring is unsubstituted or substituted by one or more R_{c} groups.

For certain embodiments, including any one of the above embodiments of Formula XXII which does not exclude this definition, R_{A-6} and R_{B-6} are each independently selected from the group consisting of:
hydrogen,
halogen,
alkyl,
alkenyl,
alkoxy,
alkylthio, and
—N(R₉)₂.

For certain embodiments, including any one of the above embodiments of Formula XXII, R_{1-5c} is selected from the group consisting of:
alkyl,
arylalkylenyl,
heterocyclylalkylenyl,
aryloxyalkylenyl,
hydroxyalkylenyl,
aminoalkylenyl,
haloalkylenyl,
alkylsulfonylalkylenyl,
—X—Y′″—R_{4c}, and
—X—R₅₋₃;

wherein:
X is alkylene;
Y′″ is selected from the group consisting of:
   —N(R₈)—C(O)—,
   —N(R₈)—S(O)₂—,
   —N(R₈)—C(O)—N(R₈)—,
   —N(R₈)—C(S)—N(R₈)—,
   —N(R₈)—S(O)₂—N(R₈)—, and 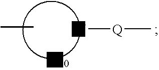
R_{4c} is selected from the group consisting of alkyl, aryl, and heteroaryl; and
R₅₋₃ is selected from the group consisting of:

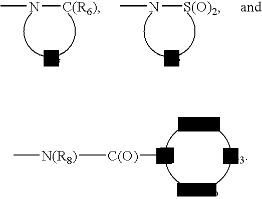

For certain embodiments, including any one of the above embodiments of Formula XXII, R_{1-5c} is alkyl or hydroxyalkylenyl. For certain embodiments, including any one of the above embodiments of Formula XXII, R_{1-5c} is 2-methylpropyl or 2-hydroxy-2-methylpropyl.

For certain embodiments, including any one of the above embodiments of Formulas XX, XXI, and XXII, Y″ is selected from the group consisting of:
—S(O)₀₋₂—,
—C(R₆)—,
—C(R₆)—O—,
—O—C(R₆)—,
—O—C(O)—O—,
—N(R₈ₐ)-Qₐ-,
—O—C(R₆)—N(R₈ₐ)—, and
—C(R₆)—N(OR₉)—.

For certain embodiments, including any one of the above embodiments of Formulas XX, XXI, and XXII, R₂₋₅ is —Ar′-Y″—R₄₋₁ or —Ar′—X′″—Y″—R₄₋₁ wherein Ar′ is phenylene, X′″ is methylene, Y″ is —NH—C(O)—, —NH—S(O)₂—, —C(O)—, —C(O)—O—, —S—, or —N(CH₃)—, and R₄₋₁ is methyl.

For certain embodiments, including any one of the above embodiments of Formulas XX, XXI, and XXII which does not exclude this definition, R₂₋₅ is selected from the group consisting of phenyl and phenyl substituted with trifluoromethyl, cyano, nitro, carboxy, dimethylamino, methylcarbonylamino, or methylsulfonylamino, or with one or more substituents selected from the group consisting of halogen, methoxy, and methyl.

For certain embodiments, including any one of the above embodiments of Formulas XX, XXI, and XXII which does not exclude this definition, R₂₋₅ is selected from the group consisting of phenyl and phenyl substituted with halogen, methoxy, methyl, dimethylamino, methylcarbonylamino, or methylsulfonylamino.

For certain embodiments, including any one of the above embodiments of Formula XXII, Xₐ is C₁₋₂ alkylene. For certain of these embodiments, Xₐ is —CH₂—.

For certain embodiments, including any one of the above embodiments of Formulas XX, XXI, and XXII which does not exclude this definition, R₄₋₁ is aryl. For certain of these embodiments, R₄₋₁ is phenyl.

For certain embodiments, including the above embodiment of Formula XXIII, R_{A-2a} and R_{B-2a} form a fused benzene ring that is unsubstituted or substituted by one or more Rₐ₁ groups.

For certain embodiments, including any one of the above embodiments of Formula XXIII, R_{A-2a} and R_{B-2a} form a fused benzene ring that is unsubstituted.

For certain embodiments, including any one of the above embodiments of Formula XXIII which does not exclude this definition, R_{A-2a} and R_{B-2a} form a fused cyclohexene ring that is unsubstituted or substituted by one or more R_{c} groups;

For certain embodiments, including any one of the above embodiments of Formula XXIII which does not exclude this definition, R_{A-2a} and R_{B-2a} are each independently selected from the group consisting of:
hydrogen,
halogen,
alkyl,
alkenyl,
alkoxy,
alkylthio, and
—N(R₉)₂;

or R_{A-2a} and R_{B-2a} taken together form a fused heteroaryl or 5 to 7 membered saturated ring, containing one heteroatom selected from the group consisting of N and S, wherein the heteroaryl ring is unsubstituted or substituted by one or more R_{b} groups, and the 5 to 7 membered saturated ring is unsubstituted or substituted by one or more R_{c} groups.

For certain embodiments, including any one of the above embodiments of Formula XXIII which does not exclude this definition, R_{A-2a} and R_{B-2a} taken together form a fused pyridine ring selected from the group consisting of: 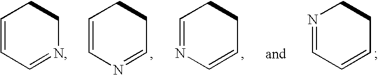
wherein the ring is unsubstituted or substituted by one or more R_{b} groups, and wherein the highlighted bond indicates the position where the ring is fused.

For certain embodiments, including any one of the above embodiments of Formula XXIII which does not exclude this definition, R_{A-2a} and R_{B-2a} taken together form a fused pyridine ring, wherein the ring is unsubstituted.

For certain embodiments, including any one of the above embodiments of Formula XXIII which does not exclude this definition, the fused pyridine ring is 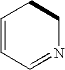
wherein the highlighted bond indicates the position where the ring is fused.

For certain embodiments, including any one of the above embodiments of Formula XXIII which does not exclude this definition, R_{A-2a} and R_{B-2a} taken together form a fused piperidine ring selected from the group consisting of: 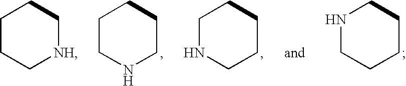
wherein the ring is unsubstituted or substituted by one or more R_{c} groups; and wherein the highlighted bond indicates the position where the ring is fused.

For certain embodiments, including any one of the above embodiments of Formula XXIII which does not exclude this definition, the fused piperidine ring is 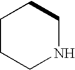
wherein the ring is unsubstituted or substituted by one or more R_{c} groups; and wherein the highlighted bond indicates the position where the ring is fused. For certain of these embodiments, the ring is unsubstituted.

For certain embodiments, including any one of the above embodiments of Formula XXIII which does not exclude this definition, R_{A-2a} and R_{B-2a} are each independently selected from the group consisting of:
hydrogen,
halogen,
alkyl,
alkenyl,
alkoxy,
alkylthio, and
—N(R₉)₂.
   For certain of these embodiments, R_{A-2a} and R_{B-2a} are each methyl.

For certain embodiments, including any one of the above embodiments of Formula XXIII, X′″ is C₁₋₄ alkylene. For certain of these embodiments, X′″ is —CH₂—.

For certain embodiments, including any one of the above embodiments of Formula XXIII, R₃ₐ is propylene.

For certain embodiments, including any one of the above embodiments of Formula XXIII, R₁₋₆ is selected from the group consisting of:
alkyl,
arylalkylenyl,
heterocyclylalkylenyl,
aryloxyalkylenyl,
hydroxyalkylenyl,
aminoalkylenyl,
haloalkylenyl,
alkylsulfonylalkylenyl,
—X₃—Yₐ—R₄ₐ, and
X₃—R₅₋₁;

wherein:
X₃ is alkylene;
Yₐ is selected from the group consisting of:
   —N(R₈)—C(O)—,
   —N(R₈)—S(O)₂—,
   —N(R₈)—C(O)—N(O)—,
   —N(R₈)—C(O)—N(R₈)—,
   —N(R₈)—C(S)—N(R₈)—,
   —N(R₈)—S(O)₂—N(R₈)—, and 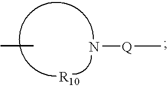
R₄ₐ is selected from the group consisting of alkyl, alkenyl substituted by aryl, aryl which is unsubstituted or substituted by one or more substituents selected from the group consisting of cyano, chloro, dimethylamino, and methoxy, arylalkylenyl, and heteroaryl which is unsubstituted or substituted by methyl; and
R₅₋₁ is selected from the group consisting of:

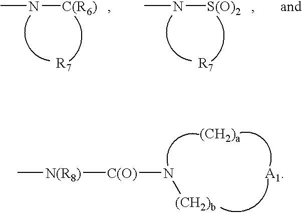

For certain embodiments, including any one of the above embodiments of Formula XXIII, R₁₋₆ is alkyl or hydroxyalkyl. For certain of these embodiments, R₁₋₆ is 2-methylpropyl, or 2-hydroxy-2-methylpropyl.

For certain embodiments, including the above embodiment of Formula XXIV, R_{A-2a} and R_{B-2a} form a fused benzene ring that is unsubstituted or substituted by one or more Rₐ₁ groups. For certain of these embodiments, R_{A-2a} and R_{B-2a} form a fused benzene ring that is unsubstituted.

For certain embodiments, including any one of the above embodiments of Formula XXIV which does not exclude this definition, R_{A-2a} and R_{B-2a} form a fused cyclohexene ring that is unsubstituted or substituted by one or more R_{c} groups.

For certain embodiments, including any one of the above embodiments of Formula XXIV which does not exclude this definition, R_{A-2a} and R_{B-2a} taken together form a fused pyridine ring selected from the group consisting of: 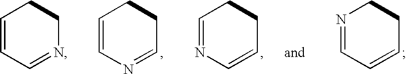
wherein the ring is unsubstituted or substituted by one or more R_{b} groups, and wherein the highlighted bond indicates the position where the ring is fused. For certain of these embodiments, the fused pyridine ring is 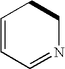
wherein the highlighted bond indicates the position where the ring is fused.

For certain embodiments, including any one of the above embodiments of Formula XXIV which does not exclude this definition, the fused pyridine ring is unsubstituted.

For certain embodiments, including any one of the above embodiments of Formula XXIV which does not exclude this definition, R_{A-2a} and R_{B-2a} taken together form a fused piperidine ring selected from the group consisting of: 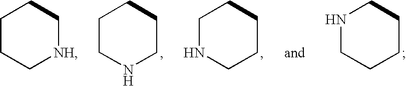
wherein the ring is unsubstituted or substituted by one or more R_{c} groups; and wherein the highlighted bond indicates the position where the ring is fused. For certain of these embodiments, the fused piperidine ring is 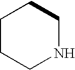
wherein the ring is unsubstituted or substituted by one or more R_{c} groups; and wherein the highlighted bond indicates the position where the ring is fused. For certain of these embodiments, the ring is unsubstituted.

For certain embodiments, including any one of the above embodiments of Formula XXIV, X′″ is C₁₋₄ alkylene. For certain of these embodiments, X′″ is —CH₂—.

For certain embodiments, including any one of the above embodiments of Formula XXIV, R₂₋₆ is 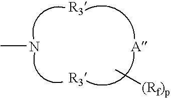
wherein R_{f} is selected from the group consisting of methyl, ethoxycarbonyl, carboxy, hydroxy, hydroxymethyl, hydroxyethyl, aminocarbonyl, diethylaminocarbonyl, methylcarbonylamino, pyrrolidinyl, and benzyl, and p is 1.

For certain embodiments, including any one of the above embodiments of Formula XXIV which does not exclude this definition, R₂₋₆ is 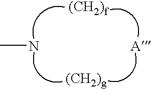
wherein A′″ is —N(-Q″—R₂₋₄ₐ)—; Q″ is bond, —C(O)—, or —S(O)₂—; and R₂₋₄ₐ is C₁₋₆ alkyl optionally substituted by one or more substituents selected from the group consisting of C₁₋₄ alkoxy, hydroxy, and C₁₋₄ alkoxycarbonyl; heteroaryl optionally substituted by one or more methyl groups; aryl optionally substituted by one or more substituents selected from the group consisting of fluoro, chloro, methoxy, cyano, and methyl; arylC₁₋₄ alkylenyl optionally substituted by one or more substituents selected from the group consisting of hydroxy and chloro; heteroarylC₁₋₄ alkylenyl; or aryloxyC₁₋₄ alkylenyl optionally substituted by one or more substituents selected from the group consisting of hydroxy, methyl, chloro, and fluoro.

For certain embodiments, including any one of the above embodiments of Formula XXIV which does not exclude this definition, R₂₋₆ is

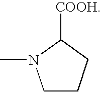

For certain embodiments, including any one of the above embodiments of Formula XXIV, R₁₋₇ is 2-methylpropyl, 2-hydroxy-2-methylpropyl, or 3-methoxypropyl.

For certain embodiments, Rₐ is selected from the group consisting of fluorine, alkyl, haloalkyl, alkoxy, and —N(R₉)₂.

For certain embodiments, Rₐ₁ is selected from the group consisting of halogen, alkyl, haloalkyl, alkoxy, and —N(R₉)₂.

For certain embodiments, Rₐ₁ is selected from the group consisting of fluorine, alkyl, haloalkyl, alkoxy, and —N(R₉)₂.

For certain embodiments, R_{b} is selected from the group consisting of halogen, hydroxy, alkyl, haloalkyl, alkoxy, and —N(R₉)₂.

For certain embodiments, R_{c} is selected from the group consisting of halogen, hydroxy, alkyl, alkenyl, haloalkyl, alkoxy, alkylthio, and —N(R₉)₂.

For certain embodiments, R_{d} and Rₑ are independently selected from the group consisting of hydrogen, halogen, hydroxy, alkyl, alkenyl, aryl, haloalkyl, alkoxy, alkylthio, and —N(R₉)₂; or R_{d} and Rₑ can join to form a fused aryl ring or fused 5-10 membered heteroaryl ring containing one to four heteroatoms.

For certain embodiments, R_{f} is selected from the group consisting of C₁₋₄ alkyl, phenyl, arylC₁₋₄ alkylenyl, hydroxy, hydroxyC₁₋₄ alkyl, C₄alkoxycarbonyl, carboxy, C₁₋₄ alkylcarbonylamino, pyrrolidinyl, and —C(O)N(R₉ₐ)₂.

For certain embodiments, R_{f} is selected from the group consisting of methyl, ethoxycarbonyl, carboxy, hydroxy, hydroxymethyl, hydroxyethyl, aminocarbonyl, diethylaminocarbonyl, methylcarbonylamino, pyrrolidinyl, and benzyl.

For certain embodiments, R′ is selected from the group consisting of hydrogen, alkyl, alkoxy, and alkoxyalkylenyl, or the R′ groups join together to form a 5 to 7 membered saturated ring optionally substituted by phenyl or phenyl substituted with one or more substituents selected from the group consisting of alkyl, alkoxy, halogen, and trifluoromethyl.

For certain embodiments, R′ is C₁₋₃ alkyl.

For certain embodiments, R″ is selected from the group consisting of alkyl, aryl, and heteroaryl wherein aryl is unsubstituted or substituted by halogen or haloalkyl.

For certain embodiments, R₁₋₄ₐ₋₁ is selected from the group consisting of alkyl, alkenyl, alkoxyalkylenyl, aryl, and arylalkylenyl, wherein the alkyl, alkenyl, alkoxyalkylenyl, aryl, and arylalkylenyl can be unsubstituted or substituted with one or more substituents selected from the group consisting of alkyl, alkoxy, hydroxyalkyl, haloalkyl, haloalkoxy, halogen, nitro, hydroxy, mercapto, cyano, amino, alkylamino, and dialkylamino.

For certain embodiments, R₂₋₄ₐ is C₁₋₆ alkyl optionally substituted by one or more substituents selected from the group consisting of C₁₋₄ alkoxy, hydroxy, and C₁₋₄ alkoxycarbonyl; heteroaryl optionally substituted by one or more methyl groups; aryl optionally substituted by one or more substituents selected from the group consisting of fluoro, chloro, methoxy, cyano, and methyl; arylC₁₋₄ alkylenyl optionally substituted by one or more substituents selected from the group consisting of hydroxy and chloro; heteroarylC₁₋₄ alkylenyl; or aryloxyC₁₋₄ alkylenyl optionally substituted by one or more substituents selected from the group consisting of hydroxy, methyl, chloro, and fluoro.

For certain embodiments, R₃ is C₃₋₅ alkylene.

For certain embodiments, R₃ₐ is C₂₋₅ alkylene.

For certain embodiments, R₃ₐ is propylene.

For certain embodiments, R₃′ is C₁₋₃ alkylene.

For certain embodiments, R₄ is alkyl, aryl, or heteroaryl.

For certain embodiments, R₄ is alkyl.

For certain embodiments, R₄, particularly in R₁₋₃, is hydrogen.

For certain embodiments, R₄ₐ is selected from the group consisting of alkyl, alkenyl substituted by aryl, aryl which is unsubstituted or substituted by one or more substituents selected from the group consisting of cyano, chloro, dimethylamino, and methoxy, arylalkylenyl, and heteroaryl which is unsubstituted or substituted by methyl; with the proviso that when R₁₋₆ is —X₃—R₄ₐ, then R₄ₐ is other than an unsubstituted or substituted isoxazolylalkylenyl, dihydroisoxazolylalkylenyl, or oxadiazolylalkylenyl group.

For certain embodiments, R_{4c} is selected from the group consisting of alkyl, aryl, and heteroaryl; with the proviso that when R_{1-5b} is —R_{4c} or —X—R_{4c}, then R_{4c} is other than an unsubstituted or substituted isoxazolylalkylenyl, dihydroisoxazolylalkylenyl, or oxadiazolylalkylenyl group.

For certain embodiments, R₅ₐ is selected from the group consisting of:

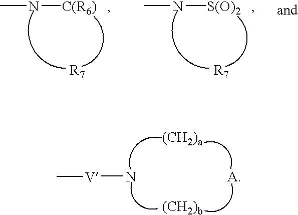

For certain embodiments, R₅ is selected from the group consisting of:

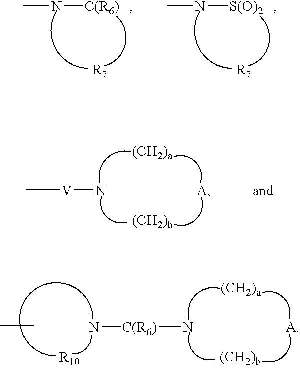

For certain embodiments, R₅ is

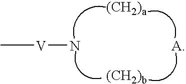

For certain embodiments, R₅ is selected from the group consisting of:

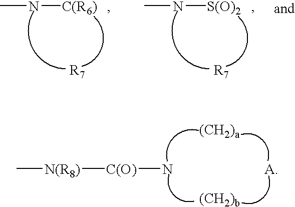

For certain embodiments, R₅₋₁ is selected from the group consisting of:

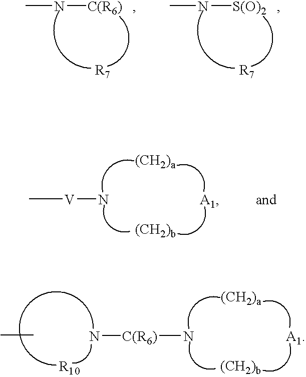

For certain embodiments, R₅₋₁ is

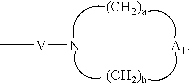

For certain embodiments, R₅₋₁ is selected from the group consisting of:

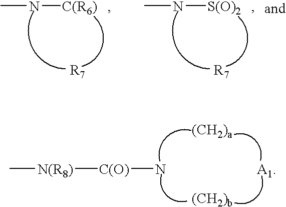

For certain embodiments, R₅₋₂ is selected from the group consisting of:

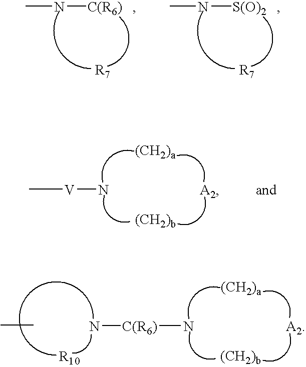

For certain embodiments, R₅₋₂ is

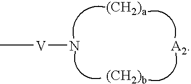

For certain embodiments, R₅₋₂ is selected from the group consisting of:

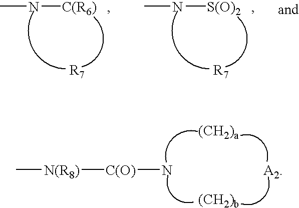

For certain embodiments, R₅₋₃ is selected from the group consisting of:

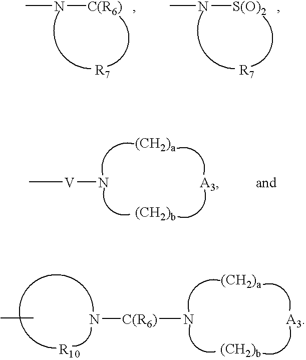

For certain embodiments, R₅₋₃ is

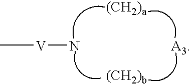

For certain embodiments, R₅₋₃ is selected from the group consisting of:

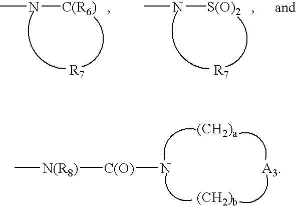

For certain embodiments, R₆ is selected from the group consisting of ═O and ═S.

For certain embodiments, R₆ is ═O.

For certain embodiments, R₇ is C₂₋₇ alkylene.

For certain embodiments, R₇ is C₂₋₄ alkylene.

For certain embodiments, R₇ is C₂₋₃ alkylene.

For certain embodiments, R₈ is selected from the group consisting of hydrogen, alkyl, alkoxyalkylenyl, and arylalkylenyl.

For certain embodiments, R₈ₐ is selected from the group consisting of hydrogen and C₁₋₄ alkyl. For certain embodiments, R₈ₐ is hydrogen.

For certain embodiments, R₉ is selected from the group consisting of hydrogen and alkyl.

For certain embodiments, R₉ₐ is selected from the group consisting of hydrogen and C₁₋₄ alkyl.

For certain embodiments, R₁₀ is C₃₋₈ alkylene.

For certain embodiments, R₁₁ is a straight chain C₂₋₃ alkylene.

For certain embodiments, R₁₂ is C₃₋₉ alkylene or C₃₋₉ alkenylene, optionally interrupted by one heteroatom.

For certain embodiments, R₁₃ is C₂₋₇ alkylene or C₂₋₇ alkenylene, optionally interrupted by one heteroatom.

For certain embodiments, A is selected from the group consisting of —O—, —C(O)—, —S(O)₀₋₂—, —CH₂—, and —N(R₄)—.

For certain embodiments, A is —O—.

For certain embodiments, A′ is selected from the group consisting of —O—, —S(O)₀₋₂—, —N(-Q-R₄)—, and —CH₂—.

For certain embodiments, A′ is —N(-Q-R₄)—.

For certain embodiments, A′ is —CH₂—.

For certain embodiments, A″ is selected from the group consisting of —O—, —NH—, and —CH₂—.

For certain embodiments, A″—NH—, or —CH₂—.

For certain embodiments, A′″ is selected from the group consisting of —S— and —N(-Q″—R₂₋₄ₐ).

For certain embodiments, A′″ is —N(-Q″-R₂₋₄ₐ)—.

For certain embodiments, A₁ is selected from the group consisting of —O—, —C(O)—, —CH₂—, —S(O)₀₋₂—, and —N(R₄ₐ)—.

For certain embodiments, A₂ is selected from the group consisting of —O—, —C(O)—, —CH₂—, —S(O)₀₋₂—, and —N(R_{4b})—.

For certain embodiments, A₃ is selected from the group consisting of —O—, —C(O)—, —S(O)₀₋₂—, —CH₂—, and —N(R_{4c})—.

For certain embodiments, Ar is phenyl or phenyl substituted with trifluoromethyl, cyano, or nitro, or with one or more substituents selected from the group consisting of halogen, methoxy, and methyl.

For certain embodiments, particularly in —Ar′—Y″—R₄₋₁ or —Ar′—X′″—Y″—R₄₋₁, Ar′ is phenylene.

For certain embodiments, Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—.

For certain embodiments, Q is —C(R₆)—, —S(O)₂—, or —C(R₆)—N(R₈)—.

For certain embodiments, particularly in —N(R₈)-Q-, Q is selected from the group consisting of a bond, —C(O)—, —S(O)₂—, and —C(O)—N(R₈)—.

For certain embodiments, Q is a bond.

For certain embodiments, Q′ is selected from the group consisting of a bond, —C(R₆)—, —C(R₆)—C(R₆)—, —S(O)₂—, and —S(O)₂—N(R₈)—.

For certain embodiments, particularly embodiments of —N(R₈)-Q′-, Q′ is —C(O)—, or —S(O)₂—.

For certain embodiments, Q″ is selected from the group consisting of a bond, —C(R₆)—, —S(O)₂—, —S(O)₂—N(R₈)—, and —C(R₆)—O—.

For certain embodiments, particularly in —N(-Q″-R₂₋₄ₐ)—, Q″ is selected from the group consisting of a bond, —C(O)—, and —S(O)₂—.

For certain embodiments, Q″ is —C(O)—.

For certain embodiments, Qₐ is selected from the group consisting of a bond, —C(R₆)—, —C(R₆)—C(R₆)—, —S(O)₂—, —C(R₆)—N(R₈ₐ)—W—, —S(O)₂—N(R₈ₐ)—, —C(R₆)—O—, and —C(R₆)—N(OR₉)—.

For certain embodiments, Qₐ is —C(R₆)—, —S(O)₂—, or —C(R₆)—N(R₈ₐ)—.

For certain embodiments, particularly —N(R₈ₐ)-Qₐ-, Qₐ is selected from the group consisting of a bond, —C(O)—, —S(O)₂—, and —C(O)—N(R₈ₐ)—.

For certain embodiments, Qₐ is a bond.

For certain embodiments, V is selected from the group consisting of —O—C(R₆)— and —N(R₈)—C(R₆)—.

For certain embodiments, V is —N(R₈)—C(R₆)—.

For certain embodiments, V′ is selected from the group consisting of —C(R₆)—, —O—C(R₆)—, and —S(O)₂—.

For certain embodiments, V′ —C(R₆)—.

For certain embodiments, W is selected from the group consisting of a bond, —C(O)—, and —S(O)₂—.

For certain embodiments, W is a bond.

For certain embodiments, X is selected from the group consisting of alkylene, alkenylene, alkynylene, arylene, heteroarylene, and heterocyclylene wherein the alkylene, alkenylene, and alkynylene groups are optionally interrupted or terminated by arylene, heteroarylene or heterocyclylene and optionally interrupted by one or more —O— groups.

For certain embodiments, particularly in —X—Y′—R_{4c} or —X—Y′″—R_{4c}, X is alkylene.

For certain embodiments, particularly in —X—R₄ or —X—Y—R₄, X is C₁₋₂ alkylene.

For certain embodiments, X₁ is selected from the group consisting of alkylene, alkenylene, alkynylene, arylene, heteroarylene, and heterocyclylene wherein the alkylene group can be optionally interrupted or terminated with arylene or heteroarylene and optionally interrupted by one or more —O— groups.

For certain embodiments, X₁ is alkylene.

For certain embodiments, X₂ is selected from the group consisting of alkylene, alkenylene, alkynylene, arylene, heteroarylene, and heterocyclylene wherein the alkylene, alkenylene, and alkynylene groups are interrupted by one or more —O— groups and can be optionally interrupted or terminated with arylene, heteroarylene, or heterocyclylene.

For certain embodiments, X₃ is selected from the group consisting of alkylene, arylene, heteroarylene, and heterocyclylene wherein the alkylene group can be optionally interrupted or terminated by arylene, heteroarylene or heterocyclylene and optionally interrupted by one or more —O— groups.

For certain embodiments, particularly in —X₃—Yₐ—R₄ₐ, X₃ is alkylene.

For certain embodiments, X₄ is selected from the group consisting of alkylene, alkenylene, alkynylene, arylene, heteroarylene, and heterocyclylene wherein the alkylene group can be optionally interrupted or terminated by arylene, heteroarylene or heterocyclylene and optionally interrupted by one or more —O— groups.

For certain embodiments, Y is selected from the group consisting of —S(O)₀₋₂—, —S(O)₂—N(R₈)—, —C(R₆)—, —C(R₆)—O—, —O—C(R₆)—, —O—C(O)—O—, —N(R₈)-Q′-, —C(R₆)—N(R₈)—, —O—C(R₆)—N(R₈)—, —C(R₆)—N(OR₉)—,

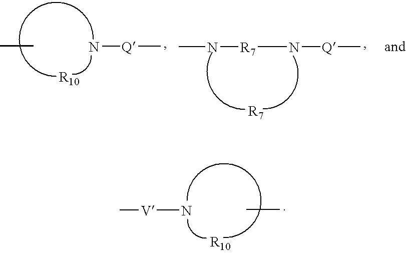

For certain embodiments, particularly in —X—Y—R₄, Y is —S(O)₀₋₂—, —S(O)₂—N(R₈)—, —C(R₆)—, —C(R₆)—O—, —O—C(R₆)—, —O—C(O)—O—, —N(R₈)-Q′-, —C(R₆)—N(R₈)—, —O—C(R₆)—N(R₈)—, or —C(R₆)—N(OR₉)—.

For certain embodiments, particularly in —X₁—Y₁—R₄, Y₁ is selected from the group consisting of —S(O)₀₋₂—, —O—C(R₆)—, —O—C(O)—O—, —N(R₈)-Q-, —O—C(R₆)—N(R₈)—, 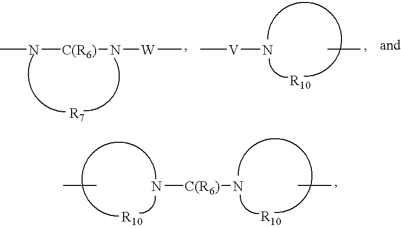
with the proviso that when X₁ is interrupted with one —O— group, then Y, is other than —S(O)₀₋₂—.

For certain embodiments, particularly in —X₁—Y—R₄, Y₁ is —N(R₈)—C(O)—, —N(R₈)—S(O)₂—, —N(R₈)—C(O)—N(R₈)—, —N(R₈)—C(S)—N(R₈)—, or —N(R₈)—S(O)₂—N(R₈)—.

For certain embodiments, Y₂ is selected from the group consisting of —S(O)₀₋₂—, —C(R₆)—O—, —O—C(R₆)—, —O—C(O)—O—, —N(R₈)-Q-, and —O—C(R₆)—N(R₈)—.

For certain embodiments, Y₃ is selected from the group consisting of

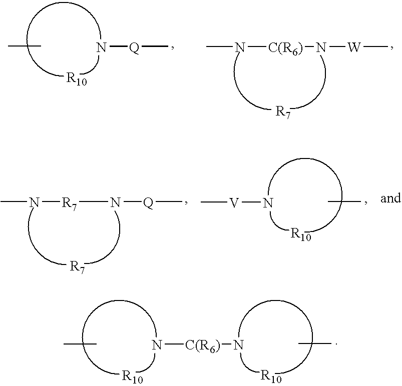

For certain embodiments, Y₄ is selected from the group consisting of:

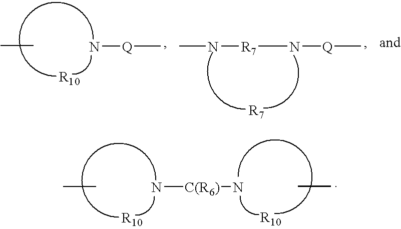

For certain embodiments, particularly in —X—Yₐ—R_{4b} or —X₃—Yₐ—R₄ₐ, Yₐ is selected from the group consisting of —S(O)₀₋₂—, —C(R₆)—, —C(R₆)—O—, —O—C(R₆)—, —O—C(O)—O—, —N(R₈)-Q-, —O—C(R₆)—N(R₈)—, —C(R₆)—N(OR₉)—, 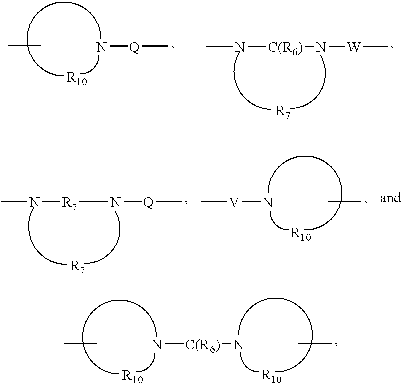
with the proviso that when X or X₃ is interrupted with one —O— group, then Yₐ is other than —S(O)₀₋₂—.

For certain embodiments, particularly in —X—Yₐ—R_{4b} or —X₃—Yₐ—R₄ₐ, Yₐ is selected from the group consisting of —S(O)₀₋₂—, —C(R₆)—, —C(R₆)—O—, —O—C(R₆)—, —O—C(O)—O—, —N(R₈)-Q-, —O—C(R₆)—N(R₈)—, —C(R₆)—N(OR₉)—, and 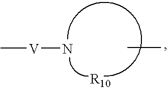
with the proviso that when X or X₃ is interrupted with one —O— group, then Yₐ is other than —S(O)₀₋₂—.

For certain embodiments, particularly in —X₃—Yₐ—R₄ₐ, Yₐ is selected from the group consisting of —N(R₈)—C(O)—, —N(R₈)—S(O)₂—, —N(R₈)—C(O)—(O)—, —N(R₈)—C(O)—N(R₈)—, —N(R₈)—C(S)—N(R₈)—, —N(R₈)—S(O)₂—N(R₈)—, and

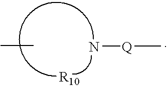

For certain embodiments, particularly in —X—Y′—R_{4c}, Y′ is selected from the group consisting of —S(O)₀₋₂—, —C(R₆)—, —C(R₆)—O—, —O—C(R₆)—, —O—C(O)—O—, —N(R₈)-Q-, —O—C(R₆)—N(R₈)—, —C(R₆)—N(OR₉)—, 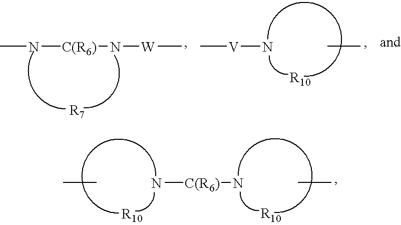
with the proviso that when X is interrupted with one —O— group, then Y′ is other than —S(O)₀₋₂—.

For certain embodiments, particularly in —X—Y′—R_{4c}, Y′ is selected from the group consisting of —N(R₈)—C(O)—, —N(R₈)—S(O)₂—, —N(R₈)—C(O)—N(R₈)—, —N(R₈)—C(S)—N(R₈)—, and —N(R₈)—S(O)₂—N(R₈)—.

For certain embodiments, Y″ is selected from the group consisting of —S(O)₀₋₂—, —S(O)₂—N(R₈ₐ)—, —C(R₆)—, —C(R₆)—O—, —O—C(R₆)—, —O—C(O)—O—, —N(R₈ₐ)-Qₐ-, —C(R₆)—N(R₈ₐ)—, —O—C(R₆)—N(R₈ₐ)—, and —C(R₆)—N(OR₉)—.

For certain embodiments, Y″ is selected from the group consisting of —S(O)₀₋₂—, —C(R₆)—, —C(R₆)—O—, —O—C(R₆)—, —O—C(O)—O—, —N(R₈ₐ)-Qₐ-, —O—C(R₆)—N(R₈ₐ)—, and —C(R₆)—N(OR₉)—.

For certain embodiments, particularly in —Ar′—Y″—R₄₋₁ or —Ar′—X′″—Y″—R₄₋₁, Y″ is —NH—C(O)—, —NH—S(O)₂—, —C(O)—, —C(O)—O—, —S—, or —N(CH₃)—.

For certain embodiments, particularly in —Ar′—Y″—R₄₋₁ or —Ar′—X′″—Y″—R₄₋₁, Y″ is —S(O)₂—N(R₈ₐ)— or —C(R₆)—N(R₈ₐ)—. For certain of these embodiments, R₄₋₁ can also be hydrogen. For certain of these embodiments, R₄₋₁ is hydrogen.

For certain embodiments, particularly in —X₃—Y′″—R_{4c}, Y′″ is selected from the group consisting of —S(O)₀₋₂—, —O—C(R₆)—, —O—C(O)—O—, —N(R₈)-Q-, —O—C(R₆)—N(R₈)—, 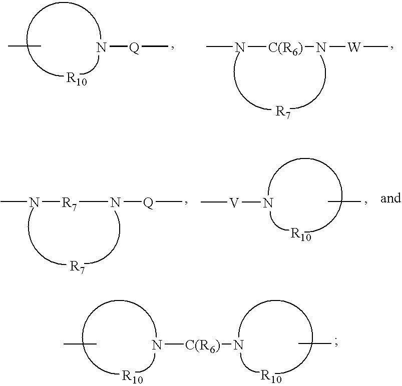
with the proviso that when X₃ is interrupted with one —O— group, then Y′″ is other than —S(O)₀₋₂—.

For certain embodiments, particularly in —X—Y′″—R_{4c}, Y′″ is selected from the group consisting of —N(R₈)—C(O)—, —N(R₈)—S(O)₂—, —N(R₈)—C(O)—N(R₈)—, —N(R₈)—C(S)—N(R₈)—, —N(R₈)—S(O)₂—N(R₈)—, and

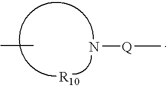

For certain embodiments, Y′″ can also be selected from the group consisting of —C(R₆)—, —C(R₆)—O—, and —C(R₆)—N(OR₉)—. For example, when R_{A-6} and R_{B-6} taken together form a fused heteroaryl or 5 to 7 membered saturated ring, containing one heteroatom selected from the group consisting of N and S, wherein the heteroaryl ring is unsubstituted or substituted by one or more R_{b} groups, and the 5 to 7 membered saturated ring is unsubstituted or substituted by one or more R_{c} groups, then Y′″ can also be selected from the group consisting of —C(R₆)—, —C(R₆)—O—, and —C(R₆)—N(OR₉)—.

For certain embodiments, 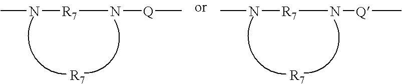
has a total number of ring atoms of 6 to 8.

For certain embodiments, a and b are independently integers from 1 to 6 with the proviso that a+b is ≦7.

For certain embodiments, a is 2.

For certain embodiments, b is 2.

For certain embodiments, m is an integer of 0 to 3.

For certain embodiments, m is 0.

For certain embodiments, p is 1 or 2.

For certain embodiments, p is 1.

For certain embodiments, f and g are independently an integer of 1 to 3.

For certain embodiments of the compounds of Formulas I through VII, VIII-1, VIII-2, VIII-3, VIII-4, IX-1, IX-2, IX-3, IX-4, X through XIV, XV-1, XV-2, XV-3, XV-4, XVI-1, XVI-2, XVI-3, XVI-4, XIXa, XIXb, XIXc, XIXd, XX, XXI, XXII, XXIII, and XXIV, the —NH₂ group can be replaced by an —NH-G group, as shown in the compounds of Formulas CI, CV, CXI, CXIX, CXX, CXXII, CXXIII, and CXXIV, to form prodrugs. In such embodiments, G is selected from the group consisting of: —C(O)—R′″, α-aminoacyl, α-aminoacyl-α-aminoacyl, —C(O)—O—R′″, —C(O)—N(R″″)R′″, —C(═NY₅)—R′″, —CH(OH)—C(O)—OY₅, —CH(OC₁₋₄ alkyl)Y₀, —CH₂Y₆, and —CH(CH₃)Y₆. For certain embodiments, G is selected from the group consisting of —C(O)—R′″, α-aminoacyl, α-aminoacyl-α-aminoacyl, and —C(O)—O—R′″. Preferably, R′″ and R″″ are independently selected from the group consisting of C₁₋₁₀ alkyl, C₃₋₇ cycloalkyl, and benzyl, each of which may be unsubstituted or substituted by one or more substitutents selected from the group consisting of halogen, hydroxy, nitro, cyano, carboxy, C₁₋₆ alkyl, C₁₋₄ alkoxy, aryl, heteroaryl, arylC₁₋₄ alkylenyl, heteroarylC₁₋₄ alkylenyl, haloC₁₋₄ alkylenyl, halo C₁₋₄ alkoxy, —O—C(O)—CH₃, —C(O)—O—CH₃, —C(O)—NH₂, —O—CH₂—C(O)—NH₂, —NH₂, and —S(O)₂—NH₂, with the proviso that R″″ can also be hydrogen. Preferably, α-aminoacyl is an acyl group derived from an amino acid selected from the group consisting of racemic, D-, and L-amino acids. Preferably, Y₅ is selected from the group consisting of hydrogen, C₁₋₆ alkyl, and benzyl. Preferably, Y₀ is selected from the group consisting of C₁₋₆ alkyl, carboxyC₁₋₆ alkylenyl, aminoC₁₋₄ alkylenyl, mono-N—C₁₋₆ alkylaminoC₁₋₄ alkylenyl, and di-N,N—C₁₋₆ alkylaminoC₁₋₄ alkylenyl. Preferably, Y₆ is selected from the group consisting of mono-N—C₁₋₆ alkylamino, di-N,N—C₁₋₆ alkylamino, morpholin-4-yl, piperidin-1-yl, pyrrolidin-1-yl, and 4-C₄ alkylpiperazin-1-yl.

In one embodiment, there is provided a pharmaceutical composition comprising a therapeutically effective amount of a compound or salt of any one of the above embodiments of Formulas I through VII, VIII-1, VIII-2, VIII-3, VIII-4, IX-1, IX-2, IX-3, IX-4, X through XIV, XV-1, XV-2, XV-3, XV-4, XVI-1, XVI-2, XVI-3, XVI-4, XIXa, XIXb, XIXc, XIXd, XX, XXI, XXII, XXIII, and XXIV, or Formulas CI, CV, CXI, CXIX, CXX, CXXII, CXXIII, and CXXIV in combination with a pharmaceutically acceptable carrier.

In another embodiment, there is provided a method of inducing cytokine biosynthesis in an animal comprising administering an effective amount of a compound or salt of any one of the above embodiments of Formulas I through VII, VIII-1, VIII-2, VIII-3, VIII-4, IX-1, IX-2, IX-3, IX-4, X through XIV, XV-1, XV-2, XV-3, XV-4, XVI-1, XVI-2, XVI-3, XVI-4, XIXa, XIXb, XIXc, XIXd, XX, XXI, XXII, XXIII, and XXIV, or Formulas CI, CV, CXI, CXIX, CXX, CXXII, CXXIII, and CXXIV or a pharmaceutical composition comprising any one of the above embodiments of Formulas I through VII, VIII-1, VIII-2, VIII-3, VIII-4, IX-1, IX-2, IX-3, IX-4, X through XIV, XV-1, XV-2, XV-3, XV-4, XVI-1, XVI-2, XVI-3, XVI-4, XIXa, XIXb, XIXc, XIXd, XX, XXI, XXII, XXIII, and XXIV, or Formulas CI, CV, CXI, CXIX, CXX, CXXII, CXXIII, and CXXIV to the animal.

In another embodiment, there is provided a method of treating a viral disease in an animal in need thereof comprising administering a therapeutically effective amount of a compound or salt of any one of the above embodiments of Formulas I through VII, VIII-1, VIII-2, VIII-3, VIII-4, IX-1, IX-2, IX-3, IX-4, X through XIV, XV-1, XV-2, XV-3, XV-4, XVI-1, XVI-2, XVI-3, XVI-4, XIXa, XIXb, XIXc, XIXd, XX, XXI, XXII, XXIII, and XXIV, or Formulas CI, CV, CXI, CXIX, CXX, CXXII, CXXIII, and CXXIV or a pharmaceutical composition comprising any one of the above embodiments of Formulas I through VII, VIII-1, VIII-2, VIII-3, VIII-4, IX-1, IX-2, IX-3, IX-4, X through XIV, XV-1, XV-2, XV-3, XV-4, XVI-1, XVI-2, XVI-3, XVI-4, XIXa, XIXb, XIXc, XIXd, XX, XXI, XXII, XXIII, and XXIV, or Formulas CI, CV, CXI, CXIX, CXX, CXXII, CXXIII, and CXXIV to the animal.

In another embodiment, there is provided a method of treating a neoplastic disease in an animal in need thereof comprising administering a therapeutically effective amount of a compound or salt of any one of the above embodiments of Formulas I through VII, VIII-1, VIII-2, VIII-3, VIII-4, IX-1, IX-2, IX-3, IX-4, X through XIV, XV-1, XV-2, XV-3, XV-4, XVI-1, XVI-2, XVI-3, XVI-4, XIXa, XIXb, XIXc, XIXd, XX, XXI, XXII, XXIII, and XXIV, or Formulas CI, CV, CXI, CXIX, CXX, CXXII, CXXIII, and CXXIV or a pharmaceutical composition comprising any one of the above embodiments of Formulas I through VII, VIII-1, VIII-2, VIII-3, VIII-4, IX-1, IX-2, IX-3, IX-4, X through XIV, XV-1, XV-2, XV-3, XV-4, XVI-1, XVI-2, XVI-3, XVI-4, XIXa, XIXb, XIXc, XIXd, XX, XXI, XXII, XXIII, and XXIV, or Formulas CI, CV, CXI, CXIX, CXX, CXXII, CXXIII, and CXXIV to the animal.

Various alternative and preferred embodiments of the compounds of Formulas (I) through (XXIV) are presented herein in the appended claims.

As used herein, the terms “alkyl”, “alkenyl”, “alkynyl”, and the prefix “alk-” are inclusive of both straight chain and branched chain groups and of cyclic groups, e.g., cycloalkyl and cycloalkenyl. Unless otherwise specified, these groups contain from 1 to 20 carbon atoms, with alkenyl groups containing from 2 to 20 carbon atoms, and alkynyl groups containing from 2 to 20 carbon atoms. In some embodiments, these groups have a total of up to 10 carbon atoms, up to 8 carbon atoms, up to 6 carbon atoms, or up to 4 carbon atoms. Cyclic groups can be monocyclic or polycyclic and preferably have from 3 to 10 ring carbon atoms. Exemplary cyclic groups include cyclopropyl, cyclopropylmethyl, cyclopentyl, cyclohexyl, adamantyl, and substituted and unsubstituted bornyl, norbornyl, and norbornenyl.

Unless otherwise specified, “alkylene”, “alkenylene”, and “alkynylene” are the divalent forms of the “alkyl”, “alkenyl”, and “alkynyl” groups defined above. The terms, “alkylenyl”, “alkenylenyl”, and “alkynylenyl” are use when “alkylene”, “alkenylene”, and “alkynylene”, respectively, are substituted. For example, an arylalkylenyl group comprises an alkylene moiety to which an aryl group is attached. In another example, hydroxyalkylenyl, haloalkylenyl, and haloalkyleneoxy have the same meaning as hydroxyalkyl, haloalkyl, and haloalkoxy, respectively.

The term “haloalkyl” is inclusive of groups that are substituted by one or more halogen atoms, including perfluorinated groups. This is also true of other groups that include the prefix “halo-”. Examples of suitable haloalkyl groups are chloromethyl, trifluoromethyl, and the like.

The term “aryl” as used herein includes carbocyclic aromatic rings or ring systems. Examples of aryl groups include phenyl, naphthyl, biphenyl, fluorenyl and indenyl.

Unless otherwise indicated, the term “heteroatom” refers to the atoms O, S, or N.

The term “heteroaryl” includes aromatic rings or ring systems that contain at least one ring heteroatom (e.g., O, S, N). In some embodiments, the term “heteroaryl” includes a ring or ring system that contains 2 to 12 carbon atoms, 1 to 3 rings, 1 to 4 heteroatoms, and O, S, and/or N as the heteroatoms. Suitable heteroaryl groups include furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, pyrazinyl, 1-oxidopyridyl, pyridazinyl, triazinyl, tetrazinyl, oxadiazolyl, thiadiazolyl, and so on.

The term “heterocyclyl” includes non-aromatic rings or ring systems that contain at least one ring heteroatom (e.g., O, S, N) and includes all of the fully saturated and partially unsaturated derivatives of the above mentioned heteroaryl groups. In some embodiments, the term “heterocyclyl” includes a ring or ring system that contains 2 to 12 carbon atoms, 1 to 3 rings, 1 to 4 heteroatoms, and O, S, and N as the heteroatoms. Exemplary heterocyclyl groups include pyrrolidinyl, tetrahydrofuranyl, morpholinyl, thiomorpholinyl, 1,1-dioxothiomorpholinyl, piperidinyl, piperazinyl, thiazolidinyl, imidazolidinyl, isothiazolidinyl, tetrahydropyranyl, quinuclidinyl, homopiperidinyl (azepanyl), 1,4-oxazepanyl, homopiperazinyl (diazepanyl), 1,3-dioxolanyl, aziridinyl, azetidinyl, dihydroisoquinolin-(1H)-yl, octahydroisoquinolin-(1H)-yl, dihydroquinolin-(2H)-yl, octahydroquinolin-(2H)-yl, dihydro-1H-imidazolyl, 3-azabicyclo[3.2.2]non-3-yl, and the like.

The term “heterocyclyl” includes bicyclic and tricyclic heterocyclic ring systems. Such ring systems include fused and/or bridged rings and spiro rings. Fused rings can include, in addition to a saturated or partially saturated ring, an aromatic ring, for example, a benzene ring. Spiro rings include two rings joined by one spiro atom and three rings joined by two spiro atoms.

When “heterocyclyl” contains a nitrogen atom, the point of attachment of the heterocyclyl group may be the nitrogen atom.

The terms “arylene”, “heteroarylene”, and “heterocyclylene” are the divalent forms of the “aryl”, “heteroaryl”, and “heterocyclyl” groups defined above. The terms, “arylenyl”, “heteroarylenyl”, and “heterocyclylenyl” are used when “arylene”, “heteroarylene,” and “heterocyclylene”, respectively, are substituted. For example, an alkylarylenyl group comprises an arylene moiety to which an alkyl group is attached.

The term “fused aryl ring” includes fused carbocyclic aromatic rings or ring systems. Examples of fused aryl rings include benzo, naphtho, fluoreno, and indeno.

Unless otherwise indicated, the term “fused heteroaryl ring” includes the fused forms of 5 or 6 membered aromatic rings that contain one heteroatom selected from S and N.

The term “fused 5 to 7 membered saturated ring” includes rings which are fully saturated except for the bond where the ring is fused.

When a group (or substituent or variable) is present more than once in any Formula described herein, each group (or substituent or variable) is independently selected, whether explicitly stated or not. For example, for the formula —N(R₉)₂ each R₉ group is independently selected. In another example, when an R_{A-2} and/or an R_{B-2} group contains an R₉ group in addition to the R₉ group in X′, each R₉ group is independently selected. In a further example, when R₁₋₃ is 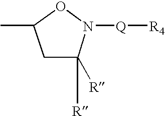
and R₂₋₃ includes and R₄ group, each R″ group is independently selected, and each R₄ group is independently selected. In another example, for the formula 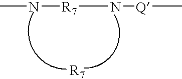
each R₇ group is independently selected.

The invention is inclusive of the compounds described herein (including intermediates) in any of their pharmaceutically acceptable forms, including isomers (e.g., diastereomers and enantiomers), salts, solvates, polymorphs, prodrugs, and the like. In particular, if a compound is optically active, the invention specifically includes each of the compound's enantiomers as well as racemic mixtures of the enantiomers. It should be understood that the term “compound” includes any or all of such forms, whether explicitly stated or not (although at times, “salts” are explicitly stated).

The term “prodrug” means a compound that can be transformed in vivo to yield an immune response modifying compound in any of the salt, solvated, polymorphic, or isomeric forms described above. The prodrug, itself, may be an immune response modifying compound in any of the salt, solvated, polymorphic, or isomeric forms described above. The transformation may occur by various mechanisms, such as through a chemical (e.g., solvolysis or hydrolysis, for example, in the blood) or enzymatic biotransformation. A discussion of the use of prodrugs is provided by T. Higuchi and W. Stella, “Pro-drugs as Novel Delivery Systems,” Vol. 14 of the A. C. S. Symposium Series, and in Bioreversible Carriers in Drug Design, ed. Edward B. Roche, American Pharmaceutical Association and Pergamon Press, 1987.

Preparation of the Compounds

Compounds of the invention may be synthesized by synthetic routes that include processes analogous to those well known in the chemical arts, particularly in light of the description contained herein. The starting materials are generally available from commercial sources such as Aldrich Chemicals (Milwaukee, Wis., USA) or are readily prepared using methods well known to those skilled in the art (e.g. prepared by methods generally described in Louis F. Fieser and Mary Fieser, Reagents for Organic Synthesis, v. 1-19, Wiley, New York, (1967-1999 ed.); Alan R. Katritsky, Otto Meth-Cohn, Charles W. Rees, Comprehensive Organic Functional Group Transformations, v 1-6, Pergamon Press, Oxford, England, (1995); Barry M. Trost and Ian Fleming, Comprehensive Organic Synthesis, v. 1-8, Pergamon Press, Oxford, England, (1991); or Beilsteins Handbuch der organischen Chemie, 4, Aufl. Ed. Springer-Verlag, Berlin, Germany, including supplements (also available via the Beilstein online database)).

For illustrative purposes, the reaction schemes depicted below provide potential routes for synthesizing the compounds of the present invention as well as key intermediates. For more detailed description of the individual reaction steps, see the EXAMPLES section below. Those skilled in the art will appreciate that other synthetic routes may be used to synthesize the compounds of the invention. Although specific starting materials and reagents are depicted in the reaction schemes and discussed below, other starting materials and reagents can be easily substituted to provide a variety of derivatives and/or reaction conditions. In addition, many of the compounds prepared by the methods described below can be further modified in light of this disclosure using conventional methods well known to those skilled in the art.

In the preparation of compounds of the invention it may sometimes be necessary to protect a particular functionality while reacting other functional groups on an intermediate. The need for such protection will vary depending on the nature of the particular functional group and the conditions of the reaction step. Suitable amino protecting groups include acetyl, trifluoroacetyl, tert-butoxycarbonyl (Boc), benzyloxycarbonyl, and 9-fluorenylmethoxycarbonyl (Fmoc). Suitable hydroxy protecting groups include acetyl and silyl groups such as the tert-butyl dimethylsilyl group. For a general description of protecting groups and their use, see T. W. Greene and P. G. M. Wuts, Protective Groups in Organic Synthesis, John Wiley & Sons, New York, USA, 1991.

Conventional methods and techniques of separation and purification can be used to isolate compounds of the invention or pharmaceutically acceptable salts thereof, as well as various intermediates related thereto. Such techniques may include, for example, all types of chromatography (high performance liquid chromatography (HPLC), column chromatography using common absorbents such as silica gel, and thin layer chromatography, recrystallization, and differential (i.e., liquid-liquid) extraction techniques.

Intermediates useful for making substituted imidazoquinolines and imidazonaphthyridines can be prepared according to Reaction Scheme I where X′″ and R₈ₐ are as defined above; Hal is chloro, bromo, or iodo; R_{Y} and R_{z} join to form a fused benzene ring optionally substituted by one or more Rₐ₂ groups or a fused pyridine ring optionally substituted by one or more R_{b} groups, wherein Rₐ₂ and R_{b} are as defined above; and R₁ₐ can be those groups included in R₁₋₄ₐ, R_{1-4b}, R_{1-4c}, R_{1-4d}, R₁₋₅ₐ, R_{1-5b}, R₁₋₆, and R₁₋₇ as defined above that do not include substituents that one skilled in the art would recognize as being susceptible to oxidation in step (2). These substituents include —S— and heteroaryl groups.

In step (1) of Reaction Scheme I, a 3,4-diamine of Formula XXX is reacted with a carboxylic acid or carboxylic acid equivalent to provide a 1H-imidazo[4,5-c]quinoline of Formula XXXI. The carboxylic acid equivalent is selected such that it will provide the desired Hal-X′″-substituent in a compound of Formula XXXI. Suitable carboxylic acid equivalents include orthoesters of Formula Hal-X′″—C(O-alkyl)₃, 1,1-dialkoxyalkyl alkanoates of Formula Hal-X′″—C(O-alkyl)₂(O—C(O)-alkyl), and acid halides of Formula Hal-X′″—C(O)Cl or Hal-X′″—C(O)Br.

The reaction with an acid halide of Formula Hal-X′″—C(O)Cl, such as chloroacetyl chloride, is conveniently carried out by combining the acid halide with a 3,4-diamine of Formula XXX in an inert solvent such as dichloromethane in the presence of a base such as triethylamine. The reaction can be carried out at ambient temperature, and the product can be isolated by conventional methods. The reaction may alternatively be carried out in two steps by first adding the acid halide of Formula Hal-X′″—C(O)Cl to a solution of the 3,4-diamine of Formula XXX in a suitable solvent such as dichloromethane at a sub-ambient temperature such as 0° C. The amide intermediate can optionally be isolated using conventional techniques and then treated with a base such as aqueous potassium carbonate or triethylamine in a suitable solvent such as dichloromethane, 1,2-dichloroethane, or ethanol or solvent system such as ethanol and water. The cyclization can be carried out at ambient temperature or at an elevated temperature such as the reflux temperature of the solvent.

Many compounds of Formula XXX, such as substituted quinolines and [1,5]naphthyridines, are known and can be readily prepared using known synthetic routes; see for example, U.S. Pat. Nos. 4,689,338 (Gerster), 4,929,624 (Gerster et al.), 5,268,376 (Gerster), 5,389,640 (Gerster et al.), 6,194,425 (Gerster et al.), 6,331,539 (Crooks et al.), 6,451,810 (Coleman et al.), 6,541,485 (Crooks et al.), 6,660,747 (Crooks et al.), 6,670,372 (Charles et al.), 6,683,088 (Crooks et al.), 6,656,938 (Crooks et al.), 6,664,264 (Dellaria et al.), and U.S. Patent Publication Application No. US 2004/0147543 (Hays et al.).

In step (2) of Reaction Scheme I an imidazoquinoline or imidazonaphthyridine of Formula XXXI is oxidized to a 5N-oxide of Formula XXXII using a conventional oxidizing agent capable of forming N-oxides. The reaction is conveniently carried out at ambient temperature by adding 3-chloroperoxybenzoic acid to a solution of a compound of Formula XXXI in a solvent such as chloroform or dichloromethane.

In step (3) of Reaction Scheme I a 5N-oxide of Formula XXXII is aminated to provide an amine of Formula XXXIII. Step (3) can be carried out by the activation of an N-oxide of Formula XXXII by conversion to an ester and then reacting the ester with an aminating agent. Suitable activating agents include alkyl- or arylsulfonyl chlorides such as benzenesulfonyl chloride, methanesulfonyl chloride, or p-toluenesulfonyl chloride. Suitable aminating agents include ammonia, in the form of ammonium hydroxide, for example, and ammonium salts such as ammonium carbonate, ammonium bicarbonate, and ammonium phosphate. The reaction is conveniently carried out by adding ammonium hydroxide to a solution of the N-oxide of Formula XXXII in a suitable solvent such as dichloromethane or chloroform and then adding p-toluenesulfonyl chloride. The reaction can be carried out at ambient temperature.

Steps (2) and (3) of Reaction Scheme I may alternatively be carried out as a one-pot procedure by adding 3-chloroperoxybenzoic acid to a solution of a compound of Formula XXXI in a solvent such as dichloromethane or chloroform and then adding ammonium hydroxide and p-toluenesulfonyl chloride without isolating the N-oxide compound of Formula XXXII.

In step (4) of Reaction Scheme I a compound of Formula XXXIII is treated with potassium phthalimide to provide a phthalimide-substituted compound of Formula XXXIV. The reaction is conveniently carried out by combining potassium phthalimide and compound of Formula XXXIII in a suitable solvent such as N,N-dimethylformamide (DMF). The reaction can be carried out at ambient temperature.

In step (5) of Reaction Scheme I a phthalimide-substituted compound of Formula XXXIV is deprotected to an aminoalkyl-substituted compound of Formula XXXV. Removal of the phthalimide group is conveniently carried out by adding hydrazine to a solution or suspension of a phthalimide-substituted compound of Formula XXXIV in a suitable solvent such as ethanol. The reaction can be carried out at ambient temperature.

Alternatively, a compound of Formula XXXIII can be converted to an aminoalkyl-substituted compound of Formula XXXVa according to step (4a) of Reaction Scheme I. The reaction can be carried out by adding an excess of an amine of Formula H₂N—R₈ₐ to solution of a compound of Formula XXXIII in a suitable solvent such as methanol. Several amines of Formula H₂N—R₈ₐ are commercially available. The reaction can be carried out at ambient temperature.

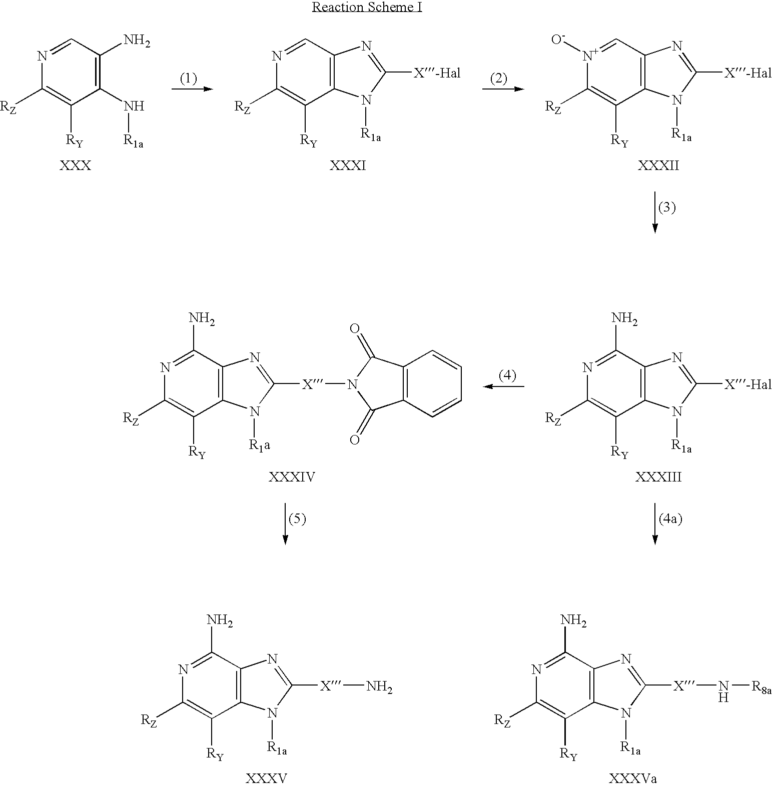

Intermediates useful for making substituted imidazopyridines can be prepared according to Reaction Scheme II, where R_{A1}, R_{B1}, X′″, and Hal are as defined above; Ph is phenyl; and R₁ includes the groups defined above in R_{1-4c}, R₁₋₅ₐ, R_{1-5c}, R₁₋₆, and R₁₋₇. In step (1) of Reaction Scheme II, a 2-phenoxypyridine-3,4-diamine of Formula XXXVI is converted to a 1H-imidazo[4,5-c]pyridine of Formula XXXVII by reaction with a halogen-substituted carboxylic acid equivalent. The reaction can be carried out as described in step (1) of Reaction Scheme I. When X′″ is methylene, the reaction is conveniently carried out by combining a 2-phenoxypyridine-3,4-diamine of Formula XXXVI with ethyl chloroacetimidate hydrochloride in a suitable solvent such as chloroform. The reaction can be carried out at an elevated temperature such as 60° C. Several 2-phenoxypyridine-3,4-diamines of Formula XXXVI are known or can be prepared by published methods. See, for example, U.S. Pat. Nos. 6,545,016 (Dellaria et al.), 6,743,920 (Lindstrom et al.), and 6,797,718 (Dellaria et al.). Ethyl chloroacetimidate hydrochloride is a known compound that can be prepared according to the literature procedure: Stillings, M. R. et al., J. Med. Chem., 29, pp. 2280-2284 (1986).

In step (2) of Reaction Scheme II, a halogen-substituted 1H-imidazo[4,5-c]pyridine of Formula XXXVII is aminated to provide an aminoalkyl-1H-imidazo[4,5-c]pyridin-4-amine of Formula XXXVIII. The reaction is conveniently carried out by adding a solution of ammonia in a suitable solvent such as methanol to a compound of Formula XXXVII and heating the reaction at an elevated temperature such as 150° C.

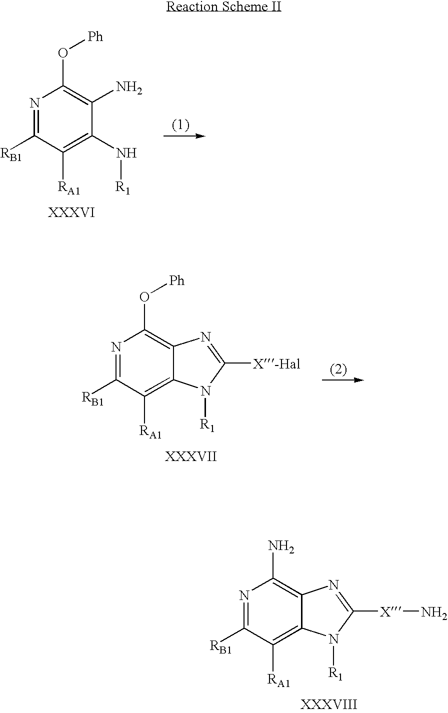

Amide-substituted compounds of the invention can be prepared according to Reaction Scheme III, wherein R₁ₐ, R₂₋₄ₐ, R₆, R₈ₐ, and X′″ are as defined above, and R_{Y2} and R_{Z2} are each independently selected from the group consisting of hydrogen, halogen, alkyl, alkenyl, alkoxy, alkylthio, and —N(R₉)₂; or R_{Y2} and R_{Z2} join to form a fused benzene ring optionally substituted by one or more Rₐ₂ groups; or R_{Y2} and R_{Z2} join to form a fused pyridine ring optionally substituted by one or more R_{b} groups, wherein Rₐ₂ and R_{b} are as defined above. Amino-substituted compounds of Formula XXXIX can be prepared according to the methods described in Reaction Schemes I and II.

In Reaction Scheme III, an amino-substituted compound of Formula XXXIX is converted to an amide-substituted compound of Formula XIXe using conventional methods. For example, an amino-substituted compound of Formula XXXIX or a salt thereof can react with an acid chloride of Formula R₂₋₄ₐC(O)Cl to provide a compound of Formula XIXe. Numerous acid chlorides of Formula R₂₋₄ₐC(O)Cl are commercially available; others can be readily prepared using known synthetic methods. The reaction is conveniently carried out by adding the acid chloride of Formula R₂₋₄ₐC(O)Cl to a solution of the aminoalkyl-substituted compound of Formula XXXIX in a suitable solvent such as chloroform, dichloromethane, DMF, or N,N-dimethylacetamide (DMA). Optionally a base such as triethylamine or N,N-diisopropylethylamine can be added. The reaction can be carried out at ambient temperature or a sub-ambient temperature such as 0° C.

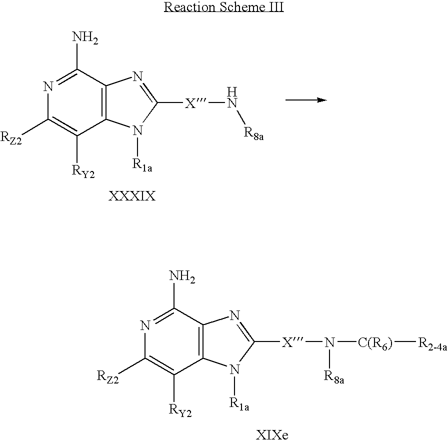

Sulfonamide-substituted compounds of the invention can be prepared according to Reaction Scheme IV, wherein R₁ₐ, R_{Y2}, R_{Z2}, R₃ₐ, and X′″ are as defined above. In Reaction Scheme IV, an amino-substituted compound of Formula XXXIXa is treated with a chloroalkanesulfonyl chloride of Formula Cl—R₃ₐS(O)₂Cl. Amino-substituted compounds of Formula XXXIXa can be prepared according to the methods described in Reaction Schemes I and II. The reaction is conveniently carried out by adding the chloroalkanesulfonyl chloride to a solution of the compound of Formula XXXIXa in a suitable solvent such as chloroform at ambient temperature. The isolable intermediate chloroalkanesulfonamide can then be treated with a base such as 1,8-diazabicyclo[5.4.0]undec-7-ene at ambient temperature in a suitable solvent such as DMF or chloroform to effect the cyclization and provide a compound of Formula XXIIIb.

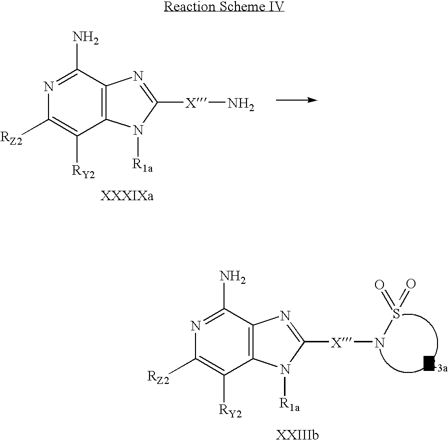

For some embodiments, substituted imidazoquinolines and substituted imidazonaphthyridines of the invention can be prepared from halo-substituted compounds of Formula XXXIII according to Reaction Scheme V, wherein R₁ₐ, R_{Y}, R_{Z}, R₂₋₅, and X′″ are as defined above. Haloalkyl-substituted compounds of Formula XXXIII can be prepared as described in Reaction Scheme I. Reaction Scheme V can be carried out by adding a substituted phenol of Formula H—O—R₂₋₅ to a compound of Formula XXXIII in a suitable solvent such as DMF to provide a compound of Formula XL. The reaction can be conveniently carried out in the presence of a base such as potassium carbonate at an elevated temperature such as 65° C. Numerous phenols of Formula H—O—R₂₋₅ are commercially available; others can be prepared using known synthetic methods.

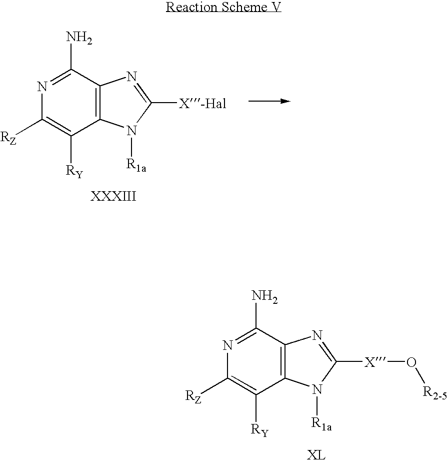

Compounds of the invention can also be prepared according to Reaction Scheme VI, wherein R₁ₐ, R_{Y}, R_{Z}, R₂₋₅, and Xₐ are as defined above. In step (1) of Reaction Scheme VI, a 3,4-diamine of Formula XXX is treated with a carboxylic acid or an equivalent thereof to provide a compound of Formula XLI. Suitable equivalents to carboxylic acid include acid anhydrides of Formula O[C(O)—Xₐ—O—(Xₐ)₀₋₁—R₂₋₅]₂ and acid chlorides of Formula Cl—C(O)—Xₐ—O—(Xₐ)₀₋₁—R₂₋₅. Some acid anhydrides and acid chlorides of these Formulas, such as phenoxyacetyl chloride and benzyloxyacetyl chloride, are commercially available; others can be prepared by known synthetic methods. The reaction is conveniently carried out according to the methods described in step (1) of Reaction Scheme I. The reaction with an acid chloride of Formula Cl—C(O)—Xₐ—O—(Xₐ)₀₋₁—R₂₋₅ can also be carried out in acetonitrile at room temperature or at an elevated temperature, such as the reflux temperature of the solvent.

In step (2) of Reaction Scheme VI, the compound of Formula XLI is oxidized and aminated to provide a compound of Formula XXIIa. This step is conveniently carried out according to the conditions described in steps (2) and (3) of Reaction Scheme I. Alternative oxidation conditions include the use of peracetic acid as the oxidizing agent in a solvent such as methyl acetate.

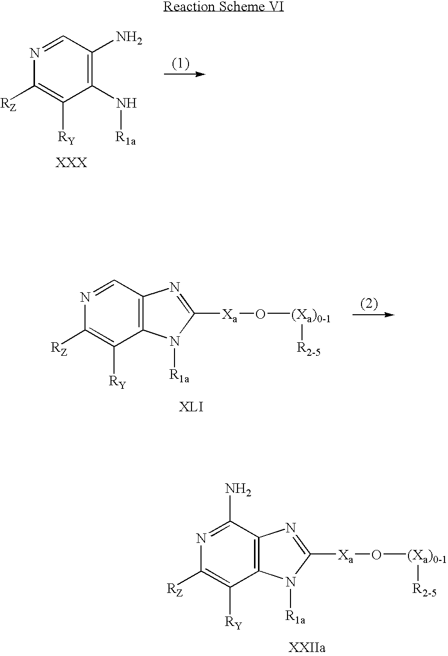

Compounds of the invention can be prepared according to Reaction Scheme VII, where R₁ₐ, R_{Y}, R_{Z}, R₂₋₄ₐ, Q″, f, g, Hal, and X′″ are as defined above.

In step (1) of Reaction Scheme VII, a haloalkyl-substituted compound of Formula XXXIII is treated with a cyclic diamine of Formula 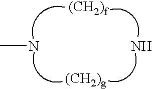
in the presence of a base such as triethylamine or N,N-diisopropylethylamine to provide a compound of Formula XLII. Such cyclic diamines, for example piperazine, are commercially available or can be readily synthesized by known methods. The reaction is conveniently carried out in a suitable solvent such as acetonitrile at an elevated temperature such as the reflux temperature of the solvent.

In step (2) of Reaction Scheme VII, a compound of Formula XLII is converted to a compound of Formula XXIVa using conventional techniques. For example, a compound of Formula XLII or a salt thereof can react with an acid chloride of Formula R₂₋₄ₐC(O)Cl or acid anhydride of Formula [R₂₋₄ₐC(O)]₂O to provide a compound of Formula XXIVa in which Q″ is —C(O)—. In addition, a compound of Formula XLII can react with sulfonyl chloride of Formula R₂₋₄ₐS(O)₂Cl or a sulfonic anhydride of Formula (R₂₋₄ₐS(O)₂)₂O to provide a compound of Formula XXIVa in which Q″ is —S(O)₂—. Numerous acid chlorides of Formula R₂₋₄ₐC(O)Cl, sulfonyl chlorides of Formula R₂₋₄ₐS(O)₂Cl, and sulfonic anhydrides of Formula (R₂₋₄ₐS(O)₂)₂O are commercially available; others can be readily prepared using known synthetic methods. The reaction is conveniently carried out by adding the acid chloride, sulfonyl chloride, or sulfonic anhydride to a solution of the compound of Formula XLII in a suitable solvent such as chloroform, dichloromethane, DMF, or DMA. Optionally a base such as triethylamine or N,N-diisopropylethylamine can be added. The reaction can be carried out at ambient temperature or a sub-ambient temperature such as 0° C.

Sulfamides of Formula XXIVa, where Q″ is —S(O)₂—N(R₈ₐ)—, can be prepared by reacting a compound or salt of Formula XLII with sulfuryl chloride to generate a sulfamoyl chloride in situ, and then reacting the sulfamoyl chloride with an amine of formula HN(R₈ₐ)R₂₋₄ₐ. Alternatively, sulfamides of Formula XXIVa can be prepared by reacting a compound of Formula XLII with a sulfamoyl chloride of Formula R₂₋₄ₐ(R₈ₐ)N—S(O)₂Cl. Many sulfonyl chlorides of Formula R₂₋₄ₐS(O)₂Cl and amines of Formula HN(R₈ₐ)R₂₋₄ₐ, and some sulfamoyl chlorides of Formula R₂₋₄ₐ(R₈ₐ)N—S(O)₂Cl are commercially available; others can be prepared using known synthetic methods.

Compounds of Formula XXIVa, wherein Q″ is a bond, can be prepared by reacting a compound or salt of Formula XLII with a variety of commercially available electrophiles, including alkyl halides and epoxides. The reaction can be carried out as described above for the reaction of a compound of Formula XLII with acid chlorides or sulfonyl chlorides.

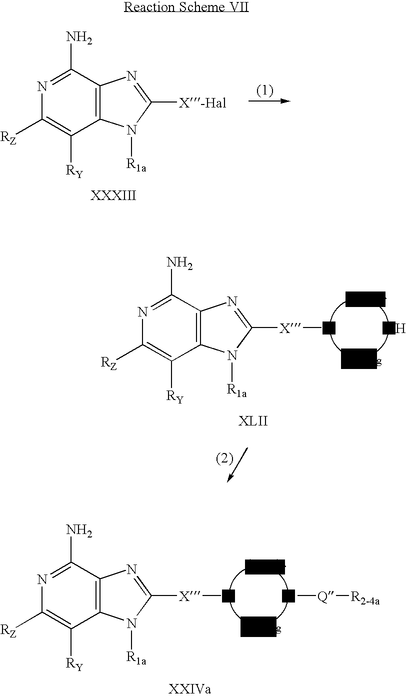

Compounds of the invention can also be prepared according to Reaction Scheme VIII, wherein R₁ₐ, R_{Y}, R_{Z}, R₃′, A″, R_{f}, p, Hal, and X′″ are as defined above. In Reaction Scheme VIII, a halogen-substituted compound of Formula XXXIII is treated with a substituted cyclic amine of Formula 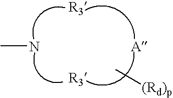
to provide a compound of Formula XXIVb. Many substituted cyclic amines are commercially available; others can be made by known methods. The reaction can be carried out according to the method described in step (1) of Reaction Scheme VII or the method described in Reaction Scheme V. These reaction conditions can also be used to treat a compound of Formula XXXIII with thiomorpholine to provide a compound of Formula XXIV wherein R₂₋₆ is 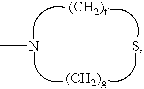
wherein f and g are as defined above.

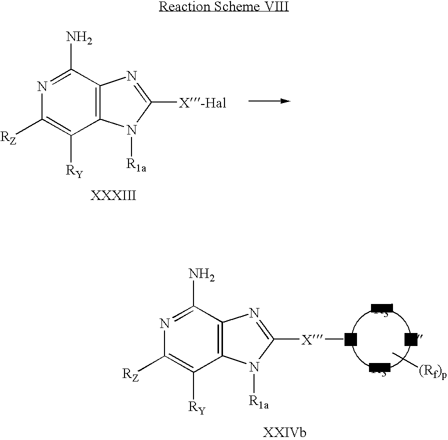

For some embodiments, compounds of the invention are prepared according to Reaction Scheme IX, wherein R_{Y}, R_{Z}, X, Q, Hal, R₈, and R₄ are as defined above; Boc is a tert-butoxycarbonyl group; and R_{2z} is selected from the group consisting of 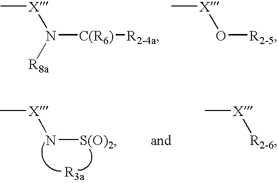
wherein X′″, R₂₋₄ₐ, R₂₋₅, R₂₋₆, R₃ₐ, R₆, and R₈ₐ are as defined above.

In steps (1) through (3) of Reaction Scheme IX, a 3,4-diamine of Formula XLIII is cyclized to a compound of Formula XLIV, which is then oxidized and aminated to a compound of Formula XLVI. Steps (1) through (3) of Reaction Scheme IX can be carried out as described for steps (1) through (3) of Reaction Scheme I. Compounds of Formula XLIII are known and can be readily prepared using known synthetic routes; see for example, U.S. Pat. Nos. 6,331,539 (Crooks et al.), 6,451,485 (Crooks et al.), 6,451,810 (Coleman et al.), and 6,677,349 (Griesgraber).

In step (4) of Reaction Scheme IX, a halogen-substituted compound of Formula XLVI is treated according to any of the methods or combination of methods described in Reaction Schemes I, III, IV, V, VII, and VIII to introduce the R₂, group and provide a compound of Formula XLVII. For example, the halogen-substituted compound of Formula XLVI can be treated according to the methods described in steps (4) and (5) of Reaction Scheme I followed by the method described in Reaction Scheme IV to provide a compound of Formula XLVII wherein R_{2z} is 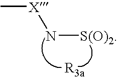
In another example, step (4) of Reaction Scheme IX can be carried out according to the method of Reaction Scheme V to provide a compound of Formula XLVII wherein R_{2z} is —X′″—O—R₂₋₅.

In step (5) of Reaction Scheme IX, the Boc group of the compound of Formula XLVII is removed to provide a 1-amino-substituted compound of Formula XLVIII. The deprotection is conveniently carried out by adding a solution of hydrogen chloride in a suitable solvent such as dioxane to a solution of the compound of Formula XLVII in a suitable solvent or solvent mixture such as methanol and dichloromethane. The reaction can be carried out at ambient temperature.

In step (6) of Reaction Scheme IX, a 1-amino-substituted compound of Formula XLVIII is converted to a compound of Formula XLIX using conventional methods. For example, a 1-amino-substituted compound of Formula XLVIII or a salt thereof can react with an acid chloride of Formula R₄C(O)Cl to provide a compound of Formula XLIX in which Q is —C(O)—. In addition, a 1-amino-substituted compound of Formula XLVIII can react with sulfonyl chloride of Formula R₄S(O)₂Cl or a sulfonic anhydride of Formula (R₄S(O)₂)₂O to provide a compound of Formula XLIX in which Q is —S(O)₂—. The reaction can be carried out according to one of the methods described in step (2) of Reaction Scheme VII.

Sulfamides of Formula XLIX, where Q is —S(O)₂—N(R₈)—, can be prepared by reacting a compound or salt of Formula XLVIII with sulfuryl chloride to generate a sulfamoyl chloride in situ, and then reacting the sulfamoyl chloride with an amine of formula HN(R₈)R₄. Alternatively, sulfamides of Formula XLIX can be prepared by reacting a compound of Formula XLVIII with a sulfamoyl chloride of formula R₄(R₈)N—S(O)₂Cl.

Compounds of Formula XLIX, wherein Q is —C(O)—N(R₈)—, —C(O)—N(R₈)—(CO)—, —C(S)—N(R₈)—, or —C(O)—N(R₈)—S(O)₂— can be prepared by reacting a compound of Formula XLVIII with an isocyanate of Formula R₄N═C═O or carbamoyl chloride of Formula R₄N—(R₈)—C(R₆)Cl, an isothiocyanate of Formula R₄N═C═S, or a sulfonyl isocyanate of Formula R₄S(O)₂N═C═O. Many compounds of these Formulas are commercially available; others can be prepared by known synthetic methods. The reaction is conveniently carried out by adding the isocyanate, isothiocyanate, carbamoyl chloride, or sulfonyl isocyanate to a solution of the compound of Formula XLVIII in a suitable solvent such as DMF or chloroform at ambient temperature. Optionally a base such as triethylamine or N,N-diisopropylethylamine can be added.

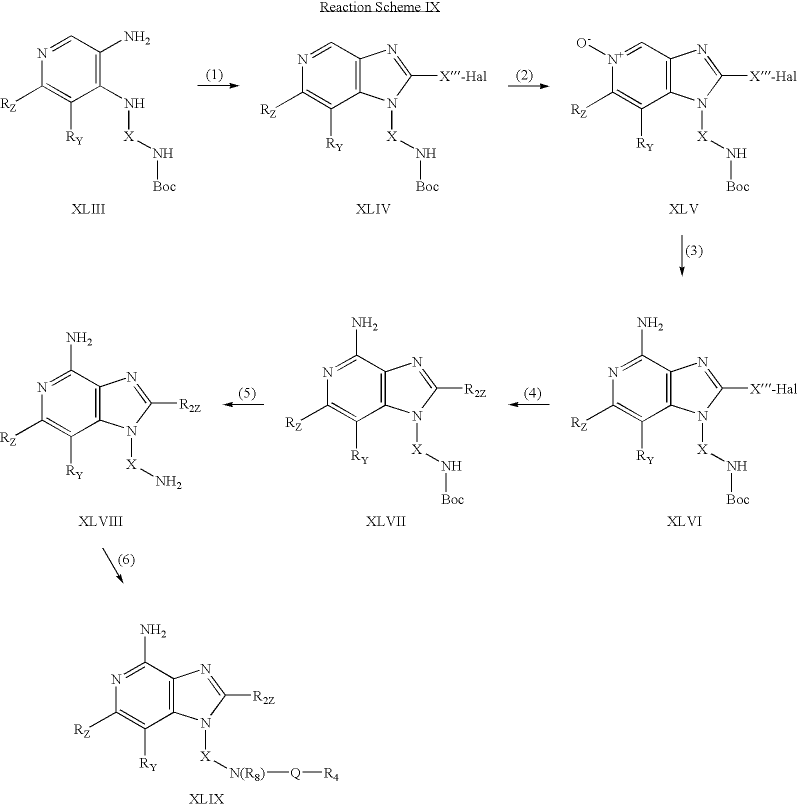

Tetrahydroquinolines and tetrahydronaphthyridines of the invention can be prepared according to Reaction Scheme X, wherein R_{Ya} and R_{Za} join to form a fused benzene ring or a fused pyridine ring, each of which is optionally substituted by one or more R_{g} groups, wherein R_{g} is alkyl, alkoxy, or —N(R₉)₂; R_{W} and R_{X} join to form a fused 5 to 7 membered saturated ring, optionally containing one heteroatom selected from the group consisting of N and S and optionally substituted by one or more R_{g} groups; R_{1d} can be those groups included in R₁₋₄ₐ, R_{1-4b}, R_{1-4c}, R_{1-4d}, R₁₋₆, and R₁₋₇ as defined above that do not include those substituents that one skilled in the art would recognize as being susceptible to reduction under the acidic hydrogenation conditions of the reaction; R_{2y} is selected from the group consisting of 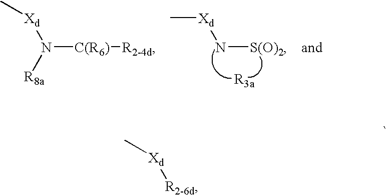
wherein R₆, R₈ₐ, and R₃ₐ are as defined above, X_{d} is C₁₋₄ alkylene, and R_{2-4d} and R_{2-6d} are subsets of R₂₋₄ and R₂₋₆ as defined above that do not include those substituents that one skilled in the art would recognize as being susceptible to reduction under the acidic hydrogenation conditions of the reaction. These susceptible groups include, for example, alkenyl, alkynyl, and aryl groups and groups bearing nitro substituents. Compounds of Formula L can be prepared according to one of the methods described in Reaction Scheme III, IV, VII, VIII, and IX.

In Reaction Scheme X, a substituted 1H-imidazo[4,5-c]quinolin-4-amine of or 1H-imidazo[4,5-c]naphthyridin-4-amine of Formula L is reduced to a 6,7,8,9-tetrahydro-1H-imidazo[4,5-c]quinolin-4-amine or 6,7,8,9-tetrahydro-1H-imidazo[4,5-c]naphthyridin-4-amine of Formula LI. The reaction is conveniently carried out under hetereogeneous hydrogenation conditions by adding platinum (IV) oxide to a solution of the compound of Formula L in trifluoroacetic acid and placing the reaction under hydrogen pressure. The reaction can be carried out on a Parr apparatus at room temperature.

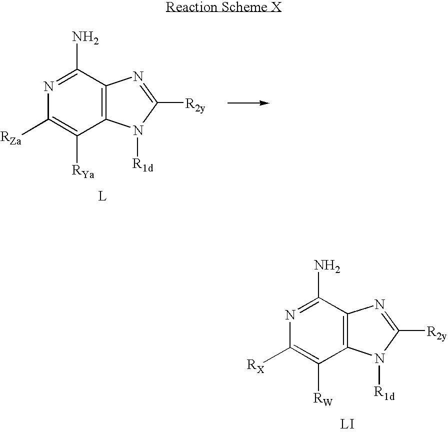

For some embodiments, imidazopyridines of the invention can be prepared according to Reaction Scheme Xa, wherein R_{A1}, R_{B1}, Ph, Hal, R₁, and X′″ are as defined above, and R₂ₓ is selected from the group consisting of 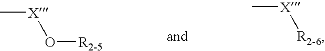
wherein X′″, R₂₋₅, and R₂₋₆ are as defined above. In step (1) of Reaction Scheme Xa, an imidazopyridine of Formula XXXVII is treated according to one of the methods described in Reaction Scheme V, VII, or VIII to provide a compound of Formula LII. In step (2) of Reaction Scheme Xa, an imidazopyridine of Formula LII is aminated according to the method described in step (2) of Reaction Scheme II. Ammonium acetate can also be used as the aminating reagent in step (2).

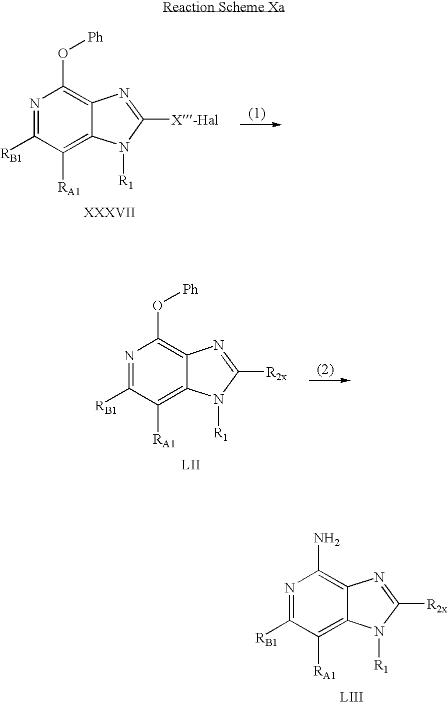

For some embodiments compounds of the invention can be prepared according to Reaction Scheme XI, wherein Rₐ, R₁₁, R₁₋₁, R₂₋₂, X′, R′, and n are as defined above. In step (1) of Reaction Scheme XI, a 4-chloro-3-nitroquinoline of Formula LIV is reacted with an amine of the Formula 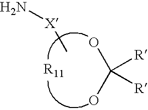
to form a compound of Formula LV. This reaction is conveniently carried out by adding the amine to a solution of a 4-chloro-3-nitroquinoline of Formula LIV in the presence of a base such as triethylamine. The reaction is carried out in a suitable solvent, such as dichloromethane or chloroform. Some amines of Formula 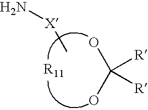
such as 2,2-dimethyl-1,3-dioxolane-4-methanamine are commercially available in both racemic and enantiomerically pure forms. Others can be prepared using known synthetic methods. Many compounds of Formula LIV are known or can be prepared using known synthetic methods, see for example, U.S. Pat. Nos. 4,689,338 (Gerster) and 4,988,815 (Andre et al.), U.S. Patent Publication Application No. US 2004/0147543 (Hays et al.), and the documents cited therein.

The resultant compound of Formula LV can be reduced in step (2) of Reaction Scheme XI using a variety of methods to provide a quinoline-3,4-diamine of Formula LVI. The reaction can be carried out by hydrogenation using a heterogeneous hydrogenation catalyst such as platinum on carbon. The hydrogenation is conveniently carried out in a Parr apparatus in a suitable solvent such as toluene or ethanol. The reaction can be carried out at ambient temperature.

Alternatively step (2) can be carried out using a one- or two-phase sodium dithionite reduction. The reaction is conveniently carried out using the conditions described by Park, K. K.; Oh, C. H.; and Joung, W. K.; Tetrahedron Lett., 34, pp. 7445-7446 (1993) by adding sodium dithionite to a compound of Formula LV in a mixture of dichloromethane and water at ambient temperature in the presence of potassium carbonate and ethyl viologen dibromide, ethyl viologen diiodide, or 1,1′-di-n-octyl-4,4′-bipyridinium dibromide.

In step (3) of Reaction Scheme XI, a quinoline-3,4-diamine of Formula LVI is treated with a carboxylic acid equivalent to provide a 1H-imidazo[4,5-c]quinoline of Formula LVII. Suitable carboxylic acid equivalents include orthoesters of Formula R₂₋₂C(O-alkyl)₃, 1,1-dialkoxyalkyl alkanoates of Formula R₂₋₂C(O-alkyl)₂(O—C(O)-alkyl), and acid chlorides of Formula R₂₋₂C(O)Cl. The selection of the carboxylic acid equivalent is determined by the desired substituent at R₂₋₂. For example, triethyl orthoformate will provide a compound where R₂₋₂ is hydrogen, and trimethyl orthovalerate will provide a compound where R₂₋₂ is a butyl group. The reaction is conveniently carried out by adding the carboxylic acid equivalent to a quinoline-3,4-diamine of Formula LVI in a suitable solvent such as toluene. Optionally, catalytic pyridine hydrochloride or pyridium p-toluenesulfonate can be added. The reaction is carried out at a temperature high enough to drive off alcohol or water formed during the reaction. Conveniently, a Dean-Stark trap can be used to collect the volatiles.

Alternatively, step (3) can be carried out in two steps when an acid chloride of Formula R₂₋₂C(O)Cl is used as the carboxylic acid equivalent. The first step is conveniently carried out by adding the acid chloride to a solution of a quinoline-3,4-diamine of Formula LVI in a suitable solvent such as dichloromethane to afford an amide. Optionally, a tertiary amine such as triethylamine, pyridine, or 4-dimethylaminopyridine can be added. The reaction can be carried out at or below ambient temperature. The amide product can be isolated and optionally purified using conventional techniques before it is heated and cyclized to provide a 1H-imidazo[4,5-c]quinoline of Formula LVII. The cyclization reaction is conveniently carried out in a solvent such as ethanol or methanol in the presence of a base such as triethylamine and may be carried out at an elevated temperature, such as the reflux temperature of the solvent.

In steps (4) and (5) of Reaction Scheme XI, a 1H-imidazo[4,5-c]quinoline of Formula LVII is first oxidized to a 5N-oxide of Formula LVIII, which is then aminated to provide a 1H-imidazo[4,5-c]quinolin-4-amine of Formula VI. Steps (4) and (5) of Reaction Scheme XI can be carried out according to the methods described in steps (2) and (3) of Reaction Scheme I.

In step (6) of Reaction Scheme XI, the ketal or acetal of Formula VI is converted to a diol of Formula IIa by acid-catalyzed hydrolysis. The reaction is conveniently carried out by adding a strong acid, such as hydrochloric acid, to a ketal or acetal of Formula VI. The reaction may be carried out at ambient temperature in a suitable solvent or solvent system such as a tetrahydrofuran/water mixture.

Conversion of a diol of Formula IIa to a ketal or acetal of Formula VI is also possible by using the method shown in step (7) of Reaction Scheme XI. In step (7), a diol of Formula IIa reacts with a ketone or aldehyde in the presence of an acid catalyst. Conditions for this reaction are well known to one skilled in the art. See, for example, T. W. Greene and P. G. M. Wuts, Protective Groups in Organic Synthesis, John Wiley & Sons, New York, USA, 1991, p. 178. Numerous ketones and aldehydes are commercially available; others can be prepared using known synthetic methods.

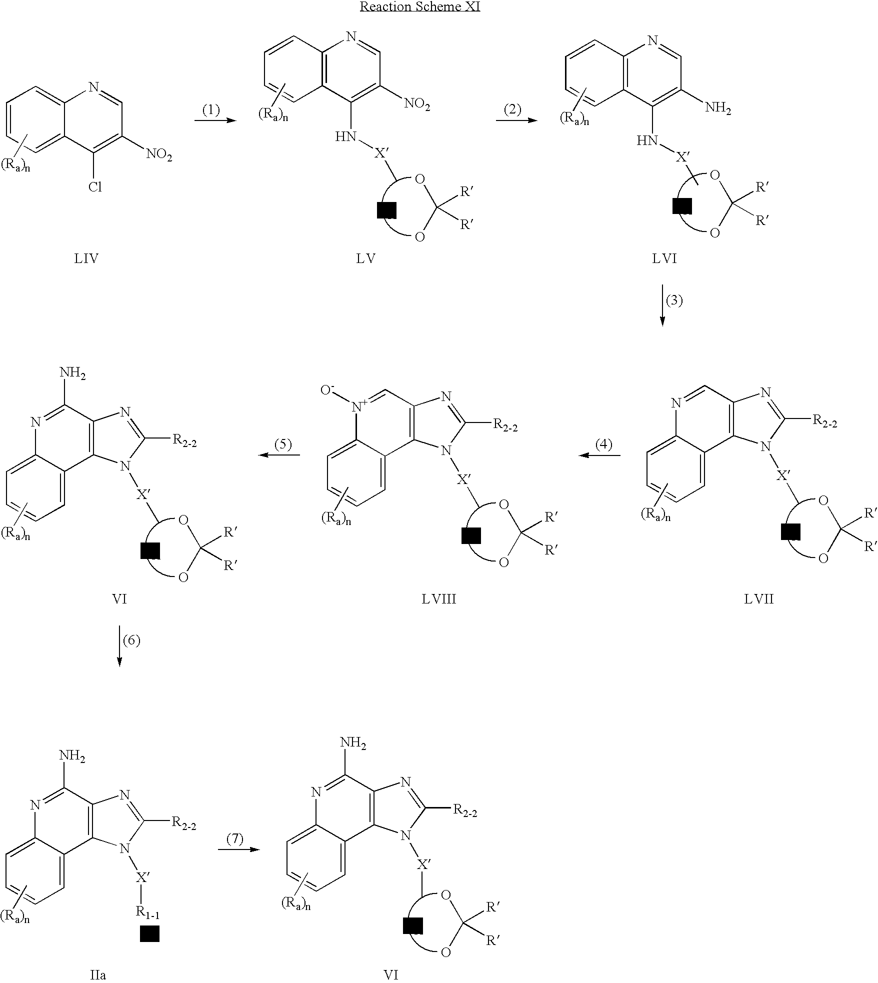

For some embodiments, tetrahydroquinolines of the invention can be prepared according to Reaction Scheme XII, wherein R_{g}, R₁₋₁, R₁₁, R′, and n are as defined above, and R_{2-2b} and X′_{b} are subsets of R₂₋₂ and X′ as defined above that do not include those substituents that one skilled in the art would recognize as being susceptible to reduction under the acidic hydrogenation conditions of step (1). These susceptible groups include, for example, alkenyl, alkynyl, and aryl groups and groups bearing nitro substituents. In step (1) of Reaction Scheme XII, an 1H-imidazo[4,5-c]quinoline of Formula IIb is reduced to a 6,7,8,9-tetrahydro-1H-imidazo[4,5-c]quinolin-4-amine of Formula IIIa. The reaction is conveniently carried out according to the method described in Reaction Scheme X.

In step (2) of Reaction Scheme XII, a diol of Formula IIIa is converted to a ketal or acetal of Formula VIIIa according to the method described in step (7) of Reaction Scheme XI.

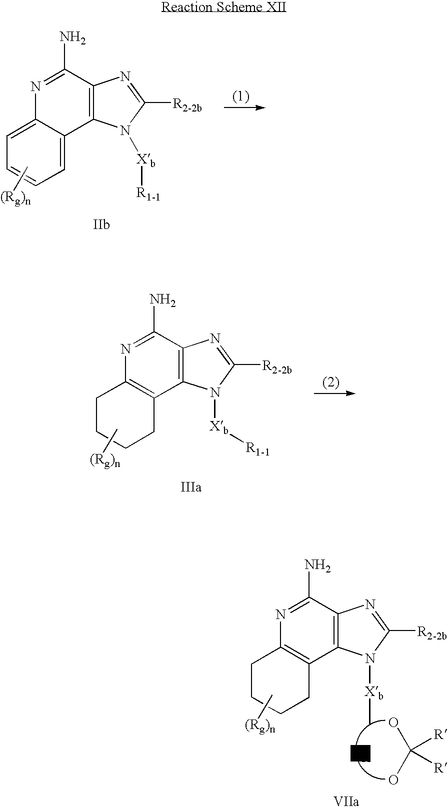

For some embodiments, compounds of the invention are prepared according to Reaction Scheme XIII, wherein Rₐ₂, R₂₋₃, X″, and n are as defined above; and R₁₋₃ₐ is a subset of R₁₋₃ that includes the rings: 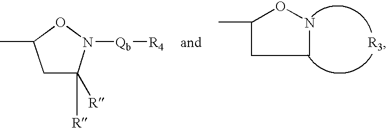
wherein Q_{b} is a bond and R″, R₃, and R₄ are defined as above. In step (1) of Reaction Scheme XIII, an amine of Formula NH₂—X″—CH═CH₂ is added to a compound of Formula LIX to provide a substituted quinoline of Formula LX. The reaction can be carried out according to the method described in step (1) of Reaction Scheme XI. Several compounds of Formula LIX are known and can be made by known methods. See, for example, U.S. Pat. No. 4,689,338 (Gerster). Amines of the Formula NH₂—X″—CH═CH₂ are commercially available or can be readily prepared by known methods.

In steps (2) and (3) of Reaction Scheme XIII, the nitro group of a compound of Formula LX is first reduced to provide a quinoline-3,4-diamine of Formula LXI, which is cyclized to provide a 1H-imidazo[4,5-c]quinoline of Formula LXII. Steps (2) and (3) of Reaction Scheme XIII can be carried out according to the methods described in steps (2) and (3) of Reaction Scheme XI.

In step (4) of Reaction Scheme XIII, the alkene group of a compound of Formula LXII reacts with a nitrone of Formula LXIII or LXIV to provide a heterocyclyl-substituted 1H-imidazo[4,5-c]quinoline of Formula LXV. Nitrones of Formula LXIII are known and can be prepared by known methods. See, for example, Dicken, C. M. and DeShong, P., J. Org. Chem., 47, pp. 2047-2051 (1982). Nitrones of Formula LXIV can be prepared according to the literature procedures: Thesing, J.; Sirrenberg, W., Chem. Ber., 92, p. 1748, (1959) and Iwashita, T. et al., J. Org. Chem., 47, p. 230, (1982). The cycloaddition reaction shown in step (4) can be carried out by combining the nitrone of Formula LXIII or LXIV with a compound of Formula LXII in a suitable solvent such as toluene and heating at an elevated temperature, for example, the reflux temperature of the solvent. Nitrones of Formula LXIII can also be prepared in situ by combining a hydroxylamine of Formula R₄—NH—OH or a hydrochloride salt thereof and an aldehyde or ketone of Formula (R″)₂C═O with a compound of Formula LXII in the presence of a base such as sodium bicarbonate and alumina. The reaction can be carried out at an elevated temperature in a suitable solvent such as toluene.

In step (5) of Reaction Scheme XIII, a heterocyclyl-substituted compound of Formula LXV is aminated to provide a heterocyclyl-substituted 1H-imidazo[4,5-c]quinolin-4-amine of Formula XIIIa, a subgenus of Formulas XI and XIII. The reaction can be carried out according to the method described in step (2) of Reaction Scheme 2.

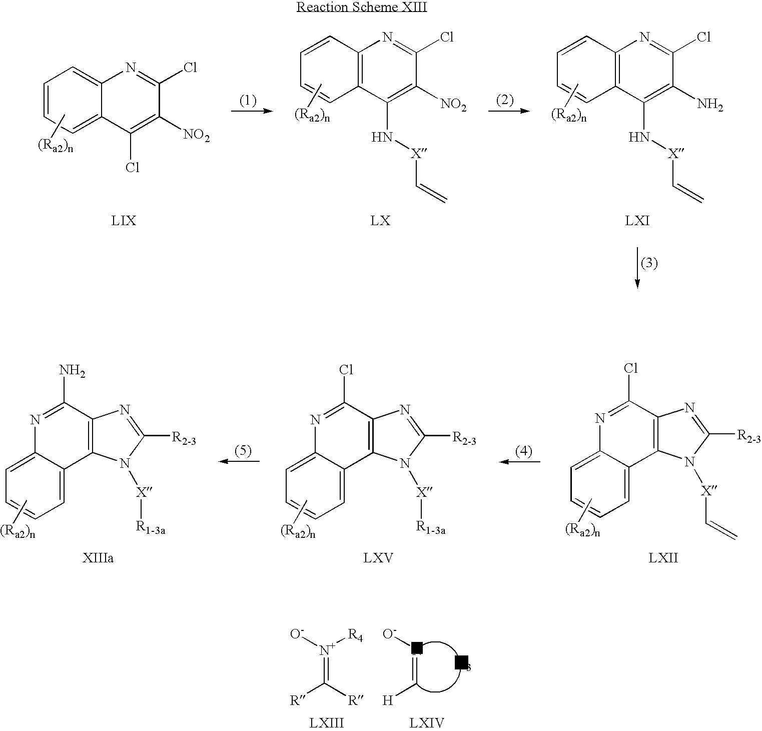

Imidazoquinolin-4-amines of the invention can also be prepared according to Reaction Scheme XIV, wherein Rₐ₂, R₂₋₃, R″, X″, Q, R₄, and n are as defined above. In step (1) of Reaction Scheme XIV, an isoxazolidine-substituted 1H-imidazo[4,5-c]quinolin-4-amine of Formula LXVI undergoes reductive cleavage to provide an amino alcohol of Formula LXVII. The reaction is conveniently carried out under heterogeneous hydrogenation conditions in the presence of a heterogeneous hydrogenation catalyst such as palladium on carbon. The reaction can be carried out in a suitable solvent or solvent combination such as methanol:acetic acid.

In step (2) of Reaction Scheme XIV, an amino alcohol of Formula LXVII is converted to an oxazolidinone of Formula LXVIII using an appropriate coupling reagent such as 1,1′-carbonyldiimidazole. The reaction is conveniently carried out by heating, for example at reflux, the amino alcohol of Formula LXVII and 1,1′-carbonyldiimidazole in a suitable solvent such as tetrahydrofuran. Steps (1) and (2) of Reaction Scheme XIV can also be carried out when the nitrogen of the oxazolidine ring is substituted by an R₄ group other than hydrogen.

In steps (1a) and (3) of Reaction Scheme XIV, compounds of the Formulas LXVI and LXVIII can be converted to substituted oxazolidines and oxazolidinones of Formulas LXVIa and LXVIIIa using the one of the methods described in step (2) of Reaction Scheme VII and step (6) of Reaction Scheme IX.

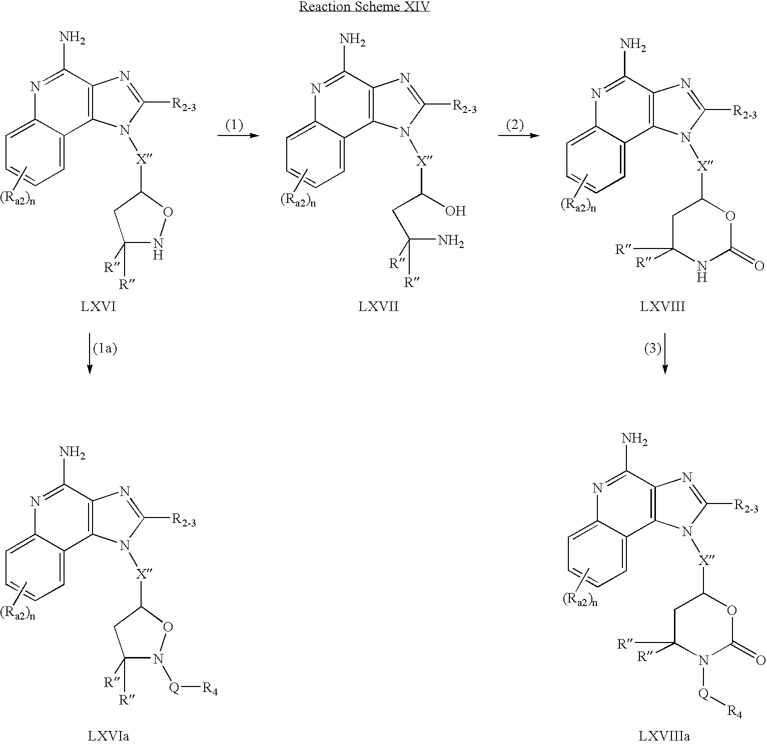

Heterocyclyl-substituted compounds of the invention can be prepared according to Reaction Scheme XV, wherein R_{A-2a}, R_{B-2b}, X″, and R₂₋₃ are as defined above, and R_{1-3b} is a subset of R₁₋₃ that includes the rings: 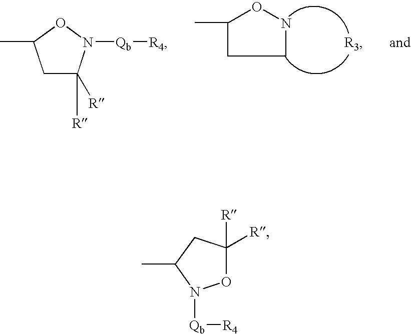
wherein Q_{b} is a bond and R″, R₃, and R₄ are defined as above. In step (1) of Reaction Scheme XV, a compound of Formula LXIX is reacted with an amino alcohol of the Formula H₂N—X″—OH to form a compound of Formula LXX. The reaction is conveniently carried out according to the method described in step (1) of Reaction Scheme XIII. Many 2,4-dichloro-3-nitropyridines of the Formula LXIX are known and can be readily prepared using known synthetic methods. See, for example, Dellaria et al, U.S. Pat. No. 6,525,064 and the references cited therein. Many 2,4-dichloro-3-nitroquinolines are also known and can be prepared by known methods; see, for example, U.S. Pat. No. 4,988,815 (Andre et al). Related routes to tetrahydroquinolines of Formula LXX are known; see, for example, U.S. Pat. Nos. 5,352,784 (Nikolaides et al) and 6,670,372 (Charles et al).

In step (2) of Reaction Scheme XV a compound of Formula LXX is reacted with an alkali metal azide to provide a tetrazole of Formula LXXI. The reaction can be carried out by combining the compound of Formula LXX with an alkali metal azide, for example, sodium azide, in a suitable solvent such as acetonitrile/water, preferably 90/10 acetonitrile/water, in the presence of cerium (III) chloride, preferably cerium (III) chloride heptahydrate. Optionally, the reaction can be carried out with heating, for example, at the reflux temperature. Alternatively, the reaction can be carried out by combining the compound of Formula LXX with an alkali metal azide, for example, sodium azide, in a suitable solvent such as DMF and heating, for example to about 50° C. to 60° C., optionally in the presence of ammonium chloride. Other related routes to imidazonaphthyridines of Formula LXXI have been reported; see, for example, U.S. Pat. No. 6,194,425 (Gerster et al).

In step (3) of Reaction Scheme XV, the nitro group of the compound of Formula LXXI is reduced to provide a diamine of Formula LXXII. The reduction can be carried out according to the methods described in step (2) of Reaction Scheme XI.

In step (4) of Reaction Scheme XV, a diamine of Formula LXXII is reacted with a carboxylic acid equivalent to provide a compound of Formula LXXIII. The reaction can be carried out as described in step (3) of Reaction Scheme XI.

In step (5) of Reaction Scheme XV, the alcohol of Formula LXXIII is oxidized to an aldehyde-substituted compound of Formula LXXIV using conventional methods, for example, Swern oxidation conditions. The Swern oxidation is conveniently carried out by adding a compound of Formula LXXIII followed by triethylamine to a mixture of oxalyl chloride and dimethylsulfoxide in a suitable solvent, such as dichloromethane. The reaction can be carried out at sub-ambient temperatures, such as −78° C.

In step (6) of Reaction Scheme XV, an aldehyde-substituted compound of Formula LXXIV is converted to an alkenyl-substituted compound of Formula LXXV. The reaction can be carried out using synthetic methods well known to those skilled in the art; such methods include the Wittig reaction.

In step (7) of Reaction Scheme XV, the alkene dipolarophile of Formula LXXV undergoes a cycloaddition reaction with a nitrone of Formula LXIII or LXIV to provide a heterocyclyl-substituted compound of Formula LXXVI wherein R_{1-3b} is 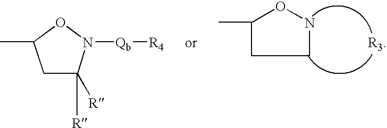
The reaction can be run according to one of the methods described in step (4) of Reaction Scheme XIII to provide a product of Formula LXXVI.

Compounds of Formula LXXVI wherein R_{1-3b} is 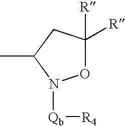
can be prepared according to step (6a) of Reaction Scheme XV. The transformation can be carried out by converting an aldehyde of Formula LXXIV to a nitrone using one of the methods described in step (4) of Reaction Scheme XIII. The nitrone can then undergo cycloaddition with an alkene of formula R′—CH═CH₂ according to one of the methods described in step (4) of Reaction Scheme XIII. Numerous alkenes of this formula are commercially available; others can be prepared by known methods. The reaction may be carried out in one step if the nitrone is generated in situ in the presence of an alkene.

In step (8) of Reaction Scheme XV, the tetrazole ring is removed from a compound of Formula LXXVI by reaction with triphenylphosphine to form an N-triphenylphosphinyl intermediate. The reaction with triphenylphosphine can be run in a suitable solvent such as toluene or 1,2-dichlorobenzene under an atmosphere of nitrogen with heating, for example at the reflux temperature. The N-triphenylphosphinyl intermediate is then hydrolyzed to provide a compound of Formula XIa, a subgenus of Formula XI.

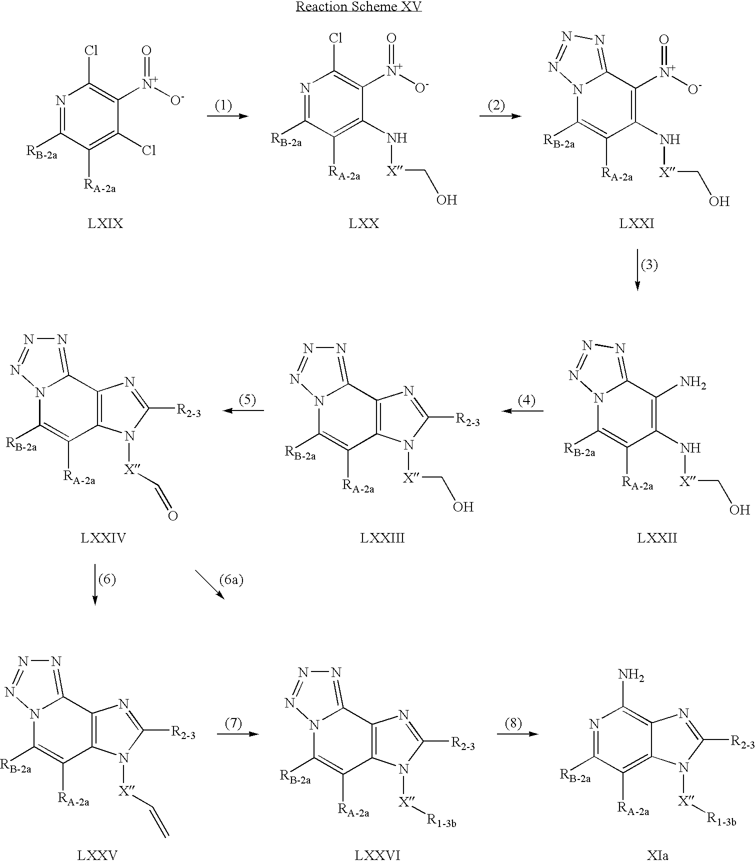

Tetrahydroquinolines and tetrahydronaphthyridines of the invention can be prepared according to Reaction Scheme XVI, wherein R_{Ya}, R_{Za}, R_{W}, R_{X}, R_{1d}, and X′_{b} are as defined above, P is a hydroxy protecting group, and R_{2z-1} is a subset of R_{2z} as defined above in which X′″ is C₁₋₄ alkylene.

In step (1) of Reaction Scheme XVI, a compound of Formula XXXa or a salt thereof is reacted with a carboxylic acid or an equivalent thereof to provide a compound of Formula LXXVII. Compounds of Formula XXXa are a subset of compounds of Formula XXX, which are shown in Reaction Scheme I. Suitable carboxylic acid equivalents that can be used to provide a compound of formula LXXVII include acid anhydrides of formula O[C(O)—X′_{b}—CH₂—O—P]₂ and acid chlorides of formula Cl—C(O)—X′_{b}—CH₂—O—P. The reaction is conveniently carried out by under the conditions described in step (1) of Reaction Scheme I for the reaction with acid chlorides of formula Hal-X′—C(O)Cl. Some compounds of formula Cl—C(O)—X′_{b}—O—P, such as acetoxyacetyl chloride, methoxyacetyl chloride, and 2-methoxypropionyl chloride, are commercially available. Others can be prepared by known synthetic methods.

Alternatively, step (1) can be carried out in two steps by first heating a quinoline-3,4-diamine of Formula XXXa with a carboxylic acid of formula HO—X′_{b}—CO₂H, with a trialkyl orthoester of formula HO—X′_{b}—C(O—C₁₋₄ alkyl)₃, or with a combination thereof to provide a hydroxy-substituted compound. The reaction is run with sufficient heating to drive off any alcohol or water formed as a byproduct of the reaction and is typically run at about 130° C. The resultant hydroxy-substituted compound is protected with a removable protecting group such as an alkanoyloxy group (e.g., acetoxy) or aroyloxy group (e.g., benzoyloxy) to provide a compound of Formula LXXVII. Suitable protecting groups and reactions for their placement and removal are well known to those skilled in the art. See, for example, U.S. Pat. No. 4,689,338 (Gerster), Examples 115 and 120 and U.S. Pat. No. 5,389,640 (Gerster et al.), Examples 2 and 3.

In steps (2) and (3) of Reaction Scheme XVI, a protected hydroxy-substituted imidazoquinoline or imidazonaphthyridine of Formula LXXVII is first oxidized to an N-oxide of Formula LXXVIII, which is then aminated to a compound of Formula LXXIX. Steps (2) and (3) of Reaction Scheme XVI can be carried out as described for steps (2) and (3) of Reaction Scheme I. Under the amination reaction conditions, some protecting groups are removed; for example, an ester group such as an acetoxy group would be hydrolyzed under these conditions. Other hydroxy protecting groups may need to be removed in a subsequent step prior to step (4) to provide a compound of Formula LXXIX. For example, a methyl ether, wherein P is methyl, can be dealkylated by treatment with boron tribromide in a suitable solvent such as dichloromethane at a sub-ambient temperature such as 0° C.

In step (4) of Reaction Scheme XVI, a compound of Formula LXXIX is reduced according to the method described in Reaction Scheme X to provide a hydroxy-substituted 6,7,8,9-tetrahydro-1H-imidazo[4,5-c]quinolin-4-amine or 6,7,8,9-tetrahydro-1H-imidazo[4,5-c]naphthyridin-4-amine of Formula LXXX.

In step (5) of Reaction Scheme XVI, a compound of Formula LXXX is halogenated using conventional methods to provide a compound of Formula LXXXI. For example, a hydroxy-substituted compound of Formula LXXX can be combined with thionyl chloride in a suitable solvent such as dichloromethane or 1,2-dichloroethane at room temperature.

In step (6) of Reaction Scheme XVI, a halogen-substituted compound of Formula LXXXI is treated according to any of the methods or combination of methods described in Reaction Schemes I, III, IV, V, VII, and VIII to introduce the R₂, group and provide a compound of Formula LXXXII. The transformation can be carried out according to one of the methods described in step (4) of Reaction Scheme IX.

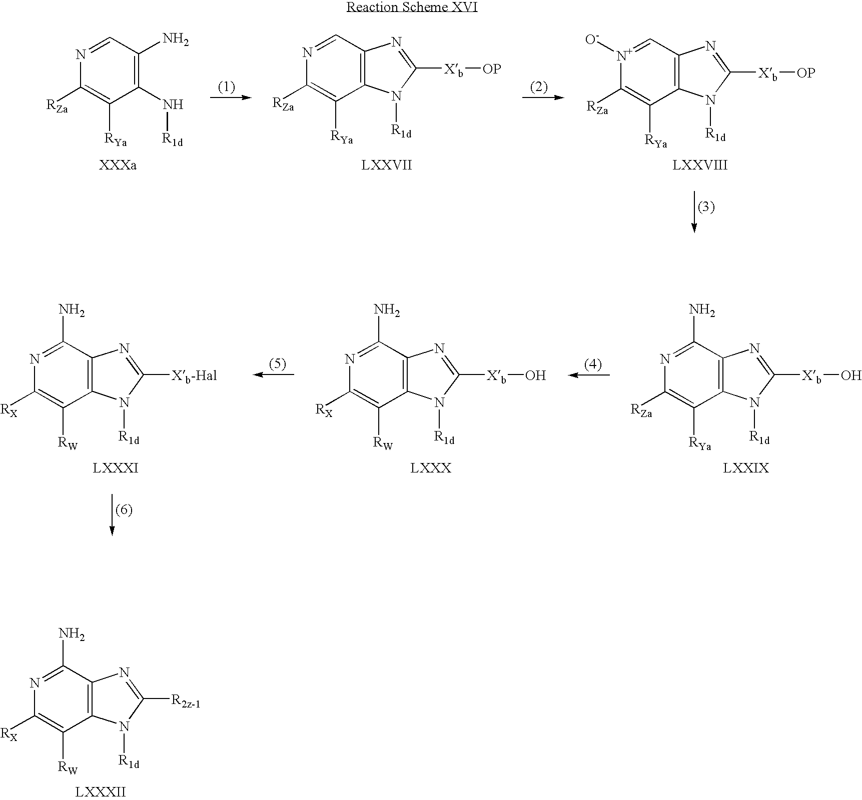

Compounds of the invention can also be prepared using variations of the synthetic routes shown in Reaction Schemes I through XVI that would be apparent to one of skill in the art. For example, the synthetic route shown in Reaction Scheme XI for the preparation of quinolines can be used to prepare [1,5]naphthyridines by starting with a 4-chloro-3-nitro[1,5]naphthyridine in lieu of the 4-chloro-3-nitroquinoline. In another example, the methods described in Reaction Scheme XVI can be used to install a hydroalkyl group at the R₂₋₂ or R₂₋₃ position shown in Reaction Scheme XI, XIII, or XV. Also, the methods shown in Reaction Scheme XIV can be carried out on a compound wherein R₁₋₃ is 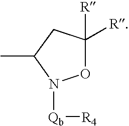
Compounds of the invention can also be prepared using the synthetic routes described in the EXAMPLES below.

Prodrugs can be prepared in a variety of ways. For example, a compound wherein R₁ or R₂ is hydroxyalkyl can be converted into a prodrug wherein R₁ or R₂ is, for example, an ester, an ether, a carbonate, or a carbamate, using methods known to one skilled in the art. In addition, a compound wherein R_{b} is hydroxy may also be converted to an ester, an ether, a carbonate, or a carbamate. For any of these compounds containing an alcohol functional group, a prodrug can be formed by the replacement of the hydrogen atom of the alcohol group with a group such as C₁₋₆ alkanoyloxymethyl, 1-(C₁₋₆ alkanoyloxy)ethyl, 1-methyl-1-(C₁₋₆ alkanoyloxy)ethyl, C₁₋₆ alkoxycarbonyloxymethyl, N—(C₁₋₆ alkoxycarbonyl)aminomethyl, succinoyl, C₁₋₆ alkanoyl, α-aminoC₁₋₄ alkanoyl, arylacyl, —P(O)(OH)₂, —P(O)(O—C₁₋₆ alkyl)₂, C₁₋₆ alkoxycarbonyl, C₁₋₆ alkylcarbamoyl, and α-aminoacyl or α-aminoacyl-α-aminoacyl, where each α-aminoacyl group is independently selected from racemic, D-, and L-amino acids. For compounds containing an alcohol functional group, particularly useful prodrugs are esters made from carboxylic acids containing one to six carbon atoms, unsubstituted or substituted benzoic acid esters, or esters made from amino acids.

Prodrugs can also be made from a compound containing an amino group by conversion of the amino group to a functional group such as an amide, carbamate, urea, amidine, or another hydroylizable group using conventional methods. A prodrug of this type can be made by the replacement of a hydrogen atom in an amino group, particularly the amino group at the 4-position, with a group such as —C(O)—R′″, α-aminoacyl, α-aminoacyl-α-aminoacyl, —C(O)—O—R′″, —C(O)—N(R″″)—R′″, —C(═NY₅)—R″″, —CH(OH)—C(O)—OY₅, —CH(OC₁₋₄ alkyl)Y₀, —CH₂Y₆, or —CH(CH₃)Y₆; wherein R′″ and R″″ are each independently C₁₋₁₀ alkyl, C₃₋₇ cycloalkyl, or benzyl, each of which may be unsubstituted or substituted by one or more substituents selected from the group consisting of halogen, hydroxy, nitro, cyano, carboxy, C₁₋₆ alkyl, C₁₋₄ alkoxy, aryl, heteroaryl, arylC₁₋₄ alkylenyl, heteroarylC₁₋₄ alkylenyl, haloC₁₋₄ alkylenyl, haloC₁₋₄ alkoxy, —O—C(O)—CH₃, —C(O)—O—CH₃, —C(O)—NH₂, —O—CH₂—C(O)—NH₂, —NH₂, and —S(O)₂—NH₂; with the proviso that R″″ may also be hydrogen; each α-aminoacyl group is independently selected from racemic, D, or L-amino acids; Y₅ is hydrogen, C₁₋₆ alkyl, or benzyl; Y₀ is C₁₋₆ alkyl, carboxyC₁₋₆ alkylenyl, aminoC₁₋₄ alkylenyl, mono-N—C₁₋₆ alkylaminoC₁₋₄ alkylenyl, or di-N,N—C₁₋₆ alkylaminoC₁₋₄ alkylenyl; and Y₆ is mono-N—C₁₋₆ alkylamino, di-N,N—C₁₋₆ alkylamino, morpholin-4-yl, piperidin-1-yl, pyrrolidin-1-yl, or 4-C₁₋₄ alkylpiperazin-1-yl. For compounds containing an amine functional group, particularly useful prodrugs are amides derived from carboxylic acids containing one to ten carbon atoms, amides derived from amino acids, and carbamates containing one to ten carbon atoms.

Pharmaceutical Compositions and Biological Activity

Pharmaceutical compositions of the invention contain a therapeutically effective amount of a compound or salt of the invention as described above in combination with a pharmaceutically acceptable carrier.

The terms “a therapeutically effective amount” and “effective amount” mean an amount of the compound or salt sufficient to induce a therapeutic or prophylactic effect, such as cytokine induction, immunomodulation, antitumor activity, and/or antiviral activity. Although the exact amount of active compound or salt used in a pharmaceutical composition of the invention will vary according to factors known to those of skill in the art, such as the physical and chemical nature of the compound or salt, the nature of the carrier, and the intended dosing regimen, it is anticipated that the compositions of the invention will contain sufficient active ingredient to provide a dose of about 100 nanograms per kilogram (ng/kg) to about 50 milligrams per kilogram (mg/kg), preferably about 10 micrograms per kilogram (μg/kg) to about 5 mg/kg, of the compound or salt to the subject. A variety of dosage forms may be used, such as tablets, lozenges, capsules, parenteral formulations, syrups, creams, ointments, aerosol formulations, transdermal patches, transmucosal patches and the like.

The compounds or salts of the invention can be administered as the single therapeutic agent in the treatment regimen, or the compounds or salts of the invention may be administered in combination with one another or with other active agents, including additional immune response modifiers, antivirals, antibiotics, antibodies, proteins, peptides, oligonucleotides, etc.

Compounds or salts of the invention have been shown to induce the production of certain cytokines in experiments performed according to the test set forth below. These results indicate that the compounds or salts are useful as immune response modifiers that can modulate the immune response in a number of different ways, rendering them useful in the treatment of a variety of disorders.

Cytokines whose production may be induced by the administration of compounds or salts of the invention generally include interferon-α (IFN-α) and/or tumor necrosis factor-α (TNF-α) as well as certain interleukins (IL). Cytokines whose biosynthesis may be induced by compounds or salts of the invention include IFN-α, TNF-α, IL-1, IL-6, IL-10 and IL-12, and a variety of other cytokines. Among other effects, these and other cytokines can inhibit virus production and tumor cell growth, making the compounds or salts useful in the treatment of viral diseases and neoplastic diseases. Accordingly, the invention provides a method of inducing cytokine biosynthesis in an animal comprising administering an effective amount of a compound or salt or composition of the invention to the animal. The animal to which the compound or salt or composition is administered for induction of cytokine biosynthesis may have a disease as described infra, for example a viral disease or a neoplastic disease, and administration of the compound or salt may provide therapeutic treatment. Alternatively, the compound or salt may be administered to the animal prior to the animal acquiring the disease so that administration of the compound or salt may provide a prophylactic treatment.

In addition to the ability to induce the production of cytokines, compounds or salts of the invention can affect other aspects of the innate immune response. For example, natural killer cell activity may be stimulated, an effect that may be due to cytokine induction. The compounds or salts may also activate macrophages, which in turn stimulate secretion of nitric oxide and the production of additional cytokines. Further, the compounds or salts may cause proliferation and differentiation of B-lymphocytes.

Compounds or salts of the invention can also have an effect on the acquired immune response. For example, the production of the T helper type 1 (T_{H}1) cytokine IFN-γ may be induced indirectly and the production of the T helper type 2 (T_{H}2) cytokines IL-4, IL-5 and IL-13 may be inhibited upon administration of the compounds or salts.

Whether for prophylaxis or therapeutic treatment of a disease, and whether for effecting innate or acquired immunity, the compound or salt or composition may be administered alone or in combination with one or more active components as in, for example, a vaccine adjuvant. When administered with other components, the compound or salt and other component or components may be administered separately; together but independently such as in a solution; or together and associated with one another such as (a) covalently linked or (b) non-covalently associated, e.g., in a colloidal suspension.

Conditions for which compounds or salts identified herein may be used as treatments include, but are not limited to:

(a) viral diseases such as, for example, diseases resulting from infection by an adenovirus, a herpesvirus (e.g., HSV-I, HSV-II, CMV, or VZV), a poxvirus (e.g., an orthopoxvirus such as variola or vaccinia, or molluscum contagiosum), a picornavirus (e.g., rhinovirus or enterovirus), an orthomyxovirus (e.g., influenzavirus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV);

(b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella;

(c) other infectious diseases, such chlamydia, fungal diseases including but not limited to candidiasis, aspergillosis, histoplasmosis, cryptococcal meningitis, or parasitic diseases including but not limited to malaria, pneumocystis carnii pneumonia, leishmaniasis, cryptosporidiosis, toxoplasmosis, and trypanosome infection;

(d) neoplastic diseases, such as intraepithelial neoplasias, cervical dysplasia, actinic keratosis, basal cell carcinoma, squamous cell carcinoma, renal cell carcinoma, Kaposi's sarcoma, melanoma, leukemias including but not limited to myelogeous leukemia, chronic lymphocytic leukemia, multiple myeloma, non-Hodgkin's lymphoma, cutaneous T-cell lymphoma, B-cell lymphoma, and hairy cell leukemia, and other cancers;

(e) T_{H}2-mediated, atopic diseases, such as atopic dermatitis or eczema, eosinophilia, asthma, allergy, allergic rhinitis, and Ommen's syndrome;

(f) certain autoimmune diseases such as systemic lupus erythematosus, essential thrombocythaemia, multiple sclerosis, discoid lupus, alopecia greata; and

(g) diseases associated with wound repair such as, for example, inhibition of keloid formation and other types of scarring (e.g., enhancing wound healing, including chronic wounds).

Additionally, a compound or salt of the present invention may be useful as a vaccine adjuvant for use in conjunction with any material that raises either humoral and/or cell mediated immune response, such as, for example, live viral, bacterial, or parasitic immunogens; inactivated viral, tumor-derived, protozoal, organism-derived, fungal, or bacterial immunogens; toxoids; toxins; self-antigens; polysaccharides; proteins; glycoproteins; peptides; cellular vaccines; DNA vaccines; autologous vaccines; recombinant proteins; and the like, for use in connection with, for example, BCG, cholera, plague, typhoid, hepatitis A, hepatitis B, hepatitis C, influenza A, influenza B, parainfluenza, polio, rabies, measles, mumps, rubella, yellow fever, tetanus, diphtheria, hemophilus influenza b, tuberculosis, meningococcal and pneumococcal vaccines, adenovirus, HIV, chicken pox, cytomegalovirus, dengue, feline leukemia, fowl plague, HSV-1 and HSV-2, hog cholera, Japanese encephalitis, respiratory syncytial virus, rotavirus, papilloma virus, yellow fever, and Alzheimer's Disease.

Compounds or salts of the present invention may be particularly helpful in individuals having compromised immune function. For example, compounds or salts may be used for treating the opportunistic infections and tumors that occur after suppression of cell mediated immunity in, for example, transplant patients, cancer patients and HIV patients.

Thus, one or more of the above diseases or types of diseases, for example, a viral disease or a neoplastic disease may be treated in an animal in need thereof (having the disease) by administering a therapeutically effective amount of a compound or salt of the invention to the animal.

An amount of a compound or salt effective to induce cytokine biosynthesis is an amount sufficient to cause one or more cell types, such as monocytes, macrophages, dendritic cells and B-cells to produce an amount of one or more cytokines such as, for example, IFN-α, TNF-α, IL-1, IL-6, IL-10 and IL-12 that is increased (induced) over a background level of such cytokines. The precise amount will vary according to factors known in the art but is expected to be a dose of about 100 ng/kg to about 50 mg/kg, preferably about 10 μg/kg to about 5 mg/kg. The invention also provides a method of treating a viral infection in an animal and a method of treating a neoplastic disease in an animal comprising administering an effective amount of a compound or salt or composition of the invention to the animal. An amount effective to treat or inhibit a viral infection is an amount that will cause a reduction in one or more of the manifestations of viral infection, such as viral lesions, viral load, rate of virus production, and mortality as compared to untreated control animals. The precise amount that is effective for such treatment will vary according to factors known in the art but is expected to be a dose of about 100 ng/kg to about 50 mg/kg, preferably about 10 μg/kg to about 5 mg/kg. An amount of a compound or salt effective to treat a neoplastic condition is an amount that will cause a reduction in tumor size or in the number of tumor foci. Again, the precise amount will vary according to factors known in the art but is expected to be a dose of about 100 ng/kg to about 50 mg/kg, preferably about 10 μg/kg to about 5 mg/kg.

In addition to the formulations and uses described specifically herein, other formulations, uses, and administration devices suitable for compounds of the present invention are described in, for example, International Publication Nos. WO 03/077944 and WO 02/036592, U.S. Pat. No. 6,245,776, and U.S. Publication Nos. 2003/0139364, 2003/185835, 2004/0258698, 2004/0265351, 2004/076633, and 2005/0009858.

### EXAMPLES

Objects and advantages of this invention are further illustrated by the following examples, but the particular materials and amounts thereof recited in these examples, as well as other conditions and details, should not be construed to unduly limit this invention.

In the examples below automated flash chromatography was carried out using a COMBIFLASH system (an automated high-performance flash purification product available from Teledyne Isco, Inc., Lincoln, Nebr., USA), a HORIZON HPFC system (an automated high-performance flash purification product available from Biotage, Inc, Charlottesville, Va., USA) or a combination thereof. For some of these purifications, either a FLASH 40+M silica cartridge or a FLASH 65I silica cartridge (both available from Biotage, Inc, Charlottesville, Va., USA) was used. The eluent used for each purification is given in the example. In some chromatographic separations, the solvent mixture 80/18/2 v/v/v chloroform/methanol/concentrated ammonium hydroxide (CMA) was used as the polar component of the eluent. In these separations, CMA was mixed with chloroform in the indicated ratio.

### Example 1

### N-{[4-Amino-1-(2-methylpropyl)-1H-imidazo[4,5-c]quinolin-2-yl]methyl}cyclopropanecarboxamide

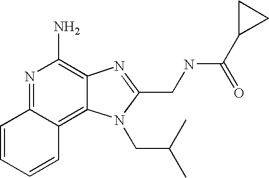 Part A

N⁴-(2-Methylpropyl)quinoline-3,4-diamine (41 g, 0.190 mol, U.S. Pat. No. 5,389,640 Example 1), dichloromethane (550 mL), triethylamine (40 mL, 0.286 mol), and chloroacetyl chloride (16.7 mL, 0.210 mol) were combined and then stirred at ambient temperature over the weekend. The reaction mixture was diluted with 1,2-dichloroethane (75 mL) and then washed with saturated aqueous sodium bicarbonate (3×400 mL). The organic layer was dried over magnesium sulfate, filtered through a layer of CELITE filter agent, and then concentrated under reduced pressure to provide 52.81 g of 2-(chloromethyl)-1-(2-methylpropyl)-1H-imidazo[4,5-c]quinoline as a brown solid.

Part B

3-Chloroperoxybenzoic acid (mCPBA) (32.7 g of 77% pure material, 146 mmol) was added over a period of five minutes to a solution of 2-(chloromethyl)-1-(2-methylpropyl)-1H-imidazo[4,5-c]quinoline (20.0 g, 73.1 mmol) in chloroform (500 mL); the reaction mixture was stirred at ambient temperature for one hour. Ammonium hydroxide (200 mL) was added, and then p-toluenesulfonyl chloride (16.7 g, 87.7 mmol) was added in portions over a period of 10 minutes. The reaction mixture was stirred at ambient temperature for one hour, and then water (200 mL) was added. The aqueous layer was separated and extracted with dichloromethane (2×200 mL). The combined organic fractions were dried over magnesium sulfate, filtered, and concentrated under reduced pressure to provide 32 g of crude product as a tan solid. The crude product was dissolved in dichloromethane (50 mL), and the resulting solution was divided into two portions. Each portion was purified by automated flash chromatography on a HORIZON HPFC system using a FLASH 651 silica cartridge (eluting with ethyl acetate:methanol in a gradient from 98:2 to 85:15) to provide 11.24 g of 2-(chloromethyl)-1-(2-methylpropyl)-1H-imidazo[4,5-c]quinolin-4-amine as a tan solid.

Part C

Potassium phthalimide (6.3 g, 34 mmol) was added to a solution of 2-(chloromethyl)-1-(2-methylpropyl)-1H-imidazo[4,5-c]quinolin-4-amine (8.2 g, 28 mmol) in N,N-dimethylformamide (DMF, 30 mL); a precipitate formed. The reaction mixture was stirred at ambient temperature overnight, and then water (300 mL) was added. The resulting mixture was stirred for 15 minutes, and the precipitate was isolated by filtration, washed with water, and dried overnight in a vacuum oven at 65° C. to provide 9.71 g of 2-{[4-amino-1-(2-methylpropyl)-1H-imidazo[4,5-c]quinolin-2-yl]methyl}-1H-isoindole-1,3(2H)-dione.

Part D

Hydrazine (1.14 mL, 36.4 mmol) was added to a stirred suspension of 2-{[4-amino-1-(2-methylpropyl)-1H-imidazo[4,5-c]quinolin-2-yl]methyl)}-1H-isoindole-1,3(2H)-dione (9.7 g, 24 mmol) in ethanol (200 mL). After 2.5 hours at ambient temperature, an analysis by liquid chromatography/mass spectrometry (LC/MS) indicated the presence of starting material. The reaction mixture was filtered to remove a precipitate, and the filter cake washed with dichloromethane. The filtrate was concentrated under reduced pressure, dissolved in methanol:dichloromethane, and purified by automated flash chromatography on a HORIZON HPFC system using a FLASH 40+M cartridge (eluting with chloroform:2 M ammonia in methanol in a gradient from 95:5 to 85:15) to provide 5.05 g of 2-(aminomethyl)-1-(2-methylpropyl)-1H-imidazo[4,5-c]quinolin-4-amine as a yellow solid.

Part E

Cyclopropanecarbonyl chloride (342 mg, 3.27 mmol) was added to a solution of 2-(aminomethyl)-1-(2-methylpropyl)-1H-imidazo[4,5-c]quinolin-4-amine (780 mg, 2.97 mmol) and triethylamine (0.62 mL, 4.5 mmol) in DMF. The solution was stirred overnight at room temperature and then was concentrated under reduced pressure. The resulting residue was dissolved in a minimal amount of methanol, and acetonitrile was added until the solution became cloudy. Upon heating the mixture, a solution formed which was allowed to stand at room temperature for two days, during which time crystals grew. The crystals were isolated by filtration and were further purified by automated flash chromatography (silica gel cartridge, gradient elution with 1% concentrated ammonium hydroxide in methanol/dichloromethane) followed by recrystallization to yield 292 mg of N-{[4-amino-1-(2-methylpropyl)-1H-imidazo[4,5-c]quinolin-2-yl)methyl]cyclopropanecarboxamide, mp 215.0-216.0° C.

Anal. calcd for C₁₉H₂₃N₅O.0.5H₂O: C, 65.87; H, 6.98; N, 20.22. Found: C, 66.20; H, 6.96; N, 20.43.

### Examples 2-20

An acid chloride (0.09 mmol, 0.9 equivalents) from the table below was added to a test tube containing 2-(aminomethyl)-1-(2-methylpropyl)-1H-imidazo[4,5-c]quinolin-4-amine (27 mg, 0.10 mmol) and N,N-diisopropylethylamine (0.022 mL, 0.12 mmol) in DMF (2 mL). The test tubes were capped and shaken overnight at ambient temperature. One drop of water was added to each test tube, and the solvent was removed by vacuum centrifugation.

The compounds were purified by reversed phase preparative high-performance liquid chromatography (prep HPLC) using a Waters Fraction Lynx automated purification system. The prep HPLC fractions were analyzed using a Micromass LC/TOF-MS, and the appropriate fractions were centrifuge evaporated to provide the trifluoroacetate salt of the desired compound. Column: Zorbax BonusRP, 21.2×50 millimeters (mm), 5 micron particle size; non-linear gradient elution from 5-95% B where A is 0.05% trifluoroacetic acid/water and B is 0.05% trifluoroacetic acid/acetonitrile; fraction collection by mass-selective triggering. The table below shows the acid chloride used for each example, the structure of the resulting compound, and the observed accurate mass for the isolated trifluoroacetate salt.

### Examples 2-20

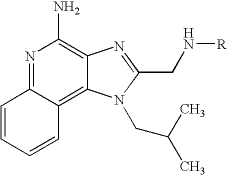

| Ex- | | | Measured |
|---|---|---|---|
| am- | | | Mass |
| ple | Acid Chloride | R | (M + H) |
| 2 | Pentanoyl chloride | 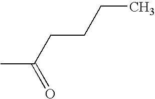 | 354.2322 |
| 3 | Cyclopentanecarbonyl chloride | 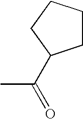 | 366.2312 |
| 4 | tert-Butylacetyl chloride | 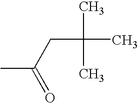 | 368.2466 |
| 5 | Cyclohexanecarbonyl chloride | 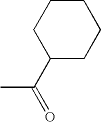 | 380.2469 |
| 6 | m-Toluoyl chloride | 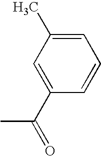 | 388.2126 |
| 7 | Phenylacetyl chloride | 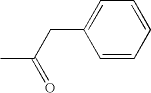 | 388.2156 |
| 8 | 2-Fluorobenzoyl chloride | 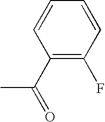 | 392.1890 |
| 9 | 2-Thiopheneacetyl chloride | 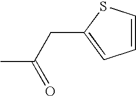 | 394.1731 |
| 10 | 3-Cyclopentylpropionyl chloride | 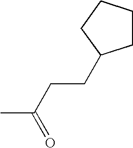 | 394.2634 |
| 12 | Hydrocinnamoyl chloride | 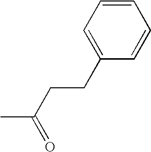 | 402.2294 |
| 13 | 3-Chlorobenzoyl chloride | 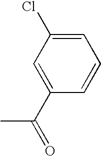 | 408.1622 |
| 14 | 4-Chlorobenzoyl chloride | 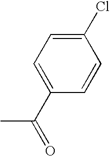 | 408.1606 |
| 15 | Isonicotinoyl chloride hydrochloride | 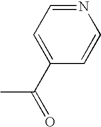 | 375.1950 |
| 16 | Nicotinoyl chloride hydrochloride | 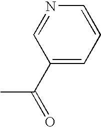 | 375.1955 |
| 17 | 2-Naphthoyl chloride | 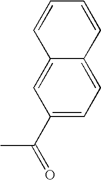 | 424.2176 |
| 18 | 3- (Trifluoromethyl)benzoyl chloride | 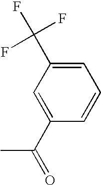 | 442.1872 |
| 19 | 4- (Trifluoromethyl)benzoyl chloride | 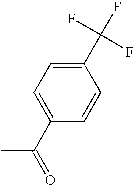 | 442.1868 |
| 20 | 4-(Tri- fluoromethoxy)benzoyl chloride | 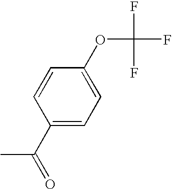 | 458.1815 |

### Examples 21-22

Part A

Triethylamine (49.0 mL, 0.350 mol) was added to a stirred suspension of 1-[(3-aminoquinolin-4-yl)amino]-2-methylpropan-2-ol (0.233 mol) in dichloromethane (0.5 L). A solution of chloroacetyl chloride (21.0 mL, 0.257 mol) in dichloromethane (50 mL) was added dropwise to the mixture at room temperature. The mixture was stirred for 2.5 days at room temperature. The resulting solution was concentrated under reduced pressure, and the residue was partitioned between ethyl acetate (0.5 L) and a solution of 1:1 saturated aqueous sodium bicarbonate/water (0.5 L). The aqueous layer was extracted with ethyl acetate (3×250 mL) and chloroform (250 mL). The combined organic layers were dried over magnesium sulfate, filtered, and concentrated to yield a light brown solid. The solid was dissolved in dichloromethane (80 mL), and crystals formed over a one hour period. The crystals were isolated by filtration, washed with dichloromethane, and dried under vacuum to afford 25.7 g of 1-[2-(chloromethyl)-1H-imidazo[4,5-c]quinolin-1-yl]-2-methylpropan-2-ol as pale yellow crystals. A second crop of crystals (3.56 g) was isolated from the mother liquor. The mother liquor was concentrated and purified by automated flash chromatography using a HORIZON HPFC system (silica cartridge, gradient elution with 3-13% methanol/ethyl acetate) to yield an additional 15.48 g of product. The total amount of 1-[2-(chloromethyl)-1H-imidazo[4,5-c]quinolin-1-yl]-2-methylpropan-2-ol isolated was 44.74 g.

Part B

mCPBA (77% pure, 24 g, 107 mmol) was added in portions to a solution of 1-[2-(chloromethyl)-1H-imidazo[4,5-c]quinolin-1-yl]-2-methylpropan-2-ol (15.5 g, 53.5 mmol) in dichloromethane (500 mL), and the reaction was stirred for 1.5 hours at room temperature. Ammonium hydroxide (200 mL) was added to the reaction mixture and stirred for 5 minutes. p-Toluenesulfonyl chloride (12.2 g, 64.2 mmol) was added over 5 minutes to the reaction mixture, which was stirred at room temperature for two hours. The phases were separated and the aqueous phase was sequentially extracted with dichloromethane (2×200 mL) and 25% methanol in dichloromethane (1×200 mL). The combined organic fractions were dried over magnesium sulfate, filtered, and concentrated under reduced pressure to afford 20 g of 1-[4-amino-2-(chloromethyl)-1H-imidazo[4,5-c]quinolin-1-yl]-2-methylpropan-2-ol as a brown solid.

Part C

Potassium phthalimide (1.46 g, 7.87 mmol) was added to a solution of 1-[4-amino-2-(chloromethyl)-1H-imidazo[4,5-c]quinolin-1-yl]-2-methylpropan-2-ol (2.0 g, 6.56 mmol) in DMF (15 mL). The reaction mixture was stirred at room temperature for two days. Water (150 mL) was added to the mixture, which was stirred for an additional 10 minutes. The solid material was filtered, washed with water, and dried overnight in a vacuum oven to afford 1.2 g of 2-{[4-amino-1-(2-hydroxy-2-methylpropyl)-1H-imidazo[4,5-c]quinolin-2-yl]methyl}-1H-isoindole-1,3(2H)-dione as a tan solid.

Part D

Hydrazine (0.181 mL, 5.78 mmol) was added to a solution of 2-{[4-amino-1-(2-hydroxy-2-methylpropyl)-1H-imidazo[4,5-c]quinolin-2-yl]methyl}-1H-isoindole-1,3(2H)-dione (1.2 g, 2.9 mmol) in ethanol (20 mL), and the reaction was stirred at room temperature for two hours. A precipitate formed and was filtered and the filter cake was washed with dichloromethane. The filtrate was concentrated to 1.2 g of brown solid. The solid was dissolved in dichloromethane and methanol and added onto silica gel. The material was purified by automated flash chromatography using a HORIZON HPFC system (silica cartridge, eluting with 10-20% (2 N ammonia in methanol) in chloroform) and concentrated under reduced pressure to afford 0.370 g of 1-[4-amino-2-(aminomethyl)-1H-imidazo[4,5-c]quinolin-1-yl]-2-methylpropan-2-ol as a light yellow solid.

Part E

An acid chloride (0.11 mmol, 1.1 equivalents) from the table below was added to a test tube containing 1-[4-amino-2-(aminomethyl)-1H-imidazo[4,5-c]quinolin-1-yl]-2-methylpropan-2-ol (28 mg, 0.10 mmol) and N,N-diisopropylethylamine (0.020 mL, 0.12 mmol) in chloroform (1 mL). The test tubes were capped and shaken overnight at room temperature. The solvent was removed by vacuum centrifugation.

The compounds were purified by prep HPLC using the method described for Examples 2-20. The table below shows the acid chloride used for each example, the structure of the resulting compound, and the observed accurate mass for the isolated trifluoroacetate salt.

### Examples 21-22

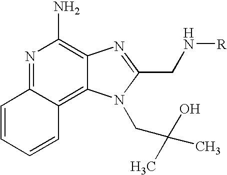

| | | | Measured Mass |
|---|---|---|---|
| Example | Reagent | R | (M + H) |
| 21 | Cyclopropanecarbonyl chloride | 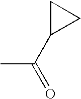 | 354.1935 |
| 22 | Benzoyl chloride | 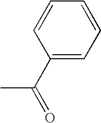 | 390.1942 |

### Example 23

### N-{[4-Amino-1-(2-methylpropyl)-6,7-dimethyl-1H-imidazo[4,5-c]pyridin-2-yl]methyl}cyclopropanecarboxamide

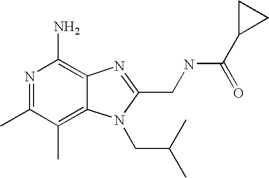 Part A

Ethyl chloroacetimidate hydrochloride (60 g, 380 mmol), prepared according to the procedure of Stillings, M. R. et al., J. Med. Chem., 29, pp. 2280-2284, (1986), was added to a solution of 5,6-dimethyl-N⁴-(2-methylpropyl)-2-phenoxypyridine-3,4-diamine (36.08 g, 126.4 mmol, see the methods in the examples of U.S. Pat. No. 6,743,920) in chloroform (520 mL), and the reaction was stirred at 60° C. overnight, allowed to cool to ambient temperature, and diluted with chloroform (400 mL). The resulting solution was washed with brine (2×500 mL), dried over magnesium sulfate, filtered through a layer of CELITE filter agent, and concentrated under reduced pressure to provide 53.17 g of a dark brown oil. The oil was purified in two portions by column chromatography on silica gel (eluting with dichloromethane:methanol in a gradient from 99.5:0.5 to 98:2) to provide 18.10 g of 2-(chloromethyl)-6,7-dimethyl-1-(2-methylpropyl)-4-phenoxy-1H-imidazo[4,5-c]pyridine as a light pink solid.

Part B

A solution of 2-(chloromethyl)-6,7-dimethyl-1-(2-methylpropyl)-4-phenoxy-1H-imidazo[4,5-c]pyridine (8.51 g, 24.7 mmol) and ammonia (300 mL of 7 N solution in methanol) was heated in a high-pressure vessel overnight at 150° C., allowed to cool to ambient temperature, and concentrated under reduced pressure to provide 9.05 g of a dark brown solid. The solid was mixed with 10.53 g of material from another run and purified by column chromatography on silica gel (eluting with dichloromethane:methanol:ammonium hydroxide in a gradient from 89.1:9.9:1 to 85.1:13.9:1) to provide 6.39 g of 2-(aminomethyl)-6,7-dimethyl-1-(2-methylpropyl)-1H-imidazo[4,5-c]pyridin-4-amine as a brown solid.

Part C

Cyclopropanecarbonyl chloride (0.83 mL, 9.1 mmol) was added to a solution of 2-(aminomethyl)-1-(2-methylpropyl)-6,7-dimethyl-1H-imidazo[4,5-c]pyridin-4-amine (1.50 g, 6.06 mmol) and triethylamine (1.70 mL, 12.2 mmol) in dichloromethane (25 mL) at room temperature. After one hour, the solution was diluted with dichloromethane (25 mL) and washed with brine (4×40 mL). The organic phase was dried over magnesium sulfate, filtered, and concentrated under reduced pressure. The crude product was purified by flash chromatography (silica gel, elution with 1:4:95 concentrated ammonium hydroxide/methanol/dichloromethane) to provide 1.13 g of N-{[4-amino-1-(2-methylpropyl)-6,7-dimethyl-1H-imidazo[4,5-c]pyridin-2-yl]methyl}cyclopropanecarboxamide as a pale brown solid, mp 182-184° C.

Anal. calcd for C₁₇H₂₅N₅O.0.75 CH₂Cl₂: C, 56.23; H, 7.05; N, 18.47. Found: C, 55.91; H, 6.98; N, 18.54.

### Examples 24-32

A reagent (0.12 mmol, 1.2 equivalents) from the table below was added to a test tube containing 2-(aminomethyl)-6,7-dimethyl-1-(2-methylpropyl)-1H-imidazo[4,5-c]pyridin-4-amine (24.3 mg, 0.098 mmol) and N,N-diisopropylethylamine (0.057 mL, 0.33 mmol) in DMF (1 mL). The test tube was capped and shaken overnight at room temperature, and then the solvent was removed by vacuum centrifugation. The compounds were purified by prep HPLC according to the method described in Examples 2-20. The table below shows the reagent added to each test tube, the structure of the resulting compound, and the observed accurate mass for the isolated trifluoroacetate salt.

### Examples 24-32

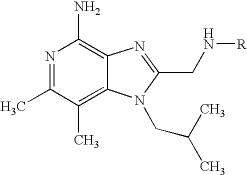

| | | | Measured |
|---|---|---|---|
| | | | Mass |
| Example | Reagent | R | (M + H) |
| 24 | Cyclopropanecarbonyl chloride | 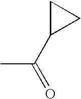 | 316.2146 |
| 25 | Benzoyl chloride | 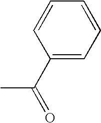 | 352.2143 |
| 26 | m-Toluoyl chloride | 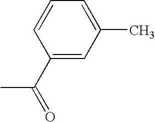 | 366.2321 |
| 27 | 3-Chlorobenzoyl chloride | 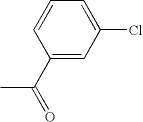 | 386.1750 |
| 28 | 4-Chlorobenzoyl chloride | 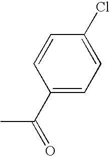 | 386.1742 |
| 29 | Isonicotinoyl chloride hydrochloride | 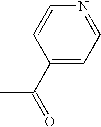 | 353.2100 |
| 30 | trans-2-Phenyl-1- cyclopropanecarbonyl chloride | 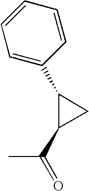 | 392.2415 |
| 31 | 2-Naphthoyl chloride | 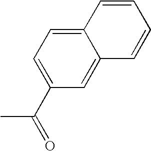 | 402.2303 |
| 32 | 3,4-Dichlorobenzoyl chloride | 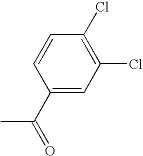 | 420.1374 |

### Examples 33-42

Part A

A solution of N⁴-(2-methylpropyl)[1,5]naphthyridine-3,4-diamine (approximately 15 g, 70 mmol, U.S. Pat. No. 6,194,425 Example 30, Part A), dichloromethane (280 mL) was cooled to 0° C.; chloroacetyl chloride (6.1 mL, 77 mmol) was added dropwise over a period of ten minutes. The reaction was allowed to warm to ambient temperature, stirred for two hours, and concentrated under reduced pressure to provide 2-chloro-N-(2-methylpropylamino)-([1,5]naphthyridin-3-yl)acetamide hydrochloride as a pale-yellow solid.

Part B

Aqueous potassium carbonate (17.5 mL of 6 M, 105 mmol) was added to a solution of the material from Part A in 3:1 ethanol:water (280 mL); the reaction was stirred for three days and concentrated under reduced pressure. The residue was partitioned between dichloromethane (200 mL) and brine (100 mL). The aqueous layer was separated and extracted with dichloromethane (2×50 mL). The combined organic fractions were dried over magnesium sulfate, filtered, and concentrated under reduced pressure to provide 19.5 g of 2-(chloromethyl)-1-(2-methylpropyl)-1H-imidazo[4,5-c][1,5]naphthyridine as a brown solid containing a small amount of dichloromethane.

Part C

mCPBA (5.38 g of 77% pure material, 31.2 mmol) was added to a solution of 2-(chloromethyl)-1-(2-methylpropyl)-1H-imidazo[4,5-c][1,5]naphthyridine (3.0 g, 11 mmol) in chloroform (45 mL); the reaction mixture was stirred at room temperature for one hour. An analysis by LC/MS indicated the reaction was incomplete, and additional mCPBA (1.8 g) was added. The reaction was stirred for one hour and diluted with dichloromethane (150 mL) and saturated aqueous sodium bicarbonate (75 mL). The organic layer was separated and washed with saturated aqueous sodium bicarbonate (75 mL). The combined aqueous fractions were extracted with dichloromethane (2×30 mL), and the combined organic layers were dried over magnesium sulfate, filtered, and concentrated under reduced pressure to provide 2-(chloromethyl)-1-(2-methylpropyl)-5-oxido-1H-imidazo[4,5-c][1,5]naphthyridine as an orange semi-solid.

Part D

A solution of the material from Part C in methanol (40 mL) was cooled to 0° C., and ammonium hydroxide (3.6 mL of 15 M) was added. Benzenesulfonyl chloride (2.9 mL, 23 mmol) was added dropwise over a period of ten minutes, and the reaction was stirred at 0° C. for one hour and then concentrated under reduced pressure. The residue was partitioned between dichloromethane (120 mL) and saturated aqueous sodium bicarbonate (80 mL). The aqueous layer was extracted with dichloromethane (2×25 mL), and the combined organic fractions were dried over magnesium sulfate, filtered, and concentrated under reduced pressure. The resulting brown solid was triturated with chloroform, isolated by filtration, and purified by automated flash chromatography on a HORIZON HPFC system using a FLASH 40+M cartridge (eluting with chloroform:CMA in a gradient from 100:0 to 75:25) to provide 1.82 g of 2-(chloromethyl)-1-(2-methylpropyl)-1H-imidazo[4,5-c][1,5]-naphthyridin-4-amine as a yellow solid.

Part E

Potassium phthalimide (1.40 g, 7.54 mmol) was added to a solution of 2-(chloromethyl)-1-(2-methylpropyl)-1H-imidazo[4,5-c][1,5]-naphthyridin-4-amine (1.82 g, 6.28 mmol) in DMF (50 mL). The reaction mixture was stirred at room temperature for three hours, and a white precipitate formed. The DMF was removed under reduced pressure, and the residue was triturated with methanol, isolated by filtration, and dried under high vacuum to provide 1.51 g of 2-{[4-amino-1-(2-methylpropyl)-1H-imidazo[4,5-c][1,5]naphthyridin-2-yl]methyl}-1H-isoindole-1,3(2H)-dione.

Part F

Hydrazine (0.59 mL, 19 mmol) was added to a stirred suspension of 2-{[4-amino-1-(2-methylpropyl)-1H-imidazo[4,5-c][1,5]naphthyridin-2-yl]methyl}-1H-isoindole-1,3(2H)-dione (1.51 g, 3.77 mmol) in ethanol (60 mL). After four hours at room temperature, an analysis by HPLC indicated the presence of starting material. Additional hydrazine (0.3 mL) was added, and the reaction was stirred at ambient temperature overnight. The ethanol was removed under reduced pressure, and the residue was sonicated in hydrochloric acid (30 mL of 1 M) for 15 minutes. The resulting mixture was filtered to remove a solid, which washed with water. The filtrate was adjusted to pH 7 with the addition of solid sodium bicarbonate. A white precipitate formed and was isolated by filtration, washed with water, and dried for three hours in a vacuum oven at 60° C. to provide 1.02 g of 2-(aminomethyl)-1-(2-methylpropyl)-1H-imidazo[4,5-c][1,5]naphthyridin-4-amine.

HRMS (EI) calcd for C₁₄H₁₈N₆: 270.1593, found: 271.1661

Part G

An acid chloride (0.11 mmol, 1.1 equivalents) from the table below was added to a test tube containing 2-(aminomethyl)-1-(2-methylpropyl)-1H-imidazo[4,5-c][1,5]naphthyridin-4-amine (27 mg, 0.10 mmol) and N,N-diisopropylethylamine (0.035 mL, 0.20 mmol) in DMF (1 mL). The test tube was capped and shaken overnight at room temperature. Two drops of water were added to each test tube, and the solvent was removed by vacuum centrifugation.

The compounds were purified by prep HPLC according to the method described in Examples 2-20. The table below shows the acid chloride added to each test tube, the structure of the resulting compound, and the observed accurate mass for the isolated trifluoroacetate salt.

### Examples 33-42

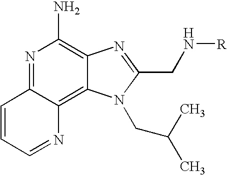

| Ex- | | | Measured |
|---|---|---|---|
| am- | | | Mass |
| ple | Reagent | R | (M + H) |
| 33 | Butyryl chloride | 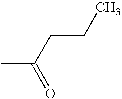 | 341.2084 |
| 34 | Methoxyacetyl chloride | 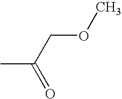 | 343.1883 |
| 35 | Cyclobutanecarbonyl chloride | 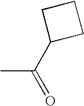 | 353.2097 |
| 36 | Benzoyl chloride | 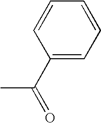 | 375.1942 |
| 37 | Hydrocinnamoylchloride | 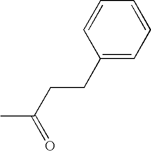 | 403.2233 |
| 38 | 3-Methoxybenzoyl chloride | 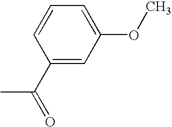 | 405.2038 |
| 39 | 3-Chlorobenzoyl chloride | 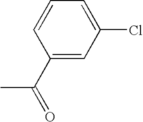 | 409.1538 |
| 40 | 4-chlorobenzoyl chloride | 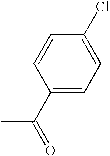 | 409.1538 |
| 41 | Isonicotinoyl chloride hydrochloride | 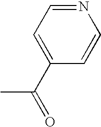 | 376.1855 |
| 42 | 3,4-dichlorobenoyl chloride | 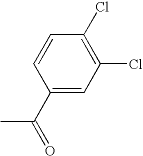 | 443.1140 |

### Examples 43-91

A phenol (1.1 eq) from the table below was added to a test tube containing 2-(chloromethyl)-1-(2-methylpropyl)-1H-imidazo[4,5-c]quinolin-4-amine (29 mg, 0.1 mmol, 1.0 eq) and potassium carbonate (41 mg) in DMF (1.5 mL). The test tubes were capped and shaken overnight at 60° C. Each reaction mixture was then filtered and concentrated by vacuum centrifugation. The compounds were purified by prep HPLC according to the method described in Examples 2-20. The table below shows the phenol added to each test tube, the structure of the resulting compound, and the observed accurate mass for the isolated product.

### Examples 43-91

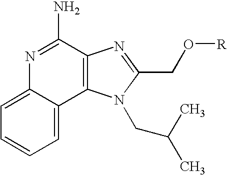

| Ex- | | | Measured |
|---|---|---|---|
| am- | | | Mass |
| ple | Phenol | R | (M + H) |
| 43 | 2′-Hydroxy- acetophenone | 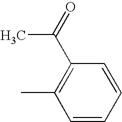 | 389.1983 |
| 44 | Ethyl salicylate | 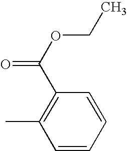 | 419.2094 |
| 45 | 3-Hydroxy-4- methoxybenzoic acid | 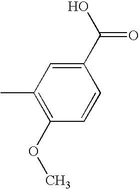 | 421.1892 |
| 46 | Phenol | 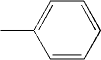 | 347.1891 |
| 47 | m-Cresol | 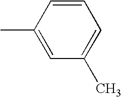 | 361.2062 |
| 48 | o-Cresol | 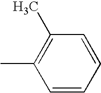 | 361.2046 |
| 49 | p-Cresol | 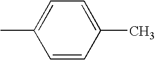 | 361.2014 |
| 50 | 2-Fluorophenol | 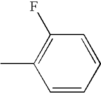 | 365.1803 |
| 51 | 3-Fluorophenol | 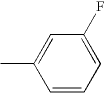 | 365.1772 |
| 52 | 4-Fluorophenol | 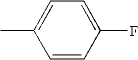 | 365.1767 |
| 53 | 2-Cyanophenol | 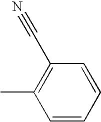 | 372.1809 |
| 54 | 4-Cyanophenol | 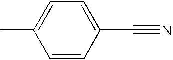 | 372.1859 |
| 55 | 2,3- Dimethylphenol | 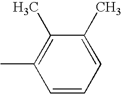 | 375.2184 |
| 56 | 2,4- Dimethylphenol | 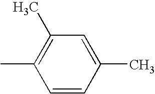 | 375.2153 |
| 57 | 2,5- Dimethylphenol | 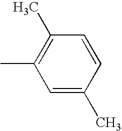 | 375.2211 |
| 58 | 3,4- Dimethylphenol | 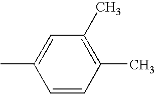 | 375.2217 |
| 59 | 3-Methoxyphenol | 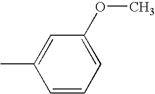 | 377.1962 |
| 60 | 4-Methoxyphenol | 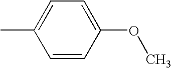 | 377.2007 |
| 61 | Guaiacol | 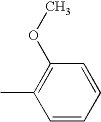 | 377.2003 |
| 62 | 2-Chlorophenol | 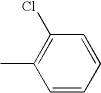 | 381.1504 |
| 63 | 3-Chlorophenol | 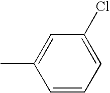 | 381.1484 |
| 64 | 4-Chlorophenol | 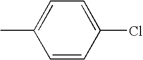 | 381.1503 |
| 65 | 4′-Hydroxy- acetophenone | 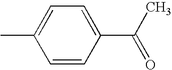 | 389.2003 |
| 66 | 3′-Hydroxy- acetophenone | 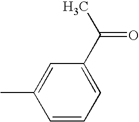 | 389.2014 |
| 67 | 3-(Dimethyl- amino)-phenol | 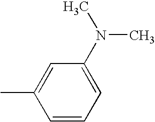 | 390.2290 |
| 68 | 3-Nitrophenol | 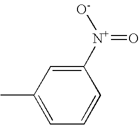 | 392.1685 |
| 69 | 4-Nitrophenol | 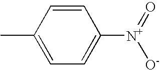 | 392.1729 |
| 70 | 2-Methyl- mercaptophenol | 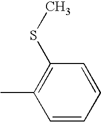 | 393.1764 |
| 71 | 4-Methyl- mercaptophenol | 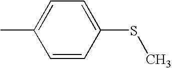 | 393.1756 |
| 72 | 3-tert-Butyl- phenol | 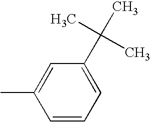 | 403.2463 |
| 73 | 2-Acetamido- phenol | 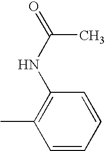 | 404.2085 |
| 74 | 3-Acetamido- phenol | 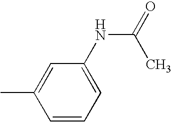 | 404.2125 |
| 75 | 4-Hydroxy- phenylacetamide | 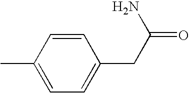 | 404.2118 |
| 76 | 4-[(Dimethyl- amino)methyl]- phenol | 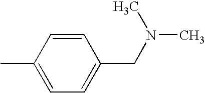 | 404.2457 |
| 77 | Methyl 3- hydroxybenzoate | 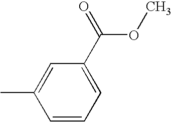 | 405.1938 |
| 78 | Methyl 4- hydroxybenzoate | 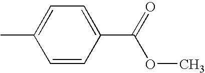 | 405.1959 |
| 79 | Methyl salicylate | 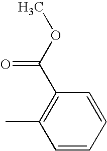 | 405.1944 |
| 80 | 2-Isopropoxy- phenol | 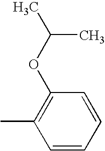 | 405.2286 |
| 81 | 2,3-Dimethoxy- phenol | 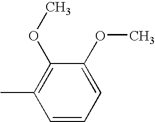 | 407.2101 |
| 82 | 3,4-Dimethoxy- phenol | 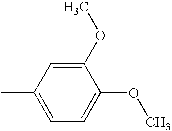 | 407.2114 |
| 83 | 2-Hydroxybenzo- trifluoride | 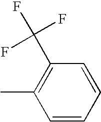 | 415.1759 |
| 84 | 3-Hydroxybenzo- trifluoride | 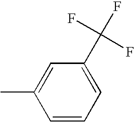 | 415.1777 |
| 85 | 4-Hydroxybenzo- trifluoride | 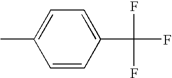 | 415.1752 |
| 86 | 2,4-Dichloro- phenol | 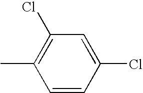 | 415.1125 |
| 87 | 2,5-Dichloro- phenol | 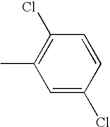 | 415.1128 |
| 88 | 3,4-Dichloro- phenol | 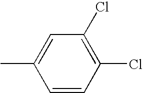 | 415.1124 |
| 89 | 3,5-Dichloro- phenol | 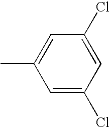 | 415.1130 |
| 90 | 3-Bromophenol | 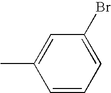 | 425.1002 |
| 91 | 4-Bromophenol | 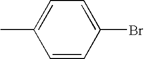 | 425.0992 |

### Examples 92-127

A phenol (1.1 eq) from the table below was added to a test tube containing 1-[4-amino-2-(chloromethyl)-1H-imidazo[4,5-c]quinolin-1-yl]-2-methylpropan-2-ol (30.4 mg, 0.100 mmol, 1.0 eq) and potassium carbonate (55 mg) in DMF (1 mL). The test tubes were capped and shaken overnight at 65° C. Each reaction mixture was then filtered and concentrated by vacuum centrifugation. The compounds were purified by prep HPLC according to the method described in Examples 2-20. The table below shows the phenol added to each test tube, the structure of the resulting compound, and the observed accurate mass for the isolated product.

### Examples 92-127

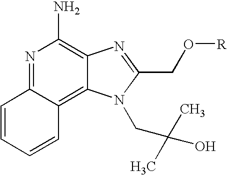

| Ex- | | | Measured |
|---|---|---|---|
| am- | | | Mass |
| ple | Reagent | R | (M + H) |
| 92 | Phenol | 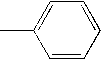 | 363.1811 |
| 93 | m-Cresol | 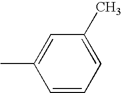 | 377.1981 |
| 94 | o-Cresol | 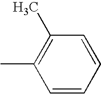 | 377.1955 |
| 95 | p-Cresol | 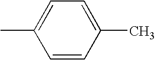 | 377.1993 |
| 96 | 4-Cyanophenol | 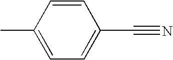 | 388.1760 |
| 97 | 2,3-Dimethylphenol | 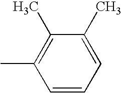 | 391.2125 |
| 98 | 2,4-Dimethylphenol | 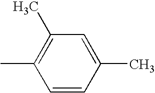 | 391.2136 |
| 99 | 2,5-Dimethylphenol | 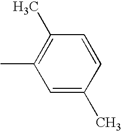 | 391.2154 |
| 100 | 3,4-Dimethylphenol | 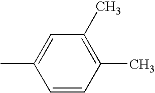 | 391.2146 |
| 101 | 3-Methoxyphenol | 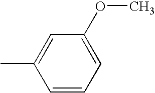 | 393.1947 |
| 102 | Guaiacol | 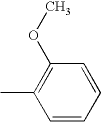 | 393.1936 |
| 103 | 4-Methoxyphenol | 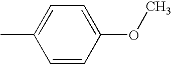 | 393.1933 |
| 104 | 2-Chlorophenol | 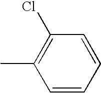 | 397.1440 |
| 105 | 3-Chlorophenol | 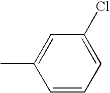 | 397.1433 |
| 106 | 4-Chlorophenol | 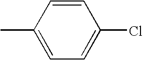 | 397.1410 |
| 107 | 2′-Hydroxy- acetophenone | 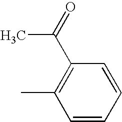 | 405.1937 |
| 108 | 3-(Dimethylamino)- phenol | 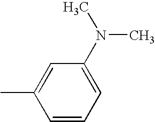 | 406.2235 |
| 109 | 2-Nitrophenol | 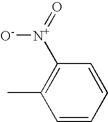 | 408.1704 |
| 110 | 3-Nitrophenol | 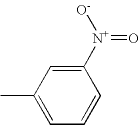 | 408.1697 |
| 111 | 2-Methyl- mercaptophenol | 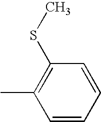 | 409.1705 |
| 112 | 4-Methyl- mercaptophenol | 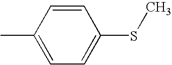 | 409.1697 |
| 113 | 3-tert-Butyl-phenol | 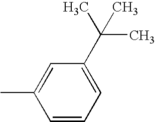 | 419.2454 |
| 114 | 4-Hydroxy- phenylacetamide | 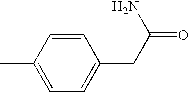 | 420.2050 |
| 115 | 4-Acetamidophenol | 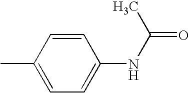 | 420.2045 |
| 116 | 2-Acetamidophenol | 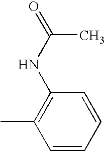 | 420.2048 |
| 117 | 3-Acetamidophenol | 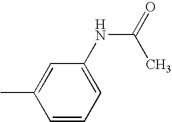 | 420.2032 |
| 118 | 3,4-Dimethoxyphenol | 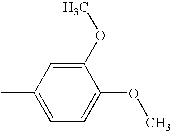 | 423.2042 |
| 119 | 2-Hydroxy- benzotrifluoride | 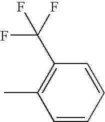 | 431.1713 |
| 120 | 3-Hydroxy- benzotrifluoride | 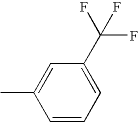 | 431.1715 |
| 121 | 4-Hydroxy- benzotrifluoride | 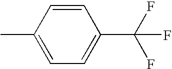 | 431.1700 |
| 122 | 2,3-Dichlorophenol | 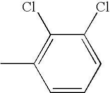 | 431.1061 |
| 123 | 2,4-Dichlorophenol | 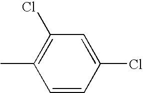 | 431.1064 |
| 124 | 2,5-Dichlorophenol | 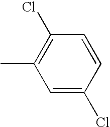 | 431.1038 |
| 125 | 3,4-Dichlorophenol | 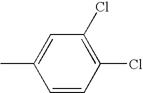 | 431.1081 |
| 126 | 3,5-Dichlorophenol | 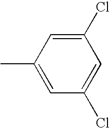 | 431.1059 |
| 127 | 3-Bromophenol | 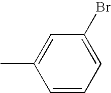 | 441.0928 |

### Examples 128-135

A phenol (1.5 eq) from the table below was added to a test tube containing 2-(chloromethyl)-1-(2-methylpropyl)-1H-imidazo[4,5-c][1,5]naphthyridin-4-amine (28 mg, 0.098 mmol, 1 eq) and potassium carbonate (55 mg, 4 eq) in DMF (1 mL). The test tube was capped and shaken for 19 hours at 65° C. Each reaction mixture was then filtered and concentrated by vacuum centrifugation. The compounds were purified by prep HPLC according to the method described for Examples 2-20. The table below shows the phenol added to each test tube, the structure of the resulting compound, and the observed accurate mass for the isolated product.

### Examples 128-135

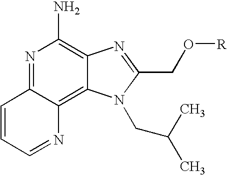

| | | | Measured |
|---|---|---|---|
| Exam- | | | Mass |
| ple | Reagent | R | (M + H) |
| 128 | 2-Fluorophenol | 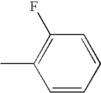 | 366.1731 |
| 129 | 4-Fluorophenol | 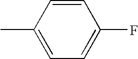 | 366.1739 |
| 130 | Guaiacol | 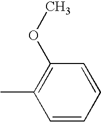 | 378.1918 |
| 131 | 4-Methoxyphenol | 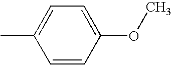 | 378.1914 |
| 132 | 3- Dimethylaminophenol | 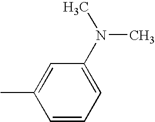 | 391.2238 |
| 133 | 3-tert-Butylphenol | 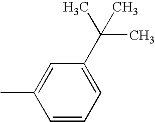 | 404.2462 |
| 134 | 2-Acetamidophenol | 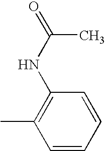 | 405.2068 |
| 135 | 3,4-Dichlorophenol | 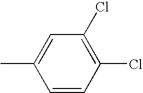 | 416.1049 |

### Example 136

### 2-[(1,1-Dioxidoisothiazolidin-2-yl)methyl]-1-(2-methylpropyl)-1H-imidazo[4,5-c][1,5]naphthyridin-4-amine

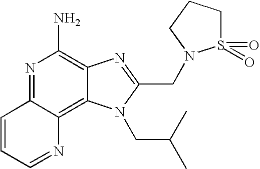

3-Chloropropylsulfonyl chloride (13 μL, 0.11 mmol) was added to a stirred solution of 2-(aminomethyl)-1-(2-methylpropyl)-1H-imidazo[4,5-c][1,5]naphthyridin-4-amine (27 mg, 0.10 mmol) in DMF (2.5 mL) at or slightly above room temperature. After the reaction was stirred at room temperature for two hours, 1,8-diazabicyclo[5.4.0]undec-7-ene (DBU) (37 μL, 0.22 mmol) was added and the solution was stirred overnight. The following morning, more 3-chloropropylsulfonyl chloride (3 μL) was added to the reaction. After four hours, water (0.5 mL) was added and the mixture was concentrated under reduced pressure. The residue was purified by prep HPLC according to the method described in Examples 2-20 to yield 2-[(1,1-dioxidoisothiazolidin-2-yl)methyl]-1-(2-methylpropyl)-1H-imidazo[4,5-c][1,5]naphthyridin-4-amine.

HRMS (EI) calcd for C₁₇H₂₂N₆O₂S: 374.1525, found: 375.1615 (M+H).

### Example 137

### 2-[(1,1-Dioxidoisothiazolidin-2-yl)methyl]-1-(2-methylpropyl)-6,7-dimethyl-1H-imidazo[4,5-c]pyridin-4-amine

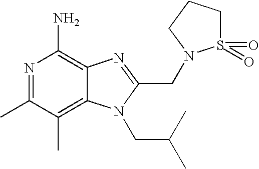

3-Chloropropylsulfonyl chloride (14 μL, 0.11 mmol) was added to a vigorously stirred suspension of 2-(aminomethyl)-1-(2-methylpropyl)-6,7-dimethyl-1H-imidazo[4,5-c]pyridin-4-amine (25 mg, 0.10 mmol) and DBU (38 μL, 0.25 mmol) in DMF (1.0 mL) at room temperature. After two hours, the reaction was concentrated under reduced pressure and was purified by prep HPLC according to the method described in Examples 2-20 to yield 2-[(1,1-dioxidoisothiazolidin-2-yl)methyl]-1-(2-methylpropyl)-6,7-dimethyl-1H-imidazo[4,5-c]pyridin-4-amine.

HRMS (EI) calcd for C₁₇H₂₂N₆O₂S: 351.1729, found: 352.1838 (M+H).

### Example 138-159

Part A

Triethylamine (58.2 g, 575 mmol) and 4-chloro-3-nitroquinoline (80.0 g, 384 mmol) were added to a solution of tert-butyl N-(2-aminoethyl)carbamate (67.6 g, 422 mmol) in DMF (300 mL), and the reaction was stirred overnight at ambient temperature. Water (600 mL) was added, and the resulting mixture was stirred for one hour. A precipitate formed and was isolated by filtration, washed with water (3×150 mL), and dried for two days in a vacuum oven at 45° C. to provide 125.36 g of tert-butyl[2-(3-nitroquinolin-4-ylamino)ethyl]carbamate as a yellow solid.

Part B

A solution of tert-butyl[2-(3-nitroquinolin-4-ylamino)ethyl]carbamate (46.46 g, 139.8 mmol) in ethyl acetate was added to a Parr vessel; 5% platinum on carbon (16.4 g, 84.0 mmol) was added. The vessel was placed under hydrogen pressure (3.0 psi, 2.1×10⁵ Pa) and shaken overnight. The reaction mixture was filtered through a layer of CELITE filter agent, and the filter cake washed with methanol and dichloromethane. The filtrate was concentrated under reduced pressure to provide 40.23 g of tert-butyl 2-[(3-aminoquinolin-4-yl)amino]ethylcarbamate.

Part C

Triethylamine (37.1 mL, 266 mmol) and chloroacetyl chloride (10.6 mL, 133 mmol) were sequentially added to a solution of tert-butyl 2-[(3-aminoquinolin-4-yl)amino]ethylcarbamate (40.23 g, 133 mmol) in dichloromethane (400 mL), and the reaction was stirred at ambient temperature for ten minutes and then concentrated under reduced pressure. The residue was further dried under high vacuum for 30 minutes and then dissolved in ethanol (1 L). The resulting solution was stirred for two days at room temperature and concentrated under reduced pressure. The residue was dissolved in dichloromethane, and the resulting solution washed sequentially with 5% aqueous ammonium chloride and water, dried over magnesium sulfate, filtered, concentrated under reduced pressure, and further dried under high vacuum to provide 50.73 g of tert-butyl 2-[2-(chloromethyl)-1H-imidazo[4,5-c]quinolin-1-yl]ethylcarbamate.

Part D

mCPBA (7.5 g of 77% pure material, 33 mmol) was added to a solution of tert-butyl 2-[2-(chloromethyl)-1H-imidazo[4,5-c]quinolin-1-yl]ethylcarbamate (10.0 g, 27.7 mmol) in chloroform; the reaction mixture was stirred at ambient temperature for one hour. Additional portions of mCPBA were added, and the reaction was stirred until analysis by thin layer chromatography (TLC) indicated that the reaction was complete. Ammonium hydroxide (100 mL) and p-toluenesulfonyl chloride (5.81 g, 30.45 mmol) were sequentially added, and the reaction mixture was stirred vigorously at ambient temperature overnight. The organic layer was separated, washed with ammonium hydroxide, and concentrated under reduced pressure. The crude product was purified by automated flash chromatography (eluting with dichloromethane:methanol:triethylamine in a gradient from 100:0:0 to 95:4.5:0.5) to provide 3.99 g of tert-butyl 2-[4-amino-2-(chloromethyl)-1H-imidazo[4,5-c]quinolin-1-yl]ethylcarbamate.

Part E

A solution of tert-butyl 2-[4-amino-2-(chloromethyl)-1H-imidazo[4,5-c]quinolin-1-yl]ethylcarbamate (3.99 g, 10.6 mmol) and ammonia (50 mL of 7 N solution in methanol) was stirred overnight at room temperature, concentrated under reduced pressure, and further dried under high vacuum to provide 3.49 g of tert-butyl 2-[4-amino-2-(aminomethyl)-1H-imidazo[4,5-c]quinolin-1-yl]ethylcarbamate.

Part F

3-Chloropropylsulfonyl chloride (0.574 mL, 4.72 mmol) was added to a stirred mixture of tert-butyl 2-[4-amino-2-(aminomethyl)-1H-imidazo[4,5-c]quinolin-1-yl]ethylcarbamate (1.40 g, 3.93 mmol) and DBU (2.35 mL, 15.7 mmol) in DMF (20 mL) at room temperature. After five hours, more DBU (2 equivalents) was added and the reaction was stirred overnight. The reaction was concentrated under reduced pressure and was purified by flash chromatography on silica gel to yield 1.63 g of tert-butyl 2-{4-amino-2-[(1,1-dioxidoisothiazolidin-2-yl)methyl]-1H-imidazo[4,5-c]quinolin-1-yl}ethylcarbamate.

Part G

A solution of tert-butyl 2-{4-amino-2-[(1,1-dioxidoisothiazolidin-2-yl)methyl]-1H-imidazo[4,5-c]quinolin-1-yl}ethylcarbamate (1.62 g, 3.5 mmol) in dichloromethane (10 mL) was treated with a solution of 4 M hydrogen chloride in dioxane (10 mL). The reaction was stirred for 19 hours at room temperature, then diethyl ether was added and a precipitate was isolated by filtration. The filter cake washed with a solution of 1:1 dichloromethane/diethyl ether and dried to yield 1.67 g of 1-(2-aminoethyl)-2-[(1,1-dioxidoisothiazolidin-2-yl)methyl]-1H-imidazo[4,5-c]quinolin-4-amine dihydrochloride.

Part H

A reagent (0.11 mmol, 1.1 equivalents) from the table below was added to a test tube containing 1-(2-aminoethyl)-2-[(1,1-dioxidoisothiazolidin-2-yl)methyl]-1H-imidazo[4,5-c]quinolin-4-amine dihydrochloride (43 mg, 0.10 mmol) and N,N-diisopropylethylamine (0.08 mL, 5 eq) in N,N-dimethylacetamide (DMA) (1 mL). The test tube was capped and shaken for four hours at 50° C. Two drops of water were then added to each test tube, and the solvent was removed by vacuum centrifugation.

The compounds were purified by prep HPLC according to the method described in Examples 2-20. The table below shows the reagent added to each test tube, the structure of the resulting compound, and the observed accurate mass for the isolated trifluoroacetate salt.

### Examples 138-159

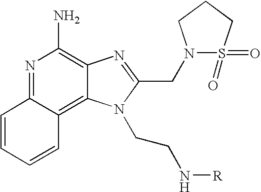

| Ex- | | | Measured |
|---|---|---|---|
| am- | | | Mass |
| ple | Reagent | R | (M + H) |
| 138 | None | 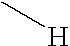 | 361.1450 |
| 139 | Methyl chloroformate | 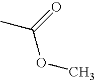 | 419.1503 |
| 140 | Cyclo- propanecarbonyl chloride | 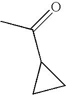 | 429.1715 |
| 141 | Benzoyl chloride | 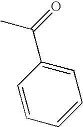 | 465.1696 |
| 142 | Phenylacetyl chloride | 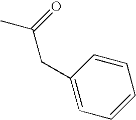 | 479.1861 |
| 143 | 3-Cyanobenzoyl chloride | 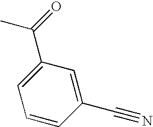 | 490.1615 |
| 144 | 3-Chlorobenzoyl chloride | 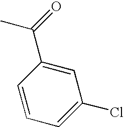 | 499.1333 |
| 145 | 4-Chlorobenzoyl chloride | 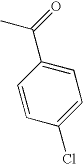 | 499.1299 |
| 146 | Nicotinoyl chloride hydrochloride | 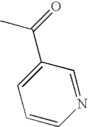 | 466.1645 |
| 147 | trans-2-Phenyl-1- cyclopropane carbonyl chloride | 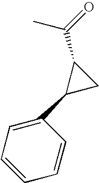 | 505.2004 |
| 148 | 3-Dimethyl- aminobenzoyl chloride | 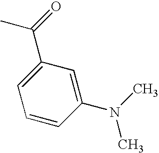 | 508.2096 |
| 149 | Methanesulfonyl chloride | 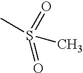 | 439.1212 |
| 150 | Isopropylsulfonyl chloride | 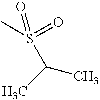 | 467.1521 |
| 151 | Dimethylsulfamoyl chloride | 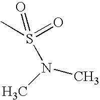 | 468.1494 |
| 152 | 1-Methylimidazole- sulfanoyl chloride | 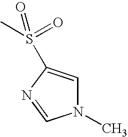 | 505.1407 |
| 153 | α-Toluenesulfonyl chloride | 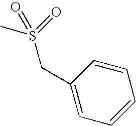 | 515.1484 |
| 154 | trans-Styrene- sulfonyl chloride | 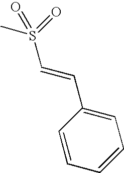 | 527.1498 |
| 155 | 3-Methoxy- benzenesulfonyl chloride | 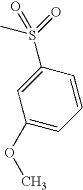 | 531.1448 |
| 156 | 3-Chlorobenzene- sulfonyl chloride | 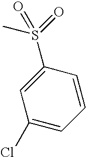 | 535.0987 |
| 157 | Methyl isocyanate | 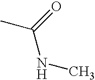 | 418.1646 |
| 158 | Benzyl isocyanate | 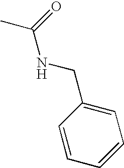 | 494.1951 |
| 159 | trans-2- Phenylcyclopropyl isocyanate | 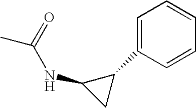 | 520.2121 |

### Example 160

### 1-{4-Amino-2-[(benzyloxy)methyl]-1H-imidazo[4,5-c]quinolin-1-yl}-2-methylpropan-2-ol

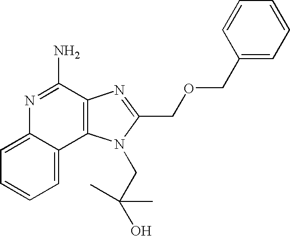 Part A

Benzyloxyacetyl chloride (0.8 g, 4.3 mmol) was added dropwise to a solution of 1-[(3-aminoquinolin-4-yl)amino]-2-methylpropan-2-ol (1.0 g, 4.3 mmol) in acetonitrile (35 mL) at 0° C. A yellow solid formed upon addition and the reaction mixture was warmed to ambient temperature and then heated at reflux overnight. The solid was filtered off, and analysis by proton nuclear magnetic resonance spectroscopy (¹H NMR) indicated the presence of intermediate. The material was dissolved in a 2N methanolic ammonia solution and heated in a sealed vessel overnight at 150° C. The mixture was concentrated under reduced pressure, extracted with dichloromethane, and washed with water. The organic layers were separated, dried over magnesium sulfate, and concentrated under reduced pressure to afford 1.5 g of 1-{2-[(benzyloxy)methyl]-1H-imidazo[4,5-c]quinolin-1-yl}-2-methylpropan-2-ol as an oil.

Part B

A mixture of peracetic acid (0.62 mL, 4.3 mmol), 1-{2-[(benzyloxy)methyl]-1H-imidazo[4,5-c]quinolin-1-yl}-2-methylpropan-2-ol (1.5 g, 4.3 mmol) and methyl acetate (35 mL, 4.3 mmol) were heated at reflux for three hours. An analysis by TLC indicated the reaction was incomplete, so additional peracetic acid (0.3 mL) was added, and the reaction was heated at reflux overnight. The reaction mixture was then concentrated under reduced pressure, dissolved in heptane, and concentrated under reduced pressure again. The product was purified by high performance liquid chromatography on silica gel (eluting with a 95:5 dichloromethane:methanol mixture) to yield 1.4 g of 1-{2-[(benzyloxy)methyl]-5-oxido-1H-imidazo[4,5-c]quinolin-1-yl}-2-methylpropan-2-ol.

Part C

Concentrated ammonium hydroxide (20 mL) was added to a solution of 1-{2-[(benzyloxy)methyl]-5-oxido-1H-imidazo[4,5-c]quinolin-1-yl}-2-methylpropan-2-ol (1.4 g, 3.7 mmol) in dichloromethane (30 mL) and cooled to 0° C. p-Toluenesulfonyl chloride (0.77 g, 4.0 mmol) was added dropwise to the mixture and stirred overnight. The resulting mixture washed with water, dried over magnesium sulfate, and concentrated under reduced pressure. The crude product was recrystallized from an isopropanol/water mixture to provide 1-{4-amino-2-[(benzyloxy)methyl]-1H-imidazo[4,5-c]quinolin-1-yl}-2-methylpropan-2-ol, mp 196-198° C.

Anal. calcd for C₂₂H₂₄N₄O₂: C, 70.19; H, 6.43; N, 14.88. Found: C, 69.74; H, 6.34; N, 14.52.

### Example 161

### 1-(3-Methoxypropyl)-2-(phenoxymethyl)-1H-imidazo[4,5-c]quinolin-4-amine

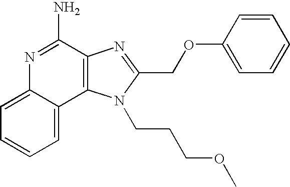 Part A

Phenoxyacetyl chloride (6.15 mL, 44.4 mmol) was added dropwise to a suspension of N⁴-(3-methoxypropyl)quinoline-3,4-diamine (10 g, 43.2 mmol, see U.S. Pat. No. 5,389,640 Example 42) in acetonitrile (150 mL) at 5° C. The reaction was allowed to warm to ambient temperature and stirred for 20 hours. A light yellow solid was isolated by filtration and washed with acetonitrile and dried in an oven.

Part B

The material from Part A was suspended in a methanolic ammonia solution (7.35%, 150 mL) and heated in a sealed reaction vessel at 160° C. for 8 hours and allowed to cool to room temperature overnight. The resulting mixture was concentrated under reduced pressure, made basic with potassium hydroxide and filtered. The filtrate was concentrated under reduced pressure, diluted with water, and the solution was made basic with sodium hydroxide. The product was extracted from the solution with ethyl acetate, dried over magnesium sulfate, and concentrated under reduced pressure to yield 7.1 g of 1-(methoxypropyl)-2-(phenoxymethyl)-1H-imidazo[4,5-c]quinoline as a yellow solid.

Part C

Peracetic acid (1.64 g, 21.6 mmol) was added to a solution of 1-(methoxypropyl)-2-(phenoxymethyl)-1H-imidazo[4,5-c]quinoline (5.0 g, 14.4 mmol) in methyl acetate (100 mL) and heated at reflux for one hour. Analysis by TLC indicated the reaction was incomplete. Additional peracetic acid (1.1 g, 14.4 mmol) was added and the reaction was heated for an additional four hours followed by cooling to room temperature. After addition of small amounts of heptane, the mixture was concentrated under reduced pressure to afford a yellow-orange solid.

Part D

Concentrated ammonium hydroxide was added to the material from Part C in dichloromethane (60 mL) and cooled to 5° C. p-Toluenesulfonyl chloride (3.29 g, 17.3 mmol) was added dropwise to the mixture, which was allowed to warm to room temperature. After 20 hours, the mixture was concentrated under reduced pressure, and the crude product was collected by filtration. The product washed with water and dried under vacuum in an oven to yield 5.2 g of 1-(3-methoxypropyl)-2-(phenoxymethyl)-1H-imidazo[4,5-c]quinolin-4-amine as an orange solid, mp 151.6-152.4° C.

Anal. calcd for C₂₁H₂₂N₄O₂: C, 69.59; H, 6.12; N, 15.46. Found: C, 69.00; H, 6.29; N, 15.31.

### Example 162

### 1-Butyl-2-(phenoxymethyl)-1H-imidazo[4,5-c]quinolin-4-amine

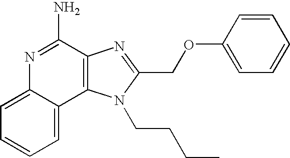 Part A

The general method of Part A of Example 161 was followed using N⁴-butyl-2-chloro-quinoline-3,4-diamine (2.5 g, 10 mmol) in lieu of N⁴-(3-methoxypropyl)quinoline-3,4-diamine as starting material. The reaction time was also limited to three hours after addition of the phenoxyacetyl chloride (1.38 mL, 10 mmol).

Part B

The material from Part A was suspended in a methanolic ammonium solution (7.55%, 30 mL) and heated in a sealed reaction vessel at 160° C. for 18 hours. The suspension was made basic with methanolic potassium hydroxide and the resulting solid was filtered, subsequently washed with methanol and water, and dried. The product was recrystallized from an ethanol/water solution and dried in a vacuum oven to yield 2.3 g of 1-butyl-2-(phenoxymethyl)-1H-imidazo[4,5-c]quinolin-4-amine as an off-white fluffy solid, mp 172.5-173.2° C.

Anal. calcd for C₂₁H₂₂N₄O: C, 72.81; H, 6.40; N, 16.17. Found: C, 73.04; H, 6.41; N, 16.12.

### Example 163

### 1-(2-Methylpropyl)-2-(phenoxymethyl)-1H-imidazo[4,5-c]quinolin-4-amine

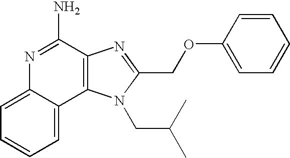

Phenoxyacetyl chloride (5.3 g, 31 mmol) was added dropwise to a stirred solution of 2-chloro-N⁴-(2-methylpropyl)quinoline-3,4-diamine (7.5 g, 30 mmol, see U.S. Pat. No. 4,988,815 Example 4) in acetonitrile (100 mL) cooled to 5° C. The reaction was allowed to warm to room temperature and stirred for an additional 18 hours. The resulting solid was filtered from the mixture, washed with acetonitrile, and dried under vacuum. The solid was then suspended in a methanolic ammonia solution (7.5%, 100 mL) and heated in a sealed reaction vessel at 150-170° C. for 8 hours. The reaction mixture was allowed to cool to room temperature. The solution was heated to reduce the remaining ammonia and made basic with methanolic potassium hydroxide. After cooling to room temperature, the resulting solid was filtered, washed sequentially with methanol and water, and dried in a vacuum oven. The product was then purified by recrystallization from an ethanol/water mixture to yield 9.3 g of 1-(2-methylpropyl)-2-(phenoxymethyl)-1H-imidazo[4,5-c]quinolin-4-amine as a fluffy white powder, mp 196.4-196.8° C.

Anal. calcd for C₂₁H₂₂N₄O: C, 72.81; H, 6.40; N, 16.17. Found: C, 72.57; H, 6.58; N, 16.24.

### Example 164

### 1-[4-Amino-2-(phenoxymethyl)-1H-imidazo[4,5-c]quinolin-1-yl]-2-methylpropan-2-ol

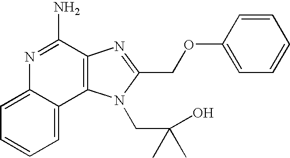

The general method of Example 163 was utilized with 1-(3-amino-2-chloroquinolin-4-ylamino)-2-methylpropan-2-ol (5 g, 19 mmol, see U.S. Pat. No. 4,988,815 Example 13) in lieu of 2-chloro-N⁴-(2-methylpropyl)quinoline-3,4-diamine as the starting material. The product was purified by two recrystallizations from an ethanol/water (90:10) mixture to yield 4.81 g of 1-[4-amino-2-(phenoxymethyl)-1H-imidazo[4,5-c]quinolin-1-yl]-2-methylpropan-2-ol as a tan powder, mp 200.0-201.3° C.

Anal. calcd for C₂₁H₂₂N₄O₂: C, 68.55; H, 6.54; N, 14.53. Found: C, 68.56; H, 14.39; N, 14.39.

### Example 165

### 2-[(4-Chlorophenoxy)methyl]-1-(2-methylpropyl)-1H-imidazo[4,5-c]quinolin-4-amine

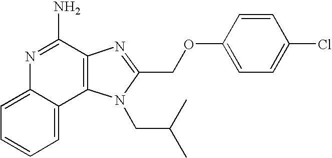

The general procedure of Example 162 was followed with 2-chloro-N⁴-(2-methylpropyl)quinoline-3,4-diamine (2.5 g, 10 mmol) as the starting material in lieu of N⁴-butyl-2-chloroquinoline-3,4-diamine. 4-Chlorophenoxyacetyl chloride (1.56 mL, 10 mmol) was substituted for phenoxyacetyl chloride. The series of reactions yielded 3.0 g of 2-[(4-chlorophenoxy)methyl]-1-(2-methylpropyl)-1H-imidazo[4,5c]quinolin-4-amine as a fluffy off-white solid, mp 209.4-210.0° C.

Anal. calcd for C₂₁H₂₁ClN₄O: C, 66.22; H, 5.55; N, 14.70. Found: C, 65.88; H, 5.25; N, 14.52.

### Example 166

### 1-(2-Methylpropyl)-2-[(4-nitrophenoxy)methyl]-1H-imidazo[4,5-c]quinolin-4-amine

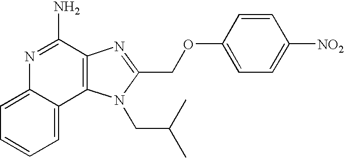

4-Nitrophenoxyacetic acid (9.9 g, 50 mmol) in dichloromethane (100 mL) was treated with a solution of thionyl chloride (8.0 mL, 110 mmol) in DMF (0.5 mL) to produce the acid chloride, which was added to a solution of 2-chloro-N⁴-(2-methylpropyl)quinoline-3,4-diamine (12.5 g, 50 mmol) in acetonitrile (300 mL). After 5 minutes, a solid crystallized from the solution, which in turn was filtered from the mixture, washed with acetonitrile, and dried under vacuum. The solid was then suspended in a methanolic ammonia solution (7.5%, 100 mL) and heated in a sealed reaction vessel at 150° C. for six hours. The reaction mixture was allowed to cool to room temperature. The solution was made basic with methanolic potassium hydroxide and diluted with water. The resulting solid was filtered, washed sequentially with methanol/water and water, and dried in a vacuum oven. The product was then purified by recrystallization from DMF to yield 19.6 g of 1-(2-methylpropyl)-2-[(4-nitrophenoxy)methyl]-1H-imidazo[4,5-c]quinolin-4-amine as a pale yellow solid.

Anal. calcd for C₂₁H₂₁N₅O₃: C, 64.44; H, 5.41; N, 17.89. Found: C, 64.24; H, 5.26; N, 17.89.

### Examples 167-195

Part A

Under a nitrogen atmosphere, a solution of 2-(chloromethyl)-1-(2-methylpropyl)-1H-imidazo[4,5-c]quinolin-4-amine (2.0 g, 6.9 mmol), piperazine (6 g, 70 mmol), and N,N-diisopropylethylamine (1.4 mL, 14 mmol) in acetonitrile (100 mL) was heated at reflux for three hours, cooled to 60° C., and stirred overnight. The solvent was removed under reduced pressure, and the residue was dissolved in chloroform. The resulting solution washed with water (4×100 mL) and concentrated under reduced pressure to provide 1.7 g of 1-(2-methylpropyl)-2-(piperazin-1-ylmethyl)-1H-imidazo[4,5-c]quinolin-4-amine.

Part B

A reagent (0.110-0.120 mmol, 0.11-0.125 equivalents) from the table below was added to a test tube containing 1-(2-methylpropyl)-2-(piperazin-1-ylmethyl)-1H-imidazo[4,5-c]quinolin-4-amine (32.6 mg, 0.096 mmol) and N,N-diisopropylethylamine (0.022 mL, 0.126 mmol) in chloroform (2 mL). The test tube was capped, shaken for four hours at room temperature, and allowed to stand at room temperature overnight. The solvent was removed by vacuum centrifugation, and the compounds were purified by prep HPLC according to the method described in Examples 2-20. The table below shows the reagent added to each test tube, the structure of the resulting compound, and the observed accurate mass for the isolated trifluoroacetate salt.

### Examples 167-195

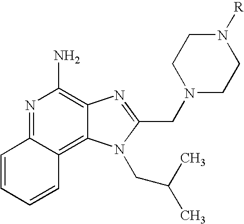

| Ex- | | | Measured |
|---|---|---|---|
| am- | | | Mass |
| ple | Reagent | R | (M + H) |
| 167 | Cyclo- propanecarbonyl chloride | 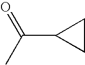 | 407.2552 |
| 168 | Methoxyacetyl chloride | 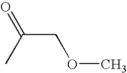 | 411.2531 |
| 169 | Methanesulfonyl chloride | 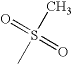 | 417.2093 |
| 170 | Isoxazole-carbonyl chloride | 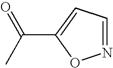 | 434.2309 |
| 171 | Cyclo- pentanecarbonyl chloride | 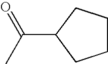 | 435.2860 |
| 172 | tert-Butylacetyl chloride | 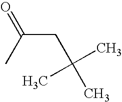 | 437.3029 |
| 174 | Benzoyl chloride | 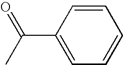 | 443.2533 |
| 175 | 1-Propanesulfonyl chloride | 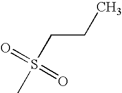 | 445.2381 |
| 176 | 2-Thio- phenecarbonyl chloride | 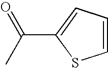 | 449.2130 |
| 177 | Phenylacetyl chloride | 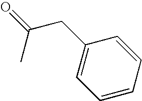 | 457.2708 |
| 178 | 2-Fluorobenzoyl chloride | 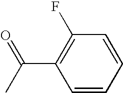 | 461.2472 |
| 179 | 3-Fluorobenzoyl chloride | 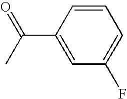 | 461.2491 |
| 180 | 4-Fluorobenzoyl chloride | 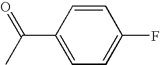 | 461.2462 |
| 181 | 2-Thio- pheneacetyl chloride | 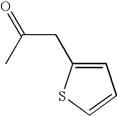 | 463.2294 |
| 183 | Hydrocinnamoyl chloride | 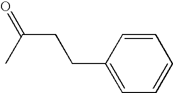 | 471.2837 |
| 184 | 2-Methoxybenzoyl chloride | 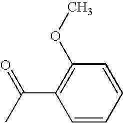 | 473.2694 |
| 185 | 3-Methoxybenzoyl chloride | 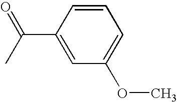 | 473.2674 |
| 186 | 4-Methoxybenzoyl chloride | 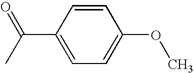 | 473.2678 |
| 187 | 2-Chlorobenzoyl chloride | 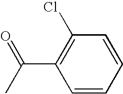 | 477.2143 |
| 188 | 3-Chlorobenzoyl chloride | 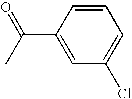 | 477.2160 |
| 189 | 4-Chlorobenzoyl chloride | 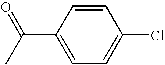 | 477.2176 |
| 190 | Benzenesulfonyl chloride | 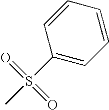 | 479.2216 |
| 191 | α-Toluenesulfonyl chloride | 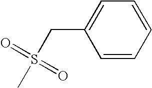 | 493.2357 |
| 192 | 3,5-Dimethyl- isoxazole-4- sulfonyl chloride | 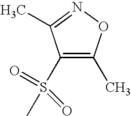 | 498.2284 |
| 193 | 2-Cyano- benzenesulfonyl chloride | 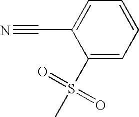 | 504.2170 |
| 194 | 3-Cyano- benzenesulfonyl chloride | 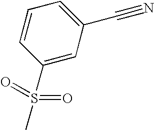 | 504.2162 |
| 195 | 4-Cyano- benzenesulfonyl chloride | 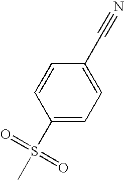 | 504.2165 |

### Examples 196-210

The procedure for Examples 43-91 was followed using a cyclic amine (1.1 equivalents) in lieu of a phenol.

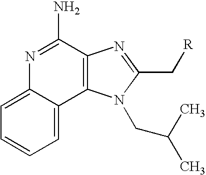

| Ex- | | | Measured |
|---|---|---|---|
| am- | | | Mass |
| ple | Cyclic Amine | R | (M + H) |
| 196 | 2-Methyl- piperazine | 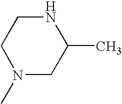 | 353.2457 |
| 197 | 2,6-Dimethyl- piperazine | 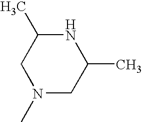 | 367.2618 |
| 198 | 1-Phenyl- piperazine | 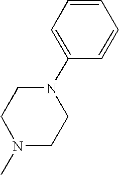 | 415.2613 |
| 199 | 1-Benzyl- piperazine | 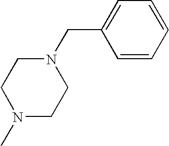 | 429.2775 |
| 200 | Ethyl nipecotate | 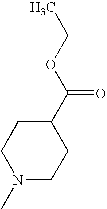 | 410.2577 |
| 201 | 4-Methyl- piperidine | 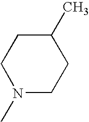 | 352.2513 |
| 202 | 1-(2-Pyridyl)- piperazine | 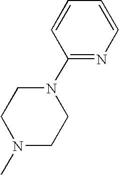 | 416.2570 |
| 203 | 1-(2,5-Di- methylphenyl)- piperazine | 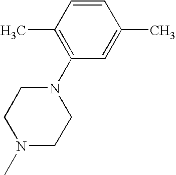 | 443.2953 |
| 204 | 1-(Cinnamyl)- piperazine | 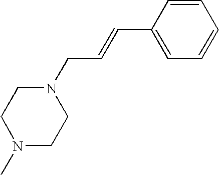 | 455.2934 |
| 205 | 1-(2,3-Di- chloro- phenyl) piperazine | 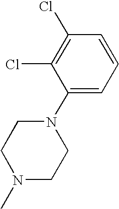 | 483.1840 |
| 206 | 1-(2-Furoyl)- piperazine | 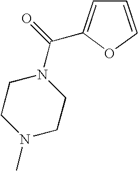 | 433.2368 |
| 207 | 1-(2-Fluoro- phenyl)- piperazine | 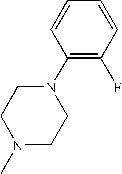 | 433.2511 |
| 208 | Ethyl 1- piperazine- acetate | 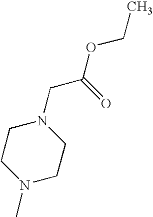 | 425.2672 |
| 209 | 1-(4-Chloro- phenyl)- piperazine | 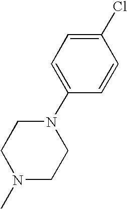 | 449.2225 |
| 210 | 1-Acetyl- piperazine | 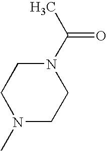 | 381.2416 |

### Examples 211-224

A cyclic amine (0.12 mmol, 1.0 eq.) from the table below was added to a test tube containing 2-(chloromethyl)-1-(2-methylpropyl)-1H-imidazo[4,5-c]quinolin-4-amine (35 mg, 0.12 mmol, 1.0 eq) and potassium carbonate (51 mg) in DMF (1 mL). The test tubes were capped and shaken overnight at 60° C. Each reaction mixture was then filtered and concentrated by vacuum centrifugation. The compounds were purified by prep HPLC according to the method described in Example 2-20. The table below shows the cyclic amine added to each test tube, the structure of the resulting compound, and the observed accurate mass for the isolated product.

### Examples 211-224

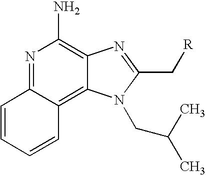

| | | | Measured |
|---|---|---|---|
| | | | Mass |
| Example | Reagent | R | (M + H) |
| 211 | (R)-3-hydroxypyrrolidine | 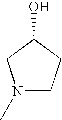 | 340.2125 |
| 212 | 1-Methylpiperazine | 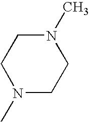 | 353.2444 |
| 213 | 3-Hydroxypiperidine | 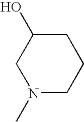 | 354.2288 |
| 214 | 4-hydroxypiperidine | 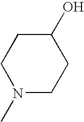 | 354.2310 |
| 215 | L-Prolinol | 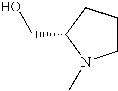 | 354.2295 |
| 216 | 2-Piperidinemethanol | 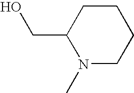 | 368.2468 |
| 217 | 3-(Hydroxymethyl)- piperidine | 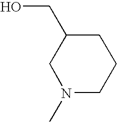 | 368.2470 |
| 218 | 4-(Hydroxymethyl)- piperidine | 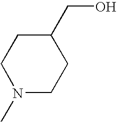 | 368.2459 |
| 219 | Nipecotamide | 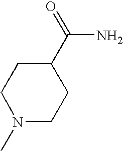 | 381.2431 |
| 220 | Isonipecotamide | 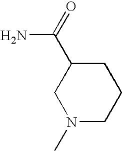 | 381.2382 |
| 221 | Nipecotic acid | 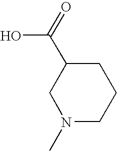 | 382.2252 |
| 222 | 2-Piperidine ethanol | 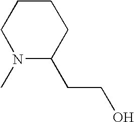 | 382.2604 |
| 223 | 2-Piperazin-1-ylethanol | 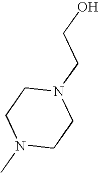 | 383.2576 |
| 224 | 4-(1-Pyrrolidinyl)- piperidine | 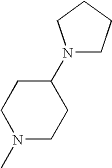 | 407.2938 |

### Examples 226-249

The procedure described in Examples 92-127 was followed using a cyclic amine in lieu of a phenol.

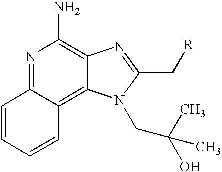

| Ex- | | | Measured |
|---|---|---|---|
| am- | | | Mass |
| ple | Reagent | R | (M + H) |
| 226 | (R)-3-hydroxy- pyrrolidine | 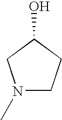 | 356.2081 |
| 227 | 1-Methylpiperazine | 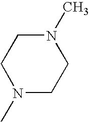 | 369.2381 |
| 228 | 4-Hydroxypiperidine | 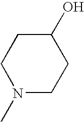 | 370.2252 |
| 229 | 3-Hydroxypiperidine | 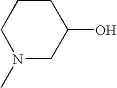 | 370.2233 |
| 230 | L-Prolinol | 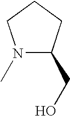 | 370.2242 |
| 231 | 3,5-Dimethyl- piperidine | 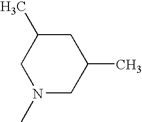 | 382.2604 |
| 232 | 1-Methylhomo- piperazine | 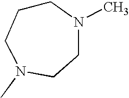 | 383.2564 |
| 233 | D-Proline | 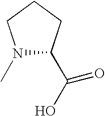 | 384.2058 |
| 234 | L-Proline | 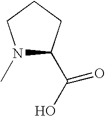 | 384.2011 |
| 235 | 2-Hydroxymethyl- piperidine | 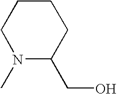 | 384.2408 |
| 236 | 3-Hydroxymethyl- piperidine | 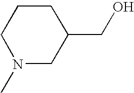 | 384.2393 |
| 237 | 4-Hydroxymethyl- piperidine | 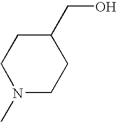 | 384.2403 |
| 238 | (R)-3-Acetamido- pyrrolidine | 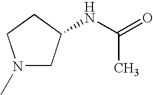 | 397.2344 |
| 239 | 1-Acetylpiperazine | 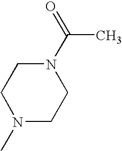 | 397.2357 |
| 240 | Nipecotamide | 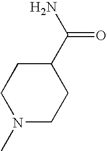 | 397.2337 |
| 241 | Isonipecotamide | 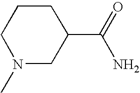 | 397.2350 |
| 242 | 2-Piperidine-ethanol | 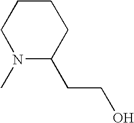 | 398.2551 |
| 243 | 4-(1-Pyrrolidinyl)- piperidine | 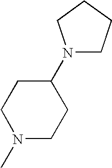 | 423.2884 |
| 244 | 1-(2-Ethoxyethyl)- piperazine | 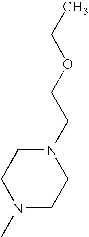 | 427.2801 |
| 245 | 1-Phenylpiperazine | 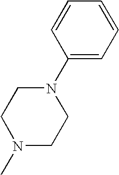 | 431.2545 |
| 246 | 1-Cyclohexyl- piperazine | 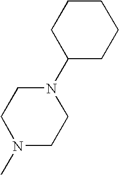 | 437.3032 |
| 247 | 1-(2-Furoyl)piperazine | 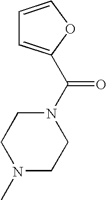 | 449.2282 |
| 248 | N,N-Diethyl- nipecotamide | 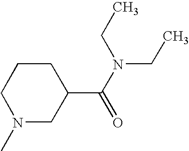 | 453.2981 |

### Examples 250-262

A reagent (0.1 mmol, 1.0 eq) from the table below was added to a test tube containing 1-(2-methylpropyl)-2-(piperazin-1-ylmethyl)-1H-imidazo[4,5-c]quinolin-4-amine (32 mg, 0.1 mmol, 1.0 eq) and triethylamine (0.016 mL, 1.25 eq) in chloroform (2 mL). The test tube was capped and shaken overnight at 60° C. Each reaction mixture was then filtered and concentrated by vacuum centrifugation. The compounds were purified as described in Examples 2-20. The table below shows the reagent added to each test tube, the structure of the resulting compound, and the observed accurate mass for the isolated product.

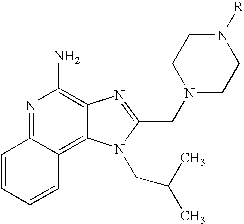

| | | | Measured |
|---|---|---|---|
| Exam- | | | Mass |
| ple | Reagent | R | (M + H) |
| 250 | Glycidyl methyl ether | 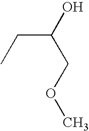 | 427.2812 |
| 252 | Methyl 2- methylglycidate | 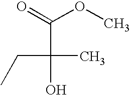 | 455.2774 |
| 253 | Isopropyl glycidyl ether | 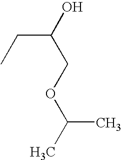 | 455.3112 |
| 254 | Styrene oxide | 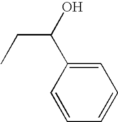 | 459.2868 |
| 257 | Glycidyl phenyl ether | 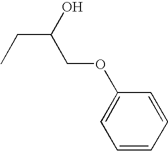 | 489.2968 |
| 258 | R-(+)-3-Chlorostyrene oxide | 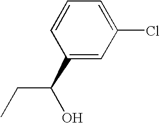 | 493.2466 |
| 259 | o-Cresyl glycidyl ether | 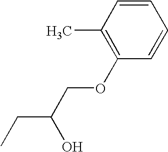 | 503.3127 |
| 261 | 4-(Fluorophenoxy)- methyloxirane | 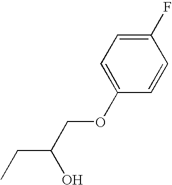 | 507.2886 |
| 262 | 4-Chlorophenyl glycidyl ether | 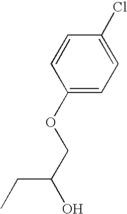 | 523.2546 |

### Examples 263-278

Part A

Catalytic 5% platinum on carbon (154 mg) was added to a mixture of (3-methoxypropyl)-(3-nitroquinolin-4-yl)amine (3.46 g, 13.24 mmol) and magnesium sulfate (˜1 g) in ethyl acetate (38.4 mL). The reaction was placed under hydrogen pressure in a sealed vessel overnight. Analysis by ¹H NMR indicated the reaction was incomplete. Additional catalytic 5% platinum on carbon (154 mg) was added and the reaction mixture was placed on the hydrogenator for an additional night. The reaction mixture was filtered through a layer of CELITE filter aid, and the filter cake washed with ethyl acetate. The filtrate was concentrated under reduced pressure to yield 2.89 g of N⁴-(3-methoxypropyl)quinoline-3,4-diamine as a brown solid.

Part B

Triethylamine (4.1 mL, 29.4 mmol) and chloroacetyl chloride (1.22 mL, 15.3 mmol) were added sequentially to a solution of the material from Part A in dichloromethane (41 mL) and stirred for 72 hours at room temperature. The reaction mixture was diluted with dichloromethane (102 mL), washed with saturated aqueous sodium bicarbonate (2×41 mL), dried over magnesium sulfate, filtered, and concentrated under reduced pressure to afford 2.92 g of 2-(chloromethyl)-1-(3-methoxypropyl)-1H-imidazo[4,5-c]quinoline as a brown solid.

Part C

mCPBA (77% pure, 2.67 g, 12 mmol) was added to a solution of 2-(2-chloromethyl)-(3-methoxypropyl)-1H-imidazo[4,5-c]quinoline (2.92 g, 10.3 mmol, 1 eq) in chloroform (30 mL), and the reaction was stirred for 2 hours at ambient temperature. The reaction mixture was diluted with dichloromethane (25 mL), washed with saturated aqueous sodium bicarbonate (3×75 mL), dried over magnesium sulfate, filtered, and concentrated under reduced pressure to afford 2.09 g of 2-(chloromethyl)-1-(3-methoxypropyl)-5-oxido-1H-imidazo[4,5-c]quinoline.

Part D

Ammonium hydroxide (15 mL) and p-toluenesulfonyl chloride (1.57 g, 8.22 mmol, 1.2 eq) were sequentially added to a mixture of 2-(chloromethyl)-1-(3-methoxypropyl)-5-oxido-1H-imidazo[4,5-c]quinoline (2.09 g, 6.85 mmol) in 1,2-dichloroethane (30 mL) cooled to 5° C. The reaction mixture was allowed to warm to room temperature and stirred for one hour. The reaction mixture was then diluted with dichloromethane (25 mL), washed with saturated aqueous sodium bicarbonate (3×75 mL), dried over magnesium sulfate, filtered, and concentrated under reduced pressure to afford 2.01 g of 2-(chloromethyl)-1-(3-methoxypropyl)-1H-imidazo[4,5-c]quinolin-4-amine as a brown solid.

Part E

A reagent (1.1 eq) from the table below was added to a test tube containing 2-(chloromethyl)-1-(3-methoxypropyl)-1H-imidazo[4,5-c]quinolin-4-amine (29 mg, 0.1 mmol, 1.0 eq) and potassium carbonate (41 mg) in DMF (1.5 mL). The test tube was capped and shaken overnight at 60° C. Each reaction mixture was then filtered and concentrated by vacuum centrifugation. The compounds were purified by prep HPLC according to the method described in Examples 2-20. The table below shows the reagent added to each test tube, the structure of the resulting compound, and the observed accurate mass for the isolated product.

### Examples 263-278

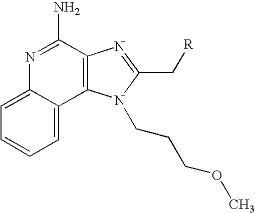

| | | | Measured |
|---|---|---|---|
| | | | Mass |
| Example | Reagent | R | (M + H) |
| 263 | 2-Methylpiperazine | 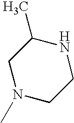 | 369.2376 |
| 264 | 2,6- Dimethylpiperzine | 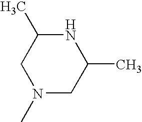 | 383.2550 |
| 265 | 1-Phenylpiperazine | 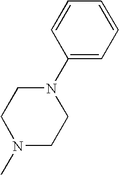 | 431.2551 |
| 266 | 1-Methylpiperazine | 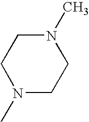 | 369.2403 |
| 268 | 2,6- Dimethylmorpholine | 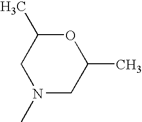 | 384.2414 |
| 269 | 4-Methylpiperidine | 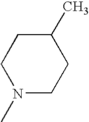 | 368.2463 |
| 270 | 4-Benzylpiperidine | 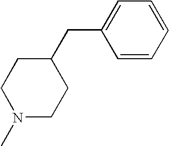 | 444.2762 |
| 271 | 1-(2-Pyridyl)- piperazine | 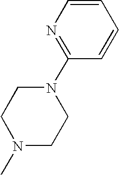 | 432.2539 |
| 272 | 1-(2,5- Dimethylphenyl)- piperazine | 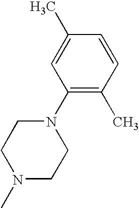 | 459.2867 |
| 274 | 1-(2,3- Dichlorophenyl)- piperazine | 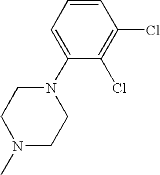 | 499.1763 |
| 275 | 1-(2-Furoyl)- piperazine | 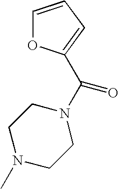 | 449.2314 |
| 276 | 1-(2-Fluorophenyl)- piperazine | 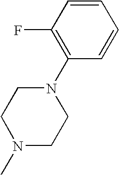 | 449.2462 |
| 277 | 1-(4-Chlorophenyl)- piperazine | 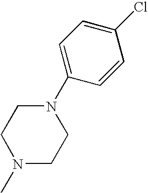 | 465.2175 |
| 278 | 1-Acetylpiperazine | 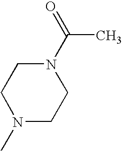 | 397.2354 |

### Example 279-285

A cyclic amine (0.25 mmol) from the table below was added to a test tube containing 2-(chloromethyl)-6,7-dimethyl-1-(2-methylpropyl)-4-phenoxy-1H-imidazo[4,5-c]pyridine (65 mg, 0.25 mmol, 1.0 eq) and potassium carbonate (80 mg) in DMF (2.5 mL). The test tube was capped and shaken overnight at 60° C. Each reaction mixture was then filtered and concentrated by vacuum centrifugation. Ammonium acetate (1 g) was added to each test tube, and the test tubes were capped and heated overnight at 150° C. and then allowed to cool to room temperature. The compounds were purified by prep HPLC according to the method described in Examples 2-20. The table below shows the cyclic amine added to each test tube, the structure of the resulting compound, and the observed accurate mass for the isolated product.

### Examples 279-285

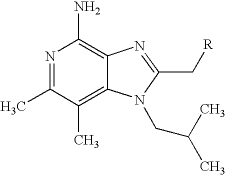

| | | | Measured |
|---|---|---|---|
| | | | Mass |
| Example | Reagent | R | (M + H) |
| 279 | 1-Phenylpiperazine | 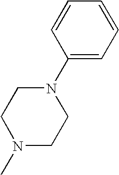 | 393.2790 |
| 280 | 1-Methylpiperazine | 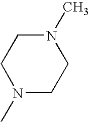 | 331.2604 |
| 281 | 1-Benzylpiperazine | 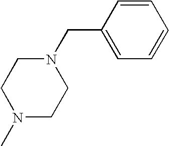 | 407.2941 |
| 282 | 4-Methylpiperidine | 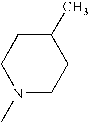 | 330.2672 |
| 283 | 1-Cyclohexyl- piperazine | 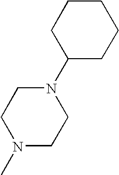 | 399.3256 |
| 284 | 1-(4-Chloro- phenyl)piperazine | 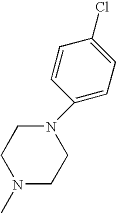 | 427.2370 |
| 285 | 1-Acetylpiperazine | 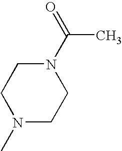 | 359.2584 |

### Examples 286-293

The procedure described in Examples 128-135 was followed using a cyclic amine shown in the table below in lieu of a phenol.

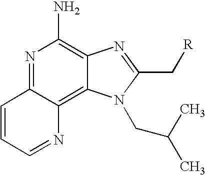

| | | | Measured Mass |
|---|---|---|---|
| Example | Reagent | R | (M + H) |
| 286 | N-Methylpiperazine | 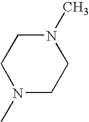 | 354.2379 |
| 287 | L-Prolinol | 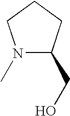 | 355.2214 |
| 288 | 2- Piperidinemethanol | 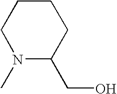 | 369.2421 |
| 289 | 3-(Hydroxymethyl) piperidine | 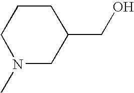 | 369.2412 |
| 290 | 4-(Hydroxymethyl) piperidine | 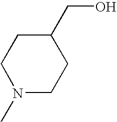 | 369.2407 |
| 291 | Isonipecotamide | 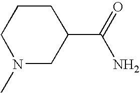 | 382.2392 |
| 292 | 2-Piperidine-ethanol | 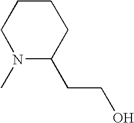 | 383.2561 |
| 293 | 1-(2-pyridyl) Piperazine | 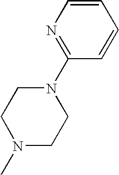 | 417.2521 |

### Example 294

### 1-{[(4R)-2,2-Dimethyl-1,3-dioxolan-4-yl]methyl}-2-(ethoxymethyl)-1H-imidazo[4,5-c]quinolin-4-amine hydrobromide

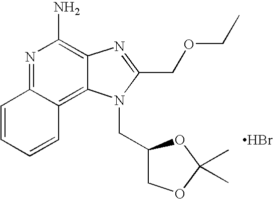 Part A

To a solution of 7-bromo-4-chloro-3-nitroquinoline (22.00 g, 76.52 mmol) in dichloromethane (250 mL) was added triethylamine (16.0 mL, 115 mmol) followed by the dropwise addition of a solution of 4R—(−)-(2,2-dimethyl)-1,3-dioxolane-4-methanamine (11.04 g, 84.16 mmol) in dichloromethane (200 mL). The reaction was monitored by TLC, and after the starting material was consumed, the reaction mixture was transferred to a separatory funnel and washed with water (200 mL) and brine (200 mL), dried over Na₂SO₄, filtered, and concentrated. The resulting yellow residue was triturated with water (200 mL) and the solid was collected by filtration and dried. The solid was sonicated in diethyl ether (100 mL) and isolated by filtration. The solid was dried under vacuum at 40° C. to yield 7-bromo-N-{[(4R)-2,2-dimethyl-1,3-dioxolan-4-yl]methyl}-3-nitroquinolin-4-amine (25.84 g) as a yellow solid, mp 136-137° C.

Part B

To a mechanically stirred mixture of 7-bromo-N-{[(4R)-2,2-dimethyl-1,3-dioxolan-4-yl]methyl}-3-nitroquinolin-4-amine (25.80 g, 67.50 mmol) and ethyl viologen dibromide (0.510 g, 1.35 mmol) in dichloromethane (300 mL) and water (50 mL), was added dropwise a solution of Na₂S₂O₄ (62.21 g, 303.8 mmol) and K₂CO₃ (46.65 g, 337.5 mmol) in water (250 mL). The reaction mixture was stirred at room temperature overnight. Water (300 mL) was added and the mixture was stirred for 10 minutes. The organic layer was separated and the aqueous layer was extracted with dichloromethane (200 mL). The combined organic layers were washed with water (800 mL) and brine (800 mL), dried over Na₂SO₄, filtered and evaporated to give 7-bromo-N⁴-{[(4R)-2,2-dimethyl-1,3-dioxolan-4-yl]methyl}quinoline-3,4-diamine as a brown foam (22.87 g).

Part C

Triethylamine (11.3 mL, 81.2 mmol) followed by ethoxyacetyl chloride (9.94 g, 81.2 mmol) were added to a 0° C. solution of 7-bromo-N⁴-{[(4R)-2,2-dimethyl-1,3-dioxolan-4-yl]methyl}quinoline-3,4-diamine (22.87 g, 64.93 mmol) in dichloromethane (250 mL). The reaction was allowed to warm to room temperature. After 4 hours, the solvent was evaporated under reduced pressure and the residue was dissolved in ethanol (200 mL). Triethylamine (27.2 mL, 195 mmol) was added to the solution and the solution was heated at reflux for 16 hours. The solvent was evaporated under reduced pressure. The residue was extracted with dichloromethane (2×300 mL). The combined organics were washed with water (300 mL) and brine (300 mL), and dried over Na₂SO₄. The crude material was purified by flash chromatography (silica gel, eluting with 5% CMA in chloroform). The appropriate fractions were combined and evaporated under reduced pressure to give a white solid that was crystallized from acetonitrile to yield 7-bromo-1-{[(4R)-2,2-dimethyl-1,3-dioxolan-4-yl]methyl}-2-(ethoxymethyl)-1H-imidazo[4,5-c]quinoline (17.37 g) as a white crystalline solid, mp 90-91° C.

Part D

To a solution of 7-bromo-1-{[(4R)-2,2-dimethyl-1,3-dioxolan-4-yl]methyl}-2-(ethoxymethyl)-1H-imidazo[4,5-c]quinoline (17.37 g, 41.22 mmol) in dichloromethane (175 mL) was added mCPBA (23.1 g, 103 mmol). The mixture was stirred for 2 hours and then was diluted with dichloromethane (150 mL), washed with 4% aqueous Na₂CO₃ (150 mL×2) and brine (150 mL), filtered, and concentrated. To the residue was added dichloromethane (200 mL) and concentrated ammonium hydroxide (80 mL) and the mixture was stirred rapidly and cooled to 4° C. p-Toluenesulfonyl chloride (9.82 g, 51.5 mmol) was added in portions. The mixture was allowed to warm to room temperature and was stirred 16 hours. The mixture was diluted with dichloromethane (200 mL) and washed with 2 M aqueous sodium carbonate (200 mL). The aqueous layer was back-extracted with dichloromethane (100 mL). The combined organic layers were washed with brine, dried over Na₂SO₄, filtered, and concentrated to afford an off-white solid that was purified by flash chromatography (silica gel, gradient elution with 0-10% CMA/chloroform). The appropriate fractions were combined and concentrated to a solid that was crystallized from acetonitrile to yield the product 7-bromo-1-{[(4R)-2,2-dimethyl-1,3-dioxolan-4-yl]methyl}-2-(ethoxymethyl)-1H-imidazo[4,5-c]quinolin-4-amine (7.48 g) as a white solid, mp 176-177° C.

Part E

A mixture of 7-bromo-1-{[(4R)-2,2-dimethyl-1,3-dioxolan-4-yl]methyl}-2-(ethoxymethyl)-1H-imidazo[4,5-c]quinolin-4-amine (2.00 g, 4.59 mmol) and 10% palladium on carbon (0.20 g) in ethyl acetate (200 mL) was hydrogenated on a Parr apparatus at 50 psi (3.5×10⁵ Pa) overnight. The mixture was diluted with methanol (100 mL) and filtered through CELITE filter agent. The filtrate was concentrated to afford a solid that was crystallized from acetonitrile to yield 1-{[(4R)-2,2-dimethyl-1,3-dioxolan-4-yl]methyl}-2-(ethoxymethyl)-1H-imidazo[4,5-c]quinolin-4-amine hydrobromide (1.92 g) as a white crystalline solid, mp 220-222° C. ¹H NMR (300 MHz, CDCl₃) δ 8.14 (d, J=8.0, 1H), 7.94 (d, J=8.1 Hz, 1H), 7.66-7.57 (m, 1H), 7.56-7.47 (m, 1H), 5.03-4.95 (m, 1H), 4.95-4.76 (m, 3H), 4.69-4.58 (m, 1H), 4.27 (dd, J=8.7, 6.4 Hz, 1H), 3.91 (dd, J=8.7, 6.3 Hz, 1H), 3.67 (q, J=7.0 Hz, 2H), 1.44 (s, 3H), 1.29 (t, J=7.0 Hz, 3H), 1.29 (s, 3H); ¹³C-NMR (75 MHz, CDCl₃) δ 152.9, 149.2, 136.3, 133.8, 130.2, 125.4, 124.5, 121.3, 119.9, 112.8, 110.7, 74.5, 67.0, 66.7, 65.0, 49.3, 26.5, 24.9, 15.0; MS (APCI) m/z 357.0 (M+H)⁺;

Anal. calcd for C₁₉H₂₄N₄O₃.HBr: C, 52.18; H, 5.76; N, 12.81; Br, 17.95. Found: C, 52.00; H, 5.64; N, 12.59; Br, 18.39.

### Example 295

### (2R)-3-[4-Amino-2-(ethoxymethyl)-1H-imidazo[4,5-c]quinolin-1-yl]propane-1,2-diol

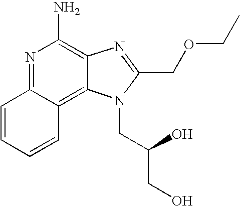

A suspension of 1-{[(4R)-2,2-dimethyl-1,3-dioxolan-4-yl]methyl}-2-(ethoxymethyl)-1H-imidazo[4,5-c]quinolin-4-amine hydrobromide (1.54 g, 3.52 mmol) in tetrahydrofuran (25 mL) was treated with 1 M HCl (25 mL) to give a clear solution that was stirred at ambient temperature overnight. The tetrahydrofuran was removed under reduced pressure and remaining aqueous solution was made basic (pH=8.5-9) with 1 M NaOH. A solid formed that was collected by filtration, rinsed with water, and dried. The solid was purified on a by automated flash chromagraphy on a HORIZON HPFC system (eluting with a CMA/chloroform gradient). The appropriate fractions were combined and evaporated to yield (2R)-3-[4-amino-2-(ethoxymethyl)-1H-imidazo[4,5-c]quinolin-1-yl]propane-1,2-diol (0.65 g) as a white powder, mp 101-102° C. ¹H NMR (300 MHz, CDCl₃) δ 8.21 (d, J=7.9 Hz, 1H), 7.65-7.58 (m, 1H), 7.48-7.38 (m, 1H), 7.27-7.17 (m, 1H), 6.57 (br s, 2H), 5.20 (d, J=5.3 Hz, 1H), 5.10 (t, J=5.5 Hz, 1H), 4.99 (d, J=12.3 Hz, 1H), 4.84 (dd, J=15.2, 2.4 Hz, 1H), 4.65 (d, J=12.3 Hz, 1H), 4.46 (dd, J=15.1, 9.5 Hz, 1H), 4.02-3.88 (m, 1H), 3.63-3.44 (m, 4H), 1.15 (t, J=7.0 Hz, 3H); ¹³C-NMR (75 MHz, CDCl₃) δ 151.9, 149.8, 145.1, 133.1, 126.5, 126.1, 120.9, 120.8, 114.8, 70.2, 65.2, 64.3, 63.6, 48.7, 14.9; MS (APCI) m/z 317.1 (M+H)⁺;

Anal. calcd for C₁₆H₂₀N₄O₃: C, 60.75; H, 6.37; N, 17.71. Found: C, 60.44; H, 6.39; N, 17.50.

### Example 296

### 1-{[(4S)-2,2-Dimethyl-1,3-dioxolan-4-yl]methyl}-2-(ethoxymethyl)-1H-imidazo[4,5-c]quinolin-4-amine

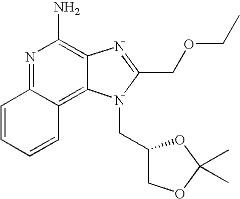 Part A

Following the method described in Part A of Example 294, a solution of 7-bromo-4-chloro-3-nitroquinoline (11.00 g, 38.3 mmol) and triethylamine (8.00 mL, 57.4 mmol) in dichloromethane (100 mL) was treated with 4S-(2,2-dimethyl)-1,3-dioxolane-4-methanamine (5.52 g, 42.1 mmol) to produce 7-bromo-N-{[(4S)-2,2-dimethyl-1,3-dioxolan-4-yl]methyl}-3-nitroquinolin-4-amine as a yellow solid (14.05 g).

Part B

A mixture of a 7-bromo-N-{[(4S)-2,2-dimethyl-1,3-dioxolan-4-yl]methyl}-3-nitroquinolin-4-amine (14.05 g, 36.8 mmol) and ethyl viologen dibromide (0.284 g, 0.735 mmol) in dichloromethane (175 mL) and water (25 mL) was treated with a solution of Na₂S₂O₄ (29.8 g, 195 mmol) and K₂CO₃ (25.4, 184 mmol) in water (150 mL) according to the method described in Part B of Example 294. The product 7-bromo-N⁴-{[(4S)-2,2-dimethyl-1,3-dioxolan-4-yl]methyl}quinoline-3,4-diamine was isolated as a pale brown solid (10.27 g).

Part C

Following the procedure described in Part C of Example 294, ethoxyacetyl chloride (4.14 g, 30.4 mmol) was reacted with 7-bromo-N⁴-{[(4S)-2,2-dimethyl-1,3-dioxolan-4-yl]methyl}quinoline-3,4-diamine (10.72 g, 30.4 mmol) in the presence of triethylamine (4.67 mL, 33.5 mmol) to yield 7-bromo-1-{[(4S)-2,2-dimethyl-1,3-dioxolan-4-yl]methyl}-2-(ethoxymethyl)-1H-imidazo[4,5-c]quinoline (8.88 g) as a white solid, mp 89-90° C.

Part D

Following the procedure described in Part D of Example 294, 7-bromo-1-{[(4S)-2,2-dimethyl-1,3-dioxolan-4-yl]methyl}-2-(ethoxymethyl)-1H-imidazo[4,5-c]quinoline (8.74 g, 20.8 mmol) was treated with mCPBA (9.58 g, 41.6 mmol). The resulting N-oxide was reacted with p-toluenesulfonyl chloride (4.96 g, 26.0 mmol) and excess ammonium hydroxide to yield 7-bromo-1-{[(4S)-2,2-dimethyl-1,3-dioxolan-4-yl]methyl}-2-(ethoxymethyl)-1H-imidazo[4,5-c]quinolin-4-amine (4.28 g) as a white solid, mp 184-185° C.

Part E

Following the procedure described in Part E of Example 294, 7-bromo-1-{[(4S)-2,2-dimethyl-1,3-dioxolan-4-yl]methyl}-2-(ethoxymethyl)-1H-imidazo[4,5-c]quinolin-4-amine (1.50 g, 3.45 mmol) was hydrogenated over 10% palladium on carbon (0.15 g). After the work up described in Part E of Example 294 yielded 1-{[(4S)-2,2-dimethyl-1,3-dioxolan-4-yl]methyl}-2-(ethoxymethyl)-1H-imidazo[4,5-c]quinolin-4-amine hydrobromide that was slightly impure, the product was dissolved in water and treated with NaOH until the pH was approximately 9. The solution was extracted with several portions of chloroform. The combined organic phases were dried over Na₂SO₄, filtered, and concentrated. The material was purified by automated flash chromatography using a HORIZON HPFC system (silica cartridge, gradient elution with CMA/chloroform) followed by crystallization from diethyl ether to give 1-{[(4S)-2,2-dimethyl-1,3-dioxolan-4-yl]methyl}-2-(ethoxymethyl)-1H-imidazo[4,5-c]quinolin-4-amine (0.99 g) as a white crystalline solid, mp 130-131° C. ¹H NMR (300 MHz, DMSO-d₆) δ 8.17 (d, J=7.7 Hz, 1H), 7.62 (dd, J=8.3 Hz, 1.0, 1H), 7.49-7.40 (m, 1H), 7.28-7.19 (m, 1H), 6.62 (br s, 2H), 4.96-4.85 (m, 2H), 4.81-4.68 (m, 2H), 4.60-4.49 (m, 1H), 4.20 (dd, J=8.7, 6.5 Hz, 1H), 3.86 (dd, J=8.7, 6.5 Hz, 1H), 3.58 (q, J=7.1 Hz, 2H), 1.34 (s, 3H), 1.19 (s, 3H), 1.16 (t, J=7.0 Hz, 3H); ¹³C NMR (75 MHz, DMSO-d₆) δ 151.9, 149.3, 145.2, 133.2, 126.8, 126.2, 120.9, 120.8, 114.6, 109.0, 74.5, 66.1, 65.3, 64.3, 47.8, 26.2, 24.9, 14.8. MS (APCI) m/z 357.1 (M+H)⁺;

Anal. calcd for C₁₉H₂₄N₄O₃.0.25H₂O: C, 63.23; H, 6.84; N, 15.52. Found: C, 62.97; H, 6.99; N, 15.50.

### Example 297

### (2S)-3-[4-Amino-2-(ethoxymethyl)-1H-imidazo[4,5-c]quinolin-1-yl]propane-1,2-diol

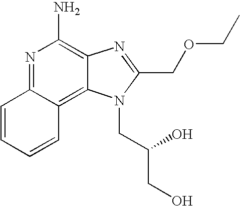

Following the procedure described in Example 295, 1-{[(4S)-2,2-dimethyl-1,3-dioxolan-4-yl]methyl}-2-(ethoxymethyl)-1H-imidazo[4,5-c]quinolin-4-amine (0.59 g, 1.66 mmol) was treated with 1 M HCl (8 mL) in tetrahydrofuran (8 mL) to yield (2S)-3-[4-amino-2-(ethoxymethyl)-1H-imidazo[4,5-c]quinolin-1-yl]propane-1,2-diol as a white solid (0.355 g), mp 99-100° C. ¹H NMR (300 MHz, DMSO-d₆) δ 8.22 (d, J=8.1 Hz, 1H), 7.66-7.58 (m, 1H), 7.48-7.39 (m, 1H), 7.27-7.17 (m, 1H), 6.60 (br s, 2H), 5.20 (d, J=5.2 Hz, 1H), 5.10 (t, J=5.4 Hz, 1H), 4.99 (d, J=12.3 Hz, 1H), 4.84 (dd, J=14.9, 2.4 Hz, 1H), 4.66 (d, J=12.3 Hz, 1H), 4.46 (dd, J=15.1, 9.5 Hz, 1H), 4.03-3.89 (m, 1H), 3.65-3.45 (m, 4H), 1.15 (t, J=7.0 Hz, 3H); ¹³C NMR (75 MHz, CDCl₃) δ 151.9, 149.9, 145.0, 133.2, 126.6, 126.1, 126.0, 120.9, 120.8, 114.8, 70.2, 65.2, 64.3, 63.6, 48.7, 14.9; MS (APCI) m/z 317.1 (M+H)⁺;

Anal. calcd for C₁₆H₂₀N₄O₃.0.25H₂O.0.05 CHCl₃: C, 59.47; H, 6.30; N, 17.29. Found: C, 59.19; H, 6.49; N, 17.37.

### Example 298

### 1-[(2,2-Dimethyl-1,3-dioxolan-4-yl)methyl]-2-(ethoxymethyl)-1H-imidazo[4,5-c]quinolin-4-amine hydrobromide

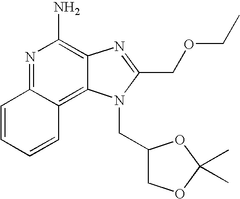 Part A

Following the method described in Part A of example 294, a solution of 7-bromo-4-chloro-3-nitroquinoline (39.85 g, 139 mmol) was reacted with racemic 4-(2,2-dimethyl)-1,3-dioxolane-4-methanamine (20.0 g, 152 mmol) to produce 7-bromo-N-[(2,2-dimethyl-1,3-dioxolan-4-yl)methyl]-3-nitroquinolin-4-amine as a yellow solid (48.4 g).

Part B

Following the method described in Part B of example 294, a mixture of 7-bromo-N-[(2,2-dimethyl-1,3-dioxolan-4-yl)methyl]-3-nitroquinolin-4-amine (48.12 g, 125.9 mmol) and 1,1′-di-N-octyl-4,4′-bipyridinium dibromide (1.37 g, 2.52 mmol) in dichloromethane (500 mL) and water (50 mL) was treated with a solution of Na₂S₂O₄ (116.1 g, 566.6 mmol) and K₂CO₃ (87.00 g, 629.5 mmol) in water (450 mL). The product, 7-bromo-N⁴-[(2,2-dimethyl-1,3-dioxolan-4-yl)methyl]quinoline-3,4-diamine, was isolated as a dark brown solid (40.1 g).

Part C

Following the procedure described in Part C of Example 294, ethoxyacetyl chloride (13.95 g, 113.8 mmol) was reacted with 7-bromo-N⁴-[(2,2-dimethyl-1,3-dioxolan-4-yl)methyl]quinoline-3,4-diamine (40.08 g, 113.8 mmol) to yield 7-bromo-1-[(2,2-dimethyl-1,3-dioxolan-4-yl)methyl]-2-(ethoxymethyl)-1H-imidazo[4,5-c]quinoline (30.23 g) as an off white solid, mp 138-140° C.

Part D

Following the procedure described in Part D of Example 294, 7-bromo-1-[(2,2-dimethyl-1,3-dioxolan-4-yl)methyl]-2-(ethoxymethyl)-1H-imidazo[4,5-c]quinoline (20.00 g, 47.58 mmol) was treated with mCPBA (16.43 g, 71.38 mmol). The resulting N-oxide was reacted with p-toluenesulfonyl chloride (9.98 g, 52.3 mmol) and excess ammonium hydroxide to yield pure 7-bromo-1-[(2,2-dimethyl-1,3-dioxolan-4-yl)methyl]-2-(ethoxymethyl)-1H-imidazo[4,5-c]quinolin-4-amine (9.00 g) as a white solid, mp 174-175° C.

Part E

Following the procedure described in Part E of Example 294, 7-bromo-1-[(2,2-dimethyl-1,3-dioxolan-4-yl)methyl]-2-(ethoxymethyl)-1H-imidazo[4,5-c]quinolin-4-amine (2.00 g, 4.59 mmol) was hydrogenated over 10% palladium on carbon (0.20 g) to yield 1-[(2,2-dimethyl-1,3-dioxolan-4-yl)methyl]-2-(ethoxymethyl)-1H-imidazo[4,5-c]quinolin-4-amine hydrobromide (1.40 g) as a white solid, mp 243-245° C. ¹H NMR (300 MHz, DMSO-d₆) δ 13.33 (s, 1H), 9.84-8.22 (br absorption, 2H), 8.44 (d, J=8.1 Hz, 1H), 7.90-7.82 (m, 1H), 7.80-7.70 (m, 1H), 7.62-7.52 (m, 1H), 5.08-4.82 (m, 2H), 4.97 (d, J=12.9 Hz, 1H), 4.80 (d, J=12.8 Hz, 1H), 4.63-4.51 (m, 1H), 4.25 (dd, J=8.7, 6.5 Hz, 1H), 3.89 (dd, J=8.7, 6.8 Hz, 1H), 3.61 (m, 2H), 1.33 (s, 3H), 1.19 (s, 3H), 1.19 (t, J=7.0 Hz, 3H); ¹³C NMR (75 MHz, DMSO-d₆) δ 152.5, 148.8, 135.8, 134.0, 129.8, 124.6, 124.3, 122.6, 118.6, 112.7, 109.2, 74.3, 66.1, 65.6, 63.9, 48.2, 26.0, 24.9, 14.8; MS (APCI) m/z 357.1 (M+H)⁺;

Anal. calcd for C₁₉H₂₄N₄O₃.HBr: C, 52.18; H, 5.76; N, 12.81. Found: C, 52.31; H, 5.83; N, 12.68.

### Example 299

### 3-[4-Amino-2-(ethoxymethyl)-1H-imidazo[4,5-c]quinolin-1-yl]propane-1,2-diol

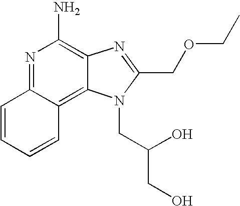

Following the procedure described in Example 295, 1-[(2,2-dimethyl-1,3-dioxolan-4-yl)methyl]-2-(ethoxymethyl)-1H-imidazo[4,5-c]quinolin-4-amine hydrobromide (0.61 g, 1.40 mmol) was treated with 1 M HCl (10 mL) in tetrahydrofuran (10 mL). Chromatographic purification was unnecessary in this case, and 3-[4-amino-2-(ethoxymethyl)-1H-imidazo[4,5-c]quinolin-1-yl]propane-1,2-diol was isolated as a white solid (0.32 g), mp 212-213° C. ¹H NMR (300 MHz, DMSO-d₆) δ 8.22 (d, J=7.9 Hz, 1H), 7.61 (dd, J=8.3, 0.9 Hz, 1H), 7.49-7.39 (m, 1H), 7.28-7.17 (m, 1H), 6.61 (br s, 2H), 5.20 (d, J=5.3 Hz, 1H), 5.10 (t, J=5.4 Hz, 1H), 4.99 (d, J=12.3 Hz, 1H), 4.84 (dd, J=15.0, 2.5 Hz, 1H), 4.65 (d, J=12.3 Hz, 1H), 4.46 (dd, J=15.1, 9.5 Hz, 1H), 4.03-3.89 (m, 1H), 3.64-3.43 (m, 4H), 1.15 (t, J=7.0 Hz, 3H); ¹³C NMR (75 MHz, DMSO-d₆) δ 152.0, 150.0, 145.0, 133.3, 126.7, 126.2, 126.1, 121.0, 120.9, 114.9, 70.3, 65.3, 64.4, 63.7, 48.8, 15.0; MS (APCI) m/z 317.1 (M+H)⁺;

Anal. calcd for C₁₆H₂₀N₄O₃: C, 60.75; H, 6.37; N, 17.7. Found: C, 60.70; H, 6.53; N, 17.48.

### Example 300

### 2-(Ethoxymethyl)-1-[(2-methylisoxazolidin-5-yl)methyl]-1H-imidazo[4,5-c]quinolin-4-amine

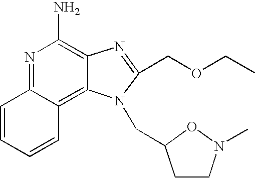 Part A

Triethylamine (17.0 mL, 123 mmol) was added to a 0° C. solution of 2,4-dichloro-3-nitroquinoline (20.0 g, 82.3 mmol) in dichloromethane (350 mL) followed by the dropwise addition of allylamine (5.90 mL, 78.2 mmol). The solution was allowed to stir and warm to room temperature overnight. The solvent was evaporated under reduced pressure and the resulting orange solid was suspended in water (300 mL). Solid sodium carbonate was added to adjust the pH to 10-11 and the suspension was stirred for 2 hours at 0° C. A yellow solid was isolated by filtration and dried under vacuum overnight to yield N-allyl-2-chloro-3-nitroquinolin-4-amine (21.7 g) that contained small amounts of an impurity and water.

Part B

An aqueous solution (200 mL) of potassium carbonate (55.3 g, 400 mmol) and sodium dithionate (62.7 g, 360 mmol) was added dropwise over 30 minutes to a mixture of N-allyl-2-chloro-3-nitroquinolin-4-amine (21.0 g, 79.9 mmol) and ethyl viologen dibromide (1.80 g, 4.80 mmol) in dichloromethane (320 mL) and water (40 mL) under a nitrogen atmosphere. The dark blue-green mixture was stirred rapidly and was heated at reflux overnight. The mixture was transferred to a separatory funnel and the layers were separated. The aqueous layer was extracted with dichloromethane. The combined organic layers were filtered through CELITE filter agent, dried over MgSO₄, filtered, and concentrated to a dark oil. The crude product was purified by suction filter chromatography (silica gel, gradient elution from 3:1 to 1:3 hexanes/ethyl acetate, then 4:1 dichloromethane/ethyl acetate) to afford pure product N⁴-allyl-2-chloroquinoline-3,4-diamine (12.06 g) along with some impure product (3.10 g).

Part C

Ethoxyacetyl chloride (8.80 g, 71.8 mmol) was added dropwise to a solution of N4-allyl-2-chloroquinoline-3,4-diamine (15.2 g, 65.3 mmol) in acetonitrile (300 mL) at room temperature. After 45 min, the reaction mixture was filtered and an orange solid (approximately 17 g) was isolated. The solid was dissolved in a solution of ethanol (240 mL) and water (80 mL). Sodium hydroxide (3.92 g, 98.0 mmol) was added and the solution was heated at reflux for 2 hours. The ethanol was removed under reduced pressure and the remaining aqueous solution was extracted several times with dichloromethane. The combined organic layers were dried over MgSO₄, filtered, and concentrated to an orange solid. The solid was triturated with ethyl acetate and isolated by filtration to yield 6 g of a pale yellow solid. The filtrate was concentrated and purified by suction filter chromatography (silica gel with 97:3 dichloromethane/methanol as the eluent) to yield an additional 5 g of product. The material was combined to provide 11 g of 1-allyl-4-chloro-2-(ethoxymethyl)-1H-imidazo[4,5-c]quinoline.

Part D

A mixture of 1-allyl-4-chloro-2-(ethoxymethyl)-1H-imidazo[4,5-c]quinoline (2.00 g, 6.63 mmol), N-methylhydroxylamine hydrochloride (609 mg, 7.29 mmol), sodium bicarbonate (949 mg, 11.3 mmol), paraformaldehyde (997 mg, 33.2 mmol) and alumina in toluene (100 mL) was heated at reflux for 5 hours. Additional N-methylhydroxylamine hydrochloride (305 mg), sodium bicarbonate (475 mg), and paraformaldehyde (500 mg) were added and the mixture was heated at reflux overnight. The mixture was filtered through CELITE filter agent and concentrated under reduced pressure. The crude product was purified by flash chromatography (silica gel, elution with 97:3 dichloromethane/methanol) to yield 1.40 g of 4-chloro-2-(ethoxymethyl)-1-[(2-methylisoxazolidin-5-yl)methyl]-1H-imidazo[4,5-c]quinoline as tan oil. Some starting material (500 mg) was also recovered.

Part E

A solution of 4-chloro-2-(ethoxymethyl)-1-[(2-methylisoxazolidin-5-yl)methyl]-1H-imidazo[4,5-c]quinoline (340 mg, 0.942 mmol) in a solution of 7 M ammonia in methanol (15 mL) was sealed in a pressure vessel and heated to 150° C. After 10 hours, the vessel was allowed to cool to room temperature, and the volatiles were removed under reduced pressure. The crude product was purified by flash chromatography (silica gel, elution with 97:3 dichloromethane/methanol) to afford 2-(ethoxymethyl)-1-[(2-methylisoxazolidin-5-yl)methyl]-1H-imidazo[4,5-c]quinolin-4-amine (310 mg) as a white solid that was crystallized from acetonitrile to give a white crystalline solid, mp 173-174° C. ¹H NMR (300 MHz, CDCl₃) δ 8.05 (d, J=8.3 Hz, 1H), 7.81 (dt, J=8.3, 0.9 Hz, 1H), 7.51 (dt, J=7.1, 1.2 Hz, 1H), 7.31 (dt, J=7.1, 1.2 Hz, 1H), 5.41 (br s, 2H), 5.07-4.53 (m, 5H), 3.62 (q, J=7.0 Hz, 2H), 3.37 (t, J=8.2 Hz, 1H), 2.65 (s, 3H), 2.61-2.43 (m, 2H), 2.21-2.14 (m, 1H), 1.25 (t, J=7.0 Hz, 3H); MS (APCI) m/z 342 (M+H⁺);

Anal. calcd for C₁₈H₂₃N₅O₂: C, 63.32; H, 6.79; N, 20.51. Found: C, 63.19; H, 6.74; N, 20.71.

### Example 301

### 1-[(2,3-Dimethylisoxazolidin-5-yl)methyl]-2-(ethoxymethyl)-1H-imidazo[4,5-c]quinolin-4-amine

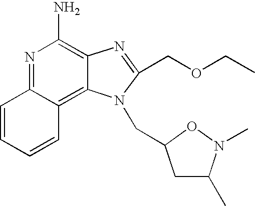 Part A

A slurry of 1-allyl-4-chloro-2-(ethoxymethyl)-1H-imidazo[4,5-c]quinoline (prepared as described in Part C of Example 300, 5.76 g, 19.1 mmol) in a solution of 7 M ammonia in methanol (25 mL) was sealed in a pressure vessel and heated to 150° C. After one day, the volatiles were removed under reduced pressure and the residue was partitioned between saturated aqueous sodium bicarbonate and dichloromethane. The aqueous layer was extracted with two additional portions of dichloromethane. The combined organic layers were washed with brine, dried over MgSO₄, filtered, and concentrated to afford 1-allyl-2-(ethoxymethyl)-1H-imidazo[4,5-c]quinolin-4-amine (3.45 g) as a pale yellow solid.

Part B

α-Methyl-N-methylnitrone was prepared at room temperature from acetaldehyde and methyl hydroxylamine hydrochloride using a modified version of a procedure published by C. M. Dicken and P. DeShong, J. Org. Chem. 47, pp. 2047-2051 (1982) that describes the synthesis of α-phenyl-N-methylnitrone. A slurry of α-methyl-N-methylnitrone (315 mg, 4.25 mmol) and 1-allyl-2-(ethoxymethyl)-1H-imidazo[4,5-c]quinolin-4-amine (1.00 g, 3.54 mmol) in toluene (25 mL) was sealed in a pressure tube and heated at 118° C. for 72 hours. The solution washed with saturated aqueous sodium bicarbonate and brine, dried over MgSO₄, filtered and concentrated. The residue was purified by flash chromatography on silica gel to a white solid. The solid was crystallized from acetonitrile to yield analytically pure 1-[(2,3-dimethylisoxazolidin-5-yl)methyl]-2-(ethoxymethyl)-1H-imidazo[4,5-c]quinolin-4-amine (0.85 g) as a mixture of diastereomers in the form of a white crystalline solid, mp 144-146° C. ¹H NMR (300 MHz, CDCl₃) δ 7.99 (d, J=7.6 Hz, 1H), 7.82 (dd, J=8.4, 1.1 Hz, 1H), 7.52 (dt, J=8.3, 1.3 Hz, 1H), 7.31 (dt, J=7.6, 1.3 Hz, 1H), 5.39 (br s, 2H), 5.07-4.51 (m, 4H), 3.61 (q, J=7.0 Hz, 2H), 2.55 (s, 3H), 2.29-2.23 (m, 1H), 2.14 (m, 1H), 1.65 (m, 2H), 1.25 (t, J=7.0 Hz, 3H), 1.12 (d, J=6.2 Hz, 3H); MS (APCI) m/z 356 (M+H⁺);

Anal. calcd for C₁₉H₂₅N₅O₂: C, 64.20; H, 7.09; N, 19.70. Found: C, 64.10; H, 6.98; N, 20.01.

The ¹H NMR data provided were obtained from the major diastereomer.

### Example 302

### 2-(Ethoxymethyl)-1-[(2-methyl-3-phenylisoxazolidin-5-yl)methyl]-1H-imidazo[4,5-c]quinolin-4-amine

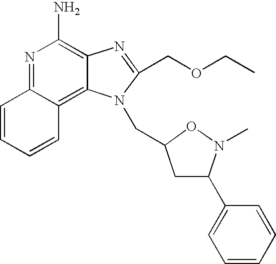 Part A

α-Phenyl-N-methylnitrone was prepared using a modified version of the procedure of C. M. Dicken and P. DeShong, J. Org. Chem. 47, pp. 2047-2051 (1982). A solution of 1-allyl-4-chloro-2-(ethoxymethyl)-1H-imidazo[4,5-c]quinoline (prepared as described in Part C of Example 300, 2.00 g, 6.63 mmol) and α-phenyl-N-methylnitrone (941 mg, 6.96 mmol) in toluene (10 mL) was heated at reflux for 60 hours. The solvent was removed under reduced pressure to afford an orange solid. The solid was triturated with ethyl acetate and isolated by filtration to provide the product 4-chloro-2-(ethoxymethyl)-1-[(2-methyl-3-phenylisoxazolidin-5-yl)methyl]-1H-imidazo[4,5-c]quinoline (1.05 g). The filtrate was concentrated, and the residue was purified by flash chromatography (silica gel, eluted with 98:2 dichloromethane/methanol) to afford additional product (360 mg). The material was combined to yield 1.41 g of 4-chloro-2-(ethoxymethyl)-1-[(2-methyl-3-phenylisoxazolidin-5-yl)methyl]-1H-imidazo[4,5-c]quinoline as an off-white solid.

Part B

4-Chloro-2-(ethoxymethyl)-1-[(2-methyl-3-phenylisoxazolidin-5-yl)methyl]-1H-imidazo[4,5-c]quinoline (750 mg, 1.72 mmol) was treated with a solution of 7 M ammonia in methanol (25 mL) according to the method described in Part A of Example 301. The crude product was purified by flash chromatography (silica gel, gradient elution using 97:3 to 96:4 dichloromethane/methanol) to afford 2-(ethoxymethyl)-1-[(2-methyl-3-phenylisoxazolidin-5-yl)methyl]-1H-imidazo[4,5-c]quinolin-4-amine (620 mg) as a white solid. The 2-(ethoxymethyl)-1-[(2-methyl-3-phenylisoxazolidin-5-yl)methyl]-1H-imidazo[4,5-c]quinolin-4-amine was crystallized from isopropanol to yield a white crystalline solid containing predominantly one diastereomer, mp 162-163° C. ¹H NMR (300 MHz, CDCl₃) δ 8.08 (dd, J=8.3, 0.9 Hz, 1H), 7.82 (dd, J=8.4, 1.0 Hz, 1H), 7.51 (dt, J=8.4, 1.3 Hz, 1H), 7.42-7.30 (m, 6H), 5.44 (br s, 2H), 5.21 (dd, J=15.2, 9.8 Hz, 1H), 5.11 (d, J=12.3 Hz, 1H), 4.77 (d, J=12.3 Hz, 1H), 4.73-4.65 (m, 1H), 4.56 (dd, J=15.2, 2.3 Hz, 1H), 3.67 (dq, J=7.0, 1.0 Hz, 2H), 3.56 (t, J=8.6 Hz, 1H), 3.03 (td, J=12.6, 8.3 Hz, 1H), 2.54 (s, 3H), 2.19 (ddd, J=12.8, 9.2, 4.8 Hz, 1H), 1.29 (t, J=7.0 Hz, 3H); MS (APCD) m/z 418 (M+H⁺);

Anal. calcd for C₂₄H₂₇N₅O₂: C, 69.04; H, 6.52; N, 16.77. Found: C, 68.82; H, 6.74; N, 16.69.

The ¹H NMR data provided were obtained from the major diastereomer.

### Example 303

### 2-(Ethoxymethyl)-1-[(2-methyl-3-pyridin-3-ylisoxazolidin-5-yl)methyl]-1H-imidazo[4,5-c]quinolin-4-amine

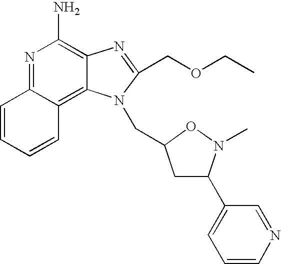 Part A

α-(3-Pyridyl)-N-methylnitrone was prepared using a modified version of a procedure published by C. M. Dicken and P. DeShong, J. Org. Chem., 47, pp. 2047-2051 (1982). A mixture of α-(3-pyridyl)-N-methylnitrone (1.06 g, 7.79 mmol) and 1-allyl-2-(ethoxymethyl)-1H-imidazo[4,5-c]quinolin-4-amine (Prepared as described in Part A of Example 301, 2.00 g, 7.08 mmol) in toluene (20 mL) was sealed in a pressure tube and heated at 118° C. for 89 hours. The solvents were removed under reduced pressure and the residue was purified by flash chromatography (silica gel, gradient elution from 98:2 to 94:6 dichloromethane/methanol). The product 2-(ethoxymethyl)-1-[(2-methyl-3-pyridin-3-ylisoxazolidin-5-yl)methyl]-1H-imidazo[4,5-c]quinolin-4-amine was obtained as a yellow solid (1.70 g) as a mixture of diastereomers that was triturated with acetonitrile to afford a white solid, mp 97-102° C. ¹H NMR (300 MHz, CDCl₃) δ 8.66-8.60 (m, 2H), 8.07 (d, J=8.0 Hz, 1H), 7.83 (dd, J=8.3, 0.9 Hz, 1H), 7.76 (dt, J=7.9, 1.9 Hz, 1H), 7.54 (dt, J=7.2, 1.1 Hz, 1H), 7.37-7.28 (m, 2H), 5.41 (br s, 2H), 5.20 (dd, J=15.2, 9.7 Hz, 1H), 5.08 (d, J=12.4 Hz, 1H), 4.77 (d, J=12.4 Hz, 1H), 4.72 (m, 1H), 4.58 (dd, J=15.2, 1.9 Hz, 1H), 3.69-3.58 (m, 3H), 3.07 (td, J=12.9, 8.3 Hz, 1H), 2.55 (s, 3H), 2.18 (ddd, J=12.9, 9.1, 4.8 Hz, 1H), 1.28 (t, J=7.0 Hz, 3H); MS (APCI) m/z 419 (M+H⁺);

Anal. calcd for C₂₃H₂₆N₆O₂.0.2H₂O: C, 65.45; H, 6.30; N, 19.91. Found: C, 65.16; H, 6.42; N, 20.06.

The ¹H NMR data provided were obtained from the major diastereomer.

### Example 304

### 1-[(2-Benzylisoxazolidin-5-yl)methyl]-2-(ethoxymethyl)-1H-imidazo[4,5-c]quinolin-4-amine

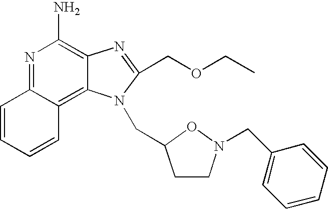 Part A

To a thick-walled glass pressure tube was added 1-allyl-4-chloro-2-(ethoxymethyl)-1H-imidazo[4,5-c]quinoline (prepared as described in Part C of Example 300, 2.50 g, 8.28 mmol), N-benzylhydroxylamine hydrochloride (1.45 g, 9.11 mmol), paraformaldehyde (1.24 g, 41.4 mmol), Al₂O₃ (3 g) and toluene (30 mL). The mixture was cooled to 0° C., and sodium bicarbonate was added (1.18 g, 14.1 mmol). After gas evolution ceased, the tube was sealed and heated with stirring in a 110° C. oil bath overnight. The following morning, additional N-benzylhydroxylamine hydrochloride (0.73 g), paraformaldehyde (0.62 g), and sodium bicarbonate (0.59 g) were added. The reaction was heated with stirring in a 110° C. oil bath for 1 day. After cooling to room temperature, the slurry was filtered through CELITE filter agent. The filtrate was concentrated to an oil that was purified by flash chromatography (silica gel, eluted with 98:2 dichloromethane/methanol) to afford 1-[(2-benzylisoxazolidin-5-yl)methyl]-4-chloro-2-(ethoxymethyl)-1H-imidazo[4,5-c]quinoline (2.80 g) as a tan oil.

Part B

1-[(2-Benzylisoxazolidin-5-yl)methyl]-4-chloro-2-(ethoxymethyl)-1H-imidazo[4,5-c]quinoline (1.00 g, 2.29 mmol) was treated with a solution of 7 M ammonia in methanol (20 mL) according to the method described in Part A of Example 301. The crude product was purified by flash chromatography (silica gel, gradient elution using 97:3 to 95:5 dichloromethane/methanol) to afford 1-[(2-benzylisoxazolidin-5-yl)methyl]-2-(ethoxymethyl)-1H-imidazo[4,5-c]quinolin-4-amine (350 mg) as a white solid that was crystallized from acetonitrile to obtain an analytically pure sample as a tan crystalline solid, mp 137-138° C. ¹H NMR (300 MHz, CDCl₃) δ 7.80 (d, J=8.2 Hz, 1H), 7.72 (dd, J=8.4, 1.0 Hz, 1H), 7.51 (ddd, J=8.4, 7.1, 1.3 Hz, 1H), 7.32-7.23 (m, 6H), 5.43 (br s, 2H), 4.86 (dd, J=15.0, 9.1 Hz, 1H), 4.65-4.53 (m, 3H), 4.32 (m, 1H), 3.95-3.81 (m, 2H), 3.51 (m, 2H), 3.30 (m, 1H), 2.58 (m, 2H), 2.17 (m, 1H), 1.20 (t, J=7.0 Hz, 3H); MS (APCI) m/z 418 (M+H⁺); Anal. calcd for C₂₄H₂₇N₅O₂: C, 69.04; H, 6.52; N, 16.77. Found: C, 69.05; H, 6.55; N, 16.99.

### Example 305

### 1-[(2-Methyl-3-phenylisoxazolidin-5-yl)methyl]-1H-imidazo[4,5-c]quinolin-4-amine

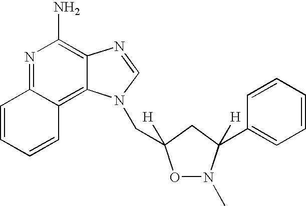 Part A

A mixture of N⁴-allyl-2-chloroquinoline-3,4-diamine (prepared as described in Part B of Example 300, 71.1 g, 304 mmol), trimethylorthoformate (75.8 mL, 456 mmol), and pyridine hydrochloride (0.70 g, 6.1 mmol) in toluene (1 L) was heated at reflux for 16 hours. The reaction was allowed to cool to room temperature, and the volatiles were removed under reduced pressure to yield a black solid that was partitioned between dichloromethane and water. The organic layer washed with water and brine, dried over MgSO₄, filtered, and concentrated to a dark brown solid. The crude material was passed through a plug of silica gel (7:3 dichloromethane/ethyl acetate as the eluent) to yield 1-allyl-4-chloro-1H-imidazo[4,5-c]quinoline as a tan solid (67.4 g).

Part B

A mixture of benzaldehyde (3.00 mL, 29.5 mmol), N-methylhydroxylamine hydrochloride (3.20 g, 38.4 mmol) and sodium bicarbonate (7.90 g, 94.4 mmol) in dichloromethane (30 mL) was heated to 50° C. under a nitrogen atmosphere for 4.5 hours. The mixture was filtered through CELITE filter agent. The filtrate was concentrated to a white solid that was triturated with diethyl ether. The solid was isolated by filtration and dried under vacuum to yield α-phenyl-N-methylnitrone as a white solid (4.13 g, 100%). A solution of the α-phenyl-N-methylnitrone (0.582 g, 4.31 mmol) and 1-allyl-4-chloro-1H-imidazo[4,5-c]quinoline from above in Part A (1.00 g, 4.10 mmol) in toluene (15 mL) was heated at reflux for 8 days. The solution was allowed to cool to room temperature and was concentrated to an oil that was purified by flash chromatography (silica gel, gradient elution from 1:1 to 4:1 ethyl acetate/hexanes to yield 4-chloro-1-[(2-methyl-3-phenylisoxazolidin-5-yl)methyl]-1H-imidazo[4,5-c]quinoline (1.02 g).

Part C

4-Chloro-1-[(2-methyl-3-phenylisoxazolidin-5-yl)methyl]-1H-imidazo[4,5-c]quinoline (0.600 g, 1.58 mmol) was treated with a solution of 7 N ammonia in methanol (6 mL), and the reaction was heated in a sealed pressure vessel of 22 hours at 150° C. and allowed to cool to room temperature. The reaction was found to be incomplete. Additional 7 N ammonia in methanol was added, and heating was resumed at 150° C. for 20 hours. The reaction was concentrated and the resulting solid was triturated with water and isolated by filtration. The solid was recrystallized from ethyl acetate and dried at 70° C. under vacuum to yield a white solid that was the major cis diastereomer of 1-[(2-methyl-3-phenylisoxazolidin-5-yl)methyl]-1H-imidazo[4,5-c]quinolin-4-amine. The reaction was repeated on smaller scale (0.250 g, 0.660 mmol of 4-chloro-1-[(2-methyl-3-phenylisoxazolidin-5-yl)methyl]-1H-imidazo[4,5-c]quinoline) and the minor trans diastereomer of 1-[(2-methyl-3-phenylisoxazolidin-5-yl)methyl]-1H-imidazo[4,5-c]quinolin-4-amine was obtained after the crude product was purified by flash chromatography (95/5 dichloromethane/methanol) and crystallized from acetonitrile to yield a white solid (49.5 mg). The major diastereomer was obtained as a white crystalline solid, mp 198.5-200.0° C. ¹H NMR (300 MHz, DMSO-d₆) δ 8.28 (d, J=8.0 Hz, 1H), 8.19 (s, 1H), 7.62 (d, J=8.0 Hz, 1H), 7.46-7.32 (m, 6H), 7.23 (t, J=7.3 Hz, 1H), 6.62 (br s, 2H), 4.90 (dd, J=14.8, 8.8 Hz, 1H), 4.74 (dd, J=14.7, 2.5 Hz, 1H), 4.60-4.54 (m, 1H), 3.59 (t, J=8.3 Hz, 1H), 2.96 (dt, J=12.4, 8.0 Hz, 1H), 2.40 (s, 3H), 2.15 (m, 1H); MS (APCI) m/z 360 (M+H)⁺;

Anal. calcd for C₂₁H₂₁N₅O.0.5H₂O: C, 68.46; H, 6.02; N, 19.01. Found: C, 68.58; H, 6.02; N, 19.04.

The minor diastereomer was obtained as a white crystalline solid, mp 190.0-192.0° C. ¹H NMR (300 MHz, DMSO-d₆) δ 8.22 (s, 1H), 8.17 (d, J=7.9 Hz, 1H), 7.62 (d, J=8.3 Hz, 1H), 7.44 (t, J=7.0 Hz, 1H), 7.24-7.33 (m, 6H), 6.59 (s, 2H), 4.75-4.91 (m, 2H), 4.57-4.61 (bs, 1H), 3.55 (s, 1H), 2.48-2.59 (m, 2H), 2.37 (s, 3H); MS (APCI) m/z 360 (M+H)⁺;

Anal. calcd for C₂₁H₂₁N₅O: C, 70.18; H, 5.89; N, 19.48. Found: C, 70.06; H, 5.98; N, 19.80.

### Example 306

### 6-{[4-Amino-2-(ethoxymethyl)-1H-imidazo[4,5-c]quinolin-1-yl]methyl}-3-methyl-1,3-oxazinan-2-one

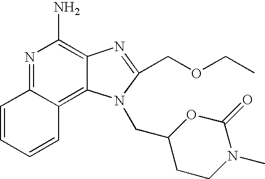 Part A

Allylamine (7.60 mL, 101 mmol) was added dropwise to a cooled solution of 4-chloro-3-nitroquinoline (20.0 g, 95.9 mmol) and triethylamine (20.0 mL, 144 mmol) in dichloromethane (350 mL). After 3 hours, the volatiles were removed under reduced pressure and the resulting yellow solid was suspended in water (300 mL). Solid sodium carbonate was added until a pH of 10-11 was reached. The suspension was stirred for 2 hours at 0° C. The yellow solid was isolated by filtration and dried under vacuum overnight to provide the product N-allyl-3-nitroquinolin-4-amine (20.0 g).

Part B

An aqueous solution (200 mL) of potassium carbonate (60.8 g, 440 mmol) and sodium dithionate (81.1 g, 396 mmol) was added dropwise over 30 minutes to a mixture of N-allyl-3-nitroquinolin-4-amine (20.0 g, 88 mmol) and ethyl viologen dibromide (1.98 g, 5.28 mmol) in dichloromethane (320 mL) and water (40 mL) under a nitrogen atmosphere. The dark blue-green mixture was stirred rapidly and was heated at reflux for 4 hours. Additional ethyl viologen dibromide (1.0 g) was added and the mixture was stirred overnight at room temperature. The mixture was transferred to a separatory funnel and the layers were separated. The aqueous layer was extracted with dichloromethane. The combined organic layers were filtered through CELITE filter agent, dried over MgSO₄, filtered, and concentrated to a dark oil. The crude product was purified by suction filter chromatography using silica gel to afford the product N⁴-allylquinoline-3,4-diamine (13.0 g) and recovered staring material (4.90 g).

Part C

Ethoxyacetyl chloride (8.84 g, 72.1 mmol) was added dropwise to a solution of N4-allylquinoline-3,4-diamine (13.0 g, 65.6 mmol) in acetonitrile (300 mL) at room temperature. After 45 minutes, the solvent was removed under reduced pressure to provide a sticky solid. The solid was dissolved in a solution of ethanol (240 mL) and water (80 mL). Sodium hydroxide (3.93 g, 98.3 mmol) was added and the solution was heated at reflux for 2 hours. The ethanol was removed under reduced pressure and the remaining aqueous layer was extracted several times with dichloromethane. The combined organic layers were dried over MgSO₄, filtered, and concentrated to an orange solid. The solid was triturated with ethyl acetate and isolated by filtration to yield 10.27 g of a pale yellow solid. The filtrate was concentrated and purified by suction filter chromatography (silica gel with 97:3 dichloromethane/methanol as the eluent) to yield an additional 4.23 g of product. The material was combined to provide 14.6 g of 1-allyl-2-ethoxymethyl-1H-imidazo[4,5-c]quinoline.

Part D

A mixture of 1-allyl-2-ethoxymethyl-1H-imidazo[4,5-c]quinoline (4.81 g, 18.0 mmol), N-methylhydroxylamine hydrochloride (1.65 g, 19.8 mmol), sodium bicarbonate (2.57 g, 30.6 mmol), paraformaldehyde (2.70 g, 90.0 mmol) and alumina (36 g) in toluene (40 mL) was heated at reflux for 18 hours. Additional N-methylhydroxylamine hydrochloride (0.83 g), sodium bicarbonate (1.29 g), and paraformaldehyde (1.35 g) were added and the mixture was heated at reflux for an additional 6 hours. The mixture was filtered through CELITE filter agent and concentrated under reduced pressure. The crude product was purified by suction filter chromatography (silica gel, gradient elution with 97:3 to 95:5 dichloromethane/methanol) to yield 5.50 g of 2-ethoxymethyl-1-[(2-methylisoxazolidin-5-yl)methyl]-1H-imidazo[4,5-c]quinoline as tan oil.

Part E

10% Palladium on carbon (200 mg) was wetted with isopropanol and added to a solution of 2-ethoxymethyl-1-[(2-methylisoxazolidin-5-yl)methyl]-1H-imidazo[4,5-c]quinoline (800 mg, 2.45 mmol) in methanol (10 mL) and acetic acid (10 mL) in a Parr vessel. The mixture was hydrogenated on a Parr apparatus at 40 psi (2.8×10⁵ Pa) overnight. The mixture was filtered through CELITE filter agent and solid potassium carbonate was added to the filtrate until the pH=11. The filtrate was extracted with chloroform. The combined chloroform layers were concentrated to an oil that was purified on a plug of silica gel (gradient elution using 4:1 to 1:1 chloroform/methanol) to afford 1-(2-ethoxymethyl-1H-imidazo[4,5-c]quinolin-1-yl)-4-(methylamino)butan-2-ol as a white solid (520 mg).

Part F

1,1′-Carbonyldiimidazole was added to a solution of 1-(2-ethoxymethyl-1H-imidazo[4,5-c]quinolin-1-yl)-4-(methylamino)butan-2-ol (520 mg, 1.58 mmol) in tetrahydrofuran (10 mL). The solution was heated at reflux for 2 days, then was partitioned between ethyl acetate and saturated aqueous sodium bicarbonate. The aqueous layer was extracted with ethyl acetate and dichloromethane. The combined organic layers were dried over MgSO₄, filtered, and concentrated to an oil. The crude product was purified by flash chromatography (silica gel, gradient elution from 97:3 to 90:10 dichloromethane/methanol) to afford 6-[(2-ethoxymethyl-1H-imidazo[4,5-c]quinolin-1-yl)methyl]-3-methyl-1,3-oxazinan-2-one as a white foam (350 mg).

Part G

To a solution of 6-[(2-ethoxymethyl-1H-imidazo[4,5-c]quinolin-1-yl)methyl]-3-methyl-1,3-oxazinan-2-one (350 mg, 0.99 mmol) in chloroform (15 mL) was added mCPBA (77% w/w, 267 mg, 1.19 mmol). After 30 minutes, the solution washed with 2% aqueous sodium carbonate solution, dried over MgSO₄, filtered, and concentrated to an orange solid. The solid was dissolved in dichloromethane (20 mL) and concentrated ammonium hydroxide (10 mL) was added. To the vigorously stirred mixture was added p-toluenesulfonyl chloride (208 mg, 1.09 mmol). After 40 minutes, the reaction mixture was partitioned between dichloromethane and 2% aqueous sodium carbonate solution. The aqueous layer was extracted with dichloromethane. The organic layers were combined, washed with brine, dried over MgSO₄, filtered, and concentrated to yield an oil. The crude product was purified by flash chromatography (silica gel) and recrystallized from acetonitrile to afford 6-[(4-amino-2-ethoxymethyl-1H-imidazo[4,5-c]quinolin-1-yl)methyl]-3-methyl-1,3-oxazinan-2-one as white crystals (200 mg), mp 192-194° C. ¹H NMR (300 MHz, CDCl₃) δ 7.91 (d, J=8.2 Hz, 1H), 7.82 (d, J=8.3 Hz, 1H), 7.52 (t, J=7.7 Hz, 1H), 7.32 (t, J=7.6 Hz, 1H), 5.63 (br s, 2H), 5.00-4.78 (m, 5H), 3.64 (q, J=6.9 Hz, 2H), 3.34-3.18 (m, 2H), 2.94 (s, 3H), 2.07-2.03 (m, 2H), 1.25 (t, J=6.9 Hz, 3H); MS (APCI) m/z 370 (M+H)⁺;

Anal. calcd for C₁₉H₂₃N₅O₃: C, 61.77; H, 6.28; N, 18.96. Found: C, 61.53; H, 6.07; N, 19.16.

### Example 307

### 5-[(4-Amino-2-propyl-1H-imidazo[4,5-c]quinolin-1-yl)methyl]-1,3-oxazolidin-2-one

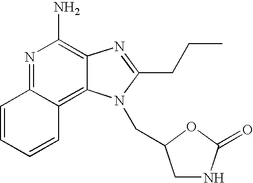 Part A

A solution of di(tert-butyl)dicarbonate (86.3 g, 0.395 mol) in 1,4-dioxane (430 mL) was added dropwise to a stirred solution of 1,3-diaminopropan-2-ol (178 g, 1.98 mol) in methanol (525 mL) at room temperature. The reaction was stirred overnight and concentrated under reduced pressure to an oil. A solution of 10% citric acid and solid citric acid was added until the pH=4 and the volume of the solution was 1.5 L. The aqueous solution washed with dichloromethane (500 mL×3). To the aqueous solution was added 50% NaOH until the pH=12. The aqueous solution was extracted with chloroform (500 mL×7). The organic layers were combined, concentrated, and dried under vacuum to provide tert-butyl 3-amino-2-hydroxypropylcarbamate as a white solid (59.63 g).

Part B

A solution of 4-chloro-3-nitroquinoline (45.7 g, 0.219 mol), tert-butyl 3-amino-2-hydroxypropylcarbamate (50 g, 0.26 mol), and triethylamine (46 mL, 0.33 mol) in DMF (800 mL) was stirred for 2 hours at room temperature. Some of the DMF was removed by evaporation under reduced pressure. The reaction mixture was poured onto water (3 L) and the mixture was stirred for 10 minutes. A bright yellow solid was collected by filtration, washed with water (800 mL×3), and dried under vacuum to provide tert-butyl 2-hydroxy-3-[(3-nitroquinolin-4-yl)amino]propylcarbamate (76.4 g).

Part C

A mixture of tert-butyl 2-hydroxy-3-[(3-nitroquinolin-4-yl)amino]propylcarbamate (55.38 g, 0.153 mol), 5% platinum on carbon (11.0 g), and two tablespoons MgSO₄ in 1,2-dichloroethane was hydrogenated on a Parr hydrogenation apparatus for 4.5 h. More 5% platinum on carbon (2.8 g) was added and the mixture was hydrogenated overnight. The mixture was filtered through CELITE filter agent and the filtrate was concentrated to provide tert-butyl 3-[(3-aminoquinolin-4-yl)amino]-2-hydroxypropylcarbamate as a yellow-brown solid (46.64 g).

Part D

A mixture of tert-butyl 3-[(3-aminoquinolin-4-yl)amino]-2-hydroxypropylcarbamate (46.64 g, 0.140 mol), trimethyl orthobutyrate (26.9 mL, 0.168 mol), and pyridine hydrochloride (1.60 g, 13.8 mmol) in toluene (360 mL) was heated at reflux. After 18 hours, additional trimethyl orthobutyrate (13.45 g×2) was added and the solution was heated at reflux for 1 more day. The solution was allowed to cool to room temperature and washed with brine (400 mL×3). The organic layer was concentrated under reduced pressure and the residue was dried under vacuum to yield tert-butyl 2-hydroxy-3-(2-propyl-1H-imidazo[4,5-c]quinolin-1-yl)propylcarbamate as a yellow solid (29.69 g).

Part E

A solution of tert-butyl 2-hydroxy-3-(2-propyl-1H-imidazo[4,5-c]quinolin-1-yl)propylcarbamate (10.00 g, 26.01 mmol), tert-butyldimethylsilyl chloride (8.63 g, 57.22 mmol), and triethylamine (14.50 mL, 104 mmol) in DMF was stirred overnight at room temperature. Additional tert-butyldimethylsilyl chloride (4.32 g), and triethylamine (8.00 mL) were added to the solution, which was then heated briefly and then allowed to stir at room temperature for 4 hours. The solvent was removed under reduced pressure. The residue was dissolved in chloroform and washed with 5% NH₄Cl (3×), 1:1 water/saturated sodium bicarbonate solution (3×), and 5% NH₄Cl. The organic layer was concentrated to provide tert-butyl 2-{[tert-butyl(dimethyl)silyl]oxy}-3-(2-propyl-1H-imidazo[4,5-c]quinolin-1-yl)propylcarbamate (12.96 g).

Part F

To a solution of tert-butyl 2-{[tert-butyl(dimethyl)silyl]oxy}-3-(2-propyl-1H-imidazo[4,5-c]quinolin-1-yl)propylcarbamate (2.00 g, 4.01 mmol) in chloroform (15 mL) at room temperature was added mCPBA (77% max., 837 mg, 4.85 mmol). The mixture was stirred overnight and concentrated ammonium hydroxide (20 mL) was added. After 1 hour, p-toluenesulfonyl chloride (841 mg, 4.41 mmol) was added slowly to the mixture, which was stirred overnight at room temperature. The mixture was transferred to a separatory funnel and the layers were separated. The aqueous layer was extracted with chloroform twice. The combined organic layers were washed with saturated NH₄Cl (3×), saturated NaHCO₃ (2×), and brine and concentrated to yield tert-butyl 3-(4-amino-2-propyl-1H-imidazo[4,5-c]quinolin-1-yl)-2-{[tert-butyl(dimethyl)silyl]oxy}propylcarbamate (2.23) which was about 70% pure and used without purification in the next step.

Part G

To a solution of the material from Part F in tetrahydrofuran at −20° C. was added a 1 M solution of tetrabutylammonium fluoride (4.77 mL, 4.77 mmol). The reaction solution was allowed to warm to room temperature over 3 hours. The solution was concentrated under reduced pressure to provide impure tert-butyl 3-(4-amino-2-propyl-1H-imidazo[4,5-c]quinolin-1-yl)-2-hydroxypropylcarbamate (1.69 g) that was used without further manipulation in the next step.

Part H

To a solution of the material from Part G in tetrahydrofuran (15 mL) at room temperature was added 1 M potassium tert-butoxide in tetrahydrofuran (10.6 mL, 10.6 mmol). The solution was stirred for 4 hours and then was concentrated in vacuo. The residue was partitioned between chloroform and 10% citric acid and the layers were separated. The aqueous layer was made basic with sodium carbonate and extracted with chloroform. The combined organic layers were washed with saturated NH₄Cl (3×), saturated NaHCO₃ (2×), and brine. The solution was concentrated and the crude residue (1.08 g) was purified by flash chromatography. The appropriate fractions were combined, concentrated, and dried under vacuum to yield a solid that was crystallized from methanol to provide 5-[(4-amino-2-propyl-1H-imidazo[4,5-c]quinolin-1-yl)methyl]-1,3-oxazolidin-2-one as white crystals. ¹H NMR (300 MHz, CD₃OD) δ 8.09 (d, J=8.3 Hz, 1H), 7.72 (d, J=8.4 Hz, 1H), 7.51 (dd, J=8.6, 7.1 Hz, 1H), 7.36 (dd, J=7.1, 2.4 Hz, 1H), 5.18-5.15 (m, 1H), 4.98-4.96 (m, 2H), 3.90 (t, J=9.0 Hz, 1H), 3.59 (dd, J=7.7, 7.7 Hz, 1H), 3.36 (s, 1H), 3.02 (t, J=9.2 Hz, 2H), 2.05-1.93 (m, 2H), 1.14 (t, J=7.3 Hz, 3H); MS (APCI) m/z 326 (M+H)⁺; HRMS (EI) calcd for C₁₇H₁₉N₅O₂: 326.1617, found: 326.1610.

### Example 308

### N-{4-[4-Amino-1-(2-methylpropyl)-1H-imidazo[4,5-c]quinolin-2-yl]butyl}benzamide

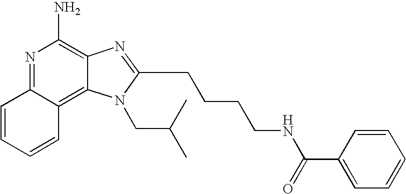 Part A

Under a nitrogen atmosphere, a solution of 5-[(tert-butoxycarbonyl)amino]pentanoic acid (Boc 5-Ava-OH, 9.50 g, 43.7 mmol) in anhydrous 1,2-dichloroethane (100 mL) was cooled to −20° C., and trimethylacetyl chloride (5.4 mL, 43.7 mmol) and triethylamine (25 mL, 0.199 mol) were sequentially added. The reaction was warmed to 0° C. and stirred for three hours. A solution of N⁴-(2-methylpropyl)quinoline-3,4-diamine (8.56 g, 39.8 mmol) in 1,2-dichloroethane (125 mL) was added, and the reaction was allowed to warm to room temperature, heated at reflux overnight, and allowed to cool to room temperature. Chloroform was added, and the resulting solution washed sequentially with water and cold saturated ammonium chloride (2×200 mL), dried over sodium sulfate, filtered, and concentrated under reduced pressure. The crude product was purified by column chromatography on silica gel (240 g, eluting with 92.5:7.5 dichloromethane:methanol). The column fractions were divided into two portions to provide two solids. Each solid was dissolved in a small volume of dichloromethane, and hexanes were added to cause a precipitate to form. The precipitate was isolated by filtration, and the filtrate was concentrated and treated again with dichloromethane and hexanes as described above. The process was repeated until no additional solid precipitated with the addition of hexanes. A mixture of tert-butyl 4-[1-(2-methylpropyl)-1H-imidazo[4,5-c]quinolin-2-yl]butylcarbamate containing a small amount of tert-butyl 5-({4-[(2-methylpropyl)amino]quinolin-3-yl}amino)-5-oxopentylcarbamate (9.26 g total) was obtained.

Part B

mCPBA (1.60 g of 60% pure material, 5 mmol) was added in one portion to a solution of the material from Part A (1.63 g, 4.11 mmol) in chloroform (50 mL); the reaction mixture was stirred at room temperature overnight. An analysis by TLC indicated the presence of starting material, and additional mCPBA (0.40 g) was added. The reaction was stirred for an additional three hours and then washed sequentially with saturated aqueous sodium bicarbonate (2×100 mL) and brine (100 mL), dried over magnesium sulfate, filtered, and concentrated under reduced pressure to provide tert-butyl 4-[1-(2-methylpropyl)-5-oxido-1H-imidazo[4,5-c]quinolin-2-yl]butylcarbamate as an orange solid.

Part C

Concentrated ammonium hydroxide (10 mL) was added to a stirred solution of the material from Part B in chloroform (50 mL). The mixture was stirred rapidly under a nitrogen atmosphere and cooled to 0° C. p-Toluenesulfonyl chloride (1.57 g, 8.23 mmol) was added in portions over a period of 45 minutes. The reaction mixture was stirred at 0° C. for 15 minutes, allowed to warm to room temperature, and stirred overnight. An analysis by HPLC indicated the presence of starting material, and the reaction was cooled to 0° C. Additional p-toluenesulfonyl chloride (0.79 g) was added, and the reaction mixture was stirred at 0° C. for 15 minutes, allowed to warm to room temperature, and stirred for two hours. The organic layer was separated and washed sequentially with 1% aqueous sodium carbonate (2×50 mL) and water (100 mL), dried over magnesium sulfate, filtered, and concentrated under reduced pressure to provide a sticky, orange solid. The solid was dissolved in a small volume of dichloromethane, and hexanes were added to cause a precipitate to form. The precipitate was isolated by filtration. A second crop of solid was isolated from the mother liquor and washed with hexanes. The two solids were combined to provide 1.62 g of tert-butyl 4-[4-amino-1-(2-methylpropyl)-1H-imidazo[4,5-c]quinolin-2-yl]butylcarbamate as a white crystalline solid, m.p. 165-167° C.

MS (APCI) m/z 412 (M+H);

Anal calcd for C₂₃H₃₃N₅O₂: C, 67.13; H, 8.08; N, 17.02. Found: C, 67.10; H, 7.93; N, 16.82.

Part D

Hydrogen chloride (15 mL of a 6 M solution in ethanol) was added to a solution of tert-butyl 4-[4-amino-1-(2-methylpropyl)-1H-imidazo[4,5-c]quinolin-2-yl]butylcarbamate (3.14 g, 7.63 mmol) in ethanol (30 mL), and the reaction was heated at reflux for one hour and allowed to cool to room temperature. Nitrogen gas was bubbled through the solution, and a precipitate formed. The solvent was removed under reduced pressure, and the residue was dissolved in deionized water and adjusted to pH 11 with the addition of ammonium hydroxide. The basic mixture was extracted twice with chloroform, and the combined extracts were concentrated under reduced pressure. Toluene was added to the residue and then removed under reduced pressure to provide 2.30 g of product. The product was dissolved in 6 N hydrochloric acid, and the resulting solution washed with dichloromethane (2×50 mL) and then made basic with the addition of 10% w/w aqueous sodium hydroxide. The resulting mixture was stirred for a few hours. A solid was present and was isolated by filtration, washed with water and diethyl ether, and dried under high vacuum for three days at 60° C. to provide 2-(4-aminobutyl)-1-(2-methylpropyl)-1H-imidazo[4,5-c]quinolin-4-amine hydrochloride as a white powder.

Part E

A solution of 2-(4-aminobutyl)-1-(2-methylpropyl)-1H-imidazo[4,5-c]quinolin-4-amine hydrochloride (0.800 g, 2.57 mmol) in dichloromethane (50 mL) was cooled to 0° C. under a nitrogen atmosphere. Triethylamine (0.400 mL, 2.83 mmol) and benzoyl chloride (0.300 mL, 2.57 mmol) were sequentially added, and the reaction was stirred for one hour at 0° C., allowed to warm to room temperature, and stirred for two hours. The reaction mixture washed sequentially with cold deionized water (100 mL) and cold dilute aqueous ammonium hydroxide (100 mL). Sodium chloride was added to break up an emulsion. The organic layer was separated and washed with cold brine (100 mL), dried over sodium sulfate, filtered, and concentrated under reduced pressure. The residue was purified by column chromatography on silica gel (35 g, eluting with 90:10 dichloromethane:methanol) and dried under high vacuum at 60° C. for two days to provide 0.43 g of N-{4-[4-amino-1-(2-methylpropyl)-1H-imidazo[4,5-c]quinolin-2-yl]butyl}benzamide as a yellow, crystalline solid, mp 95-101° C. Anal calcd for C₂₅H₂₉N₅O: C, 72.26; H, 7.03; N, 16.85. Found: C, 71.93; H, 6.92; N, 16.72.

### Example 309

### N-{[4-Amino-1-(2-hydroxy-2-methylpropyl)-1H-imidazo[4,5-c][1,5]naphthyridin-2-yl]methyl}acetamide

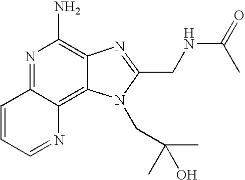 Part A

A Parr vessel was charged with 1-[(3-nitro[1,5]naphthyridin-4-yl)amino]-2-methylpropan-2-ol (11.24 g, 42.9 mmol), acetonitrile (203 mL), isopropyl alcohol (61 mL), and 5% platinum on carbon (0.9 g), and the mixture was placed under hydrogen pressure overnight and filtered through a layer of CELITE filter agent. The filtrate was concentrated under reduced pressure to provide 1-[(3-amino[1,5]naphthyridin-4-yl)amino]-2-methylpropan-2-ol.

Part B

Chloroacetyl chloride (5.4 mL, 68 mmol) was added to a solution of the material from Part A in chloroform (400 mL). The reaction was stirred for two hours at room temperature; a precipitate was present and was isolated by filtration and washed with chloroform. The solid was then triturated with acetone (2 mL/g) at 0° C. and isolated by filtration to provide 12.14 g of 2-chloro-N⁴-(2-hydroxy-2-methylpropylamino)-([1,5]naphthyridin-3-yl)acetamide hydrochloride.

Part C

Triethylamine (16 mL, 0.11 mol) was added to a suspension of the material from Part B in ethanol (80 mL/g), and the resulting solution was stirred for three days at room temperature. The volatiles were removed under reduced pressure, and the residue was recrystallized from acetonitrile, isolated by filtration, and dried overnight on the vacuum filter to provide 8.86 g of 1-[2-(chloromethyl)-1H-imidazo[4,5-c][1,5]naphthyridin-1-yl]-2-methylpropan-2-ol as a brown solid. A second crop of crystals was separated from the mother liquor by filtration, and the filtrate was concentrated under reduced pressure and recrystallized from isopropyl alcohol to provide an additional 4.78 g of product, containing two impurities.

Part D

Potassium phthalimide (4.26 g, 23.0 mmol) was added to a solution of 1-[2-(chloromethyl)-1H-imidazo[4,5-c][1,5]naphthyridin-1-yl]-2-methylpropan-2-ol (4.46 g, 15.3 mmol, containing two impurities) in DMF (90 mL), and the reaction was stirred for three hours at room temperature and then poured into deionized water (500 mL). The resulting mixture was stirred overnight. A precipitate was present and was isolated by filtration, triturated with a small volume of methanol, isolated by filtration, and dried on the vacuum filter funnel to provide 2.75 g of 2-{[1-(2-hydroxy-2-methylpropyl)-1H-imidazo[4,5-c][1,5]naphthyridin-2-yl]methyl}-1H-isoindole-1,3(2H)-dione as a white powder.

Part E

Hydrazine (0.69 mL, 21.6 mmol) was added to a stirred suspension of 2-{[1-(2-hydroxy-2-methylpropyl)-1H-imidazo[4,5-c][1,5]naphthyridin-2-yl]methyl}-1H-isoindole-1,3(2H)-dione (1.92 g, 4.80 mmol) in chloroform (77 mL), and the resulting solution was stirred overnight at room temperature. Additional hydrazine (0.11 mL, 4.7 mmol) was added, and the reaction was stirred at room temperature for an additional two hours. A solid was present and was removed by filtration, and the filtrate was concentrated under reduced pressure to provide 1-[2-(aminomethyl)-1H-imidazo[4,5-c][1,5]naphthyridin-1-yl]-2-methylpropan-2-ol.

Part F

A suspension of 1-[2-(aminomethyl)-1H-imidazo[4,5-c][1,5]naphthyridin-1-yl]-2-methylpropan-2-ol (0.65 g) in dichloromethane (13 mL) was cooled to 0° C., and triethylamine (0.7 mL, 5 mmol) and acetyl chloride (0.18 mL, 2.5 mmol) were added. The reaction was stirred for ten minutes and diluted with dichloromethane (13 mL). The resulting solution washed with 1% aqueous sodium carbonate, and the aqueous layer was extracted twice with dichloromethane. The combined organic fractions were dried over sodium sulfate, filtered, and concentrated under reduced pressure. The residue was recrystallized from acetonitrile to provide 0.43 g of N-{[1-(2-hydroxy-2-methylpropyl)-1H-imidazo[4,5-c][1,5]naphthyridin-2-yl]methyl}acetamide as off-white crystals.

Part G

mCPBA (1 g of 77% maximum purity material, 3 mmol) was added to a solution of N-{[1-(2-hydroxy-2-methylpropyl)-1H-imidazo[4,5-c][1,5]naphthyridin-2-yl]methyl}acetamide (0.31 g, 0.99 mmol) in chloroform (6.2 mL). The reaction mixture was stirred at room temperature for 30 minutes, diluted with chloroform (6.2 mL), washed with saturated aqueous sodium bicarbonate, dried over sodium sulfate, and filtered. Concentrated ammonium hydroxide (3 mL) and p-toluenesulfonyl chloride (0.37 g, 2.0 mmol) were added to the solution, and the resulting mixture was stirred for ten minutes at room temperature, diluted with chloroform (20 mL), washed with saturated aqueous sodium bicarbonate, dried over sodium sulfate, filtered, and concentrated under reduced pressure. The crude product (0.37 g) was purified by chromatography using a HORIZON HPFC system (silica cartridge, eluting with CMA:chloroform in a gradient from 2:98 to 40:60) followed by recrystallization from acetonitrile:water. The crystals were dried overnight in a vacuum oven at 90° C. to provide 20 mg of N-{[4-amino-1-(2-hydroxy-2-methylpropyl)-1H-imidazo[4,5-c][1,5]naphthyridin-2-yl]methyl}acetamide as a white solid, mp 198-200° C.

MS (APCI) m/z 329 (M+H)⁺;

Anal. calcd for C₁₆H₂₀N₆O₂: C, 58.52; H, 6.14; N, 25.59. Found: C, 58.36; H, 6.04; N, 25.72.

### Example 310

### N-{[4-Amino-1-(2-hydroxy-2-methylpropyl)-1H-imidazo[4,5-c][1,5]naphthyridin-2-yl]methyl}cyclopropanecarboxamide

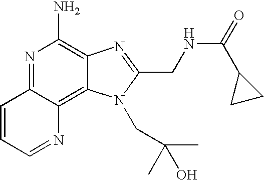 Part A

A suspension of 1-[2-(aminomethyl)-1H-imidazo[4,5-c][1,5]naphthyridin-1-yl]-2-methylpropan-2-ol (0.65 g) in dichloromethane (13 mL) was cooled to 0° C., and triethylamine (0.7 mL, 5 mmol) and cyclopropanecarbonyl chloride (0.23 mL, 2.5 mmol) were added. The reaction was stirred for ten minutes and diluted with dichloromethane (13 mL). The resulting solution washed with 1% aqueous sodium carbonate (10 mL), dried over sodium sulfate, filtered, and concentrated under reduced pressure. The residue was triturated with acetonitrile at 97° C., allowed to cool to room temperature, further cooled to 0° C., and isolated by filtration to provide 0.58 g of N-{[1-(2-hydroxy-2-methylpropyl)-1H-imidazo[4,5-c][1,5]naphthyridin-2-yl]methyl}cyclopropanecarboxyamide as off-white crystals.

Part B

The methods described in Part G of Example 309 were used to treat N-{[1-(2-hydroxy-2-methylpropyl)-1H-imidazo[4,5-c][1,5]naphthyridin-2-yl]methyl}cyclopropanecarboxyamide (0.58 g, 1.7 mmol) with mCPBA (1.5 g of 77% maximum purity material, 4.4 mmol) followed by concentrated ammonium hydroxide (6 mL) and p-toluenesulfonyl chloride (0.67 g, 3.5 mmol), isolate, and chromatographically purify the product. After chromatographic purification, the product was triturated with acetonitrile:water at 98° C., allowed to cool to room temperature, further cooled to 0° C., isolated by filtration, and dried as described in Part G of Example 309 to provide 14 mg of N-{[4-amino-1-(2-hydroxy-2-methylpropyl)-1H-imidazo[4,5-c][1,5]naphthyridin-2-yl]methyl}cyclopropanecarboxyamide as an off-white solid, mp 221-223° C.

MS (APCI) m/z 355 (M+H)⁺;

Anal. calcd for C₁₈H₂₂N₆O₂: C, 61.00; H, 6.26; N, 23.71. Found: C, 60.76; H, 6.21; N, 23.75.

### Example 311

### N-{[4-Amino-1-(2-methylpropyl)-6,7-dimethyl-1H-imidazo[4,5-c]pyridin-2-yl]methyl}acetamide

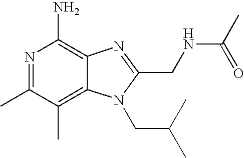

Acetyl chloride (0.517 mL, 7.27 mmol) was added to a solution of 2-(aminomethyl)-1-(2-methylpropyl)-6,7-dimethyl-1H-imidazo[4,5-c]pyridin-4-amine (1.5 g, 6.1 mmol, Example 23 Parts A and B) and triethylamine (1.7 mL, 12 mmol) in dichloromethane (25 mL). The reaction was stirred for two hours at room temperature, and an analysis by LC/MS indicated the presence of starting material. Additional acetyl chloride (0.130 mL, 1.83 mmol) was added, and the reaction was stirred for 30 minutes, diluted with dichloromethane (20 mL), and washed with brine (4×30 mL). The organic phase was dried over magnesium sulfate, filtered through a layer of CELITE filter agent, concentrated under reduced pressure, and further dried for one hour under vacuum. The crude product (1.61 g) was purified by flash chromatography (silica gel, elution with concentrated ammonium hydroxide/methanol/dichloromethane first at a ratio of 1:4:95, next at a ratio of 1:6:93, and finally in a ratio of 1:8:91) to provide 0.490 g of N-{[4-amino-1-(2-methylpropyl)-6,7-dimethyl-1H-imidazo[4,5-c]pyridin-2-yl]methyl}acetamide as brown needles, mp 162-164° C.

Anal. calcd for C₁₅H₂₃N₅O.0.1 H₂O: C, 61.87; H, 8.03; N, 24.05. Found: C, 61.60; H, 8.23; N, 23.82.

### Example 312

### N-{[4-Amino-7-bromo-1-(tetrahydro-2H-pyran-4-ylmethyl)-1H-imidazo[4,5-c]quinolin-2-yl]methyl}cyclopropanecarboxamide

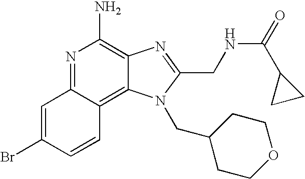 Part A

A solution of 7-bromo-N-(tetrahydro-2H-pyran-4-ylmethyl)quinoline-3,4-diamine (12.33 g, 36.67 mmol, see U.S. Patent Publication Application No. US 2004/0147543 (Hays et al.), Examples 477-480 Parts A through D) in dichloromethane (100 mL) was cooled to 0° C., and triethylamine (7.7 mL, 55 mmol) was added. A solution of chloroacetyl chloride (3.2 mL, 0.040 mol) in dichloromethane (10 mL) was added dropwise, and the reaction was allowed to warm to room temperature and stirred over the weekend. The reaction mixture was diluted with dichloromethane (200 mL) and then washed sequentially with deionized water (2×300 mL), brine (2×300 mL), and deionized water (300 mL). The organic layer was dried over magnesium sulfate, filtered, concentrated under reduced pressure, and further dried under vacuum for three hours. The residue (12.5 g) was purified by automated flash chromatography on silica gel (eluting with concentrated ammonium hydroxide/methanol/dichloromethane in a gradient from 5:0:95 to 5:6:89 to provide 5.83 g of 7-bromo-2-(chloromethyl)-1-(tetrahydro-2H-pyran-4-ylmethyl)-1H-imidazo[4,5-c]quinoline.

Part B

A solution of 7-bromo-2-(chloromethyl)-1-(tetrahydro-2H-pyran-4-ylmethyl)-1H-imidazo[4,5-c]quinoline (5.83 g, 14.8 mmol) in DMF (250 mL) was purged with gaseous ammonia for five minutes. The purging with ammonia was repeated three additional times over the course of 18 hours while the reaction was stirred at room temperature. The solvent was removed under reduced pressure to provide 1-[7-bromo-1-(tetrahydro-2H-pyran-4-ylmethyl)-1H-imidazo[4,5-c]quinolin-2-yl]methanamine.

Part C

Triethylamine (2.99 g, 29.5 mmol) was added to a solution of the material from Part B in chloroform (45 mL), and then cyclopropanecarbonyl chloride (1.54 g, 14.8 mol) was slowly added. The reaction was stirred overnight at room temperature and then concentrated under reduced pressure. The residue was triturated with acetonitrile. The resulting solid was isolated by filtration and recrystallized from isopropyl alcohol to provide 1.14 g of N-{[7-bromo-1-(tetrahydro-2H-pyran-4-ylmethyl)-1H-imidazo[4,5-c]quinolin-2-yl]methyl}cyclopropanecarboxamide as white crystals, mp 140° C.

Anal. calcd for C₂₁H₂₃BrN₄O₂: C, 56.89; H, 5.23; N, 12.64. Found: C, 57.14; H, 5.33; N, 13.02.

The filtrate from the trituration was concentrated under reduced pressure to provide an additional 4.08 g of product, which was used in the next step.

Part D

mCPBA (5.1 g of 77% pure material, 23 mmol) was added to a solution of N-{[7-bromo-1-(tetrahydro-2H-pyran-4-ylmethyl)-1H-imidazo[4,5-c]quinolin-2-yl]methyl}cyclopropanecarboxamide (4.08 g, 9.20 mmol) in chloroform (25 mL). The reaction mixture was stirred at room temperature overnight, washed twice with aqueous ammonium hydroxide, and diluted with chloroform (50 mL). Ammonium hydroxide (25 mL) and p-toluenesulfonyl chloride (2.1 g, 11 mmol) were added to the solution, and the resulting mixture was stirred overnight at room temperature and diluted with ammonium hydroxide. The organic layer was separated and washed with ammonium hydroxide and concentrated under reduced pressure. The crude product was purified by automated flash chromatography on silica gel (silica cartridge, eluting with ethanol:dichloromethane in a gradient from 0:100 to 7:93 over 42 minutes) to provide 158 mg of N-{[4-amino-7-bromo-1-(tetrahydro-2H-pyran-4-ylmethyl)-1H-imidazo[4,5-c]quinolin-2-yl]methyl}cyclopropanecarboxamide as a white solid, mp 240° C.

Anal. calcd for C₂₁H₂₄BrN₅O₂: C, 55.03; H, 5.28; N, 15.28. Found: C, 55.13; H, 5.19; N, 15.51.

### Example 313

### 2-[(1,1-Dioxidoisothiazolidin-2-yl)methyl]-1-(2-methylpropyl)-1H-imidazo[4,5-c]quinolin-4-amine

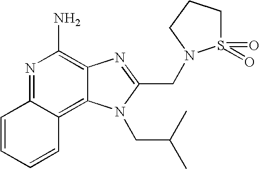 Part A

BOC-glycine N-hydroxysuccinimide ester (Boc-Gly-OSu) (30.0 g, 0.110 mmol) and pyridine hydrochloride (2.1 g) were added sequentially to a stirred suspension of N4-(2-methylpropyl)quinoline-3,4-diamine (21.5 g, 0.100 mol) in anhydrous pyridine (600 mL). The reaction mixture was heated at 40° C. for several hours and then heated at reflux overnight. An analysis by HPLC indicated the presence of starting material. The reaction was cooled to 40° C., and additional Boc-Gly-OSu (3.8 g) was added. The reaction mixture was heated at reflux for four hours, and then the solvent volume was reduced to 250 mL using a Dean-Stark trap. The reaction mixture was cooled to room temperature, and a precipitate formed, which was isolated by filtration and washed with pyridine (50 mL). The resulting light yellow solid was dissolved in dichloromethane. Deionized water (300 mL) was added, and the mixture was adjusted to pH 10 with the addition of solid sodium carbonate. The aqueous layer was separated and extracted once with dichloromethane (200 mL). The combined organic fractions were dried with sodium sulfate, filtered, and concentrated under reduced pressure. The resulting off-white solid was purified by column chromatography on silica gel (eluting with methanol:chloroform in a gradient from 1:99 to 3:97) to provide 13.5 g of tert-butyl[1-(2-methylpropyl)-1H-imidazo[4,5-c]quinolin-2-yl]methylcarbamate as a light green foam that was dried under high vacuum overnight.

MS (APCI) m/z 355 (M+H)+

Part B

mCPBA (11 g of 60% pure material, 38 mmol) was added in portions to a solution of tert-butyl[1-(2-methylpropyl)-1H-imidazo[4,5-c]quinolin-2-yl]methylcarbamate (13.5 g, 38.1 mmol) in chloroform (300 mL), and the reaction was stirred at room temperature for 30 minutes. Concenrated ammonium hydroxide (100 mL) was slowly added, and the mixture was stirred for ten minutes followed by the addition of p-toluenesulfonyl chloride (8.0 g, 42 mmoL) in portions. The reaction mixture was stirred for 60 minutes and then washed with 1% aqueous sodium carbonate (3×350 mL). The combined washings were extracted with dichloromethane (3×200 mL). The combined organic fractions were dried over sodium sulfate, filtered, and concentrated under reduced pressure to provide tert-butyl[4-amino-1-(2-methylpropyl)-1H-imidazo[4,5-c]quinolin-2-yl]methylcarbamate as a yellow-brown solid.

Part C

The material from Part B was dissolved in concentrated hydrochloric acid (100 mL). The resulting brown solution was treated with a heaping tablespoon of activated charcoal. The mixture was swirled and allowed to stand for 20 minutes. The charcoal was removed by filtration through a fritted glass disc, and the filter cake washed with deionized water until the filtrate was colorless. A precipitate formed in the filtrate and was isolated by filtration and dried in a vacuum oven to provide 9 g of 2-(aminomethyl)-1-(2-methylpropyl)-1H-imidazo[4,5-c]quinolin-4-amine hydrochloride as a white solid. The filtrate from the filtration of the salt was adjusted to pH 14 with the addition of solid sodium hydroxide. A precipitate formed and was isolated by filtration to provide 1 g of 2-(aminomethyl)-1-(2-methylpropyl)-1H-imidazo[4,5-c]quinolin-4-amine as a brown powder.

MS (APCI) m/z 270 (M+H)+.

Part D

DBU (2.5 mL) was added to a suspension of 2-(aminomethyl)-1-(2-methylpropyl)-1H-imidazo[4,5-c]quinolin-4-amine hydrochloride (1.5 g, 4.9 mmol) in chloroform (100 mL) at room temperature. 3-Chloropropanesulfonyl chloride (956 mg, 5.40 mmol) was added dropwise to the resulting mixture, and the solution was stirred at room temperature for 45 minutes. An analysis by HPLC indicated the reaction was incomplete, and additional DBU and 3-chloropropanesulfonyl chloride were added. The reaction was stirred for a total of two days and then washed with saturated aqueous ammonium chloride (3×100 mL). The combined aqueous fractions were extracted with chloroform (2×50 mL). The combined organic fractions were concentrated under reduced pressure. The resulting yellow oil was dissolved in DMF (10-20 mL), and deionized water (200 mL) was added. A white precipitate formed, and the mixture was stirred for three hours. The water was decanted away, and the resulting yellow solid was dissolved in dichloromethane (100 mL). The solution was dried with magnesium sulfate, filtered, and concentrated under reduced pressure. The resulting yellow solid (1.5 g) was purified by column chromatography on silica gel followed by recrystallization from acetonitrile. The crystals were dried in an oven overnight to provide 660 mg of 2-[(1,1-dioxidoisothiazolidin-2-yl)methyl]-1-(2-methylpropyl)-1H-imidazo[4,5-c]quinolin-4-amine as a white, crystalline solid, mp 230.5-232.5° C.

MS (APCI) m/z 374 (M+H)⁺;

Anal. calcd for C₁₈H₂₃N₅O₂S: C, 57.89; H, 6.21; N, 18.75. Found: C, 57.62; H, 6.26; N, 18.74.

### Example 314

### 2-[(1,1-Dioxidoisothiazolidin-2-yl)methyl]-1-(2-methylpropyl)-6,7,8,9-tetrahydro-1H-imidazo[4,5-c]quinolin-4-amine

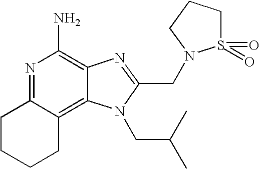

The mother liquor from the recrystallization in Part D of Example 313 was concentrated under reduced pressure to provide 2-[(1,1-dioxidoisothiazolidin-2-yl)methyl]-1-(2-methylpropyl)-1H-imidazo[4,5-c]quinolin-4-amine (150 mg, 0.4 mmol), which was dissolved in trifluoroacetic acid (25 mL). Platinum (IV) oxide (100 mg) was added, and the mixture was placed under hydrogen pressure (50 psi, 3.4×10⁵ Pa) for three days. The catalyst was removed by filtration, and the trifluoroacetic acid was removed under reduced pressure. The residue was dissolved in 3M hydrochloric acid, and the solution was adjusted to pH 13-14 with the addition of solid sodium hydroxide. The mixture was extracted with chloroform (5×25 mL), and the combined organic fractions were dried over magnesium sulfate, filtered, and concentrated under reduced pressure. The residue was purified by column chromatography on silica gel (eluting with 5:95 methanol:chloroform) followed by recrystallization from acetonitrile. The resulting light yellow crystals were dried in vacuum oven overnight to provide 35 mg of 2-[(1,1-dioxidoisothiazolidin-2-yl)methyl]-1-(2-methylpropyl)-6,7,8,9-tetrahydro-1H-imidazo[4,5-c]quinolin-4-amine as off-white crystals, mp 200-203° C.

MS (APCI) m/z 378 (M+H)⁺;

Anal. calcd for C₁₈H₂₇N₅O₂S: C, 57.27; H, 7.21; N, 18.55. Found: C, 57.50; H, 7.16; N, 18.42.

### Examples 315-335

Part A

A mixture of [4-amino-1-(2-methylpropyl)-1H-imidazo[4,5-c]quinolin-2-yl]methanol (10.9 g, 40.3 mmol, U.S. Pat. No. 5,389,640 Example 9), platinum (IV) oxide (5.5 g), and trifluoroacetic acid (75 mL) was placed under hydrogen pressure (50 psi, 3.4×10⁵ Pa) on a Parr apparatus for two days. The mixture was diluted with dichloromethane (200 mL) and filtered through CELITE filter agent; the filter cake washed with dichloromethane. The filtrate was concentrated under reduced pressure, and the residue was partitioned between dichloromethane (200 mL) and water (200 mL). The mixture was adjusted to pH 10 with the addition of solid sodium carbonate. The aqueous layer was separated and extracted with dichloromethane (2×200 mL). A solid was present in the aqueous layer and was isolated by filtration, washed with water, and combined with the organic fractions. The combined organic fractions were concentrated under reduced pressure and purified by automated flash chromatography using a HORIZON HPFC system (silica gel, eluting with dichloromethane: 1 M ammonia in methanol in a gradient from 95:5 to 80:20) to afford 4.92 g of [4-amino-1-(2-methylpropyl)-6,7,8,9-tetrahydro-1H-imidazo[4,5-c]quinolin-2-yl]methanol as a grey solid.

Part B

Thionyl chloride (1.56 mL, 21.4 mmol) was added to a stirred suspension of [4-amino-1-(2-methylpropyl)-6,7,8,9-tetrahydro-1H-imidazo[4,5-c]quinolin-2-yl]methanol (4.92 g, 17.9 mmol) in 1,2-dichloroethane (180 mL). The reaction became homogeneous, and then a precipitate formed after five minutes. The reaction mixture was stirred at room temperature for 1.5 hours and concentrated under reduced pressure to yield 2-(chloromethyl)-1-(2-methylpropyl)-6,7,8,9-tetrahydro-1H-imidazo[4,5-c]quinolin-4-amine hydrochloride as a tan solid.

Part C

A cyclic amine (0.15 mmol, 1.5 equivalents) from the table below was added to a test tube containing 2-(chloromethyl)-1-(2-methylpropyl)-6,7,8,9-tetrahydro-1H-imidazo[4,5-c]quinolin-4-amine hydrochloride (33 mg, 0.10 mmol), potassium carbonate (0.055 g, 0.40 mmol), and DMA (1 mL). The test tube was capped and heated for ten hours at 70° C. Each reaction mixture was allowed to cool to room temperature and filtered, and the filter cake washed with DMA (0.200 mL). The solvent was removed from each filtrate by vacuum centrifugation.

The compounds were purified by reversed phase prep HPLC using a Waters FractionLynx automated purification system. The prep HPLC fractions were analyzed using a Waters LC/TOF-MS, and the appropriate fractions were centrifuge evaporated to provide the trifluoroacetate salt of the desired compound. Reversed phase preparative liquid chromatography was performed with non-linear gradient elution from 5-95% B where A is 0.05% trifluoroacetic acid/water and B is 0.05% trifluoroacetic acid/acetonitrole. Fractions were collected by mass-selective triggering. The table below shows the reagent added to each test tube, the structure of the resulting compound, and the observed accurate mass for the isolated trifluoroacetate salt.

### Examples 315-335

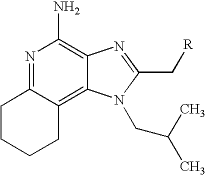

| | | | Measured |
|---|---|---|---|
| | | | Mass |
| Example | Reagent | R | (M + H) |
| 315 | 2-Methylpyrrolidine | 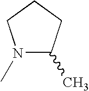 | 342.2664 |
| 316 | (R)-3-Hydroxy- pyrrolidine | 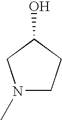 | 344.2443 |
| 317 | 4-Methylpiperidine | 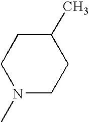 | 356.2797 |
| 318 | 2-Methylpiperidine | 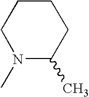 | 356.2813 |
| 319 | 1-Methylpiperazine | 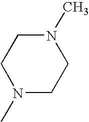 | 357.2769 |
| 320 | 3-Hydroxypiperidine | 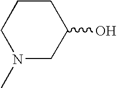 | 358.2614 |
| 321 | 4-Hydroxypiperidine | 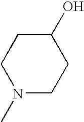 | 358.2592 |
| 322 | Thiomorpholine | 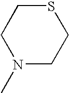 | 360.2228 |
| 323 | 1-Piperazine- carboxaldehyde | 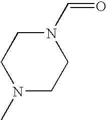 | 371.2567 |
| 324 | N-Ethylpiperazine | 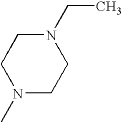 | 371.2915 |
| 325 | N-Methyl- homopiperazine | 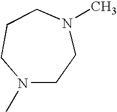 | 371.2895 |
| 326 | 2-Piperidinemethanol | 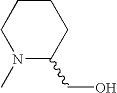 | 372.2767 |
| 327 | 3-(Hydroxy- methyl)piperidine | 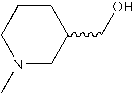 | 372.2769 |
| 328 | 4-(Hydroxy- methyl)piperidine | 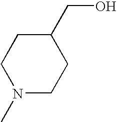 | 372.2752 |
| 329 | Isonipecotamide | 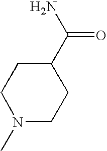 | 385.2723 |
| 330 | Nipecotamide | 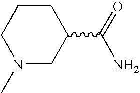 | 385.2737 |
| 331 | (3S)-(−)-3- Acetamidopyrrolidine | 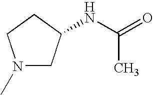 | 385.2729 |
| 332 | 1-Acetylpiperazine | 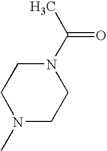 | 385.2706 |
| 333 | 2-Piperidinethanol | 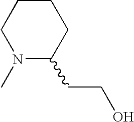 | 386.2928 |
| 334 | 4-Piperidineethanol | 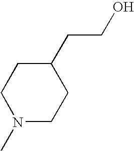 | 386.2922 |
| 335 | N-(2-Hydroxy- ethyl)piperazine | 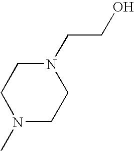 | 387.2881 |

### Examples 336-348

Part A

A mixture of the material from Part B of Examples 315-335 (3.75 g, 11.4 mmol), potassium phthalimide (2.53 g, 13.7 mmol), potassium carbonate (4.72 g, 34.2 mmol), and DMF (75 mL) was stirred at room temperature overnight. Water (300 mL) was added. A solid was present and was isolated by filtration and washed with water and diethyl ether to provide 3.1 g of 2-{[4-amino-1-(2-methylpropyl)-6,7,8,9-tetrahydro-1H-imidazo[4,5-c]quinolin-2-yl]methyl)}-1H-isoindole-1,3(2H)-dione as a yellow solid. Part B

Hydrazine (0.745 mL, 15.4 mmol) was added to a stirred suspension of 2-{[4-amino-1-(2-methylpropyl)-6,7,8,9-tetrahydro-1H-imidazo[4,5-c]quinolin-2-yl]methyl)}-1H-isoindole-1,3(2H)-dione (3.1 g, 7.7 mmol) in ethanol (35 mL). After 2.5 hours at room temperature, the reaction became homogeneous. The reaction was stirred at room temperature overnight, concentrated under reduced pressure, dissolved in methanol, and purified by automated flash chromatography using a HORIZON HPFC system (FLASH 40+M cartridge, eluting sequentially with 90:10 chloroform:methanol and dichloromethane: 1 M ammonia in methanol in a gradient from 90:10 to 80:20) to provide 1.77 g of 2-(aminomethyl)-1-(2-methylpropyl)-6,7,8,9-tetrahydro-1H-imidazo[4,5-c]quinolin-4-amine as a yellow solid.

Part C

An acid chloride (0.11 mmol, 1.1 equivalents) from the table below was added to a test tube containing 2-(aminomethyl)-1-(2-methylpropyl)-6,7,8,9-tetrahydro-1H-imidazo[4,5-c]quinolin-4-amine (27 mg, 0.10 mmol) and N,N-diisopropylethylamine (0.034 mL, 0.20 mmol) in DMA (1 mL). The test tube was capped and vortexed overnight at ambient temperature. Two drops of water were added to each test tube, and the solvent was removed by vacuum centrifugation.

The compounds were purified according to the method described in Part C of Examples 315-335. The table below shows the reagent added to each test tube, the structure of the resulting compound, and the observed accurate mass for the isolated trifluoroacetate salt.

### Examples 336-348

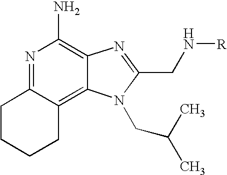

| | | | Measured |
|---|---|---|---|
| | | | Mass |
| Example | Reagent | R | (M + H) |
| | None | —H | 274.2017 |
| 336 | Butyryl chloride | 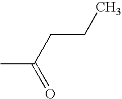 | 344.2430 |
| 337 | Isobutyryl chloride | 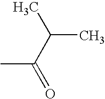 | 344.2427 |
| 338 | Methoxyacetyl chloride | 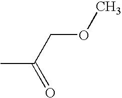 | 346.2211 |
| 339 | Cyclobutanecarbonyl chloride | 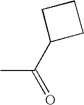 | 356.2419 |
| 340 | DL-2-Methylbutyryl chloride | 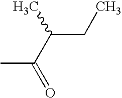 | 358.2572 |
| 341 | Pivaloyl chloride | 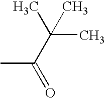 | 358.2593 |
| 342 | Cyclopentanecarbonyl chloride | 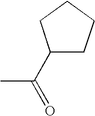 | 370.2597 |
| 343 | tert-Butylacetyl chloride | 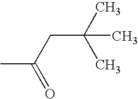 | 372.2786 |
| 344 | Benzoyl chloride | 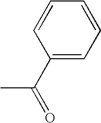 | 378.2288 |
| 345 | 2-Chlorobenzoyl chloride | 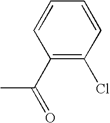 | 412.1880 |
| 346 | 3-Chlorobenzoyl chloride | 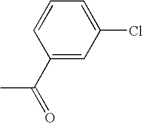 | 412.1911 |
| 347 | Nicotinoyl chloride hydrochloride | 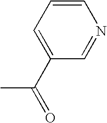 | 379.2234 |
| 348 | 3,4-Dichlorobenzoyl chloride | 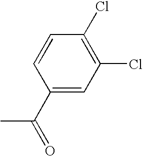 | 446.1481 |

Compounds of the invention have been found to modulate cytokine biosynthesis by inducing the production of interferon α and/or tumor necrosis factor α in human cells when tested using the methods described below.

### Cytokine Induction in Human Cells

An in vitro human blood cell system is used to assess cytokine induction. Activity is based on the measurement of interferon (α) and tumor necrosis factor (α) (IFN-α and TNF-α, respectively) secreted into culture media as described by Testerman et. al. in “Cytokine Induction by the Immunomodulators Imiquimod and S-27609”, Journal of Leukocyte Biology, 58, 365-372 (September, 1995).

Blood Cell Preparation for Culture

Whole blood from healthy human donors is collected by venipuncture into vacutainer tubes or syringes containing EDTA. Peripheral blood mononuclear cells (PBMC) are separated from whole blood by density gradient centrifugation using HISTOPAQUE-1077 (Sigma, St. Louis, Mo.) or Ficoll-Paque Plus (Amersham Biosciences Piscataway, N.J.). Blood is diluted 1:1 with Dulbecco's Phosphate Buffered Saline (DPBS) or Hank's Balanced Salts Solution (HBSS). Alternately, whole blood is placed in Accuspin (Sigma) or LeucoSep (Greiner Bio-One, Inc., Longwood, Fla.) centrifuge frit tubes containing density gradient medium. The PBMC layer is collected and washed twice with DPBS or HBSS and re-suspended at 4×10⁶ cells/mL in RPMI complete. The PBMC suspension is added to 96 well flat bottom sterile tissue culture plates containing an equal volume of RPMI complete media containing test compound.

Compound Preparation

The compounds are solubilized in dimethyl sulfoxide (DMSO). The DMSO concentration should not exceed a final concentration of 1% for addition to the culture wells. The compounds are generally tested at concentrations ranging from 30-0.014 μM. Controls include cell samples with media only, cell samples with DMSO only (no compound), and cell samples with reference compound.

Incubation

The solution of test compound is added at 60 μM to the first well containing RPMI complete and serial 3 fold dilutions are made in the wells. The PBMC suspension is then added to the wells in an equal volume, bringing the test compound concentrations to the desired range (usually 30-0.014 μM). The final concentration of PBMC suspension is 2×10⁶ cells/mL. The plates are covered with sterile plastic lids, mixed gently and then incubated for 18 to 24 hours at 37° C. in a 5% carbon dioxide atmosphere.

Separation

Following incubation the plates are centrifuged for 10 minutes at 1000 rpm (approximately 200×g) at 4° C. The cell-free culture supernatant is removed and transferred to sterile polypropylene tubes. Samples are maintained at −30 to −70° C. until analysis. The samples are analyzed for IFN-α by ELISA and for TNF-α by IGEN/BioVeris Assay.

Interferon (α) and Tumor Necrosis Factor (α) Analysis

IFN-α concentration is determined with a human multi-subtype colorimetric sandwich ELISA (Catalog Number 41105) from PBL Biomedical Laboratories, Piscataway, N.J. Results are expressed in pg/mL.

The TNF-α concentration is determined by ORIGEN M-Series Immunoassay and read on an IGEN M-8 analyzer from BioVeris Corporation, formerly known as IGEN International, Gaithersburg, Md. The immunoassay uses a human TNF-α capture and detection antibody pair (Catalog Numbers AHC3419 and AHC3712) from Biosource International, Camarillo, Calif. Results are expressed in pg/mL.

Assay Data and Analysis

In total, the data output of the assay consists of concentration values of TNF-α and IFN-α (y-axis) as a function of compound concentration (x-axis).

Analysis of the data has two steps. First, the greater of the mean DMSO (DMSO control wells) or the experimental background (usually 20 pg/mL for IFN-α and 40 pg/mL for TNF-α) is subtracted from each reading. If any negative values result from background subtraction, the reading is reported as “*”, and is noted as not reliably detectable. In subsequent calculations and statistics, “*”, is treated as a zero. Second, all background subtracted values are multiplied by a single adjustment ratio to decrease experiment to experiment variability. The adjustment ratio is the area of the reference compound in the new experiment divided by the expected area of the reference compound based on the past 61 experiments (unadjusted readings). This results in the scaling of the reading (y-axis) for the new data without changing the shape of the dose-response curve. The reference compound used is 2-[4-amino-2-ethoxymethyl-6,7,8,9-tetrahydro-α,α-dimethyl-1H-imidazo[4,5-c]quinolin-1-yl]ethanol hydrate (U.S. Pat. No. 5,352,784; Example 91) and the expected area is the sum of the median dose values from the past 61 experiments.

The minimum effective concentration is calculated based on the background-subtracted, reference-adjusted results for a given experiment and compound. The minimum effective concentration (μmolar) is the lowest of the tested compound concentrations that induces a response over a fixed cytokine concentration for the tested cytokine (usually 20 pg/mL for IFN-α and 40 pg/mL for TNF-α). The maximal response is the maximal amount of cytokine (pg/ml) produced in the dose-response.

### Cytokine Induction in Human Cells

### High Throughput Screen

The CYTOKINE INDUCTION IN HUMAN CELLS test method described above was modified as follows for high throughput screening.

Blood Cell Preparation for Culture

Whole blood from healthy human donors is collected by venipuncture into vacutainer tubes or syringes containing EDTA. Peripheral blood mononuclear cells (PBMC) are separated from whole blood by density gradient centrifugation using HISTOPAQUE-1077 (Sigma, St. Louis, Mo.) or Ficoll-Paque Plus (Amersham Biosciences Piscataway, N.J.). Whole blood is placed in Accuspin (Sigma) or LeucoSep (Greiner Bio-One, Inc., Longwood, Fla.) centrifuge frit tubes containing density gradient medium. The PBMC layer is collected and washed twice with DPBS or HBSS and re-suspended at 4×10⁶ cells/mL in RPMI complete (2-fold the final cell density). The PBMC suspension is added to 96-well flat bottom sterile tissue culture plates.

Compound Preparation

The compounds are solubilized in dimethyl sulfoxide (DMSO). The compounds are generally tested at concentrations ranging from 30-0.014 μM. Controls include cell samples with media only, cell samples with DMSO only (no compound), and cell samples with a reference compound 2-[4-amino-2-ethoxymethyl-6,7,8,9-tetrahydro-α,α-dimethyl-1H-imidazo[4,5-c]quinolin-1-yl]ethanol hydrate (U.S. Pat. No. 5,352,784; Example 91) on each plate. The solution of test compound is added at 7.5 mM to the first well of a dosing plate and serial 3 fold dilutions are made for the 7 subsequent concentrations in DMSO. RPMI Complete media is then added to the test compound dilutions in order to reach a final compound concentration of 2-fold higher (60-0.028 μM) than the final tested concentration range.

Incubation

Compound solution is then added to the wells containing the PBMC suspension bringing the test compound concentrations to the desired range (usually 30-0.014 μM) and the DMSO concentration to 0.4%. The final concentration of PBMC suspension is 2×10⁶ cells/mL. The plates are covered with sterile plastic lids, mixed gently and then incubated for 18 to 24 hours at 37° C. in a 5% carbon dioxide atmosphere.

Separation

Following incubation the plates are centrifuged for 10 minutes at 1000 rpm (approximately 200 g) at 4° C. 4-plex Human Panel MSD MULTI-SPOT 96-well plates are pre-coated with the appropriate capture antibodies by MesoScale Discovery, Inc. (MSD, Gaithersburg, Md.). The cell-free culture supernatants are removed and transferred to the MSD plates. Fresh samples are typically tested, although they may be maintained at −30 to −70° C. until analysis.

Interferon-α and Tumor Necrosis Factor-α Analysis

MSD MULTI-SPOT plates contain within each well capture antibodies for human TNF-α and human IFN-α that have been pre-coated on specific spots. Each well contains four spots: one human TNF-α capture antibody (MSD) spot, one human IFN-α capture antibody (PBL Biomedical Laboratories, Piscataway, N.J.) spot, and two inactive bovine serum albumin spots. The human TNF-α capture and detection antibody pair is from MesoScale Discovery. The human IFN-α multi-subtype antibody (PBL Biomedical Laboratories) captures all IFN-α subtypes except IFN-α F (IFNA21). Standards consist of recombinant human TNF-α (R&D Systems, Minneapolis, Minn.) and IFN-α (PBL Biomedical Laboratories). Samples and separate standards are added at the time of analysis to each MSD plate. Two human IFN-α detection antibodies (Cat. Nos. 21112 & 21100, PBL) are used in a two to one ratio (weight:weight) to each other to determine the IFN-α concentrations. The cytokine-specific detection antibodies are labeled with the SULFO-TAG reagent (MSD). After adding the SULFO-TAG labeled detection antibodies to the wells, each well's electrochemoluminescent levels are read using MSD's SECTOR HTS READER. Results are expressed in pg/mL upon calculation with known cytokine standards.

Assay Data and Analysis

In total, the data output of the assay consists of concentration values of TNF-α or IFN-α (y-axis) as a function of compound concentration (x-axis).

A plate-wise scaling is performed within a given experiment aimed at reducing plate-to-plate variability associated within the same experiment. First, the greater of the median DMSO (DMSO control wells) or the experimental background (usually 20 pg/mL for IFN-α and 40 pg/mL for TNF-α) is subtracted from each reading. Negative values that may result from background subtraction are set to zero. Each plate within a given experiment has a reference compound that serves as a control. This control is used to calculate a median expected area under the curve across all plates in the assay. A plate-wise scaling factor is calculated for each plate as a ratio of the area of the reference compound on the particular plate to the median expected area for the entire experiment. The data from each plate are then multiplied by the plate-wise scaling factor for all plates. Only data from plates bearing a scaling factor of between 0.5 and 2.0 (for both cytokines IFN-α, TNF-α) are reported. Data from plates with scaling factors outside the above mentioned interval are retested until they bear scaling factors inside the above mentioned interval. The above method produces a scaling of the y-values without altering the shape of the curve. The reference compound used is 2-[4-amino-2-ethoxymethyl-6,7,8,9-tetrahydro-α,α-dimethyl-1H-imidazo[4,5-c]quinolin-1-yl]ethanol hydrate (U.S. Pat. No. 5,352,784; Example 91). The median expected area is the median area across all plates that are part of a given experiment.

A second scaling may also be performed to reduce inter-experiment variability (across multiple experiments). All background-subtracted values are multiplied by a single adjustment ratio to decrease experiment-to-experiment variability. The adjustment ratio is the area of the reference compound in the new experiment divided by the expected area of the reference compound based on an average of previous experiments (unadjusted readings). This results in the scaling of the reading (y-axis) for the new data without changing the shape of the dose-response curve. The reference compound used is 2-[4-amino-2-ethoxymethyl-6,7,8,9-tetrahydro-α,α-dimethyl-1H-imidazo[4,5-c]quinolin-1-yl]ethanol hydrate (U.S. Pat. No. 5,352,784; Example 91) and the expected area is the sum of the median dose values from an average of previous experiments.

The minimum effective concentration is calculated based on the background-subtracted, reference-adjusted results for a given experiment and compound. The minimum effective concentration (μmolar) is the lowest of the tested compound concentrations that induces a response over a fixed cytokine concentration for the tested cytokine (usually 20 pg/mL for IFN-α and 40 pg/mL for TNF-α). The maximal response is the maximal amount of cytokine (pg/ml) produced in the dose-response.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1-11. (canceled)

12. A compound of the following formula (V) 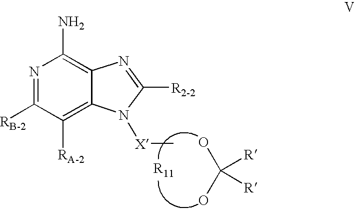
wherein:
R′ is selected from the group consisting of hydrogen, alkyl, alkoxy, and alkoxyalkylenyl, or the R′ groups join together to form a 5 to 7 membered saturated ring optionally substituted by phenyl or phenyl substituted with one or more substituents selected from the group consisting of alkyl, alkoxy, halogen, and trifluoromethyl;
X′ is selected from the group consisting of —CH(R₉)—, —CH(R₉)-alkylene-, and —CH(R₉)-alkenylene-; wherein the alkylene and alkenylene are optionally interrupted with one or more —O— groups;
R₁₁ is a straight chain C₂₋₃ alkylene;
R_{A-2} and R_{B-2} are each independently selected from the group consisting of:
hydrogen,
halogen,
alkyl,
alkenyl,
alkoxy,
alkylthio, and
—N(R₉)₂;
or R_{A-2} and R_{B-2} taken together form either a fused aryl ring that is unsubstituted or substituted by one or more Rₐ groups, or a fused 5 to 7 membered saturated ring that is unsubstituted or substituted by one or more R_{c} groups:
or R_{A-2} and R_{B-2} taken together form a fused heteroaryl or 5 to 7 membered saturated ring, containing one heteroatom selected from the group consisting of N and S, wherein the heteroaryl ring is unsubstituted or substituted by one or more R_{b} groups, and the 5 to 7 membered saturated ring is unsubstituted or substituted by one or more R_{c} groups;
Rₐ is selected from the group consisting of fluorine, alkyl, haloalkyl, alkoxy, and —N(R₉)₂;
R_{b} is selected from the group consisting of halogen, hydroxy, alkyl, haloalkyl, alkoxy, and —N(R₉)₂;
R_{c} is selected from the group consisting of halogen, hydroxy, alkyl alkenyl, haloalkyl, alkoxy, alkylthio, and —N(R₉)₂;
R₂₋₂ is selected from the group consisting of
—R₄,
—X—R₄,
—X—Y—R₄, and
—X—R₅ₐ;
X is selected from the group consisting of alkylene, alkenylene, alkynylene, arylene, heteroarylene, and heterocyclylene wherein the alkylene, alkenylene, and alkynylene groups are optionally interrupted or terminated by arylene, heteroarylene or heterocyclylene and optionally interrupted by one or more —O— groups;
Y is selected from the group consisting of:
—S(O)₀₋₂—,
—S(O)₂—N(R₈)—,
—C(R₆)—,
—C(R₆)—O—,
—O—C(R₆)—,
—O—C(O)—O—.
—N(R₉)-Q′-,
—C(R₆)—N(Rₐ)—,
—O—C(R₆)—N(R₈)—,
—C(R₆)—N(OR₉)—, 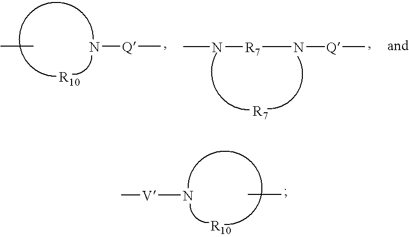
R₄ is selected from the group consisting of hydrogen, alkyl, alkenyl, alkynyl, aryl, arylalkylenyl, aryloxyalkylenyl, alkylarylenyl, heteroaryl, heteroaryalkylenyl, heteroaryloxyalkylenyl, alkylheteroarylenyl, and heterocyclyl wherein the alkyl, alkenyl, alkynyl, aryl, arylalkylenyyl, aryloxyalkylenyl, alkylarylenyl, heteroaryl, heteroarylalkylenyl, heteroaryloxyalkylenyl, alkylheteroarylenyl, and heterocycyl groups are unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;
R_{5c} is selected from the group consisting of: 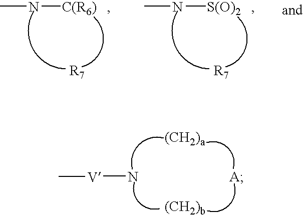
R₆ is selected from the group consisting of ═O and ═S;
R₇ is C₂₋₇ alkylene:
R₈ is selected from the group consisting of hydrogen, alkyl, alkoxyalkylenyl, and arylalkylenyl;
R₉ is selected from the group consisting of hydrogen and alkyl;
R₁₀ is C₃₋₈ alkylene;
A is selected from the group consisting of —O—, —C(O)—, —S(O)₀₋₂—, —CH₂—, and —N(R₄)—;
Q′ is selected from the group consisting of a bond, —C(R₆)—, —C(R₆)—C(R₆)—, —S(O)₂—, and —S(O)₂—N(R₈)—;
V′ is selected from the group consisting of —C(R₆)—, —O—C(R₆)—, and —S(O)₂—; and
a and b are each independently an integer from 1 to 6 with the proviso that a+b is ≦7;
or a pharmaceutically acceptable salt thereof.

13-21. (canceled)

22. A compound of the following formula (XI): 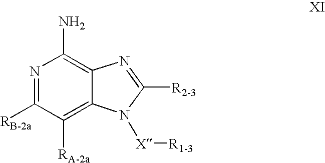
wherein:
R₁₋₃ is selected from the group consisting of: 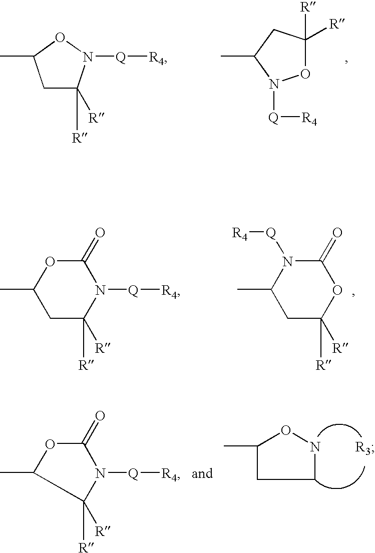
R₃ is C₃₋₅ alkylene;
R″ is selected from the group consisting of:
hydrogen,
alkyl,
alkenyl,
aryl,
arylalkylenyl,
heteroaryl,
heteroarylalkylenyl,
heterocyclyl,
heterocyclylalkylenyl, and
alkyl, alkenyl, aryl arylalkylenyl, heteroaryl, heteroarylalkylenyl, heterocyclyl, or heterocyclylalkylenyl, substituted by one or more substituents selected from the group consisting of:
hydroxy,
alkyl,
haloalkyl,
hydroxyalkyl,
alkoxy,
dialkylamino,
—S(O)₀₋₂-alkyl,
—S(O)₀₋₂-aryl,
—NH—S(O)₂-alkyl,
—NH—S(O)₂-aryl,
haloalkoxy,
halogen,
nitrile,
nitro,
aryl,
heteroaryl,
heterocyclyl,
aryloxy,
arylalkyleneoxy,
—C(O)—O-alkyl,
—C(O)—N(R₈)₂,
—N(R₈)—C(O)-alkyl,
—O—(CO)-alkyl, and
—C(O)-alkyl;
or two R″ groups on the same carbon atom can join together to form a ring system selected from the group consisting of 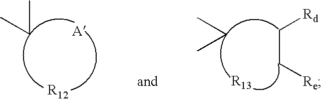
R_{d} and Rₑ are independently selected from the group consisting of hydrogen, halogen, hydroxy, alkyl, alkenyl, aryl, haloalkyl, alkoxy, alkylthio, and —N(R₉)₂; or R_{d} and Rₑ can join to form a fused aryl ring or fused 5-10 membered heteroaryl ring containing one to four heteroatoms;
A′ is selected from the group consisting of —O—, —S(O)₀₋₂—, —N(-Q-R₄)—, and —CH₂—;
R₁₂ is C₃₋₉ alkylene or C₃₋₉ alkenylene, optionally interrupted by one heteroatom;
R₁₃ is C₂₋₇ alkylene or C₂₋₇ alkenylene, optionally interrupted by one heteroatom;
X″ is selected from the group consisting of —CH(R₉)—, —CH(R₉)-alkylene-, and —CH(R₉)-alkenylene-;
R_{A-2a}, and R_{B-2a}, are each independently selected from the group consisting of:
hydrogen,
halogen,
alkyl,
alkenyl,
alkoxy,
alkylthio, and
—N(R₉)₂;
or R_{A-2a} and R_{B-2a} taken together form either a fused aryl ring that is unsubstituted or substituted by one or more 11 groups, or a fused 5 to 7 membered saturated ring that is unsubstituted or substituted by one or more R_{c} groups;
or R_{A-2a} and R_{B-2a} taken together form a fused heteroaryl or 5 to 7 membered saturated ring containing one heteroatom selected from the group consisting of N and S, wherein the heteroaryl ring is unsubstituted or substituted by one or more R_{b} groups, and the 5 to 7 membered saturated ring is unsubstituted or substituted by one or more R_{c} groups;
Rₐ₁ is selected from the group consisting of halogen, alkyl, haloalkyl, alkoxy, and —N(R₉)₂;
R_{b} is selected from the group consisting of halogen, hydroxy, alkyl, haloalkyl, alkoxy, and —N(R₉)₂;
R_{c} is selected from the group consisting of halogen, hydroxy, alkyl, alkenyl, haloalkyl, alkoxy, alkylthio, and —N(R₉)₂;
R₂₋₃ is selected from the group consisting of:
—R₄,
—X—R₄,
—X—Y—R₄, and
—X—R₅ₐ;
X is selected from the group consisting of alkylene, alkenylene, alkynylene, arylene, heteroarylene, and heterocyclylene wherein the alkylene, alkenylene, and alkynylene groups are optionally interrupted or terminated by arylene, heteroarylene or heterocyclylene and optionally interrupted by one or more —O— groups;
Y is selected from the group consisting of:
—S(O)₀₋₂—,
—S(O)₂—N(R₈)—,
—C(R₆)—,
—C(R₆)—O—,
—O—C(R₆)—,
—O—C(O)—O—,
—N(R₈)-Q′-,
—C(R₆)—N(R₈)—,
—O—C(R₆)—N(R₈)—,
—C(R₆)—N(OR₉)—, 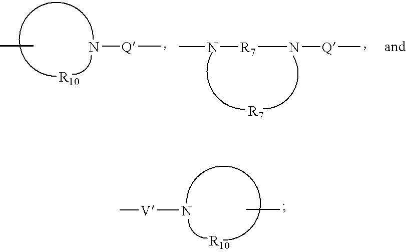
R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups are unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;
R₅ₐ is selected from the group consisting of: 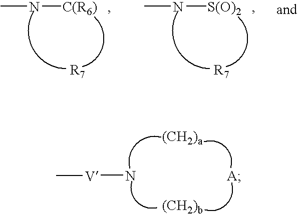
R₆ is selected from the group consisting of ═O and ═S;
R₇ is C₂₋₇ alkylene;
R₈ is selected from the group consisting of hydrogen, alkyl, alkoxyalkylenyl, and arylalkylenyl;
R₉ is selected from the group consisting of hydrogen and alkyl;
R₁₀ is C₃₋₈ alkylene;
A is selected from the group consisting of —O—, —C(O)—, —S(O)₀₋₂—, —CH₂—, and —N(R₄)—;
Q is selected from the group consisting of a bond, —C(R₆)—, —C(R₆)—C(R₆)—, —S(O)₂—, —C(R₆)—N(R₈)—W—, —S(O)₂—N(R₈)—, and —C(R₆)—O—, and —C(R₆)—N(OR₉)—;
Q′ is selected from the group consisting of a bond, —C(R₆)—, —C(R₆)—C(R₆)—, —S(O)₂—, and —S(O)₂—N(R₈)—;
V′ is selected from the group consisting of —C(R₆)—, —O—C(R₆)—, and —S(O)₂—;
W is selected from the group consisting of a bond, —C(O)—, and —S(O)₂—; and
a and b are each independently an integer from 1 to 6 with the proviso that a+b is ≦7;
or a pharmaceutically acceptable salt thereof.

23-31. (canceled)

32. A compound of the following formula (XIXa): 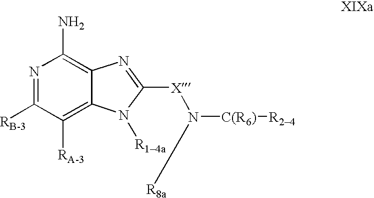
wherein:
X′″ is selected from the group consisting of C₁₋₄ alkylene and C₂₋₄ alkenylene;
R₂₋₄ is selected from the group consisting of C₃₋₆ alkyl, C₂₋₆ alkenyl, C₂₋₆ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl wherein the C₃₋₆ alkyl, C₂₋₆ alkenyl, C₂₋₆ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl groups are unsubstituted or substituted by one or more substituents independently selected from the group consisting of C₁₋₄ alkyl, C₁₋₄ alkoxy, C₁₋₄ alkanoyl, C₁₋₄ alkoxycarbonyl, hydroxyC₁₋₄ alkyl, haloC₁₋₄ alkyl, haloC₁₋₄ alkoxy, halogen, nitro, hydroxy, mercapto, cyano, amino, C₁₋₄ alkylamino, di(C₁₋₄ alkyl)amino, and in the case of C₃₋₆ alkyl, C₂₋₆ alkenyl, C₂₋₆ alkynyl, and heterocyclyl, oxo;
R_{A-3} and R_{B-3} form a fused aryl ring that is unsubstituted or substituted by one or more Rₐ₁ groups, or R_{A-3} and R_{B-3} form a fused 5 to 7 membered saturated ring that is unsubstituted or substituted by one or more R_{c} groups;
Rₐ₁ is selected from the group consisting of halogen, alkyl, haloalkyl, alkoxy, and —N(R₉)₂;
R_{c} is selected from the group consisting of halogen, hydroxy, alkyl, alkenyl, haloalkyl, alkoxy, alkylthio, and —N(R₉)₂,
R₁₋₄ₐ is selected from the group consisting of:
hydrogen,
alkyl,
alkenyl,
alkoxyalkylenyl,
aryl,
arylalkylenyl, 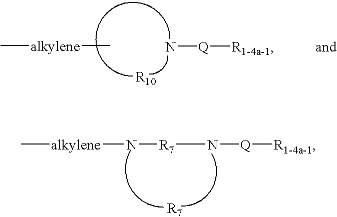
wherein the alkyl, alkenyl, alkoxyalkylenyl, aryl, and arylalkylenyl can be unsubstituted or substituted with one or more substituents selected from the group consisting of alkyl, alkoxy, hydroxyalkyl, haloalkyl, haloalkoxy, halogen, nitro, hydroxy, mercapto, cyano, amino, alkylamino, and dialkylamino;
with the proviso that when R₁₋₄ₐ includes a carbocyclic ring, then the ring carbon atom by which the ring is attached is otherwise unsubstituted or substituted by an atom other than O, S, or N;
R₁₋₄ₐ₋₁ is selected from the group consisting of alkyl, alkenyl, alkoxyalkylenyl, aryl, and arylalkylenyl, wherein the alkyl, alkenyl, alkoxyalkylenyl, aryl, and arylalkylenyl can be unsubstituted or substituted with one or more substituents selected from the group consisting of alkyl, alkoxy, hydroxyalkyl, haloalkyl, haloalkoxy, halogen, nitro, hydroxy, mercapto, cyano, amino, alkylamino, and dialkylamino;
Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—;
W is selected from the group consisting of a bond, —C(O)—, and —S(O)₂—;
R₆ is selected from the group consisting of ═O and ═S;
R₇ is C₂₋₇ alkylene;
R₈ₐ is selected from the group consisting of hydrogen and C₁₋₄ alkyl;
R₈ is selected from the group consisting of hydrogen, alkyl, alkoxyalkylenyl, and arylalkylenyl;
R₉ is selected from the group consisting of hydrogen and alkyl; and
R₁₀ is C₃₋₈ alkylene;
or a pharmaceutically acceptable salt thereof.

33-43. (canceled)

44. A compound of the following formula (XIXc): 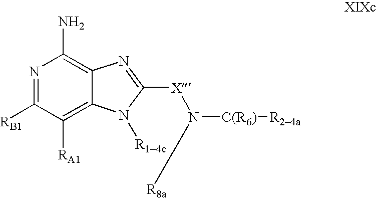
wherein:
X″ is selected from the group consisting of C₁₋₄ alkylene and C₂₋₄ alkenylene;
R₂₋₄ₐ is selected from the group consisting of C₁₋₆ alkyl, C₂₋₆ alkenyl, C₂₋₆ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl wherein the C₁₋₆alkyl, C₂₋₆alkenyl, C₂₋₆alkynyl, aryl, arylC₁₋₄alkylenyl, aryloxyC₁₋₄ alkylenyl, C₁₋₄ alkylarylenyl, heteroaryl, heteroarylC₄ alkylenyl, heteroaryloxyC₁₋₄alkylenyl, C₁₋₄alkylheteroarylenyl, and heterocyclyl groups are unsubstituted or substituted by one or more substituents independently selected from the group consisting of C₁₋₄ alkyl, C₁₋₄ alkoxy, C₁₋₄ alkanoyl, C₁₋₄ alkoxycarbonyl, hydroxyC₁₋₄ alkyl, haloC₁₋₄ alkyl, haloC₁₋₄ alkoxy, halogen, nitro, hydroxy, mercapto, cyano, amino, C₁₋₄ alkylamino, di(C₁₋₄ alkyl)amino, and in the case of C₂₋₆ alkyl, C₂₋₆ alkenyl, C₂₋₆ alkynyl, and heterocyclyl, oxo;
R_{A1} and R_{B1} are each independently selected from the group consisting of:
hydrogen,
halogen,
alkyl,
alkenyl,
alkoxy,
alkylthio, and
—N(R₉)₂;
R_{1-4c} is selected from the group consisting of:
—R₁ₐ,
—X₃—Y₃—R₄ₐ,
—X₂—R₄ₐ,
—X₂—Y₂—R₄ₐ, and
—X₂—R₅₋₁;
X₂ is selected from the group consisting of alkylene, alkenylene, alkynylene, arylene, heteroarylene, and heterocyclylene wherein the alkylene, alkenylene, and alkynylene groups are interrupted by one or more —O— groups and can be optionally interrupted or terminated with arylene, heteroarylene, or heterocyclylene;
X₃ is selected from the group consisting of alkylene, arylene, heteroarylene, and heterocyclylene wherein the alkylene group can be optionally interrupted or terminated by arylene, heteroarylene or heterocyclylene and optionally interrupted by one or more —O— groups;
Y₂ is selected from the group consisting of
—S(O)₀₋₂—,
—C(R₆)—O—,
—O—C(O)—O—,
—N(R₈)-Q-, and
—O—C(R₆)—N(R₈)—;
Y₃ is selected from the group consisting of: 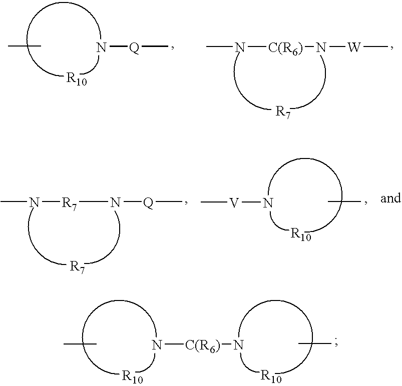
R₄ₐ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkylenyl, haloalkylenyl, haloalkyleneoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the ease of heterocyclyl, oxo;
with the proviso that when R_{1-4c} is —R₄ₐ, and R₄ₐ includes a carbocyclic ring or heterocyclic ring containing one heteroatom, then the ring carbon atom by which the ring is attached is otherwise unsubstituted or substituted by an atom other than O, S, or N;
with the further proviso that R_{1-4c} is other than an unsubstituted or substituted isoxazolylalkylenyl, dihydroisoxazolylalkylenyl, or oxadiazolylalkylenyl group;
R₅₋₁ is selected from the group consisting of: 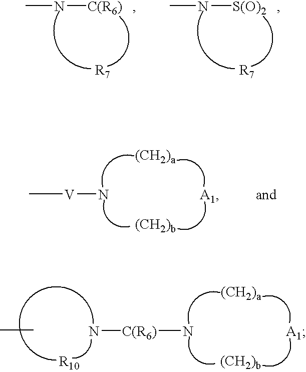
R₆ is selected from the group consisting of ═O and ═S;
R₇ is C₂₋₇ alkylene;
R₈ₐ is selected from the group consisting of hydrogen and C₁₋₄ alkyl;
R₈ is selected from the group consisting of hydrogen, alkyl, alkoxyalkylenyl, and arylalkylenyl;
R₉ is selected from the group consisting of hydrogen and alkyl;
R₁₀ is independently C₃₋₈ alkylene;
A₁ is selected from the group consisting of —O—, —C(O)—, —CH₂—, —S(O)₀₋₂—, and —N(R₄ₐ)—;
Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—;
V is selected from the group consisting of —O—C(R₆)— and —N(R₈)—C(R₆)—;
W is selected from the group consisting of a bond, —C(O)—, and —S(O)₂—; and
a and b are each independently an integer from 1 to 6 with the proviso that a+b is ≦7;
or a pharmaceutically acceptable salt thereof.

45-47. (canceled)

48. A compound of the following formula (XIXd): 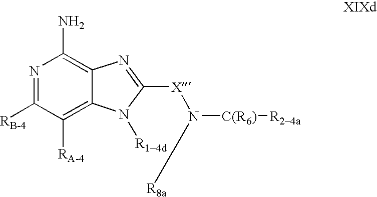
wherein:
X′″ is selected from the group consisting of C₁₋₄ alkylene and C₂₋₄ alkenylene;
R₂₋₄ₐ is selected from the group consisting of C₁₋₆ alkyl, C₂₋₆ alkenyl, C₂₋₆ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl wherein the C₁₋₆ alkyl, C₂₋₆ alkenyl, C₂₋₆ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl groups are unsubstituted or substituted by one or more substituents independently selected from the group consisting of C₁₋₄ alkyl, C₁₋₄ alkoxy, C₁₋₄ alkanoyl, C₁₋₄ alkoxycarbonyl, hydroxyC₁₋₄ alkyl, haloC₁₋₄ alkyl, haloC₁₋₄ alkoxy, halogen, nitro, hydroxy, mercapto, cyano, amino, C₁₋₄ alkylamino, di(C₁₋₄ alkyl)amino, and in the case of C₂₋₆ alkyl, C₂₋₆ alkenyl, C₂₋₆ alkynyl, and heterocyclyl, oxo;
R_{A-4} and R_{B-1} taken together form a fused heteroaryl or 5 to 7 membered saturated ring containing one heteroatom selected from the group consisting of N and S, wherein the heteroaryl ring is unsubstituted or substituted by one or more R_{b} groups, and the 5 to 7 membered saturated ring is unsubstituted or substituted by one or more R_{c} groups;
R_{b} is selected from the group consisting of halogen, hydroxy, alkyl, haloalkyl, alkoxy, and —N(R₉)₂;
R_{c} is selected from the group consisting of halogen, hydroxy, alkyl, alkenyl, haloalkyl, alkoxy, alkylthio, and —N(R₉)₂;
R_{1-4d} is selected from the group consisting of:
—R_{4b},
—X—R_{4b},
—X—Yₐ—R_{4b}, and
—X—R₅₋₂;
X is selected from the group consisting of alkylene, alkenylene, alkynylene, arylene, heteroarylene, and heterocyclylene wherein the alkylene, alkenylene and alkynylene groups can be optionally interrupted or terminated by arylene, heteroarylene or heterocyclylene and optionally interrupted by one or more —O— groups;
Yₐ is selected from the group consisting of:
—S(O)₀₋₂—,
—C(R₆)—,
—C(R₆)—O—,
—O—C(R₆)—,
—O—C(O)—O—.
—N(R₈)-Q-,
—O—C(R₆)—N(R₈)—,
—C(R₆)—N(OR₉)—, 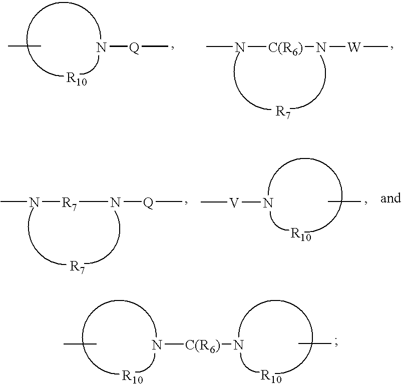
R_{4b} is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, aryl, arylalkylenyl, aryloxyalkylenyl, alkylarylenyl heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkylenyl, haloalkylenyl, haloalkyleneoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl and heterocyclyl, oxo;
with the proviso that when R_{1-4d} is —R_{4b} or —X—R_{4b}, and R_{4b} or —X—R_{4b} includes a carbocyclic ring or heterocyclic ring containing one heteroatom, then the ring carbon atom by which the ring is attached is otherwise unsubstituted or substituted by an atom other than O, S, or N;
with the further proviso that R_{1-4d} is other than an unsubstituted or substituted isoxazolylalkylenyl, dihydroisoxazolylalkylenyl, or oxadiazolylalkylenyl group;
R₅₋₂ is selected from the group consisting of: 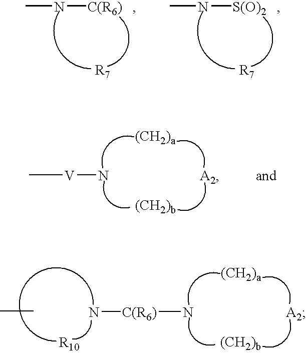
R₆ is selected from the group consisting of ═O and ═S;
R₇ is C₂₋₇ alkylene;
R₈ₐ is selected from the group consisting of hydrogen and C₁₋₄ alkyl;
R₈ is selected from the group consisting of hydrogen, alkyl, alkoxyalkylenyl, and arylalkylenyl;
R₉ is selected from the group consisting of hydrogen and alkyl;
R₁₀ is C₃₋₈ alkylene;
A₂ is selected from the group consisting of —O—, —C(O)—, —CH₂—, —S(O)₀₋₂—, and —N(R_{4b})—;
Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—;
V is selected from the group consisting of —O—C(R₆)— and —N(R₈)—C(R₆)—;
W is selected from the group consisting of a bond, —C(O)—, and —S(O)₂—; and
a and b are each independently an integer from 1 to 6 with the proviso that a+b is ≦7;
with the proviso that when X is interrupted with one —O— group, then Yₐ is other than —S(O)₀₋₂—;
or a pharmaceutically acceptable salt thereof.

49-59. (canceled)

60. A compound of the Formula (XX): 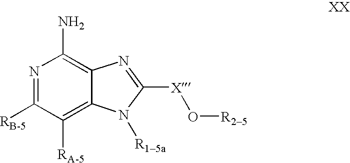
wherein:
X′″ is selected from the group consisting of C₁₋₄alkylene and C₂₋₄ alkenylene;
R₁₋₅ₐ is selected from the group consisting of:
hydrogen,
alkyl,
alkoxyalkylenyl,
hydroxyalkoxyalkylenyl,
alkenyl,
alkynyl,
aryl,
arylalkylenyl,
alkylarylenyl,
heteroaryl.
heteroarylalkylenyl,
alkylheteroarylenyl,
heterocyclyl,
—X₃—O—C(R₆)—R₁₋₄ₐ₋₁,
—X₃—O—C(R₆)—O—R₁₋₄ₐ, and
—X₃—O—C(R₆)—N(R₈)—R₁₋₄ₐ—,
wherein the alkyl, aryl, arylalkylenyl, alkylarylenyl, heteroaryl, heteroarylalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, heteroaryl, tetrahydropyranyl, amino, alkylamino, dialkylamino, and in the case of heterocyclyl, oxo;
with the proviso that when R₁₋₅ₐ includes a carbocyclic ring or heterocyclic ring containing one heteroatom, then the ring carbon atom by which the ring is attached is otherwise unsubstituted or substituted by an atom other than O, S, or N;
with the further proviso that R₁₋₅ₐ is other than an unsubstituted or substituted isoxazolylalkylenyl, dihydroisoxazolylalkylenyl, or oxadiazolylalkylenyl group;
R₂₋₅ is selected from the group consisting of:
—Ar,
—Ar′—Y″—R₁₋₁, and
—Ar′—X′″—Y″—R₄₋₁;
R_{A-5} and R_{B-5} are each independently selected from the group consisting of:
hydrogen,
halogen,
alkyl.
alkenyl,
alkoxy,
alkylthio, and
—N(R₉)₂,
or R_{A-5} and R_{B-5} taken together form a fused aryl ring that is unsubstituted or substituted by one or more Rₐ₁ groups,
or R_{A-5} and R_{B-5} taken together form a fused 5 to 7 membered saturated ring, unsubstituted or substituted by one or more R_{c} groups;
Rₐ₁ is selected from the group consisting of halogen, alkyl, haloalkyl, alkoxy, and —N(R₉)₂;
R_{c} is selected from the group consisting of halogen, hydroxy, alkyl, alkenyl, haloalkyl, alkoxy, alkylthio, and —N(R₉)₂;
Ar is selected from the group consisting of aryl and heteroaryl both of which can be unsubstituted or can be substituted by one or more substituents independently selected from the group consisting of alkyl, alkoxy, haloalkyl, haloalkoxy, halogen, nitro, hydroxy, hydroxyalkyl, mercapto, cyano, carboxy, formyl, aryl, aryloxy, arylalkyleneoxy, heteroaryl, heteroaryloxy, heteroarylalkyleneoxy, heterocyclyl, heterocyclylalkylenyl, amino, alkylamino, and dialkylamino;
Ar′ is selected from the group consisting of arylene and heteroarylene both of which can be unsubstituted or can be substituted by one or more substituents independently selected from the group consisting of alkyl, alkoxy, haloalkyl, haloalkoxy, halogen, nitro, hydroxy, hydroxyalkyl, mercapto, cyano, carboxy, formyl, aryl, aryloxy, arylalkyleneoxy, heteroaryl, heteroaryloxy, heteroarylalkyleneoxy, heterocyclyl, heterocyclylalkylenyl, amino, alkylamino, and dialkylamino;
X₃ is selected from the group consisting of alkylene, arylene, heteroarylene, and heterocyclylene wherein the alkylene group can be optionally interrupted or terminated by arylene, heteroarylene or heterocyclylene and optionally interrupted by one or more —O— groups;
Y″ is selected from the group consisting of:
—S(O)₀₋₂—,
—S(O)₂—N(R₈ₐ)—,
—C(R₆)—,
—C(R₆)—O—,
—O—C(R₆)—,
—O—C(O)—O—,
—N(R₈ₐ)-Qₐ-,
—C(R₆)—N(R₈ₐ)—,
—O—C(R₆)—N(R₈ₐ)—, and
—C(R₆)—N(OR₉)—;
R₁₋₄ₐ₋₁ is selected from the group consisting of alkyl, alkenyl, alkoxyalkylenyl, aryl, and arylalkylenyl, wherein the alkyl, alkenyl, alkoxyalkylenyl, aryl, and arylalkylenyl can be unsubstituted or substituted with one or more substituents selected from the group consisting of alkyl, alkoxy, hydroxyalkyl, haloalkyl, haloalkoxy, halogen, nitro, hydroxy, mercapto, cyano, amino, alkylamino, and dialkylamino;
R₄₋₁ is selected from the group consisting of C₁₋₆ alkyl, C₂₋₆ alkenyl, C₂₋₆ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl wherein the C₁₋₆ alkyl, C₂₋₆ alkenyl, C₂₋₆ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl groups are unsubstituted or substituted by one or more substituents independently selected from the group consisting of C₁₋₄ alkyl, C₁₋₄ alkoxy, hydroxyC₁₋₄ alkyl, haloC₁₋₄ alkyl, haloC₁₋₄ alkoxy, halogen, nitro, hydroxy, mercapto, cyano, amino, C₁₋₄ alkylamino, di(C₁₋₄ alkyl)amino, and in the case of C₂₋₆ alkyl, C₂₋₆ alkenyl, C₂₋₆ alkynyl, and heterocyclyl, oxo;
with the proviso that when Y″ is —S(O)₂—N(R₈ₐ)— or —C(R₆)—N(R₈ₐ)—, then R₄₋₁ can also be hydrogen;
R₆ is selected from the group consisting of ═O and —S;
R₈ₐ is selected from the group consisting of hydrogen and C₁₋₄ alkyl.
R₈ is selected from the group consisting of hydrogen, alkyl, alkoxyalkylenyl, and arylalkylenyl;
R₉ is selected from the group consisting of hydrogen and alkyl;
Qₐ is selected from the group consisting of a bond, —C(R₆)—, —C(R₆)—C(R₆)—, —S(O)₂—, —C(R₆)—N(R₈ₐ)—W—, —S(O)₂—N(R₈ₐ)—, —C(R₆)—O—, and —C(R₆)—N(OR₉)—; and
W is selected from the group consisting of a bond, —C(O)—, and —S(O)₂—;
or a pharmaceutically acceptable salt thereof.

61-76. (canceled)

77. A compound of the Formula (XXII): 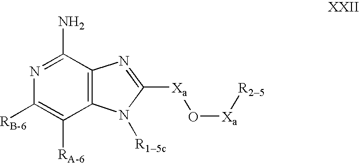
wherein:
Xₐ is C₁₋₂ alkylene;
R_{1-5c} is selected from the group consisting of:
—R_{4c},
—X₃—R_{4c},
—X₃—Y′″—R_{4c}, and
—X₃—R₅₋₃;
R₂₋₅ is selected from the group consisting of:
—Ar,
—Ar′—Y″—R₄₋₁, and
—Ar′—X′″—Y″—R₄₋₁;
R_{A-6} and R_{B-6} are each independently selected from the group consisting of:
hydrogen,
halogen,
alkyl,
alkenyl,
alkoxy,
alkylthio, and
—N(R₉)—;
or R_{A-6} and R_{B-6} taken together form either a fused aryl ring that is unsubstituted or substituted by one or more Rₐ₁ groups, or a fused 5 to 7 membered saturated ring that is unsubstituted or substituted by one or more R_{c} groups;
or R_{A-6} and R_{B-6} taken together form a fused heteroaryl or 5 to 7 membered saturated ring, containing one heteroatom selected from the group consisting of N and S, wherein the heteroaryl ring is unsubstituted or substituted by one or more R_{b} groups, and the 5 to 7 membered saturated ring is unsubstituted or substituted by one or more R_{b} groups;
Rₐ₁ is selected from the group consisting of halogen, alkyl haloalkyl, alkoxy, and —N(R₉)₂;
R_{b} is selected from the group consisting of halogen, hydroxy, alkyl, haloalkyl, alkoxy, and —N(R₉)₂;
R_{c} is selected from the group consisting of halogen, hydroxy, alkyl, alkenyl, haloalkyl, alkoxy, alkylthio, and —N(R₉)₂;
Ar is selected from the group consisting of aryl and heteroaryl both of which can be unsubstituted or can be substituted by one or more substituents independently selected from the group consisting of alkyl, alkoxy, haloalkyl, haloalkoxy, halogen, nitro, hydroxy, hydroxyalkyl, mercapto, cyano, carboxy, formyl, aryl, aryloxy, arylalkyleneoxy, heteroaryl, heteroaryloxy, heteroarylalkyleneoxy, heterocyclyl, heterocyclylalkylenyl, amino, alkylamino, and dialkylamino;
Ar′ is selected from the group consisting of arylene and heteroarylene both of which can be unsubstituted or can be substituted by one or more substituents independently selected from the group consisting of alkyl, alkoxy, haloalkyl, haloalkoxy, halogen, nitro, hydroxy, hydroxyalkyl, mercapto, cyano, carboxy, formyl, aryl, aryloxy, arylalkyleneoxy, heteroaryl, heteroaryloxy, heteroarylalkyleneoxy, heterocyclyl, heterocyclylalkylenyl, amino, alkylamino, and dialkylamino;
X₃ is selected from the group consisting of alkylene, arylene, heteroarylene, and heterocyclylene wherein the alkylene group can be optionally interrupted or terminated by arylene, heteroarylene or heterocyclylene and optionally interrupted by one or more —O— groups;
Y′″ is selected from the group consisting of:
—S(O)₀₋₂—,
—O—C(R₆)—,
—O—C(O)—O—.
—N(R₈)-Q-,
—O—C(R₆)—N(R₈)—, 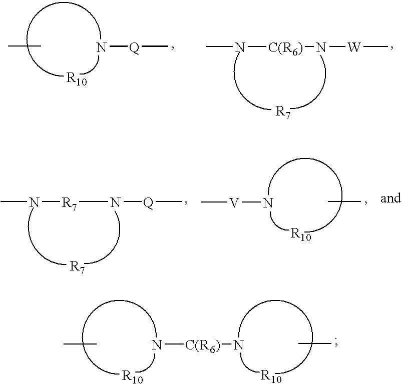
with the proviso that when X₃ is interrupted with one —O— group, then Y″ is other than —S(O)₀₋₂—;
with the further proviso that when R_{A-6} and R_{B-6} taken together form a fused heteroaryl or 5 to 7 membered saturated ring, containing one heteroatom selected from the group consisting of N and S, wherein the heteroaryl ring is unsubstituted or substituted by one or more R_{b} groups, and the 5 to 7 membered saturated ring is unsubstituted or substituted by one or more R_{c} groups, then Y′″ can also be selected from the group consisting of —C(R₆)—, —C(R₆)—O—, and —C(R₆)—N(OR₉)—;
X′″ is selected from the group consisting of a C₁₋₄ alkylene and C₂₋₄ alkenylene;
Y″ is selected from the group consisting of:
—S(O)₀₋₂—,
—S(O)₂—N(R₈ₐ)—,
—C(R₆)—,
—C(R₆)—O—,
—O—C(R₆)—.
—O—C(O)—O—,
—N(R₈ₐ)-Qₐ-,
—C(R₆)—N(R₈ₐ)—,
—O—C(R₆)—N(R₈ₐ)—, and
—C(R₆)—N(OR₉)—,
R_{4c} is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, and in the case of heterocyclyl, oxo;
R₄₋₁ is selected from the group consisting of C₁₋₆ alkyl, C₂₋₆ alkenyl, C₂₋₆ alkynyl, aryl, arylC₁₋₄ alkylenyl, aryloxyC₁₋₄ alkylenyl, C₁₋₄ alkylarylenyl heteroaryl, heteroarylC₁₋₄ alkylenyl, heteroaryloxyC₁₋₄ alkylenyl, C₁₋₄ alkylheteroarylenyl, and heterocyclyl wherein the C₁₋₆ alkyl, C₂₋₆ alkenyl, C₂₋₆ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl groups are unsubstituted or substituted by one or more substituents independently selected from the group consisting of C₁₋₄ alkyl, C₁₋₄ alkoxy, hydroxyC₁₋₄ alkyl, haloC₁₋₄ alkyl, haloC₁₋₄ alkoxy, halogen, nitro, hydroxy, mercapto, cyano, amino, C₁₋₄ alkylamino, di(C₁₋₄ alkyl)amino, and in the ease of C₂₋₆ alkyl, C₂₋₆ alkenyl, C₂₋₆ alkynyl, and heterocyclyl, oxo;
with the proviso that when Y″ is —S(O)₂—N(R₈ₐ)— or —C(R₆)—N(R₈ₐ)—, then R₄₋₁ can also be hydrogen;
R₅₋₃ is selected from the group consisting of: 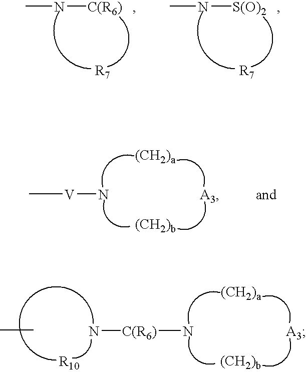
R₆ is selected from the group consisting of ═O and ═S;
R₇ is C₂₋₇ alkylene;
R₈ₐ is selected from the group consisting of hydrogen and C₁₋₄ alkyl;
R₈ is selected from the group consisting of hydrogen, alkyl, alkoxyalkylenyl, and arylalkylenyl;
R₉ is selected from the group consisting of hydrogen and alkyl,
R₁₀ is C₃₋₈ alkylene;
A₃ is selected from the group consisting of —O—, —C(O)—, —S(O)₀₋₂—, —CH₂—, and —N(R_{4c})—;
Qₐ is selected from the group consisting of a bond, —C(R₆)—, —C(R₆)—C(R₆)—, —S(O)₂—, —C(R₆)—N(R₈ₐ)—W—, —S(O)₂—N(R₈ₐ)—, —C(R₆)—O—, and —C(R₆)—N(OR₉)—;
Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—;
V is selected from the group consisting of —O—C(R₆)— and —N(R₈)—C(R₆)—;
W is selected from the group consisting of a bond, —C(O)—, and —S(O)₂—; and
a and b are each independently an integer from 1 to 6 with the proviso that a+b is ≦7;
with the proviso that when R_{1-5c} includes a carbocyclic ring or heterocyclic ring containing one heteroatom, then the ring carbon atom by which the ring is attached is otherwise unsubstituted or substituted by an atom other than O, S, or N; and
with the further proviso that R_{1-5c} is other than an unsubstituted or substituted isoxazolylalkylenyl, dihydroisoxazolylalkylenyl, or oxadiazolylalkylenyl group;
or a pharmaceutically acceptable salt thereof.

78-87. (canceled)

88. A compound of the following formula (XXIII): 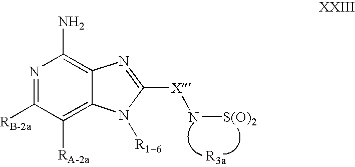
wherein:
X′″ is selected from the group consisting of C₁₋₄ alkylene and C₂₋₄ alkenylene;
R₃ₐ is C₂₋₅ alkylene;
R_{A-2a} and R_{B-2a} are each independently selected from the group consisting of:
hydrogen,
halogen,
alkyl.
alkenyl,
alkoxy,
alkylthio, and
—N(R₉)₂;
or R_{A-2a} and R_{B-2a} taken together form either a fused aryl ring that is unsubstituted or substituted by one or more Rₐ₁ groups, or a fused 5 to 7 membered saturated ring that is unsubstituted or substituted by one or more R_{c} groups;
or R_{A-2a} and R_{B-2a} taken together form a fused heteroaryl or 5 to 7 membered saturated ring, containing one heteroatom selected from the group consisting of N and S, wherein the heteroaryl ring is unsubstituted or substituted by one or more R_{b} groups, and the 5 to 7 membered saturated ring is unsubstituted or substituted by one or more R_{c} groups:
Rₐ₁ is selected from the group consisting of halogen, alkyl, haloalkyl, alkoxy, and —N(R₉)₂;
R_{b} is selected from the group consisting of halogen, hydroxy, alkyl, haloalkyl, alkoxy, and —N(R₉)₂;
R_{c} is selected from the group consisting of halogen, hydroxy, alkyl, alkenyl, haloalkyl, alkoxy, alkylthio, and —N(R₉)₂;
R₁₋₆ is selected from the group consisting of:
—R₄ₐ,
—X₃—R₄ₐ,
—X₃—Yₐ—R₄ₐ, and
—X₃—R₅₋₁;
X₃ is selected from the group consisting of alkylene, arylene, heteroarylene, and heterocyclylene wherein the alkylene group can be optionally interrupted or terminated by arylene, heteroarylene or heterocyclylene and optionally interrupted by one or more —O— groups;
Yₐ is independently selected from the group consisting of:
—S(O)₅₋₂—,
—C(R₆)—,
—C(R₆)—O—,
—O—C(R₆)—,
—O—C(O)—O—.
—N(R₈)-Q-,
—O—C(R₆)—N(R₈)—,
—C(R₆)—N(OR₉)—, 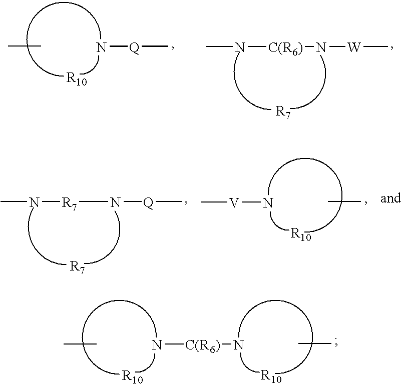
R₄ₐ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of heterocyclyl, oxo;
R₅₋₁ is selected from the group consisting of: 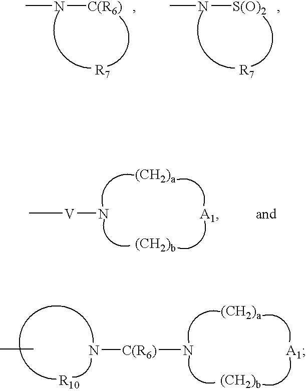
R₆ is selected From the group consisting of ═O and ═S;
R₇ is C₂₋₇ alkylene;
R₈ is selected from the group consisting of hydrogen, alkyl, alkoxyalkylenyl, and arylalkylenyl;
R₉ is selected from the group consisting of hydrogen and alkyl;
R₁₀ is C₃₋₈ alkylene;
A₁ is selected from the group consisting of —O—, —C(O)—, —CH₂—, —S(O)₀₋₂—, and —N(R₄ₐ)—;
Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—;
V is selected from the group consisting of —O—C(R₆)— and —N(R₈)—C(R₆)—;
W is selected from the group consisting of a bond, —C(O)—, and —S(O)₂—; and
a and b are each independently an integer from 1 to 6 with the proviso that a+b is ≦7;
with the proviso that when X₃ is interrupted with one —O— group, then Yₐ is other than —S(O)₀₋₂—;
with the further proviso that when R₁₋₆ includes a carbocyclic ring or heterocyclic ring containing one heteroatom, then the ring carbon atom by which the ring is attached is otherwise unsubstituted or substituted by an atom other than O, S, or N; and
with the further proviso that R₁₋₆ is other than an unsubstituted or substituted isoxazolylalkylenyl, dihydroisoxazolylalkylenyl, or oxadiazolylalkylenyl group;
or a pharmaceutically acceptable salt thereof.

89-116. (canceled)

117. A pharmaceutical composition comprising a therapeutically effective amount of a compound or salt of claim 12 in combination with a pharmaceutically acceptable carrier.

118. A method of inducing cytokine biosynthesis in an animal comprising administering an effective amount of a compound or salt of claim 12 to the animal.

119-120. (canceled)

121. A pharmaceutical composition comprising a therapeutically effective amount of a compound or salt of claim 22 in combination with a pharmaceutically acceptable carrier.

122. A method of inducing cytokine biosynthesis in an animal comprising administering an effective amount of a compound or salt of claim 22 to the animal.

123. A pharmaceutical composition comprising a therapeutically effective amount of a compound or salt of claim 32 in combination with a pharmaceutically acceptable carrier.

124. A method of inducing cytokine biosynthesis in an animal comprising administering an effective amount of a compound or salt of claim 32 to the animal.

125. A pharmaceutical composition comprising a therapeutically effective amount of a compound or salt of claim 44 in combination with a pharmaceutically acceptable carrier.

126. A method of inducing cytokine biosynthesis in an animal comprising administering an effective amount of a compound or salt of claim 44 to the animal.

127. A pharmaceutical composition comprising a therapeutically effective amount of a compound or salt of claim 48 in combination with a pharmaceutically acceptable carrier.

128. A method of inducing cytokine biosynthesis in an animal comprising administering an effective amount of a compound or salt of claim 48 to the animal.

129. A pharmaceutical composition comprising a therapeutically effective amount of a compound or salt of claim 60 in combination with a pharmaceutically acceptable carrier.

130. A method of inducing cytokine biosynthesis in an animal comprising administering an effective amount of a compound or salt of claim 60 to the animal.

131. A pharmaceutical composition comprising a therapeutically effective amount of a compound or salt of claim 77 in combination with a pharmaceutically acceptable carrier.

132. A method of inducing cytokine biosynthesis in an animal comprising administering an effective amount of a compound or salt of claim 77 to the animal.

133. A pharmaceutical composition comprising a therapeutically effective amount of a compound or salt of claim 88 in combination with a pharmaceutically acceptable carrier.

134. A method of inducing cytokine biosynthesis in an animal comprising administering an effective amount of a compound or salt of claim 88 to the animal.
